(19) United States
(12) Patent Application Publication
(10) Pub. No.: US 2020/0303037 A1
(43) Pub. Date: Sep. 24, 2020

(54) THE PRIMARY SITE OF METASTATIC CANCER IDENTIFICATION METHOD AND SYSTEM THEREOF

(71) Applicant: Mao Ying Genetech Inc., Taipei City (TW)

(72) Inventor: PEI-ING HWANG, Taipei City (TW)

(21) Appl. No.: 16/341,438

(22) PCT Filed: Oct. 27, 2017
(86) PCT No.: PCT/CN2017/107952

§ 371 Date: Apr. 11, 2019

Related U.S. Application Data
(60) Provisional application No. 62/414,228, filed on Oct. 28, 2016.

(51) Int. Cl. G16B 25/10; C12Q 1/6886; C12N 15/10; G16B 25/20; G16B 25/30

(52) U.S. Cl. CPC G16B 25/10; C12Q 1/6886; C12Q 2600/158; G16B 25/20; G16B 25/30; C12N 15/1089

(57) ABSTRACT

The present disclosure is related to a developing method of candidate probes and a using method thereof. Specifically, the candidate probes are capable binding specific genes and further identifying the primary site of a metastatic cancer in a subject in need thereof. Briefly, the developing method comprises the steps of: (a) using a chip to generate gene expressions of metastasis cancer samples with well known primary sites; (b) using a processing module to compare the gene expressions of metastasis cancer samples; and (c) developing candidate probes based on the previous comparing results. The using method comprises the steps of: (a′) using the previous candidate probes to detect the relative gene expression in a test sample with unknown primary site; and (b′) using a processing module to predict the primary site of the test sample. Moreover, the present disclosure further provides a system used to conduct the above method, and the system comprises a detecting chip including an array with the candidate probes and a processing module.

### FIELD

The present disclosure relates to a method and a system for identifying a metastatic cancer, and more particularly to a method and a system for identifying a primary site of metastatic cancer.

### BACKGROUND

Finding the primary site for metastatic cancers was mandatory and is still necessary for physicians to prescribe proper treatment for their patients. However, identifying the primary site for some of the poorly developed cancers or the so-called “cancer of unknown primary” (CUP) can sometimes be challenging.

For the CUPs where it is difficult to identify the primary site under the currently available technologies, patients will resort to additional procedures such as random biopsies in the hope to find the origin of the metastatic cancer. The chances of finding the primary site of the metastatic tumor after all such procedures, however, remain relatively unoptimistic.

Accordingly, it is desirable to develop a method to accurately and efficiently identify the primary site of a metastatic cancer.

### SUMMARY

The present disclosure provides a method for developing a plurality of candidate probes to identify at least one primary site of a selected disease, disorder or genetic disorder in a mammalian subject. The method comprises the following steps: (a) generating a plurality of gene expression from a standard sample of a subject having a selected disease, disorder or genetic disorder by using a detecting chip; (b) comparing the plurality of gene expression to generate a comparison result by using a processing module; and (c) developing an array containing the plurality of candidate probes based on the comparison result. The standard sample is diagnosed with a metastasis cancer with at least one known primary site. The detecting chip is electrically connected to the processing module. The plurality of candidate probes in the array are capable of binding a plurality of polynucleotide sequences selected from any one of SEQ ID No.1 to 695 or from any fragment of SEQ ID No.1 to 695.

In one embodiment, the number of the candidate probes is about 650.

In one embodiment, the number of the candidate probes is about 100.

In one embodiment, the number of the candidate probes is about 50.

In one embodiment, the detecting chip includes a microarray, a next-generation sequencing device, quantitative PCR and magnetic beads.

In one embodiment, the processing module is a central processing unit (CPU).

In one embodiment, the standard sample includes blood, blood plasma, serum, urine, tissue, cells, organs, seminal fluids or any combination thereof.

In one embodiment, the selected disease, disorder or genetic pathology includes hematologic malignancies or solid tumors.

In one embodiment, a length of the candidate probes is at least 20 nucleotides.

In one embodiment, the candidate probes are approximately 695 genes selected from the group consisting of those given in Table 1, and more preferably 50 genes or less.

The present disclosure further provides a method for identifying a primary site of a selected disease, disorder or genetic disorder in a mammalian subject. The method comprises the following steps: (a) analysing expression levels of an array of a test sample by using a detecting chip that contains a plurality of candidate probes developed by the procedures described above; and (b) predicting a primary site of the test sample based on the array's expression levels by using a processing module. The test sample is diagnosed with a metastasis cancer with at least one unknown primary site, and the plurality of candidate probes are capable of binding the plurality of polynucleotide sequence selected from any one of SEQ ID No.1 to 695 or from any fragment of SEQ ID No.1 to 695.

In one embodiment, the test sample includes blood, blood plasma, serum, urine, tissue, cells, organs, seminal fluids or any combination thereof.

The present disclosure also provides a system for identifying a primary site of a selected disease, disorder or genetic disorder in a mammalian subject. The system comprises a detecting chip that contains a plurality of candidate probes and a processing module. The detecting chip and the processing module are electrically connected to each other. The plurality of candidate probes are capable of binding a plurality of polynucleotide sequence selected from any one of SEQ ID No.1 to 695 or from any fragment of SEQ ID No.1 to 695.

In some embodiments of the present disclosure, the tissue or organ may be any tissue or organ, for example, breast, stomach, colon, pancreas, bladder, thyroid, prostate, kidney, liver, ovary, germ cell, soft tissue, skin, lymph node or lung.

Those and other aspects of the present disclosure may be further clarified by the following descriptions and drawings of preferred embodiments. Although there may be changes or modifications therein, they would not betray the spirit and scope of the novel ideas disclosed in the present disclosure.

### BRIEF DESCRIPTION OF THE DRAWINGS

One or more embodiments are illustrated by way of examples, and not by limitation, in the FIGURES of the accompanying drawings, wherein elements having the same reference numeral designations represent like elements throughout.

FIG. 1 illustrates the hierarchical clustering result of metastatic cancers with various primary sites using the expression profiles of the genes, which is acquired by using a microarray gene expression dataset.

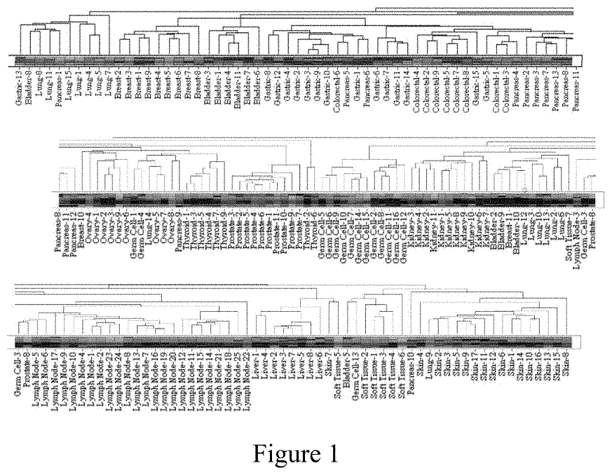

The drawings are only schematic and are non-limiting. Any reference signs in the claims shall not be construed as limiting the scope. Like reference symbols in the various drawings indicate like elements

### DETAILED DESCRIPTION

Unless defined otherwise, all technical and scientific terms used herein have the same meanings as commonly understood by one of skill in the art to which this disclosure belongs. It will be further understood that terms; such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Definition

Unless clearly specified herein, meanings of the articles “a,” “an,” and “said” all include the plural form of “more than one.” Therefore, for example, when the term “a component” is used, it includes multiple said components and equivalents known to those of common knowledge in said field.

The term “about,” as used herein, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “cancer” and “tumor” as used herein are both defined as a disease characterized by the rapid and uncontrolled growth of aberrant cells. Therefore, the terms of “cancer” and “tumor” are interchangeable. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “origin,” “originate” and “primary site” as used herein are all defined as the first location (i.e., tissue or organ) where a tumor/cancer developed. Therefore, the terms of “origin,” “originate” and “primary site” are interchangeable.

In the context of the present invention, the following abbreviations for the commonly occurring “nucleic acid bases” or “nucleotides” are used, “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

TABLE 1
“Genes used as probes for identification”

| SEQ ID | | | |
|---|---|---|---|
| No. | Gene_Sym | GENE_ID | Gene_Title |
| 103 | — | — | immunoglobulin kappa light chain variable |
| | | | region |
| 105 | — | — | immunoglobulin heavy chain variable |
| | | | region |
| 271 | ABAT | 18 | 4-aminobutyrate aminotransferase |
| 488 | ABCA8 | 10351 | ATP-binding cassette, sub-family A |
| | | | (ABC1), member 8 |
| 44 | ACE2 | 59272 | angiotensin I converting enzyme (peptidyl- |
| | | | dipeptidase A) 2 |
| 512 | ACPP | 55 | acid phosphatase, prostate |
| 583 | ACTG2 | 72 | actin, gamma 2, smooth muscle, enteric |
| 303 | ADAM28 | 10863 | ADAM metallopeptidase domain 28 |
| 377 | ADAMDEC1 | 27299 | ADAM-like, decysin 1 |
| 260/261 | ADH1B | 125 | alcohol dehydrogenase 1B (class I), beta |
| | | | polypeptide |
| 365 | ADH1C | 126 | alcohol dehydrogenase 1C (class I), gamma |
| | | | polypeptide |
| 288 | AGR2 | 10551 | anterior gradient homolog 2 (Xenopus |
| | | | laevis) |
| 626 | AGTR2 | 186 | angiotensin II receptor, type 2 |
| 181 | AHNAK2 | 113146 | AHNAK nucleoprotein 2 |
| 210 | AHSG | 197 | alpha-2-HS-glycoprotein preproprotein |
| 344 | AKR1B10 | 57016 | aldo-keto reductase family 1, member B10 |
| | | | (aldose reductase) |
| 197 | AKR1C2 | 1646 | aldo-keto reductase family 1, member C2 |
| | | | (dihydrodiol dehydrogenase 2; bile acid |
| | | | binding protein; 3-alpha hydroxysteroid |
| | | | dehydrogenase, type III) |
| 292 | AKR1C3 | 8644 | aldo-keto reductase family 1, member C3 |
| | | | (3-alpha hydroxysteroid dehydrogenase, |
| | | | type II) |
| 131/206 | ALB | 213 | albumin |
| 189 | ALDH1A1 | 216 | aldehyde dehydrogenase 1 family, member |
| | | | A1 |
| 40 | ALDH8A1 | 64577 | aldehyde dehydrogenase 8 family, member |
| | | | A1 |
| 97 | ALDOB | 229 | fructose-bisphosphate aldolase B |
| 205/491 | ALDOB | 229 | aldolase B, fructose-bisphosphate |
| 510 | ALOX5 | 240 | arachidonate 5-lipoxygenase |
| 272 | AMACR /// | 23600 /// | alpha-methylacyl-CoA racemase isoform 3 |
| | C1QTNF3- | 100534612 | /// alpha-methyl acyl-CoA racemase |
| | AMACR | | isoform 1 /// alpha-methylacyl-CoA |
| | | | racemase isoform 2 /// |
| 424 | AMBP | 259 | alpha-1-microglobulin/bikunin precursor |
| 298 | AMY1A /// | 276 /// 277 /// | pancreatic alpha-amylase precursor /// |
| | AMY1B /// | 278 /// 279 /// | alpha-amylase 1 precursor /// alpha-amylase |
| | AMY1C /// | 280 /// 281 | 1 precursor /// alpha-amylase 1 precursor /// |
| | AMY2A /// | | alpha-amylase 1 precursor /// alpha-amylase |
| | AMY2B /// | | 2B precursor /// /// |
| | AMYP1 | | |
| 354 | ANK3 | 288 | ankyrin 3, node of Ranvier (ankyrin G) |
| 79 | ANO1 | 55107 | anoctamin-1 |
| 573 | ANPEP | 290 | alanyl (membrane) aminopeptidase |
| | | | (aminopeptidase N, aminopeptidase M, |
| | | | microsomal aminopeptidase, CD13, p150) |
| 226 | ANXA10 | 11199 | annexin A10 |
| 277 | ANXA3 | 306 | annexin A3 |
| 554 | AOC1 | 26 | amiloride-sensitive amine oxidase [copper- |
| | | | containing] isoform 2 precursor /// |
| | | | amiloride-sensitive amine oxidase [copper- |
| | | | containing] isoform 1 precursor |
| 454 | AOX1 | 316 | aldehyde oxidase 1 |
| 620 | AP3B2 | 8120 | adaptor-related protein complex 3, beta 2 |
| | | | subunit |
| 358 | APCS | 325 | amyloid P component, serum |
| 99/509 | APOA1 | 335 | apolipoprotein A-I |
| 68/69 | APOA2 | 336 | apolipoprotein A-II |
| 453 | APOB | 338 | apolipoprotein B (including Ag(x) antigen) |
| 342 | APOBEC3B | 9582 | apolipoprotein B mRNA editing enzyme, |
| | | | catalytic polypeptide-like 3B |
| 398 | APOC3 | 345 | apolipoprotein C-III |
| 448 | APOH | 350 | apolipoprotein H (beta-2-glycoprotein I) |
| 4 | AQP3 | 360 | aquaporin 3 (Gill blood group) |
| 445 | AREG | 374 | amphiregulin (schwannoma-derived growth |
| | | | factor) |
| 372 | ARG1 | 383 | arginase, liver |
| 538 | ARG2 | 384 | arginase, type II |
| 374 | ARHGAP6 | 395 | Rho GTPase activating protein 6 |
| 35 | ARL14 | 80117 | ADP-ribosylation factor-like 14 |
| 238/239 | ASCL1 | 429 | achaete-scute complex homolog 1 |
| | | | (Drosophila) |
| 75 | ASPN | 54829 | asporin |
| 179 | ATP8A1 | 10396 | ATPase, aminophospholipid transporter |
| | | | (APLT), class I, type 8A, member 1 |
| 279 | AZGP1 | 563 | alpha-2-glycoprotein 1, zinc-binding |
| 57 | BANK1 | 55024 | B-cell scaffold protein with ankyrin repeats |
| | | | 1 |
| 433 | BBOX1 | 8424 | butyrobetaine (gamma), 2-oxoglutarate |
| | | | dioxygenase (gamma-butyrob etaine |
| | | | hydroxylase) 1 |
| 144 | BCAT1 | 586 | branched chain aminotransferase 1, |
| | | | cytosolic |
| 429 | BCHE | 590 | butyrylcholinesterase |
| 408 | BCL2A1 | 597 | BCL2-related protein A1 |
| 602 | BCLAF1 | 9774 | BCL2-associated transcription factor 1 |
| 85 | BEX1 | 55859 | brain expressed, X-linked 1 |
| 48 | BHMT2 | 23743 | betaine-homocysteine methyltransferase 2 |
| 213 | BIRC3 | 330 | baculoviral IAP repeat-containing 3 |
| 319 | BLNK | 29760 | B-cell linker |
| 42 | C14orf105 | 55195 | chromosome 14 open reading frame 105 |
| 67 | C1orf116 | 79098 | chromosome 1 open reading frame 116 |
| 14 | C1orf186 /// | 440712 /// | uncharacterized protein C1orf186 |
| | LOC100505650 | 100505650 | |
| 567 | C7 | 730 | complement component 7 |
| 82 | C8orf4 | 56892 | chromosome 8 open reading frame 4 |
| 332 | C9 | 735 | complement component 9 |
| 280 | CA2 | 760 | carbonic anhydrase II |
| 412/413 | CALB1 | 793 | calbindin 1, 28 kDa |
| 90/211 | CALCA | 796 | calcitonin/calcitonin-related polypeptide, |
| | | | alpha |
| 632 | CAPN11 | 11131 | calpain 11 |
| 140 | CAPN3 | 825 | calpain 3, (p94) |
| 569 | CAPN6 | 827 | calpain 6 |
| 561 | CAV2 | 858 | caveolin 2 |
| 216 | CCL15 /// | 6359 /// | C—C motif chemokine ligand 15 |
| | CCL15- | 348249 | |
| | CCL14 | | |
| 12 | CCL18 | 6362 | chemokine (C—C motif) ligand 18 |
| | | | (pulmonary and activation-regulated) |
| 231 | CCL19 | 6363 | chemokine (C—C motif) ligand 19 |
| 425 | CCL20 | 6364 | chemokine (C—C motif) ligand 20 |
| 359 | CCR7 | 1236 | chemokine (C—C motif) receptor 7 |
| 94 | CD22 | 933 | CD22 molecule |
| 13 | CD24 | 100133941 | signal transducer CD24 isoform a |
| | | | preproprotein /// signal transducer CD24 |
| | | | isoform a preproprotein /// signal transducer |
| | | | CD24 isoform b /// signal transducer CD24 |
| | | | isoform a preproprotein /// /// /// |
| 296 | CD24 | 934 | CD24 molecule |
| 267 | CD36 | 948 | CD36 molecule (thrombospondin receptor) |
| 527 | CD37 | 951 | CD37 molecule |
| 10 | CD52 | 1043 | CAMPATH-1 antigen precursor |
| 252 | CD69 | 969 | CD69 molecule |
| 594 | CDH1 | 999 | cadherin 1, type 1, E-cadherin (epithelial) |
| 248 | CDH17 | 1015 | cadherin 17, LI cadherin (liver-intestine) |
| 328 | CDH19 | 28513 | cadherin 19, type 2 |
| 557 | CDH2 | 1000 | cadherin 2, type 1, N-cadherin (neuronal) |
| 528 | CDO1 | 1036 | cysteine dioxygenase, type I |
| 589 | CEACAM5 | 1048 | carcinoembryonic antigen-related cell |
| | | | adhesion molecule 5 |
| 196/551 | CEACAM6 | 4680 | carcinoembryonic antigen-related cell |
| | | | adhesion molecule 6 (non-specific cross |
| | | | reacting antigen) |
| 371 | CEACAM7 | 1087 | carcinoembryonic antigen-related cell |
| | | | adhesion molecule 7 |
| 388 | CEL | 1056 | carboxyl ester lipase (bile salt-stimulated |
| | | | lipase) |
| 308 | CFHR5 | 81494 | complement factor H-related 5 |
| 273/274 | CHI3L1 | 1116 | chitinase 3-like 1 (cartilage glycoprotein- |
| | | | 39) |
| 498 | CHL1 | 10752 | cell adhesion molecule with homology to |
| | | | L1CAM (close homolog of L1) |
| 92 | CLCA2 | 9635 | chloride channel, calcium activated, family |
| | | | member 2 |
| 685 | CLDN16 | 10686 | claudin 16 |
| 29/30/151 | CLDN18 | 51208 | claudin 18 |
| 537 | CLDN3 | 1365 | claudin-3 |
| 137 | CLDN8 | 9073 | claudin 8 |
| 41 | CLEC2D | 29121 | C-type lectin domain family 2, member D |
| 396 | CLGN | 1047 | calmegin |
| 65 | CLIC3 | 9022 | chloride intracellular channel 3 |
| 176 | CLIC5 | 53405 | chloride intracellular channel 5 |
| 130 | CNIH3 | 149111 | cornichon homolog 3 (Drosophila) |
| 173 | CNR1 | 1268 | cannabinoid receptor 1 (brain) |
| 93 | COL10A1 | 1300 | collagen, type X, alpha 1(Schmid |
| | | | metaphyseal chondrodysplasia) |
| 5/517/ | COL11A1 | 1301 | collagen, type XI, alpha 1 |
| 183 | COL14A1 | 7373 | collagen, type XIV, alpha 1 (undulin) |
| 581 | COL1A1 | 1277 | collagen, type I, alpha 1 |
| | | | collagen, type II, alpha 1 (primary |
| 171 | COL2A1 | 1280 | osteoarthritis, spondyloepiphyseal |
| | | | dysplasia, congenital) |
| 15 | COL4A3 | 1285 | collagen, type IV, alpha 3 (Goodpasture |
| | | | antigen) |
| 178 | COL4A5 | 1287 | collagen, type IV, alpha 5 (Alport |
| | | | syndrome) |
| 405 | COMP | 1311 | cartilage oligomeric matrix protein |
| 481 | CP | 1356 | ceruloplasmin (ferroxidase) |
| 422 | CPB1 | 1360 | carboxypeptidase B1 (tissue) |
| 338 | CPB2 | 1361 | carboxypeptidase B2 (plasma) |
| 595 | CPE | 1363 | carboxypeptidase E |
| 379 | CPM | 1368 | carboxypeptidase M |
| 89/476 | CPS1 | 1373 | carbamoyl-phosphate synthetase 1, |
| | | | mitochondrial |
| 419 | CR2 | 1380 | complement component (3d/Epstein Barr |
| | | | virus) receptor 2 |
| 316 | CRISP3 | 10321 | cysteine-rich secretory protein 3 |
| 7 | CRP | 1401 | C-reactive protein, pentraxin-related |
| 451 | CSF2RB | 1439 | colony stimulating factor 2 receptor, beta, |
| | | | low-affinity (granulocyte-macrophage) |
| 367 | CST1 | 1469 | cystatin SN |
| 465 | CSTA | 1475 | cystatin A (stefin A) |
| 195/212 | CTAG1A/// | 246100///1485 | cancer/testis antigen 1A///cancer/testis |
| | CTAG1B | | antigen 1B |
| 633 | CTNND1 /// | 1500 /// | catenin delta-1 isoform 1ABC /// catenin |
| | TMX2- | 100528016 | delta-1 isoform 1AB /// catenin delta-1 |
| | CTNND1 | | isoform 1A /// catenin delta-1 isoform 1A /// |
| | | | catenin delta-1 isoform 1A /// catenin delta- |
| | | | 1 isoform 3ABC /// catenin delta-1 isoform |
| | | | 3AB /// catenin delta-1 isoform 3B /// |
| | | | catenin delta-1 isoform 3AC /// catenin |
| | | | delta-1 isoform 3A /// catenin delta-1 |
| | | | isoform 3A /// catenin delta-1 isoform 3A/// |
| | | | catenin delta-1 isoform 2ABC /// catenin |
| | | | delta-1 isoform 2AC /// catenin delta-1 |
| | | | isoform 1AC /// catenin delta-1 isoform |
| | | | 2AB /// catenin delta-1 isoform 2B /// |
| | | | catenin delta-1 isoform 2A /// catenin delta- |
| | | | 1 isoform 2A /// catenin delta-1 isoform 3A |
| | | | /// catenin delta-1 isoform 2A /// catenin |
| | | | delta-1 isoform 1B /// |
| 604 | CTR9 | 9646 | Ctr9, Paf1/RNA polymerase II complex |
| | | | component, homolog (S. cerevisiae) |
| 385 | CTSE | 1510 | cathepsin E |
| 630 | CUL1 | 8454 | cullin 1 |
| 161 | CUX2 | 23316 | cut-like homeobox 2 |
| 32 | CWH43 | 80157 | PGAP2-interacting protein isoform 2 /// |
| | | | PGAP2-interacting protein isoform 1 |
| 505 | CXCL1 | 2919 | chemokine (C-X-C motif) ligand 1 |
| | | | (melanoma growth stimulating activity, |
| | | | alpha) |
| 207/224/641 | CXCL11 | 6373 | chemokine (C-X-C motif) ligand 11 |
| 257 | CXCL12 | 6387 | stromal cell-derived factor 1 isoform beta |
| | | | precursor /// stromal cell-derived factor 1 |
| | | | isoform gamma precursor /// stromal cell- |
| | | | derived factor 1 isoform delta precursor /// |
| | | | stromal cell-derived factor 1 isoform 5 |
| | | | precursor /// stromal cell-derived factor 1 |
| | | | isoform alpha precursor |
| 444 | CXCL13 | 10563 | chemokine (C-X-C motif) ligand 13 (B-cell |
| | | | chemoattractant) |
| 88 | CXCL14 | 9547 | chemokine (C-X-C motif) ligand 14 |
| 253 | CXCL2 | 2920 | chemokine (C-X-C motif) ligand 2 |
| 314 | CXCL3 | 2921 | chemokine (C-X-C motif) ligand 3 |
| 127/129 | CXCL5 | 6374 | chemokine (C-X-C motif) ligand 5 |
| 202/574 | CXCL8 | 3576 | interleukin-8 precursor |
| 578 | CYP1B1 | 1545 | cytochrome P450, family 1, subfamily B, |
| | | | polypeptide 1 |
| 307 | CYP2C8 | 1558 | cytochrome P450 2C8 isoform a precursor |
| | | | /// cytochrome P450 2C8 isoform b /// |
| | | | cytochrome P450 2C8 isoform c /// |
| | | | cytochrome P450 2C8 isoform b |
| 240/241/597 | CYP2E1 | 1571 | cytochrome P450, family 2, subfamily E, |
| | | | polypeptide 1 |
| 148/149/401 | CYP3A5 | 1577 | cytochrome P450, family 3, subfamily A, |
| | | | polypeptide 5 |
| | CYP3A5P2 | 79424 | cytochrome P450, family 3, subfamily A, |
| | | | polypeptide 5 pseudogene 2 |
| 230 | CYP4B1 | 1580 | cytochrome P450, family 4, subfamily B, |
| | | | polypeptide 1 |
| 643 | CYP4F8 | 11283 | cytochrome P450, family 4, subfamily F, |
| | | | polypeptide 8 |
| 302/313 | DAZ1 /// | 1617 /// | deleted in azoospermia protein 4 isoform 1 |
| | DAZ2 /// | 57054 /// | /// deleted in azoospermia protein 2 isoform |
| | DAZ3 /// | 57055 /// | 2 /// deleted in azoospermia protein 2 |
| | DAZ4 | 57135 | isoform 3 /// deleted in azoospermia protein |
| | | | 1 /// deleted in azoospermia protein 2 |
| | | | isoform 1 /// deleted in azoospermia protein |
| | | | 3 /// deleted in azoospermia protein 4 |
| | | | isoform 2 |
| 106 | DCT | 1638 | L-dopachrome tautomerase isoform 2 |
| | | | precursor /// L-dopachrome tautomerase |
| | | | isoform 1 precursor |
| 107 | DCT | 1638 | L-dopachrome tautomerase isoform 2 |
| | | | precursor /// L-dopachrome tautomerase |
| | | | isoform 1 precursor |
| 437 | DCT | 1638 | dopachrome tautomerase (dopachrome |
| | | | delta-isomerase, tyrosine-related protein 2) |
| 438/440 | DDC | 1644 | dopa decarboxylase (aromatic L-amino acid |
| | | | decarboxylase) |
| 463 | DDX3Y | 8653 | DEAD (Asp-Glu-Ala-Asp) box polypeptide |
| | | | 3, Y-linked |
| 215 | DEFB1 | 1672 | defensin, beta 1 |
| 154 | DHRS2 | 10202 | dehydrogenase/reductase (SDR family) |
| | | | member 2 |
| 497 | DKK1 | 22943 | dickkopf homolog 1 (Xenopus laevis) |
| | | | dickkopf-related protein 3 precursor /// |
| 667 | DKK3 | 27122 | dickkopf-related protein 3 precursor /// |
| | | | dickkopf-related protein 3 precursor /// |
| 266 | DLK1 | 8788 | delta-like 1 homolog (Drosophila) |
| 545 | DMD | 1756 | dystrophin (muscular dystrophy, Duchenne |
| | | | and Becker types) |
| 612 | DMXL1 | 1657 | Dmx-like 1 |
| 203/552 | DPP4 | 1803 | dipeptidyl-peptidase 4 (CD26, adenosine |
| | | | deaminase complexing protein 2) |
| 180 | DPT | 1805 | dermatopontin |
| 508 | DST | 667 | dystonin |
| 532 | DUSP4 | 1846 | dual specificity phosphatase 4 |
| 300 | EDN3 | 1908 | endothelin 3 |
| 334/520/522 | EDNRB | 1910 | endothelin receptor type B |
| 50 | EHF | 26298 | ets homologous factor |
| 511 | EIF1AY | 9086 | eukaryotic translation initiation factor 1A, |
| | | | Y-linked |
| 678 | EIF4G2 | 1982 | eukaryotic translation initiation factor 4 |
| | | | gamma 2 isoform 2 /// eukaryotic translation |
| | | | initiation factor 4 gamma 2 isoform 1 /// |
| | | | eukaryotic translation initiation factor 4 |
| | | | gamma 2 isoform 1 |
| 66 | ELL3 | 80237 | elongation factor RNA polymerase II-like 3 |
| 168 | ELOVL2 | 54898 | elongation of very long chain fatty acids |
| | | | (FEN1/Elo2, SUR4/Elo3, yeast)-like 2 |
| 17 | EMX2 | 2018 | empty spiracles homeobox 2 |
| 482/483 | ENPEP | 2028 | glutamyl aminopeptidase (aminopeptidase |
| | | | A) |
| 591 | EPCAM | 4072 | epithelial cell adhesion molecule precursor |
| 380 | EPHA3 | 2042 | EPH receptor A3 |
| 348 | EPYC | 1833 | epiphycan |
| 446 | ESR1 | 2099 | estrogen receptor 1 |
| 610 | ETFB | 2109 | electron-transfer-flavoprotein, beta |
| | | | polypeptide |
| 19 | ETV1 | 2115 | ets variant gene 1 |
| 192 | EVI2B | 2124 | ecotropic viral integration site 2B |
| 170 | F2RL1 | 2150 | proteinase-activated receptor 2 precursor |
| 489 | F5 | 2153 | coagulation factor V (proaccelerin, labile |
| | | | factor) |
| 325 | F9 | 2158 | coagulation factor IX (plasma |
| | | | thromboplastic component, Christmas |
| | | | disease, hemophilia B) |
| 390 | FABP1 | 2168 | fatty acid binding protein 1, liver |
| 534 | FABP4 | 2167 | fatty acid binding protein 4, adipocyte |
| 460/461 | FABP7 | 2173 | fatty acid binding protein 7, brain |
| 249 | FAM65B | 9750 | protein FAM65B isoform 3 /// protein |
| | | | FAM65B isoform 4 /// protein FAM65B |
| | | | isoform 5 /// protein FAM65B isoform 1 /// |
| | | | protein FAM65B isoform 2 |
| 566 | FBLN1 | 2192 | fibulin 1 |
| 563 | FBN2 | 2201 | fibrillin 2 (congenital contractural |
| | | | arachnodactyly) |
| 533/615 | FCGR3B | 2215 | Fc fragment of IgG, low affinity IIIb, |
| | | | receptor (CD16b) |
| 1 | FERMT1 | 55612 | fermitin family homolog 1 |
| 410/411 | FGA | 2243 | fibrinogen alpha chain |
| 112/464 | FGB | 2244 | fibrinogen beta chain |
| 515 | FGFR3 | 2261 | fibroblast growth factor receptor 3 |
| | | | (achondroplasia, thanatophoric dwarfism) |
| 59 | FGG | 2266 | fibrinogen gamma chain |
| 218 | FHL1 | 2273 | four and a half LIM domains 1 |
| 526 | FLI1 | 2313 | Friend leukemia virus integration 1 |
| 72 | FLRT3 | 23767 | fibronectin leucine rich transmembrane |
| | | | protein 3 |
| 3/347 | FMO3 | 2328 | flavin containing monooxygenase 3 |
| 121/393 | FOLH1 /// | 2346 /// | folate hydrolase 1 |
| | FOLH1B | 219595 | |
| 492 | FOXA1 | 3169 | forkhead box A1 |
| 599 | FOXE1 | 2304 | forkhead box E1 (thyroid transcription |
| | | | factor 2) |
| 553 | FRZB | 2487 | frizzled-related protein |
| 156 | FUT9 | 10690 | fucosyltransferase 9 (alpha (1,3) |
| | | | fucosyltransferase) |
| 27 | FZD5 | 7855 | frizzled-5 precursor /// |
| 391 | GABBR1 /// | 2550 /// | gamma-aminobutyric acid type B receptor |
| | UBD | 10537 | subunit 1 isoform a precursor /// ubiquitin D |
| | | | /// gamma-aminobutyric acid type B |
| | | | receptor subunit 1 isoform b precursor /// |
| | | | gamma-aminobutyric acid type B receptor |
| | | | subunit 1 isoform c precursor |
| 237 | GABBR2 | 9568 | gamma-aminobutyric acid (GABA) B |
| | | | receptor, 2 |
| 457 | GABRP | 2568 | gamma-aminobutyric acid (GABA) A |
| | | | receptor, pi |
| 326 | GAGE1 /// | 2543 /// 2574 | G antigen 1 /// G antigen 12F///G antigen |
| | GAGE12B | /// 2576 /// | 12J /// G antigen 2D /// G antigen |
| | /// | 2577 /// 2578 | 12B/C/D/E /// G antigen 12G /// G antigen |
| | GAGE12C | /// 2579 /// | 12H///G antigen 2B/2C///G antigen 13 /// |
| | /// | 26748 /// | G antigen 12B/C/D/E /// G antigen |
| | GAGE12D | 26749 /// | 12B/C/D/E /// G antigen 2E /// G antigen |
| | /// | 645037 /// | 2A/2B /// G antigen 12B/C/D/E /// /// G |
| | GAGE12E | 645051 /// | antigen 2B/2C ///G antigen 4 /// G antigen |
| | /// | 645073 /// | 5 /// G antigen 6 /// G antigen 12I /// G |
| | GAGE12F | 729396 /// | antigen 2D /// G antigen 12G |
| | /// | 729408 /// | |
| | GAGE12G | 729422 /// | |
| | /// | 729428 /// | |
| | GAGE12H | 729431 /// | |
| | /// | 729442 /// | |
| | GAGE12I | 729447 /// | |
| | /// | 100008586 /// | |
| | GAGE12J | 100101629 /// | |
| | /// GAGE13 | 100132399 | |
| | /// GAGE2A | | |
| | /// GAGE2B | | |
| | /// GAGE2C | | |
| | /// GAGE2D | | |
| | /// GAGE2E | | |
| | /// GAGE4 | | |
| | /// GAGE5 | | |
| | /// GAGE6 | | |
| | /// GAGE7 | | |
| | /// GAGE8 | | |
| 318 | GAGE1 /// | 2543 /// 2574 | G antigen 1 /// G antigen 12F /// G antigen |
| | GAGE12D | /// 2575 /// | 12J /// G antigen 2D /// G antigen 12G /// G |
| | /// | 2576 /// 2577 | antigen 2B/2C///G antigen 13 /// G antigen |
| | GAGE12F | /// 2578 /// | 12B/C/D/E /// G antigen 2E /// G antigen |
| | /// | 2579 /// | 2A/2B /// /// G antigen 2B/2C /// G antigen |
| | GAGE12G | 26748 /// | 4 /// G antigen 5 /// G antigen 6 /// G antigen |
| | /// | 26749 /// | 12I /// G antigen 2D /// G antigen 12G |
| | GAGE12I | 645037 /// | |
| | /// | 645051 /// | |
| | GAGE12J | 645073 /// | |
| | /// GAGE13 | 729396 /// | |
| | /// GAGE2A | 729408 /// | |
| | /// GAGE2B | 729447 /// | |
| | /// GAGE2C | 100008586 /// | |
| | /// GAGE2D | 100101629 /// | |
| | /// GAGE2E | 100132399 | |
| | /// GAGE3 | | |
| | /// GAGE4 | | |
| | /// GAGE5 | | |
| | /// GAGE6 | | |
| | /// GAGE7 | | |
| | /// GAGE8 | | |
| 306 | GAGE1 /// | 2543 /// 2576 | G antigen 1 /// G antigen 12F /// G antigen |
| | GAGE12D | ///2577 /// | 12J /// G antigen 2D /// G antigen 12G /// G |
| | /// | 2578 /// 2579 | antigen 2B/2C /// G antigen 13 /// G antigen |
| | GAGE12F | ///26748 /// | 12B/C/D/E /// G antigen 2E /// /// G antigen |
| | /// | 26749 /// | 4 /// G antigen 5 /// G antigen 6 /// G antigen |
| | GAGE12G | 645037 /// | 12I /// G antigen 12G |
| | /// | 645051 /// | |
| | GAGE12I | 645073 /// | |
| | /// | 729396 /// | |
| | GAGE12J | 729408 /// | |
| | /// GAGE13 | 100008586 /// | |
| | /// GAGE2B | 100132399 | |
| | /// GAGE2D | | |
| | /// GAGE2E | | |
| | /// GAGE4 | | |
| | /// GAGE5 | | |
| | /// GAGE6 | | |
| | /// GAGE7 | | |
| 340 | GAGE12B | 2574 /// 2576 | G antigen 12F /// G antigen 2D /// G antigen |
| | /// | /// 2577 /// | 12B/C/D/E /// G antigen 12G /// G antigen |
| | GAGE12C | 2578///2579 | 12H /// G antigen 12B/C/D/E /// G antigen |
| | /// | /// 26748 /// | 12B/C/D/E /// G antigen 2E /// G antigen |
| | GAGE12D | 26749 /// | 2A/2B /// G antigen 12B/C/D/E /// G antigen |
| | /// | 645073 /// | 2B/2C /// G antigen 4 /// G antigen 5 /// G |
| | GAGE12E | 729408 /// | antigen 6 /// G antigen 12I /// G antigen 2D |
| | /// | 729422 /// | /// G antigen 12G /// /// |
| | GAGE12F | 729428 /// | |
| | /// | 729431 /// | |
| | GAGE12G | 729442 /// | |
| | /// | 729447 /// | |
| | GAGE12H | 100008586 /// | |
| | /// | 100101629 /// | |
| | GAGE12I | 100132399 | |
| | /// GAGE2A | | |
| | /// GAGE2C | | |
| | /// GAGE2D | | |
| | /// GAGE2E | | |
| | /// GAGE4 | | |
| | /// GAGE5 | | |
| | /// GAGE6 | | |
| | /// GAGE7 | | |
| | /// GAGE8 | | |
| 304 | GAGE7 | 2579 | G antigen 7 |
| 560 | GALNT3 | 2591 | UDP-N-acetyl-alpha-D- |
| | | | galactosamine: polypeptide N- |
| | | | acetylgalactosaminyltransferase 3 |
| | | | (GalNAc-T3) |
| 504 | GAP43 | 2596 | growth associated protein 43 |
| 263 | GATA3 | 2625 | GATA binding protein 3 |
| 236 | GATA6 | 2627 | GATA binding protein 6 |
| 564 | GATM | 2628 | glycine amidinotransferase (L- |
| | | | arginine:glycine amidinotransferase) |
| 466 | GC | 2638 | group-specific component (vitamin D |
| | | | binding protein) |
| 351 | GCG | 2641 | glucagon |
| 25 | GDF15 | 9518 | growth differentiation factor 15 |
| 661 | GDPD5 | 81544 | glycerophosphodiester phosphodiesterase |
| | | | domain containing 5 |
| 649 | GGA3 | 23163 | golgi associated, gamma adaptin ear |
| | | | containing, ARF binding protein 3 |
| 423 | GHR | 2690 | growth hormone receptor |
| 54 | GIMAP6 | 474344 | GTPase, IMAP family member 6 |
| 663 | GLB1L2 | 89944 | beta-galactosidase-1-like protein 2 |
| | | | precursor |
| 623 | GNAL | 2774 | guanine nucleotide binding protein (G |
| | | | protein), alpha activating activity |
| | | | polypeptide, olfactory type |
| 289 | GPM6B | 2824 | neuronal membrane glycoprotein M6-b |
| | | | isoform 4 /// neuronal membrane |
| | | | glycoprotein M6-b isoform 1 /// neuronal |
| | | | membrane glycoprotein M6-b isoform 2 /// |
| | | | neuronal membrane glycoprotein M6-b |
| | | | isoform 3 |
| 290 | GPM6B | 2824 | neuronal membrane glycoprotein M6-b |
| | | | isoform 4 /// neuronal membrane |
| | | | glycoprotein M6-b isoform 1 /// neuronal |
| | | | membrane glycoprotein M6-b isoform 2 /// |
| | | | neuronal membrane glycoprotein M6-b |
| | | | isoform 3 |
| 291 | GPM6B | 2824 | glycoprotein M6B |
| 336 | GPR143 | 4935 | G protein-coupled receptor 143 |
| 220 | GPR18 | 2841 | G protein-coupled receptor 18 |
| 259 | GPR37 | 2861 | prosaposin receptor GPR37 precursor |
| 141 | GPR65 | 8477 | G protein-coupled receptor 65 |
| 47 | GPR87 | 53836 | G protein-coupled receptor 87 |
| 369 | GRB14 | 2888 | growth factor receptor-bound protein 14 |
| 392 | GREB1 | 9687 | GREB1 protein |
| 83/84/680 | GREM1 | 26585 | gremlin 1, cysteine knot superfamily, |
| | | | homolog (Xenopus laevis) |
| 434 | GRIA2 | 2891 | glutamate receptor, ionotropic, AMPA 2 |
| 646 | GRM1 | 2911 | glutamate receptor, metabotropic 1 |
| 689 | GRWD1 | 83743 | glutamate-rich WD repeat containing 1 |
| 539 | GSTA2 | 2939 | glutathione S-transferase A2 |
| 525 | GULP1 | 51454 | GULP, engulfment adaptor PTB domain |
| | | | containing 1 |
| 223 | GZMB | 3002 | granzyme B (granzyme 2, cytotoxic T- |
| | | | lymphocyte-associated serine esterase 1) |
| 682 | HEATR3 | 55027 | HEAT repeat-containing protein 3 |
| 543 | HEPH | 9843 | hephaestin |
| 447 | HGD | 3081 | homogentisate 1,2-dioxygenase |
| | | | (homogentisate oxidase) |
| 113 | HHEX | 3087 | hematopoietically expressed homeobox |
| 165/562 | HLA-DQA1 | 3117 | major histocompatibility complex, class II, |
| | | | DQ alpha 1 |
| 185 | HLA-DQA1 | 3117 /// 3118 | HLA class II histocompatibility antigen, |
| | /// HLA- | | DQ alpha 1 chain precursor /// HLA class II |
| | DQA2 | | histocompatibility antigen, DQ alpha 2 |
| | | | chain precursor |
| 269 | HLA-DQB1 | 3119 | major histocompatibility complex, class II, |
| | | | DQ beta 1 |
| 26 | HLA-DQB1 | 3119 /// 3123 | HLA class II histocompatibility antigen, |
| | /// HLA- | /// 3124 /// | DQ beta 1 chain isoform 2 precursor /// |
| | DRB1 /// | 3125 /// 3126 | HLA class II histocompatibility antigen, |
| | HLA-DRB2 | /// 3127 /// | DQ beta 1 chain isoform 1 precursor /// |
| | /// HLA- | 3128 /// 3129 | major histocompatibility complex, class II, |
| | DRB3 /// | /// 3130 /// | DR beta 1 precursor /// HLA class II |
| | HLA-DRB4 | 105369230 | histocompatibility antigen, DQ beta 1 chain |
| | /// HLA- | | isoform 1 precursor /// major |
| | DRB5 /// | | histocompatibility complex, class II, DR |
| | HLA-DRB6 | | beta 1 precursor /// major histocompatibility |
| | /// HLA- | | complex, class II, DR beta 5 precursor /// |
| | DRB7 /// | | major histocompatibility complex, class II, |
| | HLA-DRB8 | | DR beta 4 precursor /// major |
| | /// | | histocompatibility complex, class II, DR |
| | LOC105369 | | beta 3 precursor |
| | 230 | | |
| 309 | HMGA2 | 8091 | high mobility group AT-hook 2 |
| 496 | HMGCS2 | 3158 | 3-hydroxy-3-methylglutaryl-Coenzyme A |
| | | | synthase 2 (mitochondrial) |
| 627 | HMX1 | 3166 | H6 family homeobox 1 |
| 133 | HOXA9 | 3205 | homeobox A9 |
| 335 | HP | 3240 | haptoglobin |
| 299 | HP /// HPR | 3240 /// 3250 | haptoglobin isoform 2 preproprotein /// |
| | | | haptoglobin isoform 1 preproprotein /// |
| | | | haptoglobin-related protein precursor |
| 383 | HPD | 3242 | 4-hydroxyphenylpyruvate dioxygenase |
| 201 | HPGD | 3248 | 15-hydroxyprostaglandin dehydrogenase |
| | | | [NAD(+)] isoform 1 /// 15- |
| | | | hydroxyprostaglandin dehydrogenase |
| | | | [NAD(+)] isoform 2 /// 15- |
| | | | hydroxyprostaglandin dehydrogenase |
| | | | [NAD(+)] isoform 3 /// 15- |
| | | | hydroxyprostaglandin dehydrogenase |
| | | | [NAD(+)] isoform 4 /// 15- |
| | | | hydroxyprostaglandin dehydrogenase |
| | | | [NAD(+)] isoform 5 /// 15- |
| | | | hydroxyprostaglandin dehydrogenase |
| | | | [NAD(+)] isoform 3 |
| 540/541 | HPGD | 3248 | hydroxyprostaglandin dehydrogenase 15- |
| | | | (NAD) |
| 484 | HSD17B2 | 3294 | hydroxysteroid (17-beta) dehydrogenase 2 |
| 6/406 | HSD17B6 | 8630 | hydroxysteroid (17-beta) dehydrogenase 6 |
| | | | homolog (mouse) |
| 639 | HSF2 | 3298 | heat shock transcription factor 2 |
| 608 | HSPA13 | 6782 | heat shock 70 kDa protein 13 precursor |
| 281/282 | ID4 | 3400 | inhibitor of DNA binding 4, dominant |
| | | | negative helix-loop-helix protein |
| 607 | IFI27 | 3429 | interferon, alpha-inducible protein 27 |
| 268 | IGF1 | 3479 | insulin-like growth factor I isoform 4 |
| | | | preproprotein /// insulin-like growth factor I |
| | | | isoform 1 preproprotein /// insulin-like |
| | | | growth factor I isoform 2 precursor /// /// /// |
| | | | /// |
| 547 | IGF2BP3 | 10643 | insulin-like growth factor 2 mRNA-binding |
| | | | protein 3 |
| 548 | IGF2BP3 | 10643 | insulin-like growth factor 2 mRNA binding |
| | | | protein 3 |
| 441 | IGFBP1 | 3484 | insulin-like growth factor binding protein 1 |
| 125 | IGH | 3492 | immunoglobulin heavy locus |
| 100/108 | IGHAl /// | 3493 /// 3500 | zinc finger CW-type PWWP domain |
| | IGHG1 /// | /// 3507 /// | protein 2 |
| | IGHM /// | 28396 /// | |
| | IGHV3-23 | 28442 /// | |
| | /// IGHV4- | 50802 /// | |
| | 31 /// IGK | 152098 | |
| | /// | | |
| | ZCWPW2 | | |
| 109/276 | IGHM | 3507 | immunoglobulin heavy constant mu |
| 200 | IGHM /// | 3507 /// | immunoglobulin heavy constant mu |
| | IGHV1-69 | 28458 /// | |
| | /// IGHV1- | 28461 | |
| | 69-2 | | |
| 692 | IGHMBP2 | 3508 | immunoglobulin mu binding protein 2 |
| 199 | IGKC | 3514 | immunoglobulin kappa constant |
| 198 | IGKV1-17 | 28937 | immunoglobulin kappa variable 1-17 |
| 110 | IGKV1-37 | 28894 /// | immunoglobulin kappa variable 1D- |
| | /// | 28931 | 37///immunoglobulin kappa variable 1-37 |
| | IGKV1D-37 | | |
| 123 | IGKV1-39 | 28893 /// | immunoglobulin kappa variable 1D-39 |
| | /// | 28930 | |
| | IGKV1D-39 | | |
| 122 | IGLV3-25 | 28793 | immunoglobulin lambda variable 3-25 |
| 373 | IL13RA2 | 3598 | interleukin 13 receptor, alpha 2 |
| 674 | IL9R | 3581 | interleukin-9 receptor isoform 1 precursor |
| | | | /// interleukin-9 receptor isoform 2 |
| 690 | IMP3 | 55272 | IMP3, U3 small nucleolar |
| | | | ribonucleoprotein, homolog (yeast) |
| 343 | INS | 3630 | insulin |
| 378 | ISL1 | 3670 | ISL LIM homeobox 1 |
| 402 | ITIH3 | 3699 | inter-alpha (globulin) inhibitor H3 |
| 576 | ITM2A | 9452 | integral membrane protein 2A |
| 186 | JCHAIN | 3512 | immunoglobulin J chain precursor |
| 658 | JMJD6 | 23210 | jumonji domain containing 6 |
| 228 | KCNJ15 | 3772 | potassium inwardly-rectifying channel, |
| | | | subfamily J, member 15 |
| 63 | KCNJ16 | 3773 | potassium inwardly-rectifying channel, |
| | | | subfamily J, member 16 |
| 34 | KHDC1L | 100129128 | putative KHDC1-like protein |
| 2 | KIAA0226L | 80183 | uncharacterized protein KIAA0226-like |
| | | | isoform a /// uncharacterized protein |
| | | | KIAA0226-like isoform b /// |
| | | | uncharacterized protein KIAA0226-like |
| | | | isoform c /// uncharacterized protein |
| | | | KIAA0226-like isoform d /// |
| | | | uncharacterized protein KIAA0226-like |
| | | | isoform e /// uncharacterized protein |
| | | | KIAA0226-like isoform f /// |
| | | | uncharacterized protein KIAA0226-like |
| | | | isoform a |
| 670 | KIAA1024 | 23251 | KIAA1024 protein |
| 659 | KIAA1109 | 84162 | KIAA1109 |
| 611 | KIF3C | 3797 | kinesin family member 3C |
| 287 | KLF5 | 688 | Kruppel-like factor 5 (intestinal) |
| 426/600 | KLK2 | 3817 | kallikrein-related peptidase 2 |
| 499/500 | KLK3 | 354 | kallikrein-related peptidase 3 |
| 382 | KNG1 | 3827 | kininogen 1 |
| 311 | KRT13 | 3860 | keratin 13 |
| 278 | KRT14 | 3861 | keratin 14 (epidermolysis bullosa simplex, |
| | | | Dowling-Meara, Koebner) |
| 487 | KRT15 | 3866 | keratin 15 |
| 452 | KRT17 | 3872 | keratin 17 |
| 592 | KRT19 | 3880 | keratin 19 |
| 159 | KRT20 | 54474 | keratin 20 |
| 77 | KRT23 | 25984 | keratin 23 (histone deacetylase inducible) |
| 293 | KRT6A | 3853 | keratin 6A |
| 295 | KRT7 | 3855 | keratin 7 |
| 96 | KYNU | 8942 | kynureninase (L-kynurenine hydrolase) |
| 45 | L1TD1 | 54596 | LINE-1 type transposase domain containing |
| | | | 1 |
| 143 | LBP | 3929 | lipopolysaccharide binding protein |
| 188 | LCN2 | 3934 | lipocalin 2 (oncogene 24p3) |
| 442 | LCP2 | 3937 | lymphocyte cytosolic protein 2 (SH2 |
| | | | domain containing leukocyte protein of |
| | | | 76 kDa) |
| 605 | LDLR | 3949 | low density lipoprotein receptor (familial |
| | | | hypercholesterolemia) |
| 364 | LEFTY1 | 10637 | left-right determination factor 1 |
| 244 | LEPR | 3953 | leptin receptor |
| 609 | LEPROTL1 | 23484 | leptin receptor overlapping transcript-like 1 |
| 521 | LGALS4 | 3960 | lectin, galactoside-binding, soluble, 4 |
| | | | (galectin 4) |
| 164 | LGR5 | 8549 | leucine-rich repeat-containing G protein- |
| | | | coupled receptor 5 |
| 52 | LIN28A | 79727 | protein lin-28 homolog A |
| 360 | LIPF | 8513 | lipase, gastric |
| 102 | LOC100126 | 100126583/// | hypothetical |
| | 583///IGHA | 3494///3493 | LOC100126583///immunoglobulin heavy |
| | 2///IGHA1 | | constant alpha 2 (A2m |
| | | | marker)///immunoglobulin heavy constant |
| | | | alpha 1 |
| 117 | LOC101929 | 101929272 | LOC101929272 |
| | 272 | | |
| 636 | LOC103021 | 7326 /// | ubiquitin-conjugating enzyme E2 G1 |
| | 295 /// | 103021295 | |
| | UBE2G1 | | |
| 118 | LOX | 4015 | lysyl oxidase |
| 555 | LPL | 4023 | lipoprotein lipase |
| 55 | LRAP | 64167 | leukocyte-derived arginine aminopeptidase |
| 9 | LRMP | 4033 | lymphoid-restricted membrane protein |
| 587 | LTF | 4057 | lactotransferrin |
| 409 | LY75 | 4065 | lymphocyte antigen 75 |
| 323 | MAGEA1 | 4100 | melanoma antigen family A, 1 (directs |
| | | | expression of antigen MZ2-E) |
| 134 | MAGEA12 | 4111 | melanoma antigen family A, 12 |
| 214 | MAGEA2B | 266740///139 | melanoma antigen family A, |
| | ///psMAGE | 041///4101 | 2B///melanoma antigen pseudogene, family |
| | A///MAGE | | A///melanoma antigen family A, 2 |
| | A2 | | |
| 136 | MAGEA3 | 4102 | melanoma antigen family A, 3 |
| 242 | MAGEA4 | 4103 | melanoma antigen family A, 4 |
| 147 | MAGEA5 | 4104 | melanoma antigen family A, 5 |
| 135 | MAGEA6 | 4105 | melanoma antigen family A, 6 |
| 368 | MAGEB2 | 4113 | melanoma antigen family B, 2 |
| 485 | MAL | 4118 | mal, T-cell differentiation protein |
| 513/514 | MAOA | 4128 | monoamine oxidase A |
| 572 | MAP7 | 9053 | microtubule-associated protein 7 |
| 579 | MATN2 | 4147 | matrilin 2 |
| 644 | MAX | 4149 | MYC associated factor X |
| 324 | MBL2 | 4153 | mannose-binding lectin (protein C) 2, |
| | | | soluble (opsonic defect) |
| 294 | MBP | 4155 | myelin basic protein |
| 672 | MCM5 | 4174 | minichromosome maintenance complex |
| | | | component 5 |
| 20 | MECOM | 2122 | MDS1 and EVI1 complex locus |
| 370 | MEOX2 | 4223 | mesenchyme homeobox 2 |
| 427 | MFAP3L | 9848 | microfibrillar-associated protein 3-like |
| 167 | MFAP5 | 8076 | microfibrillar associated protein 5 |
| 345 | MIA | 8190 | melanoma inhibitory activity |
| 652 | MKI67 | 4288 | antigen identified by monoclonal antibody |
| | | | Ki-67 |
| 349/350 | MLANA | 2315 | melan-A |
| 558 | MME | 4311 | membrane metallo-endopeptidase |
| 503 | MMP1 | 4312 | matrix metallopeptidase 1 (interstitial |
| | | | collagenase) |
| 501 | MMP12 | 4321 | matrix metallopeptidase 12 (macrophage |
| | | | elastase) |
| 524 | MMP7 | 4316 | matrix metallopeptidase 7 (matrilysin, |
| | | | uterine) |
| 468 | MNDA | 4332 | myeloid cell nuclear differentiation antigen |
| 430 | MPPED2 | 744 | metallophosphoesterase domain containing |
| | | | 2 |
| 550 | MPZL2 | 10205 | myelin protein zero-like 2 |
| 95/217 | MS4A1 | 931 | membrane-spanning 4-domains, subfamily |
| | | | A, member 1 |
| 60 | MS4A4A | 51338 | membrane-spanning 4-domains, subfamily |
| | | | A, member 4 |
| 660 | MSH5- | 401251 /// | suppressor APC domain-containing protein |
| | SAPCD1 /// | 100532732 | 1 /// |
| | SAPCD1 | | |
| 479 | MSLN | 10232 | mesothelin |
| 219/321 | MSMB | 4477 | microseminoprotein, beta- |
| 91 | MT1M | 4499 | metallothionein 1M |
| 647 | MTAP | 4507 | methylthioadenosine phosphorylase |
| 315 | MUC1 | 4582 | mucin 1, cell surface associated |
| 81 | MUC13 | 56667 | mucin 13, cell surface associated |
| 38 | MUC16 | 94025 | mucin 16, cell surface associated |
| 98 | MUC4 | 4585 | mucin 4, cell surface associated |
| 162 | MYBL1 | 4603 | v-myb myeloblastosis viral oncogene |
| | | | homolog (avian)-like 1 |
| 153 | MYBPC1 | 4604 | myosin binding protein C, slow type |
| 653 | MYH10 | 4628 | myosin, heavy chain 10, non-muscle |
| 593 | MYH11 | 4629 | myosin, heavy chain 11, smooth muscle |
| 677 | MYRF | 745 | myelin regulatory factor isoform 2 |
| | | | precursor /// myelin regulatory factor |
| | | | isoform 1 |
| 39 | NANOG | 79923 | Nanog homeobox |
| 467 | NCF1 /// | 653361 /// | neutrophil cytosol factor 1 |
| | NCF1B /// | 654816 /// | |
| | NCF1C | 654817 | |
| 535/536 | NEBL | 10529 | nebulette |
| 11 | NEFH | 4744 | neurofilament, heavy polypeptide 200 kDa |
| 22 | NEFL | 4747 | neurofilament, light polypeptide 68 kDa |
| 657 | NEMP1 | 23306 | nuclear envelope integral membrane protein |
| | | | 1 isoform a precursor /// nuclear envelope |
| | | | integral membrane protein 1 isoform b |
| 208 | NKX2-1 | 7080 | NK2 homeobox 1 |
| 256 | NKX3-1 | 4824 | NK3 homeobox 1 |
| 389 | NLGN1 | 22871 | neuroligin 1 |
| 18 | NLGN4X | 57502 | neuroligin 4, X-linked |
| 146 | NOV | 4856 | nephroblastoma overexpressed gene |
| 622 | NOVA1 | 4857 | neuro-oncological ventral antigen 1 |
| 352 | NOX1 | 27035 | NADPH oxidase 1 |
| 28 | NPL | 80896 | N-acetylneuraminate pyruvate lyase |
| | | | (dihydrodipicolinate synthase) |
| 172 | NPTX2 | 4885 | neuronal pentraxin II |
| 428 | NPY1R | 4886 | neuropeptide Y receptor Y1 |
| 111 | NR4A2 | 4929 | nuclear receptor subfamily 4, group A, |
| | | | member 2 |
| 264/265 | NSG1 | 27065 | neuron-specific protein family member 1 |
| | | | isoform a /// neuron-specific protein family |
| | | | member 1 isoform a /// neuron-specific |
| | | | protein family member 1 isoform b /// |
| | | | neuron-specific protein family member 1 |
| | | | isoform a |
| 23/625 | NTRK2 | 4915 | neurotrophic tyrosine kinase, receptor, type |
| | | | 2 |
| 362 | NTS | 4922 | neurotensin |
| 664 | NUP210 | 23225 | nucleoporin 210 kDa |
| 638 | NXT2 | 55916 | nuclear transport factor 2-like export factor |
| | | | 2 |
| 80 | OGN | 4969 | osteoglycin |
| 645 | OLFM1 | 10439 | olfactomedin 1 |
| 184 | OLFM4 | 10562 | olfactomedin 4 |
| 459 | ORM1 | 5004 | orosomucoid 1 |
| 142 | ORM1 /// | 5004 /// 5005 | alpha-1-acid glycoprotein 1 precursor /// |
| | ORM2 | | alpha-1-acid glycoprotein 2 precursor |
| 458 | ORM2 | 5005 | orosomucoid 2 |
| 341 | P2RY14 | 9934 | purinergic receptor P2Y, G-protein coupled, |
| | | | 14 |
| 404 | PAH | 5053 | phenylalanine hydroxylase |
| 16 | PAX5 | 5079 | paired box 5 |
| 138 | PAX8 | 7849 | paired box 8 |
| 73 | PBK | 55872 | PDZ binding kinase |
| 64 | PBLD | 64081 | phenazine biosynthesis-like protein domain |
| | | | containing |
| 683 | PCDH12 | 51294 | protocadherin 12 |
| 420 | PCDH7 | 5099 | protocadherin 7 |
| 301 | PCK1 | 5105 | phosphoenolpyruvate carboxykinase 1 |
| | | | (soluble) |
| 418 | PCP4 | 5121 | Purkinje cell protein 4 |
| 397 | PCSK1 | 5122 | proprotein convertase subtilisin/kexin type |
| | | | 1 |
| 417 | PCSK5 | 5125 | proprotein convertase subtilisin/kexin type |
| | | | 5 |
| 654 | PDCD11 | 22984 | programmed cell death 11 |
| 432 | PDZK1 | 5174 | PDZ domain containing 1 |
| 58 | PDZK1IP1 | 10158 | PDZK1 interacting protein 1 |
| 182 | PDZRN3 | 23024 | PDZ domain containing RING finger 3 |
| 190 | PEG10 | 23089 | paternally expressed 10 |
| 285/286 | PEG3 | 5178 | paternally expressed 3 |
| 631 | PFKFB2 | 5208 | 6-phosphofructo-2-kinase/fructose-2,6- |
| | | | biphosphatase 2 |
| 621 | PGAM2 | 5224 | phosphoglycerate mutase 2 (muscle) |
| 169 | PHACTR1 | 221692 | phosphatase and actin regulator 1 |
| 320 | PIR | 8544 | pirin (iron-binding nuclear protein) |
| 61 | PLA1A | 51365 | phospholipase A1 member A |
| 225 | PLA2G4A | 5321 | phospholipase A2, group IVA (cytosolic, |
| | | | calcium-dependent) |
| 76 | PLAC8 | 51316 | placenta-specific 8 |
| 327 | PLAGL1 | 5325 | pleiomorphic adenoma gene-like 1 |
| 590 | PLAT | 5327 | plasminogen activator, tissue |
| 614 | PLCB4 | 5332 | phospholipase C, beta 4 |
| 470/471/472 | PLN | 5350 | phospholamban |
| 222 | PLP1 | 5354 | proteolipid protein 1 (Pelizaeus- |
| | | | Merzbacher disease, spastic paraplegia 2, |
| | | | uncomplicated) |
| 449 | PLS1 | 5357 | plastin 1(1 isoform) |
| 688 | PLXNA1 | 5361 | plexin A1 |
| 519 | PMAIP1 | 5366 | phorbol-12-myristate-13-acetate-induced |
| | | | protein 1 |
| 247 | PMEL | 6490 | melanocyte protein PMEL isoform 2 |
| | | | precursor /// melanocyte protein PMEL |
| | | | isoform 1 precursor /// melanocyte protein |
| | | | PMEL isoform 3 preproprotein |
| 387 | PNLIP | 5406 | pancreatic lipase |
| 191 | PNLIPRP2 | 5408 | pancreatic lipase-related protein 2 |
| 679 | POMGNT1 | 55624 | protein O-linked mannose beta1,2-N- |
| | | | acetylglucosaminyltransferase |
| 443 | POU2AF1 | 5450 | POU class 2 associating factor 1 |
| 628 | PPP1R2P9 | 80316 | protein phosphatase 1 regulatory inhibitor |
| | | | subunit 2 pseudogene 9 |
| 529 | PRAME | 23532 | preferentially expressed antigen in |
| | | | melanoma |
| 310 | PRKCB1 | 5579 | protein kinase C, beta 1 |
| 650 | PRLR | 5618 | prolactin receptor |
| 518 | PROM1 | 8842 | prominin 1 |
| 431 | PRS S2 | 5645 | protease, serine, 2 (trypsin 2) |
| 439 | PSCA | 8000 | prostate stem cell antigen |
| 262 | PSCDBP | 9595 | pleckstrin homology, Sec7 and coiled-coil |
| | | | domains, binding protein |
| 456 | PSPH | 5723 | phosphoserine phosphatase |
| 648 | PTGDS | 5730 | prostaglandin D2 synthase 21 kDa (brain) |
| 486 | PTGS2 | 5743 | prostaglandin-endoperoxide synthase 2 |
| | | | (prostaglandin G/H synthase and |
| | | | cyclooxygenase) |
| 193/270 | PTN | 5764 | pleiotrophin (heparin binding growth factor |
| | | | 8, neurite growth-promoting factor 1) |
| 187 | PTPRC | 5788 | protein tyrosine phosphatase, receptor type, |
| | | | C |
| 506 | PTPRZ1 | 5803 | protein tyrosine phosphatase, receptor-type, |
| | | | Z polypeptide 1 |
| 375 | PTX3 | 5806 | pentraxin-related gene, rapidly induced by |
| | | | IL-1 beta |
| 450 | QPCT | 25797 | glutaminyl-peptide cyclotransferase |
| | | | (glutaminyl cyclase) |
| 86 | RAB25 | 57111 | RAB25, member RAS oncogene family |
| 70 | RAB38 | 23682 | RAB38, member RAS oncogene family |
| 21/353 | RARRES1 | 5918 | retinoic acid receptor responder (tazarotene |
| | | | induced) 1 |
| 415 | RASGRP1 | 10125 | RAS guanyl releasing protein 1 (calcium |
| | | | and DAG-regulated) |
| 695 | RASSF4 | 83937 | Ras association (RalGDS/AF-6) domain |
| | | | family 4 |
| 666 | RBM8A | 9939 | RNA-binding protein 8A |
| 74 | RBP4 | 5950 | retinol binding protein 4, plasma |
| 254 | REG1A | 5967 | regenerating islet-derived 1 alpha |
| | | | (pancreatic stone protein, pancreatic thread |
| | | | protein) |
| 399 | REG3A | 5068 | regenerating islet-derived 3 alpha |
| 568 | RGS1 | 5996 | regulator of G-protein signaling 1 |
| 221 | RGS13 | 6003 | regulator of G-protein signaling 13 |
| 227 | RGS20 | 8601 | regulator of G-protein signaling 20 |
| 174 | RNASE4 | 6038 | ribonuclease 4 precursor /// ribonuclease 4 |
| | | | precursor /// ribonuclease 4 precursor /// /// |
| | | | ribonuclease 4 precursor |
| 71 | RNF128 | 79589 | ring finger protein 128 |
| 36 | ROPN1 | 54763 | ropporin, rhophilin associated protein 1 |
| 588 | RPS4Y1 | 6192 | ribosomal protein S4, Y-linked 1 |
| 687 | RRAGD | 58528 | Ras-related GTP binding D |
| 606 | RSRC2 | 65117 | arginine/serine-rich coiled-coil 2 |
| 673 | RTEL1///ST | 51750///50861 | regulator of telomere elongation helicase |
| | MN3///ARF | ///10139/// | 1///stathmin-like 3///ADP-ribosylation |
| | RP1///TNF | 8771 | factor related protein 1///tumor necrosis |
| | RSF6B | | factor receptor superfamily, member 6b, |
| | | | decoy |
| 523 | S100A2 | 6273 | S100 calcium binding protein A2 |
| 386 | S100A7 | 6278 | S100 calcium binding protein A7 |
| 571 | S100A8 | 6279 | S100 calcium binding protein A8 |
| 258 | S100B | 6285 | S100 calcium binding protein B |
| 516 | S100P | 6286 | S100 calcium binding protein P |
| 329 | SALL1 | 6299 | sal-like 1 (Drosophila) |
| 37 | SAMSN1 | 64092 | SAM domain, SH3 domain and nuclear |
| | | | localization signals 1 |
| 635 | SAP18 | 10284 | Sin3A-associated protein, 18 kDa |
| 330 | SCEL | 8796 | sciellin |
| 531 | SCG2 | 7857 | secretogranin II (chromogranin C) |
| 544 | SCG5 | 6447 | secretogranin V (7B2 protein) |
| 331 | SCGB1D2 | 10647 | secretoglobin, family 1D, member 2 |
| 384 | SCGB2A1 | 4246 | secretoglobin, family 2A, member 1 |
| 355 | SCGB2A2 | 4250 | secretoglobin, family 2A, member 2 |
| 556/676 | SCNN1A | 6337 | sodium channel, nonvoltage-gated 1 alpha |
| 426 | SCRG1 | 11341 | scrapie responsive protein 1 |
| 549 | SEMA3C | 10512 | sema domain, immunoglobulin domain (Ig), |
| | | | short basic domain, secreted, (semaphorin) |
| | | | 3C |
| 128 | SEMA6A | 57556 | sema domain, transmembrane domain |
| | | | (TM), and cytoplasmic domain, |
| | | | (semaphorin) 6A |
| 691 | SENP5 | 205564 | SUMO1/sentrin specific peptidase 5 |
| 575 | SERPINA1 | 5265 | serpin peptidase inhibitor, clade A (alpha-1 |
| | | | antiproteinase, antitrypsin), member 1 |
| 495 | SERPINB2 | 5055 | serpin peptidase inhibitor, clade B |
| | | | (ovalbumin), member 2 |
| 255 | SERPINB3 | 6317 | serpin peptidase inhibitor, clade B |
| | | | (ovalbumin), member 3 |
| 480 | SERPINB5 | 5268 | serpin peptidase inhibitor, clade B |
| | | | (ovalbumin), member 5 |
| 235 | SERPINC1 | 462 | serpin peptidase inhibitor, clade C |
| | | | (antithrombin), member 1 |
| 416 | SERPIND1 | 3053 | serpin peptidase inhibitor, clade D (heparin |
| | | | cofactor), member 1 |
| 613 | SF3A3 | 10946 | splicing factor 3a, subunit 3, 60 kDa |
| 584/585/586 | SFRP1 | 6422 | secreted frizzled-related protein 1 |
| 530/616 | SFRP4 | 6424 | secreted frizzled-related protein 4 |
| 601 | SFRS11 | 9295 | splicing factor, arginine/serine-rich 11 |
| 78 | SFTPA2 | 729238 | pulmonary surfactant-associated protein A2 |
| | | | precursor |
| 8/251 | SFTPB | 6439 | surfactant, pulmonary-associated protein B |
| 229 | SH2D1A | 4068 | SH2 domain protein 1A, Duncan's disease |
| | | | (lymphoproliferative syndrome) |
| 675 | SH3BP2 | 6452 | SH3 domain-binding protein 2 isoform a |
| | | | SH3 domain-binding protein 2 isoform c |
| | | | SH3 domain-binding protein 2 isoform b /// |
| | | | SH3 domain-binding protein 2 isoform a |
| 640 | SH3 GLB 1 | 51100 | SH3-domain GRB2-like endophilin B1 |
| 629 | SHE | 6469 | sonic hedgehog homolog (Drosophila) |
| 337 | SI | 6476 | sucrase-isomaltase (alpha-glucosidase) |
| 394 | SLC14A1 | 6563 | solute carrier family 14 (urea transporter), |
| | | | member 1 (Kidd blood group) |
| 634 | SLC14A2 | 8170 | solute carrier family 14 (urea transporter), |
| | | | member 2 |
| 376 | SLC26A3 | 1811 | solute carrier family 26, member 3 |
| 31 | SLC38A4 | 55089 | solute carrier family 38, member 4 |
| 400 | SLC3A1 | 6519 | solute carrier family 3 (cystine, dibasic and |
| | | | neutral amino acid transporters, activator of |
| | | | cystine, dibasic and neutral amino acid |
| | | | transport), member 1 |
| 414 | SLC44A4 | 80736 | solute carrier family 44, member 4 |
| 542 | SLC4A4 | 8671 | solute carrier family 4, sodium bicarbonate |
| | | | cotransporter, member 4 |
| 53 | SLC6A14 | 11254 | solute carrier family 6 (amino acid |
| | | | transporter), member 14 |
| 356 | SLC6A15 | 55117 | solute carrier family 6, member 15 |
| 565 | SLPI | 6590 | secretory leukocyte peptidase inhibitor |
| 507 | SNCA | 6622 | synuclein, alpha (non A4 component of |
| | | | amyloid precursor) |
| 102 | SOD2 | 6648 | superoxide dismutase 2, mitochondrial |
| 87 | SORBS1 | 10580 | sorbin and SH3 domain containing 1 |
| 477/478 | SOX11 | 6664 | transcription factor SOX-11 |
| 570 | SOX9 | 6662 | transcription factor SOX-9 |
| 317 | SP140 | 11262 | SP140 nuclear body protein |
| 681 | SPATA5L1 | 79029 | spermatogenesis associated 5-like 1 |
| 366 | SPINK1 | 6690 | serine peptidase inhibitor, Kazal type 1 |
| 158 | SPON1 | 10418 | spondin 1, extracellular matrix protein |
| 245 | SPP1 | 6696 | secreted phosphoprotein 1 (osteopontin, |
| | | | bone sialoprotein I, early T-lymphocyte |
| | | | activation 1) |
| 166 | SPRR1A | 6698 | small proline-rich protein 1A |
| 455 | SPRR1B | 6699 | small proline-rich protein 1B (cornifin) |
| 469 | SRPX | 8406 | sushi-repeat-containing protein, X-linked |
| 160 | SST | 6750 | somatostatin |
| 175/209 | ST3GAL6 | 10402 | ST3 beta-galactoside alpha-2,3- |
| | | | sialyltransferase 6 |
| 43 | STAP1 | 26228 | signal transducing adaptor family member 1 |
| 618 | STC1 | 6781 | stanniocalcin-1 precursor |
| 619 | STK4 | 6789 | serine/threonine kinase 4 |
| 204/436 | SULT1C2 | 6819 | sulfotransferase family, cytosolic, 1C, |
| | | | member 2 |
| 361 | SULT2A1 | 6822 | sulfotransferase family, cytosolic, 2A, |
| | | | dehydroepiandrosterone (DHEA)- |
| | | | preferring, member 1 |
| 582 | TACSTD2 | 4070 | tumor-associated calcium signal transducer |
| | | | 2 |
| 101 | TARP | 445347 | TCR gamma alternate reading frame protein |
| 114 | TARP /// | 6966 /// 6967 | |
| | TRCTC1 /// | /// 6983 /// | TCR gamma alternate reading frame protein |
| | TRGC2 /// | 445347 | isoform 1 /// TCR gamma alternate reading |
| | TRGV9 | | frame protein isoform 2 |
| 250 | TARP /// | 6966 /// 6967 | TCR gamma alternate reading frame protein |
| | TRCTC1 /// | /// 6983 /// | isoform 1 /// TCR gamma alternate reading |
| | TRGC2 /// | 445347 | frame protein isoform 2 /// /// |
| | TRGV9 | | |
| 56 | TBX3 | 6926 | T-box 3 (ulnar mammary syndrome) |
| 475 | TCF21 | 6943 | transcription factor 21 |
| 686 | TCF7L1 | 83439 | transcription factor 7-like 1 (T-cell specific, |
| | | | HMG-box) |
| 421 | TCN1 | 6947 | transcobalamin I (vitamin B12 binding |
| | | | protein, R binder family) |
| 363 | TDGF1 | 6997 | teratocarcinoma-derived growth factor 1 |
| 403 | TENM1 | 10178 | teneurin-1 isoform 1 /// teneurin-1 isoform |
| | | | 2 /// teneurin-1 isoform 3 |
| 155/559 | TF | 7018 | transferrin |
| 493 | TFAP2A | 7020 | transcription factor AP-2 alpha (activating |
| | | | enhancer binding protein 2 alpha) |
| 145 | TFAP2B | 7021 | transcription factor AP-2 beta (activating |
| | | | enhancer binding protein 2 beta) |
| 333 | TFEC | 22797 | transcription factor EC |
| 462 | TFF1 | 7031 | trefoil factor 1 |
| 139 | TFF2 | 7032 | trefoil factor 2 (spasmolytic protein 1) |
| 283/284 | TFPI2 | 7980 | tissue factor pathway inhibitor 2 |
| 596 | THB S1 | 7057 | thrombospondin 1 |
| 275 | TM4SF1 | 4071 | transmembrane 4 L six family member 1 |
| 33 | TM4SF20 | 79853 | transmembrane 4 L six family member 20 |
| 243 | TM4SF4 | 7104 | transmembrane 4 L six family member 4 |
| 62 | TMC5 | 79838 | transmembrane channel-like 5 |
| 49 | TMEM255 | 55026 | transmembrane protein 255A isoform 2 /// |
| | A | | transmembrane protein 255A isoform 3 /// |
| | | | transmembrane protein 255A isoform 1 |
| 177 | TMEM30B | 161291 | transmembrane protein 30B |
| 194 | TMPRSS2 | 7113 | transmembrane protease, serine 2 |
| 435 | TMSB15A | 11013 | thymosin beta-15A |
| 473/474 | TNFRSF11 | 4982 | tumor necrosis factor receptor superfamily, |
| | B | | member 11b (osteoprotegerin) |
| 339 | TNFRSF17 | 608 | tumor necrosis factor receptor superfamily, |
| | | | member 17 |
| 502 | TOX | 9760 | thymocyte selection-associated high |
| | | | mobility group box |
| 104/126/132 | TOX3 | 27324 | TOX high mobility group box family |
| | | | member 3 |
| 163 | TRAF3IP3 | 80342 | TRAF3-interacting JNK-activating |
| | | | modulator isoform 2 /// TRAF3-interacting |
| | | | JNK-activating modulator isoform 1 |
| 693 | TRAFD1 | 10906 | TRAF-type zinc finger domain containing 1 |
| 580 | TRIM2 | 23321 | tripartite motif-containing 2 |
| 305 | TRIM31 | 11074 | tripartite motif-containing 31 |
| 655 | TRIM33 | 51592 | tripartite motif-containing 33 |
| 624 | TRPC3 | 7222 | transient receptor potential cation channel, |
| | | | subfamily C, member 3 |
| 119/120/ | TSHR | 7253 | thyroid stimulating hormone receptor |
| 234/671 | | | |
| 668 | TSPAN2 | 10100 | tetraspanin 2 |
| 546 | TSPAN8 | 7103 | tetraspanin 8 |
| 312 | TSPY1 | 7258 | testis specific protein, Y-linked 1 |
| 157 | TUBB2B | 347733 | tubulin, beta 2B |
| 665 | TWF1 | 5756 | twinfilin, actin-binding protein, homolog 1 |
| | | | (Drosophila) |
| 603 | TWF2 | 11344 | twinfilin, actin-binding protein, homolog 2 |
| | | | (Drosophila) |
| 152 | TXLNGY | 246126 | taxilin gamma pseudogene, Y-linked |
| 407 | TYRP1 | 7306 | tyrosinase-related protein 1 |
| 124/297 | UGT1A1 /// | 54575 /// | UDP-glucuronosyltransferase 1-1 precursor |
| | UGT1A10 | 54576 /// | /// UDP-glucuronosyltransferase 1-6 |
| | /// UGT1A3 | 54577 /// | isoform 1 precursor /// UDP- |
| | /// UGT1A4 | 54578 /// | glucuronosyltransferase 1-4 precursor /// |
| | ///UGT1A5 | 54579 /// | UDP-glucuronosyltransferase 1-10 |
| | /// UGT1A6 | 54600 /// | precursor /// UDP-glucuronosyltransferase |
| | /// UGT1A7 | 54657 /// | 1-8 precursor /// UDP- |
| | /// UGT1A8 | 54658 /// | glucuronosyltransferase 1-7 precursor /// |
| | /// UGT1A9 | 54659 | UDP-glucuronosyltransferase 1-5 precursor |
| | | | /// UDP-glucuronosyltransferase 1-3 |
| | | | precursor /// UDP-glucuronosyltransferase |
| | | | 1-9 precursor /// UDP- |
| | | | glucuronosyltransferase 1-6 isoform 2 |
| 46 | UGT2A3 | 79799 | UDP glucuronosyltransferase 2 family, |
| | | | polypeptide A3 |
| 322 | UGT2B15 | 7366 | UDP glucuronosyltransferase 2 family, |
| | | | polypeptide B15 |
| 346 | UGT2B4 | 7363 | UDP glucuronosyltransferase 2 family, |
| | | | polypeptide B4 |
| 232/233 | UPK1B | 7348 | uroplakin 1B |
| 656 | USP33 | 23032 | ubiquitin specific peptidase 33 |
| 662 | VASH1- | 100506603 | VASH1 antisense RNA 1 |
| | AS1 | | |
| 116/494 | VCAN | 1462 | versican |
| 115 | VGLL1 | 51442 | vestigial like 1 (Drosophila) |
| 395 | VNN1 | 8876 | vanin 1 |
| 598 | VTN | 7448 | vitronectin |
| 637 | WDR46 | 9277 | WD repeat domain 46 |
| 694 | WDTC1 | 23038 | WD and tetratricopeptide repeats 1 |
| 490 | WIF1 | 11197 | WNT inhibitory factor 1 |
| 577 | WIPF1 | 7456 | WAS/WASL interacting protein family, |
| | | | member 1 |
| 381 | WT1 | 7490 | Wilms tumor 1 |
| 24 | XIST | 7503 | X inactive specific transcript |
| 150 | XIST | 7503 | X (inactive)-specific transcript |
| 684 | YIF1B | 90522 | protein YIF1B isoform 3 /// protein YIF1B |
| | | | isoform 5 /// protein YIF1B isoform 4 /// |
| | | | protein YIF1B isoform 6 /// protein YIF1B |
| | | | isoform 2 /// protein YIF1B isoform 7 |
| 51 | ZBED2 | 79413 | zinc finger, BED-type containing 2 |
| 357 | ZIC1 | 7545 | Zic family member 1 (odd-paired homolog, |
| | | | Drosophila) |
| 285/286 | ZIM2///PEG3 | 23619///5178 | zinc finger, imprinted 2///paternally |
| | | | expressed 3 |
| 642 | ZNF174 | 7727 | zinc finger protein 174 |
| 669 | ZNF266 | 10781 | zinc finger protein 266 /// zinc finger protein |
| | | | 266 |
| 651 | ZNF471 | 57573 | zinc finger protein 471 |
| 617 | EBAG9 | 9166 | estrogen receptor binding site associated, |
| | | | antigen, 9 |
| 55 | ERAP2 | 6414767 | endoplasmic reticulum aminopeptidase 2 |

### DESCRIPTION

The present disclosure relates to a method for developing candidate probes to identify at least one primary site of a selected disease, disorder or genetic disorder in a mammalian subject. The method includes steps (a) to (c). In step (a), a detecting chip generates a plurality of gene expressions from a standard sample of a subject having a selected disease, disorder or genetic disorder, and the standard sample is diagnosed with a metastasis cancer with at least one known primary site. In step (b), a processing module compares the plurality of gene expression by using a meta-data analysis to generate a comparison result. In step (c), the processing module further develops an array that contains a plurality of candidate probes based on the comparison result. Moreover, the plurality of candidate probes are capable of binding a plurality of polynucleotide sequences selected from any one of SEQ ID No.1 to 695 or from any fragment of SEQ ID No.1 to 695. The detecting chip and the processing module are electrically connected to each other. Individually, the plurality of polynucleotides are the genes in Table 1.

In one embodiment, the number of the candidate probes used to identify primary site is about 650. In another embodiment, the number of the candidate probes is about 100. In one preferred embodiment, the number of the candidate probes is about 50.

In another embodiment, the length of the candidate probes is at least 20 nucleotides.

In one embodiment, the detecting chip used to identify the primary sites is a microarray chip or magnetic beads. In another embodiment, the processing module used to compare the plurality gene expressions or to develop the array containing the candidate probes is a central processing unit (CPU).

In one embodiment, the standard sample used to develop the candidate probes includes blood, blood plasma, serum, urine, tissue, cells, organs, seminal fluids or any combination thereof. In another embodiment, the selected disease, disorder or genetic disorder includes hematologic malignancies or solid tumors.

The present disclosure further relates to a method for identifying a primary site of a selected disease, disorder or genetic disorder in a mammalian subject. Specifically, the selected disease, disorder or genetic pathology in a mammalian subject may be a tumor. The method includes step (a′) and (b′). In step (a′), a detection chip containing the plurality of candidate probes developed by the method previously described is provided to analyse and measure the expression levels of an array of a test sample. The test sample may be obtained from a subject having a selected disease, disorder or genetic disorder. Such test sample is further diagnosed with a metastasis cancer with at least one unknown primary site.

In one embodiment, the test sample used to develop the candidate probes includes blood, blood plasma, serum, urine, tissue, cells, organs, seminal fluids or any combination thereof. In another embodiment, the selected disease, disorder or genetic disorder includes hematologic malignancies or solid tumors.

The present disclosure also related to a system for identifying a primary site of a selected disease, disorder or genetic disorder in a mammalian subject. The system includes a detecting chip and a processing module electrically connected to each other. The detecting chip contains a plurality of candidate probes for primary sites, and the candidate probes are capable of binding a plurality of polynucleotide sequence selected from any one of SEQ ID No.1 to 695 or from any fragment of SEQ ID No.1 to 695. Specifically, the plurality of polynucleotide are the genes list in the Table 1. That is, the candidate probes are capable of binding and further recognizing the genes in the Table 1.

### Example 1

In the following content, all the statistical calculations are conducted through a processing module, which is a central processing unit (CPU). The candidate genes probes in Table 1 are hereinafter referred as “PH2”, “PH2 probes” or “the 695-gene transcription profiles.”

Developing the PH2 Probes

Step (a) of the present disclosure is to generate the whole genome expression profile of the cancer sample. Specifically, a group of transcriptomic microarray datasets derived from the metastatic cancer samples of different primary sites are collected from the public database Gene Expression Omnibus (GEO, https://www.ncbi.nlm.nih.gov/geo/). As seen in Table 2, a total of more than five hundreds samples of metastatic cancers originated from fifteen primary sites are used for probes finding and validation.

TABLE 2

| | | Number | | | |
|---|---|---|---|---|---|
| | Sample | of correct | | | |
| Datasets | number | results | Metastatic_site | Cancer_type | Reference |
| GSE12630 | 187 | 276 | See Note 1 | metastatic | J Clin Oncol. |
| | | | | cancers from 15 | 2009 May 20; |
| | | | | different origins | 27(15):2503-8. |
| GSE14095 | 189 | 190 | liver metastasis | colorectal cancer | Clin Transl |
| | | | | | Oncol. 2011 Jun; |
| | | | | | 13(6):419-25. |
| GSE14108 | 28 | 9 | Brain | lung | Not Available |
| | | | metastasized | adenocarcinoma | |
| | | | from lung | | |
| | | | adenocarcinoma | | |
| GSE14378 | 20 | 19 | lung | clear-cell renal | Wuttig et al. Int. |
| | | | | cell carcinoma | J. Cancer: 125, |
| | | | | | 474-482(2009) |
| GSE15605 | 12 | 11 | lymph node, | melanoma | Raskin et al |
| | | | subcutaneous | | (2013), J Invest |
| | | | soft tissue, | | Dermatol, |
| | | | spleen or small | | 133(11):2585-92 |
| | | | instestine | | |
| GSE19949 | 15 | 15 | metastasis of | renal cell | Beleut M et al. |
| | | | RCC to other | carcinoma | (2012), BMC |
| | | | site | | Cancer1 |
| | | | | | 23; 12:310 |
| GSE20565 | 44 | 43 | ovary | breast | Meyniel et al. |
| | | | | | (2010) BMC |
| | | | | | Cancer |
| | | | | | 21; 10:222 |
| GSE22541 | 44 | 41 | lung | clear-cell renal | Wuttig et al. |
| | | | | cell carcinoma | (2012) Int J |
| | | | | | Cancer |
| | | | | | 131(5):E693-704 |
| Total | 539 | 1070 | | | |

Note 1:
bladder, breast, colon, stomach, germ cell, kidney, liver, lung, lymph node, ovary, pancreas, prostate, skin, soft tissue, and thyroid.

For the purpose of generating the candidate probes of the present invention, 186 samples of distant metastasis originated from fifteen different tissue origins are first selected from the dataset GSE12630 to construct a training dataset. For this training dataset, the CEL files are acquired from GEO and then subjected to quality assessment by AffyQualityReport to remove the poor quality arrays. The data passing quality-control is then subjected to the Robust Multichip Average (RMA, Irizarry R et al. Biostatistics 2003, 4(2):249-264) processing for data normalization. Both AffyQualityReport and RMA are obtained from the Bioconductor package in the R package (http://www.r-project.org/). Following the standard preprocessing procedure, the transcriptomic data is subjected to further statistical and bioinformatics analyses.

TABLE 3
“The Example of the Expression Array of Training Gene Dataset”

| | | Sample | | | | | | | |
|---|---|---|---|---|---|---|---|---|---|
| | Gene | Liver | Liver | Breast | Colon | Colon | | | CV |
| No. | Name | 1 | 2 | 1 | 1 | 2 | . . . | others | value |
| 1 | | | | | | | | | |
| 2 | | | | | | | | | |
| 3 | | | | | | | | | |
| . | . | . | . | . | . | . | . | . | . |
| . | . | . | . | . | . | . | . | . | . |
| . | . | . | . | . | . | . | . | . | . |

Step (b) involves comparing the expression levels across different tumor samples for each gene. According to step (a), the expression levels for each gene in different tumor tissues are provided. To compare, the coefficients of variation (CV) value of the expression level in each tumor samples is obtained based on the following formula:

The coefficients of variation (CV) is defined as the ratio of the standard deviation σ to the mean μ: C_{V}=σ/μ

Accordingly, the gene expression array which Table 3 is the exemplary format is developed. In Table 3, each row represents the expression levels of a specific gene in different tumor samples (e.g., Liver 1, Liver 2, etc.), while each column represents the different genes in the tumor samples.

More specifically, gene filtration is carried out by firstly selecting from the training dataset obtained in step (a) the genes whose CV value appeared in the top 5% of the entire transcriptome across different tissue types. The resulted highly variably expressed genes then becomes the set of candidate tissue-classifier genes which are later subjected to data redundancy elimination through hierarchical clustering against the 15 tissues using the open-source computer software MeV v4.8.1 (https://sourceforge.net/projects/mev-tm4/) where Pearson correlation and average linkage were chosen for Distance Metric and for Linkage method, respectively.

Following the hierarchical cluster analysis, one representative gene for each cluster is selected and additional genes with highly similar expression profiles are removed. Such procedure results the candidate genes as provided in Table 1.

The hierarchical cluster method (Pearson's correlation):

$$r = \frac{{\sum }_{i = 1}^{n} ( {X}_{i} - \overline{X} ) ( {Y}_{i} - \overline{Y} )}{\sqrt{{\sum }_{i = 1}^{n} {( {X}_{i} - \overline{X} )}^{2}} \sqrt{{\sum }_{i = 1}^{n} {( {Y}_{i} - \overline{Y} )}^{2}}}$$

Step (c) involves further developing the candidate probes of the present invention based on the previous candidate genes in Table 1. That is, the probe sequence is designed as the complementary sequence to SEQ ID No.1 to 695. Furthermore, the candidate probes sequence can be a long sequence that is entirely complementary to SEQ ID No.1 to 695, or a short sequence complementary only to a fragment of SEQ ID No.1 to 695.

Validation of the PH2 Probes on the Metastatic Cancerous Samples with the Oligonucleotide Microarrays

To validate the effects of the PH2 probes in identifying the primary sites of metastatic cancers, more of the whole-genome gene expression datasets with samples from metastatic cancers were collected from public database GEO. (See Table 2.)

The dataset GSE20565 (Meyniel et al. BMC Cancer 2010 May 21; 10: 222) contained 44 samples of ovarian cancers metastasized from breast. Applying the expression profiles of PH2, 43 out of 44 samples were correctly predicted with breast as their primary sites—reaching an accuracy of 97.7%. The dataset GSE22541 (Wuttig et al. Int. J. Cancer, 2009; 125: 474-482) contained 30 samples which were found in lung but metastasized from the clear-cell renal cell carcinoma. Among the 30 samples, 27 were correctly predicted to be originated from the kidney primary site, attaining a 90% of prediction accuracy. The dataset GSE15605 (Raskin L. et al. J Invest Dermatol 2013 November; 133(11): 2585-92) was predicted correctly on 11 of the 12 metastasized melanoma samples which were punch-biopsied at spleen, small intestine, lymph nodes and subcutaneous soft tissue. All of the 15 metastatic renal cell carcinoma from the dataset GSE19949 (Beleut M. et al. BMC Cancer 2012 Jul. 23; 12: 310) were successfully mapped to kidney by the PH2 probes. The lung metastasis of the renal cell carcinoma from the dataset GSE14378 19/20 (Wuttig et al. Int. J. Cancer 2009; 125: 474-482) was also confirmed by the 600-gene transcription profiles.

The Number of Genes was Reduced to Fit Different Experimental Platforms

To adapt to various experimental platforms such as using magnetic beads to identify of primary site of a metastatic cancer, the 695-gene transcription profiles may be reduced by eliminating genes with alike expression profiles. Particularly, further elimination by reducing the number of clusters at step (b) described above may result in a smaller group of classifier genes. Following validation on the test dataset with the computational process of primary-tissue-prediction, the present invention is able to reduce the gene set down to as small as 53 genes which were later proved to work efficiently on magnetic beads. As shown in Table 5 which provides the results of the validation tests, the prediction of the primary sites of metastatic cancers using a subset of the PH2 probes was highly satisfied.

TABLE 5
“Prediction of the primary site of a metastatic
cancer with different versions of PH2”

| | Samples | | | correct_Q | correct_Q |
|---|---|---|---|---|---|
| Datasets | (N) | correct_600 | correct_100 | G (k = 1) | G (k = 2) |
| GSE14095 | 189 | 169 | 178 | 177 | 187 |
| GSE14108 | 28 | 24 | 24 | 18 | 28 |
| GSE14378 | 20 | 19 | 19 | 19 | 20 |
| GSE15605 | 12 | 11 | 8 | 11 | 12 |
| GSE19949 | 15 | 15 | 15 | 14 | 15 |
| GSE20565 | 44 | 43 | 42 | 43 | 43 |
| GSE22541 | 44 | 41 | 39 | 42 | 43 |

For example, 42 out of 44 samples from the dataset GSE 20565 were correctly predicted, 15 out of 15 samples from the dataset GSE19949 were correctly predicted.

In some experimental platforms, smaller gene numbers is preferred. In one example, a group of around 53 genes (the subset of the PH2 probes) can be used to identify the primary site. While performing the validation method as described above with a larger group of genes, it was found that prediction accuracy using a subset of PH2 probes significantly dropped to 64% (18/28) from 86% (24/28) with the dataset GSE14108. However, if the parameter k of the KNN used in the prediction model changes from 1 to 2, the accuracy increases to 100% (28/28) for all test datasets. Such result suggests that a subset of the PH2 probes, if selected properly, can perform the primary site identification for metastatic cancers just as accurate as if using the entire PH2 markers.

Clinical Validation of QG on Primary Site Prediction for Metastatic Cancers

Patients and Samples:

The metastatic tumor specimens were taken from the cancer patients whose tumors were diagnosed as metastatic cancer by both oncologists and pathologists at the Tzu-Chi Hospital in Hualian, Taiwan. All the donors have signed informed consent forms before the tumors were removed at the surgery. The tissue samples (Table 6) extracted from the tumors were immersed into liquid nitrogen followed by RNAlater processing for later usage of PH2-QuantiGene assays.

TABLE 6
Anatomic and Metastatic Sites of the Clinical Samples

| Anatomic site | Number of Samples |
|---|---|
| breast | 2 |
| Colon/rectum | 1 |
| liver | 7 |
| gastric | 1 |
| others | 4 |
| Total | 15 |

Assay Kit and Signal Detection

The PH2-QuantiGene assay kit was custom-made by Affymetrix Inc. Affymetrix Inc. (the carrier of Panomics beads) designed the PH2 probes, conjugated the probes to the magnetic beads, assembled the necessary reagents and performed quality control on the final products. At the end of each assay, Luminex® 100/200™ is used to detect the hybridization signals.

The Quantigene assays on PH2 were performed in two separate experiments. The first experiment was carried out using the Luminex® 200™ to detect hybridization signals while the second experiment was performed using Luminex® 100™. Each sample was assayed in duplicates in both experiments for confirmation. For each assay, about a rice-grain size of sample was used. The Panomics-provided protocol was followed in order to measure the expression levels of each of the probes whose probes have been conjugated on the magnetic beads.

Analysis and Statistics

The data of the expression levels of each gene on the PH2-Quantigene beads output from the Luminex fluorescence reader was preprocessed and analyzed. The model then computes the probability for each of the 15 candidate tissues to become the primary site using k-nearest neighbor method (hereinafter “KNN”) as following mathematical equation at k=1, k=2 or k=3. It compares the c.f. (coefficient of correlation by Pearson's correlation) of the 600-gene profiles between a test tissue and each of our 15 tissue-specific gene expression profiles, one for each tissue type. The tissue type with highest correlation was nominated as our prediction.

The k-nearest neighbor method:

$$Sim ( {d}_{i} , {d}_{j} ) = \frac{{\sum }_{k = 1}^{M} {W}_{ik} \times {W}_{jk}}{\sqrt{( {\sum }_{k = 1}^{M} {W}_{ik}^{2} ) ( {\sum }_{k = 1}^{M} {W}_{jk}^{2} )}}$$

According to the present disclosure, the PH2 probes can identify the primary site of a metastatic cancer/tumor if the cancer/tumor originates from one of the tissues/organs including breast, stomach, colon, pancreas, bladder, thyroid, prostate, kidney, liver, ovary, germ cell, soft tissue, skin, lymph node and lung. The meta-data analysis demonstrated that a portion or an entire set of PH2 probes may perform the function with high accuracy. Clinical samples were used by some experiments to further validate the gene markers.

In the test using the magnetic-beads which had been conjugated with the oligonucleotides representing each of the PH2 probes, the magnetic beads were purchased from QuantiGene, which was developed by Panomics and distributed by eBioscience of Affymetrix Inc. Before applying to the clinical samples, PH2 probes have been validated on the transcriptomic datasets obtained from the public database GEO at NCBI (http://www.ncbi.nlm.nih.gov/geo/). The positive results (Tables 4, 5) from these analyses indicated the PH2 probes are applicable to real clinical samples.

A total of fifteen specimens from cancer patients were used. All the clinical information of the specimens and that of the donor patients were kept confidential. The pathological features and the diagnosis of each specimen had been confirmed by the pathologists and the surgeons. The fifteen specimens were dissected from various organs, including liver, colon, breast, spleen, pancreas, perineum etc. during a necessary surgery. Among the specimens, fourteen of them were confirmed as metastatic tumors while one of the specimen was found to be a benign tumor originated from soft tissue. Three of the fourteen metastatic specimens have primary sites other than the fifteen tissues/organs so were dropped from the study.

To perform the PH2/Quantigene analysis on the clinical specimens, the frozen tissue was firstly cut, thawed, and manually homogenized with micro pestles. Then the RNA was extracted and hybridized to the PH2/Quantigene beads. The manufacturer-provided standard protocol was followed until signal was acquired with the Luminex machine. The data output from the Luminex was then subjected to computer analysis with the PH2 probes which incorporates KNN method as the final step for the prediction.

A total of eleven specimens whose primary sites fell into the fifteen candidate primary sites were included for the final computing. For these eleven metastatic specimens, the primary site was predicted at k=1, k=2 and k=3 (that is, their correct primary site was ranked within one, two, or three highest scored tissues, respectively.) The overall accuracy of primary site prediction by PH2 probes in this study was 100% at k=3, see Tables 7 and 8.

TABLE 7
“PH2 on Agilent: Tested with Clinical Specimens; Accuracy: 80%
when k = 1 or k = 2; 100% when k = 3”

| | | Agilent_PH2 | Agilent_PH2 | Agilent_PH2 |
|---|---|---|---|---|
| Primary site | Anatomic | Rank_1 | Rank_2 | Rank_3 |
| answer¹ | Site² | (k = 1) | (k = 2) | (k = 3) |
| colon | liver | Colorectal | | |
| colon | liver | Colorectal | | |
| breast | breast | Breast | | |
| recurrence | | | | |
| gastric | liver | Liver | Pancreas | Gastric |
| colon | liver | Colorectal | | |

¹The primary site of the tumor sample.
²The organ where the tumor sample is taken.

TABLE 8
“PH2 on Clinical specimen using Quantigene or Agilent”

| | Test-1 | | | Test-2 | | | Agilent | | |
|---|---|---|---|---|---|---|---|---|---|
| K value | 1 | 2 | 3 | 1 | 2 | 3 | 1 | 2 | 3 |
| accuracy | 7/12 | 9.5/12 | 12/12 | 5/8 | 7.5/8 | 8/8 | 4/5 | 4/5 | 5/5 |
| (number) | | | | | | | | | |
| accuracy | 58% | 79% | 100% | 63% | 94% | 100% | 80% | 80% | 100% |
| (%) | | | | | | | | | |

The PH2 probes were confirmed by three platforms. A comparison between the results using three platforms is provided in Table 9.

TABLE 9
“Comparison of PH2 prediction on three platforms”

| | K | Affymetrix | Agilent | Magnetic |
|---|---|---|---|---|
| | value | array | array | beads (QGP) |
| Accuracy | K = 1 | >90 | 80% | ~60% |
| | K = 2 | | 80% | >80% |
| | K = 3 | | 100% | 100% |

| | | | |
|---|---|---|---|
| Price | ~30000 NT | ~20000 NT | <3000~10000 NTD |
| Sample amount | ug | ug | ng |
| Processing time | >5 days | >5 days | 1.5 days |

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the appended claims.

1. A method for developing a plurality of candidate probes to identify at least one primary site of a selected disease, disorder or genetic disorder in a mammalian subject, comprising:
(a) generating, by a detecting chip, a plurality of gene expression obtained from a standard sample of a subject having a selected disease, disorder or genetic pathology, wherein the standard sample is diagnosed with a metastasis cancer with at least one known primary site;
(b) comparing, by a processing module, the plurality of gene expression to generate a comparison result; and
(c) developing, based on the comparison result, an array containing the plurality of candidate probes, wherein the plurality of candidate probes are capable of binding a plurality of polynucleotide sequences selected from any one of SEQ ID No.1 to 695 or from any fragment of SEQ ID No.1 to 695,
wherein the detecting chip is electrically connected to the processing module.

2. The method according to claim 1, wherein a number of the plurality of candidate probes is about 650.

3. The method according to claim 1, wherein a number of the plurality of candidate probes is about 100.

4. The method according to claim 1, wherein a number of the plurality of candidate probes is about 50.

5. The method according to claim 1, wherein the detecting chip includes a microarray, a next-generation sequencing device, a quantitative PCR and magnetic beads.

6. The method according to claim 1, wherein the processing module is a central processing unit (CPU).

7. The method according to claim 1, wherein the standard sample includes blood, blood plasma, serum, urine, tissue, cells, organs, seminal fluids or any combination thereof.

8. The method according to claim 1, wherein the selected disease, disorder or genetic disorder includes hematologic malignancies or solid tumors.

9. The method according to claim 1, wherein a length of the candidate probes is at least 20 nucleotides.

10. A method for identifying a primary site of a selected disease, disorder or genetic disorder in a mammalian subject, comprising:
(a′) analysing, by a detection chip that contains the plurality of candidate probes as in claim 1, expression levels of an array of a test sample obtained from a subject having a selected disease, disorder or genetic disorder,
wherein the test sample is diagnosed with a metastasis cancer with at least one unknown primary site, and the plurality of candidate probes are capable of binding the plurality of polynucleotide sequence selected from any one of SEQ ID No.1 to 695 or from any fragment of SEQ ID No.1 to 695 as in claim 1;
(b′) predicting, by a processing module, a primary site of the test sample based on the array's expression levels.

11. The method according to claim 10, wherein the test sample includes blood, blood plasma, serum, urine, tissue, cells, organs, seminal fluids or any combination thereof.

12. A system for identifying a primary site of a selected disease, disorder or genetic disorder in a mammalian subject, comprising:
a detecting chip that contains a plurality of candidate probes wherein the plurality of candidate probes are capable of binding a plurality of polynucleotide sequence selected from any one of SEQ ID No.1 to 695 or from any fragment of SEQ ID No.1 to 695; and
a processing module, electrically connected to the detecting chip,
wherein the detecting chip analyses expression levels of an array of a test sample obtained from a subject having a selected disease, disorder or genetic disorder,
wherein the processing module predicts a primary site of the test sample based on the expression levels of the array of the test sample.

13. The system according to claim 12, wherein a number of the plurality of candidate probes is about 650.

14. The system according to claim 12, wherein a number of the plurality of candidate probes is about 100.

15. The system according to claim 12, wherein a number of the plurality of candidate probes is about 50.

16. The system according to claim 12, wherein the detecting chip includes a microarray, a next-generation sequencing device, a quantitative PCR and magnetic beads.

17. The system according to claim 12, wherein the processing module is a central processing unit (CPU).

18. The system according to claim 12, wherein the test sample include blood, blood plasma, serum, urine, tissue, cells, organs, seminal fluids or any combination thereof.

19. The system according to claim 12, wherein the selected disease, disorder or genetic disorder includes hematologic malignancies or solid tumors.

20. The system according to claim 12, wherein a length of the candidate probes is at least 20 nucleotides.


SEQUENCE LISTING (695 sequences)

SEQ ID NO: 1 - DNA, 457 bp, Artificial Sequence
  Synthetic
  misc_feature (64)..(95): n is a, c, g, or t
  misc_feature (104)..(104): n is a, c, g, or t
  misc_feature (226)..(226): n is a, c, g, or t
  misc_feature (344)..(344): n is a, c, g, or t
  misc_feature (387)..(402): n is a, c, g, or t
acagacttggcaagggaccccctggttctgagccagtagctgccatctggaaattcctcttttnnnnnnnnnnnnnnnnnnnnnnnnnnnnnnnnctcccaggnacccgctgaatttctgaggccttgcttaaagctcagaagtggtttaggcatttggaaaatctggttcacatcataaagaacttgatttgaaatgttttctatagaaacaagtgctaagtgtnaccgtattatacttgatgttggtcatttctcagtcctatttctcagttctattattttagaacctagtcagttctttaagattataactggtcctacattaaaataatgcttctcgangtcagattttacctgtttgctgctgagaacatctctgcctaannnnnnnnnnnnnnnncttcagttcaacatgcttccttagcttttcatagttgtctgacatttccatgaaa

SEQ ID NO: 2 - DNA, 298 bp, Artificial Sequence
  Synthetic
ccgatttgtgtctattattggtgacattgttttagatattgggtattgtatattaaggaaaaagatggtctatattctctttattgcatatacttaatgtttcaaaagaatgcagattctgtgtttaagcacagggctgatagttgtggttttgtttacaaatgttctgttttggctgctattggttttttaaagaggttttttatacttttgtatttgaatagttatgtttcactgatgctgagccagtttgtatgtgtgtgcatatatgtgaactgtaactgacaagatgaattac

SEQ ID NO: 3 - DNA, 374 bp, Artificial Sequence
  Synthetic
  misc_feature (40)..(96): n is a, c, g, or t
  misc_feature (99)..(99): n is a, c, g, or t
  misc_feature (115)..(115): n is a, c, g, or t
  misc_feature (245)..(245): n is a, c, g, or t
  misc_feature (261)..(261): n is a, c, g, or t
agtccctaccagtttaggctggtgggcccagggcagtggnnnnnnnnnnnnnnnnnnnnnnnnnnnnnnnnnnnnnnnnnnnnnnnnnnnnnnnnnacncgagtggtcgggaganttcagaagccttgcttctttttccattggctgaagctctttgcaattcctattctgttaatcgctgttttccttgtgttgacctaatcatcattttctctaggatttctgaaagttactgacaatacccnagacaggggctttgcntatttaaaaattaaaattttcacaccacctgcttttctattcagcatcttttgcagtactctgtagacattagtcagtaatacagtgttatttctaggctctgaaatagccac

SEQ ID NO: 4 - DNA, 279 bp, Artificial Sequence
  Synthetic
cttctacaggcttttgggaagtagggtggatgtgggtagggctgggaggagggggccacagcttaggtttggagctctggatgtacatacataagtaggagcagtgggacgtgtttctgtcataatgcaggcatgaagggtggagtgaagtcaggtcataagtttcatgtttgcttttgttttgttttgtttttaatgtatgtagcagatgttacagtcttagggatccgggatgggagaccccactttagaaagggtcgtcactcctttaatcctcta

SEQ ID NO: 5 - DNA, 398 bp, Artificial Sequence
  Synthetic
  misc_feature (80)..(80): n is a, c, g, or t
  misc_feature (82)..(83): n is a, c, g, or t
  misc_feature (85)..(85): n is a, c, g, or t
  misc_feature (87)..(87): n is a, c, g, or t
  misc_feature (97)..(97): n is a, c, g, or t
caacccattttgtgccacatgcaagttttgaataaggatggtatagaaaacaacgctgcatatacaggtaccatttaggnannancngatgcctttntgggggcagaatcacatggcaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattcc

SEQ ID NO: 6 - DNA, 442 bp, Artificial Sequence
  Synthetic
  misc_feature (319)..(334): n is a, c, g, or t
agatcttggcccaaaccagcccaggcagtctaaagaaaactgggttggtgcttcttggaatgaaggcaaaaatctgaaattgttagtgtctcagtaatcctgatttagaacccaggctttttgtaacaatgtgttttcttgcctaaattcatttatctggcatcatcagagtactaacatgtttatatttcagatatccaaagcttaccactttaggtgatgaatctttactattttagcccttttttgatgagactatttgtctaaagtgaatcatttgttcttgccttattaaacagagtagatggaaaacaatttnnnnnnnnnnnnnnnnatttctttatgaatatgaataattgttctatgctttaataatctattgtgaggaaactactaagaaatatgttggtgtgtttgtccttacttgaaatgggtctgtatt

SEQ ID NO: 7 - DNA, 608 bp, Artificial Sequence
  Synthetic
  misc_feature (46)..(47): n is a, c, g, or t
  misc_feature (118)..(118): n is a, c, g, or t
  misc_feature (131)..(131): n is a, c, g, or t
  misc_feature (142)..(142): n is a, c, g, or t
  misc_feature (155)..(155): n is a, c, g, or t
  misc_feature (186)..(186): n is a, c, g, or t
  misc_feature (193)..(193): n is a, c, g, or t
  misc_feature (211)..(211): n is a, c, g, or t
  misc_feature (221)..(221): n is a, c, g, or t
  misc_feature (246)..(246): n is a, c, g, or t
  misc_feature (248)..(248): n is a, c, g, or t
  misc_feature (258)..(258): n is a, c, g, or t
  misc_feature (273)..(273): n is a, c, g, or t
  misc_feature (281)..(281): n is a, c, g, or t
  misc_feature (290)..(290): n is a, c, g, or t
  misc_feature (298)..(298): n is a, c, g, or t
  misc_feature (332)..(332): n is a, c, g, or t
  misc_feature (348)..(348): n is a, c, g, or t
  misc_feature (432)..(450): n is a, c, g, or t
  misc_feature (476)..(476): n is a, c, g, or t
  misc_feature (489)..(503): n is a, c, g, or t
  misc_feature (582)..(582): n is a, c, g, or t
ttgccccagcagagcagatctgctttttttccagagcaaaatgaanncactaggtataaatatgttgttactgccaagaacttaaatgactggtttttgtttgcttgcagtgctttcnttaattttatggnctcttctgggnaaactcctccccnttttccacacgaaccttgtggggctgtgaantctttcnttcatccccgcattcccnaatatacccnaggccacaagagtggacgtgaaccnanagggtgtccntgtcagaggagcccnatctcccnatctccccnagctcccntatctggaggatagttggatagttacgtgttccntagcaggaccaactanagtcttcccaaggattgagttatggactttgggagtgagacatcttcttgctgctggatttccaagctgagaggacgtgaaccnnnnnnnnnnnnnnnnnnncttgtttgccacatggagagagactntgaggacagaagnnnnnnnnnnnnnnnaggagccaagggattgacaaacaacagagccttgaccacgtggagtctctgaatcagccttgtctggaaccagatctanacctggactgcccaggtctataagcc

SEQ ID NO: 8 - DNA, 383 bp, Artificial Sequence
  Synthetic
ccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagcttttgattgaaagtcctagggtgattctatttctgc

SEQ ID NO: 9 - DNA, 440 bp, Artificial Sequence
  Synthetic
  misc_feature (265)..(265): n is a, c, g, or t
  misc_feature (267)..(321): n is a, c, g, or t
  misc_feature (324)..(329): n is a, c, g, or t
  misc_feature (331)..(335): n is a, c, g, or t
  misc_feature (337)..(338): n is a, c, g, or t
  misc_feature (340)..(342): n is a, c, g, or t
  misc_feature (344)..(344): n is a, c, g, or t
  misc_feature (346)..(346): n is a, c, g, or t
  misc_feature (349)..(349): n is a, c, g, or t
  misc_feature (352)..(354): n is a, c, g, or t
  misc_feature (356)..(357): n is a, c, g, or t
  misc_feature (359)..(359): n is a, c, g, or t
  misc_feature (361)..(361): n is a, c, g, or t
ggatgtctcttcagtttatgacacaatagcttcctgggcaacaaatctcaagtcctccatcagaaaggctaataaggccctctggctctctattgcattcattgtactgtttgcagctttgatgagcttcctcacaggccaattattccagaagtctgtggatgccgctcccacacagcaagaggactcatggacgtctctagaacatatcttgtggccatttaccagactccgacacaatgggccaccaccagtgtgacagcangnnnnnnnnnnnnnnnnnnnnnnnnnnnnnnnnnnnnnnnnnnnnnnnnnnnnnnntannnnnntnnnnntnngnnngnanagnatnnnannanancagaggttctcagaatgactgtaagatagcttacatttcctctttttgcctttatctccccaactaaaatacaatgggg

SEQ ID NO: 10 - DNA, 548 bp, Artificial Sequence
  Synthetic
  misc_feature (26)..(26): n is a, c, g, or t
  misc_feature (43)..(43): n is a, c, g, or t
  misc_feature (49)..(51): n is a, c, g, or t
  misc_feature (370)..(388): n is a, c, g, or t
  misc_feature (468)..(468): n is a, c, g, or t
taatcggctcactataggaatttgcntcgaggccaagattcgnacgagnnngttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcagcatgagcggaggcattttccttttcttcgtggccaatgccataatccacctcttctgcttcagttgaggtgacacgtctcagccttagccctgtgccccctgaaacannnnnnnnnnnnnnnnnnnagagaatcccctccatctttgggaggggttgatgccagacatcaccaggttgtagaagttgacaggcagtgccatggggncaacagccaaaataggggggtaatgatgtaggggccaagcagtgcccagctgggggtcaataaagttacccttgtacttg

SEQ ID NO: 11 - DNA, 366 bp, Artificial Sequence
  Synthetic
ccccaggcgatggacaattatgatagcttatgtagctgaatgtgatacatgccgaatgccacacgtaaacacttgactataaaaactgcccccctcctttccaaataagtgcatttattgcctctatgtgcaactgacagatgaccgcaataatgaatgagcagttagaaatacattatgcttgagatgtcttaacctattcccaaatgccttctgttttccaaaggagtggtcaagcccttgcccagagctctctattctggaagagcggtccaggtggggccgggcactggccactgaattatgccagggcgcactttccactggagttcactttcaattgcttctgtgcaataaaaccaagtg

SEQ ID NO: 12 - DNA, 519 bp, Artificial Sequence
  Synthetic
  misc_feature (362)..(389): n is a, c, g, or t
atagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggccacctcttctaagagtcccatctgctatgcccagccacattaactaactttaatcttagtttatgcatcatatttcattttgaaattgatttctattgttgagctgcattatgaaattagtattttcnnnnnnnnnnnnnnnnnnnnnnnnnnnnatcctttcccctttcccttcaactcttcgtacattcaatgcatggatcaatcagtgtgattagctttctcagcagacattgtgccatatgtatcaaatgacaaatctttattgaatggttttgctcagca

SEQ ID NO: 13 - DNA, 478 bp, Artificial Sequence
  Synthetic
ttcacaaacttttatactctttctgtatatacattttttttctttaaaaaacaactatggatcagaatagcaacatttagaacactttttgttatcagtcaatatttttagatagttagaacctggtcctaagcctaaaagtgggcttgattctgcagtaaatcttttacaactgcctcgacacacataaacctttttaaaaatagacactccccgaagtcttttgtttgtatggtcacacactgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaatgctgtacatctattttggattgtatattgtggttgtgtatttacgctttgattcatagtaacttcttatggaattgat

SEQ ID NO: 14 - DNA, 231 bp, Artificial Sequence
  Synthetic
tgccatgctgatgccatatattccaacgtgatcaacctggctccccagaaggaggacgactttgctgtctacaccaacatgcccccttttcatcaccccaggaggacattgccagaccaagtggaatatgtctccattgtattccactgatgggaagctaatgagatgctcagagtgggggtcagacctggccccagctgaatcttggcataccctttgctttagatttat

SEQ ID NO: 15 - DNA, 346 bp, Artificial Sequence
  Synthetic
  misc_feature (277)..(277): n is a, c, g, or t
agctctaaaatctgcttgtattccaagcatataaaattttcccccttagtgaattagttttaaaatgatattgttatatacatactatgaaatatgtataactttaacttctgttttaccagcatacccacacaaataacaagaatactacttatgaaatgtgcactttatcctcattccataaatgttggtgcataccttatgtaagggagcagttcaataatccatgaaagaacttaaggcatttgttggtttatcagactcggaatctattttntcattgctctgaatatgtcatcactctaggttttacagatttattcctttgttacttctctaattcttc

SEQ ID NO: 16 - DNA, 452 bp, Artificial Sequence
  Synthetic
ctgctgtcttcgacttgggtcagagaaggtgagcagacccggcagcagcctgtcccggggctcaggaagaggcaggcccatcccctggccccaagcacccagcacaacagagggtggcgggcagtgagggcctggcgttgcctgggccccacttctcagccccagctgctgggcctccaaggttgggctgaggatggagttttggctctgggtttgccctgactcctgctggaagacgctgccctggtttttcaccctctagtggccttggacattgagtatttgtagaaatgcagattacattgcaaatggaaacctttgccaggaagacacatgcattttgcttttaattctttgagacatttgattttgtcttagggactgacctttcagcatcaaagaaatacatatctactgtatccgccaaagtttgtgatgcctgcatagacgct

SEQ ID NO: 17 - DNA, 480 bp, Artificial Sequence
  Synthetic
  misc_feature (57)..(57): n is a, c, g, or t
  misc_feature (65)..(65): n is a, c, g, or t
gtaggctcagcgatagtggtcctcttacagagaaacggggagcaggacgacgggggngctggggntggcgggggagggtgcccacaaaaagaatcaggacttgtactgggaaaaaaacccctaaattaattatatttcttggacattccctttcctaacatcctgaggcttaaaaccctgatgcaaacttctcctttcagtggttggagaaattggccgagttcaaccattcactgcaatgcctattccaaactttaaatctatctattgcaaaacctgaaggactgtagttagcggggatgatgttaagtgtggccaagcgcacggcggcaagttttcaagcactgagtttctattccaagatcatagacttactaaagagagtgacaaatgcttccttaatgtcttctataccagaatgtaaatatttttgtgttttgtgttaatttgttagaattctaacacactatatacttccaa

SEQ ID NO: 18 - DNA, 392 bp, Artificial Sequence
  Synthetic
aaaggaagttatgtgtggcatccatgttggtttcgtctgtctgtaatgtgaattccagtatttgtttagtatttccagttgtctcctgctagcaatatgtacagtaacgcgtcaggcttgtgacatttgaataaggaaaaacagagttcctgttaagtgaataactttagcttttacaggggattatgatcaaaagtgattttagtacatcttaaatgatatcttatttctacatggaaagaagttatagaatcttcatagagttctatgagaaaaaatatacttgctatctataaaaaagagaaaaaagaaaaaaaatgagaaaaaagtaagaaaaaaaaaaatcctgtcctaggcttttactcttgatcttcaaaggcacgcagggttta

SEQ ID NO: 19 - DNA, 486 bp, Artificial Sequence
  Synthetic
taatattctctttcatgctgaatttactatgaccatttataagcagtgcagttaactacagatagcatttcaggacaaaatagatgactcaaaccatttattgcttaaaaaatagcttacgccatgctatgctataagcagcttttatgcacattgacaaatgaagagtaagcttcagcttgctaaaggaaactgtggaaccttttgtaacttttggtgatatggaaaattatttacaaaccgtcaaagaatatgaggaagttgctgtatgacatagtgctggcactgatattatccatcatctctttttggacacttctgtaaatgtgattggattgtttgaaagaagatttaaagtttcaaagttttttgttctgtttttgctttgcatttggagaaaatattgaaagcagggtatgttgtttcattcaccttgaaaaaaccatgagtaaatggggatatagaatctctgaatagctcgctaaa

SEQ ID NO: 20 - DNA, 473 bp, Artificial Sequence
  Synthetic
accagtgccctggagatgagttgcaagacatccccagtgaggtataaagaggaagaatataaaagtggactttctgctctagatcatataaggcacttcacagatagcctcaaaatgaggaaaatggaagataatcaatattctgaagctgagctgtcttcttttagtacttcccatgtgccagaggaacttaagcagccgttacacagaaagtccaaatcgcaggcatatgctatgatgctgtcactgtctgacaaggagtccctccattctacatcccacagttcttccaacgtgtggcacagtatggccagggctgcggcggaatccagtgctatccagtccataagccacgtatgacgttatcaaggttgaccagagtgggaccaagtccaacagtagcatggctctttcatataggactatttacaagactgctgagcagaatgccttataaacctgcagggtcactc

SEQ ID NO: 21 - DNA, 534 bp, Artificial Sequence
  Synthetic
  misc_feature (53)..(55): n is a, c, g, or t
  misc_feature (114)..(114): n is a, c, g, or t
  misc_feature (292)..(292): n is a, c, g, or t
atgatctttttccgacttctaaacaagtgactatactagcataaatcattctnnnagtaaaacagctaaggtatagacattctaataatttgggaaaacctatgattacaagtnaaaactcagaaatgcaaagatgttggttttttgtttctcagtctgctttagcttttaactctggaagcgcatgcacactgaactctgctcagtgctaaacagtcaccagcaggttcctcagggtttcagccctaaaatgtaaaacctggataatcagtgtatgttgcaccagaatcancattttttttttaactgcaaaaaatgatggtctcatctctgaatttatatttctcattcttttgaacatactatagctaatatattttatgttgctaaattgcttctatctagcatgttaaacaaagataatatactttcgatgaaagtaaattataggaaaaaaattaactgttttaaaaagaacttgattatgttttatgatttcaggcaagtattcatttttaacttgctacctacttt

SEQ ID NO: 22 - DNA, 420 bp, Artificial Sequence
  Synthetic
  misc_feature (378)..(378): n is a, c, g, or t
taagaagaaagattgaacccccatttccttaattatttcaggaataattctcccgaaatcaggtcaaccccatcaccaaccaaccaaccagttgagttccagattctatgtgaattaaaaagtcaatatatgtataattctgagatgacttaggttggacattcaatgttgtgctatgaatttcctctttatgcagagtatctgtttgcttgcagagtggctttctggcttgctgccagcctgtgcatggtccacgcttatgagttcaggatctacggcaatgtgaatcattcagatgtttacaataaaaaacaccacatgagtaaatgaattcactaatgttaatgttaaacttcatggaaaaatagtcctttgaancttcggtggttagcaattaaagaccctgagttatgtgcaata

SEQ ID NO: 23 - DNA, 402 bp, Artificial Sequence
  Synthetic
aaagtcagtccattttcaagttttggtcttcagagacaaaagaacgtcccagccacctgattttgatggtgaggtaactctaagttgaattcaggctagtgttgcagtatagctttggcatgttcatgagtgagcacccagaatgtgttgaaccaacccccacccctaactactgactatgactgcagtgggtttttatggggaaaaaaagtgtgaaaagcaaaaagaaaggaacagagattttttatcacctttattgtaagacagtccatttatgaattgagtataaacacatacaaagtaacaagagattcctaagaaacgcaaatccttgagtttcacgcacttcatgttcaaccatttgctgtaatccagaggcagcctgtgaatcattctcatgcc

SEQ ID NO: 24 - DNA, 466 bp, Artificial Sequence
  Synthetic
  misc_feature (103)..(103): n is a, c, g, or t
attttctagttttcattctgtacatttttagttagacatcagatttgaaatattaatgtttacctttcaatgtgtggtatcagctggactcagtaacaccccntttcttcagctggggatggggaatggattattggaaaatggaaagaagaaagtaactaaaagccttcctttcacagtttctggcatcactaccactactgattaaacaagaataagagaacattttatcatcatctgctttattcacataaatgaagttgtgatgaataaatctgcttttatgcagacacaaggaattaagtggcttcgtcattgtccttctacctcaaagataatttattccaaaagctaagataaatggaagactcttgaacttgtgaactgatgtgaaatgcagaatctcttttgagtctttgctgtttggaagattgaaaaatattgttcagcatgggtgaccaccaga

SEQ ID NO: 25 - DNA, 536 bp, Artificial Sequence
  Synthetic
gacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgca

SEQ ID NO: 26 - DNA, 508 bp, Artificial Sequence
  Synthetic
  misc_feature (299)..(299): n is a, c, g, or t
  misc_feature (447)..(447): n is a, c, g, or t
  misc_feature (470)..(470): n is a, c, g, or t
ttttcaccccgtctacaatattttgtatttataactgtaaattagtgtatagtgtttcactccagagacttcaataatatagtgttatcaaaggacttgtacagatttcagagaaagacaaatttagaagacggaggattctctattatgtgctatctgagagtcagtatgaaatgtcaaatccaaaagtacataatttagaggtctatttcaaagtaatcatttgagcatagtttctccactgtcagagacgactgttattttattttcaatcaaattaaaacttgtttttatgcatntcttatttttagttatatgttacttgtacataagtagcagcacaatacgtacatataaaccctatgagtataaatcctatagaaatatattaagttgataattatgtctgttctgtttgatcacagagttgcaacaaagaggcctcanttcctaagttgaggaaatgattntctcattttatatgagactcgtggtgtggaattacaag

SEQ ID NO: 27 - DNA, 394 bp, Artificial Sequence
  Synthetic
ggtgacttttgatacgggtttggtgcaggtgaatttattactgagccaaatgaggcacataccgagtcagtagttgaagtccagggcattcgatactgtttatgatttccatatatgtatagtgcctatcccatgctgtagtcactgttatgttaaatccagaagttacactagagccagcgatactttatttgtagacaatcaatttgaatccatatgttattactggcagatgatacatgattacagttctgaatctgtaacacttacaaaaggaaacccagagcagcttgatgagtttttgtttctgcttcgttcctgggagtcagtagaaacagcagttgtatgtggttatgttagtctcaagatacttaatttgttgaccttacttcag

SEQ ID NO: 28 - DNA, 530 bp, Artificial Sequence
  Synthetic
tggaagctggtagctagtgcctctctatcaaatcagggtttgcaccttgagacataatctaccttaaatagtgcatttttttctcagggaattttagatgaacttgaataaactctcctagcaaatgaaatctcacaataagcattgaggtaccttttgtgagccttaaaaagtcttattttgtgaaggggcaaaaactctaggagtcacaactctcagtcattcatttcacagatttttttgtggagaaatttctgtttatatggatgaaatggaatcaagaggaaaattgtaattgattaattccatctgtctttaggagctctcattatctcggtctctggttcctaatcctattttaaagttgtctaattttaaaccactataatatgtcttcattttaataaatattcatttggaatctaggaaaactctgagctactgcatttaggcaggcactttaataccaaactgtaacatgtctcaactgtatacaactcaaaatacaccagctcatttggctgctcagt

SEQ ID NO: 29 - DNA, 161 bp, Artificial Sequence
  Synthetic
tacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacga

SEQ ID NO: 30 - DNA, 333 bp, Artificial Sequence
  Synthetic
gtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggt

SEQ ID NO: 31 - DNA, 543 bp, Artificial Sequence
  Synthetic
gtgttgagtggttctccgtaaaagtataagtgctaacactacgaagaaatgcacacgatcattcttgctcacttctataacaaacttacataaaatggatttaaaaattcctactcacagcctaaaacttctggagttcactacctttttttcaaatcatagtaagatcacttgtgtattttatattttagtaaagccaattatgaagtacaagtatcatacacgtacttttgagctactattatttgaaaaaaatctgccaaatagcatctttaggatatatttacattttcactcatctaaaaagtatacaaaaataaaaagtggaaaaaggtatcttctgaatgttcaagagcatcctatagtgccaaataataaagcaccatttttttcttcataaccaggattaaaattcatatatactgcagggcagacatacatatgatagcttgtgctgattaatttaaccccatttgtaaacagatgaaaattttattttcttatttcatttataagatggctcaatgtattgggaggcttctt

SEQ ID NO: 32 - DNA, 163 bp, Artificial Sequence
  Synthetic
gaaaagtagctctaatcaagtgatatttctgggatatatcacttcagcacctggctccagagattatctacagctcactgaacatggcaatgtgaaggatatcgacagcactgatcatgacagatggtgtgaatacattatgtatcgagggctgatcaggttg

SEQ ID NO: 33 - DNA, 486 bp, Artificial Sequence
  Synthetic
ggggatgggatgcaagtctaaccacaaaattcatttatgtctcatacacactttgaacacctggcctgaaggtaatttcacacaatattttaaataactttgtgcatgaaacacaattttgactgcattttgactgcaactcatcacatgaggtcaggtatggaattttccacttgtggtgttacgttactggctcaaaaagttttggatctcggagcattctggattttgaatttttggattagtgatgctcaacctgtatacagaaatgtcctcatttttaaaaaaagaaatgcatatttatatgttttaaaattacttcaaccaaaagcaacggggagatgtttactgttatatttaggtgacaggtacatggcaattcattataccctcctattttcctatgtttacattattcattaattaaaaaacaatacctagaaaaacccaagactttcaaaagctattttctatatgtgccaatct

SEQ ID NO: 34 - DNA, 465 bp, Artificial Sequence
  Synthetic
tttcattctccaatggtgttccacatggaagaggaccaggaggagctcatcttcggacttgatgacacgtaccttcgctgcattgagctgcacagccacacccttattcagctggagaggtgtttcacagctacaggccagacacgtgtgactgtagtcggaccaccaatggcaaagcagtggctgctgctcatgttccattgcgtggggagccaggactccaagtgtcacgctcgaggtctgaagatgctagagcgtgtccgaagccagcccctgaccaatgatgacctggtcacctccgttagcctgccaccgtacaccggagactgatctcccctgggaagaggggatgagaggagaagtcttggcttcgcttttggtgagaaagacatgaaaaacactgaagaaagtttgaatttgtggccagacctgcccctctcaggccttctaggcatgtgtctgagg

SEQ ID NO: 35 - DNA, 533 bp, Artificial Sequence
  Synthetic
agaggagacactttggcgttcttcaagcagaactgaggctgcgaaaaatccaagtctctacagagacactgatgaagttgaaagggtaattgtttttccatgccaaatgaggaaatcaaattaatgagttgacaaacttttcctgagatgttatttcatctacatttagttaaacaacttagaatgatatctagaaaatatttattttcagccaggaactttagcaaactgtggcaatgatcactgggaatgaaaacgtataatgttctacaatttttgttatcacattggatgactttgaatatagtagtgacacttgggaccaaataaattattttatatgactactagaacaaagttttagtaagctgtctgctaatagagtcagaattcttccagattgtttccagctgtaacaggtattgaatttccaccgtgctcctatgtaacagctgggtggtgtaaaaaatgaacttcaaattatggagtggaagaagcgtaatgttaatatcttgtaaattcgtattccctat

SEQ ID NO: 36 - DNA, 411 bp, Artificial Sequence
  Synthetic
gggcagagctaacacctgagctgttaaagatcctgcattctcaggttgctggcagactgatcatccgtgcagaggagctggcccagatgtggaaagtggtgaatctcccaacagatctgtttaatagtgtgatgaatgtgggtcgcttcacggaggagatcgagtggctgaagtttttagcccttgcttgcagcgctctgggagttactattaccaaaactctcaagatagtgtgtgaggtcttatcatgtgaccacaatggtgggttgccccgaatcccattcagcaccttccagtttctctacacgtatattgccgaagtggatggggagatctgtgcatcacatgtcagcaggatgctaaactacattgaacaggaagtaattggtcctgatggtttaatcacggtga

SEQ ID NO: 37 - DNA, 419 bp, Artificial Sequence
  Synthetic
gtatctctaacattccaaattactgtcaataaaatatatatttattattttaaatgctatgtgttaatatttcacttgcttgtattagaaaggcaaaatgtaagactttggtatgtgtgacatatgctttatttggctttattttacaagtacagtatctgcaaaaaacaaagtaaccttttttcatacctgccagttttgaatttatatatgttattgaacaaatagtaatagaggattcgctgttgaaacaagttgtccaagcaatgttatattcatttttatacttattgggaaagtgtgagttaatattggacacattttatcctgatccacagtggagttttagtaattatattttgttgatttcttcattttgttttctggtataaaagtagagataatgtgtagtcacttct

SEQ ID NO: 38 - DNA, 485 bp, Artificial Sequence
  Synthetic
ttgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattctcccaacagaaatgagcccttaactgggaattctgaccttcccttctgggctgtcatcttcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcc

SEQ ID NO: 39 - DNA, 422 bp, Artificial Sequence
  Synthetic
gacattttaataaccttggctgctaaggacaacattgatagaagccgtctctggctatagataagtagatctaatactagtttggatatctttagggtttagaatctaacctcaagaataagaaatacaagtacgaattggtgatgaagatgtattcgtattgtttgggattgggaggctttgcttatttttttaaaactattgaggtaaagggttaagctgtaacatacttaattgatttcttaccgtttttggctctgttttgctatatcccctaatttgttggttgtgctaatctttgtagaaagaggtcttgtatttgctgcatcgtaatgacatgagtactactttagttggtttaagttcaaatgaatgaaacaaatatttttcctttagttgattttaccctgatttcaccgagt

SEQ ID NO: 40 - DNA, 321 bp, Artificial Sequence
  Synthetic
atagccataatttgccatctttggagagattatggagataatctccttgtctattagccactagcatggctaattgatcattgattggacctcattacataagggggctgtggacgtctttggaatttgttaggggaaggctatgtggtaagctctactaaatgtatctgtacaaggatgaagaaaaacaaaataactactacttttggaggaaacttggaacaagaagagtacatagtttgatccattcctacggtcgagtctaatcaattccatttcttagtgtatgcatctaatgaggcttacaacttgctaacccca

SEQ ID NO: 41 - DNA, 185 bp, Artificial Sequence
  Synthetic
accaagaactggacatcaagtcagaggttttgtgactcacaagatgctgatcttgctcaggttgaaagcttccaggaactgaatttcctgttgagatataaaggcccatctgatcactggattgggctgagcagagaacaaggccaaccatggaaatggataaatggtactgaatggacaagaca

SEQ ID NO: 42 - DNA, 533 bp, Artificial Sequence
  Synthetic
atcccagctacttgatgcgaaggctgaggcaaaagaattgcttgaacccaggagatagaggttgcaatgagccaagatcactccactgcactccagcctgggcgacagaatgagactctgtctcaaaattaaaaaacaaaaaaccaaaaacaaatagatgaaaaagtagactggagacaaataaaagtgagtttctaaaggaaattcacagtaatgctgcattaaacactaagctcacttaggtcactttctagtgagctaaccgtaacagagagcctacaggatacacgtgagataatgtcacgtgtagaagatcgttgtgaattaaagttcaaaattaagacttcttagattatgatgtagattttagagctccttaaaacataaagcgaatcttataaatgttcaattctaaagttattccacttggaaaaattagcttttgggacaatttttaagaacttttgtgtaaatgcagctccatgtttagcataatctaaaaataatttcaagcaatccagaatcttccaaga

SEQ ID NO: 43 - DNA, 410 bp, Artificial Sequence
  Synthetic
attacctatattctcctgatactgattaccaagtcattcacctgaacaccagaattagaattaaacattgtaccatacaatttcattatcttatcagaatgaaaccaattcacagggaaactagagactacggctgtggtggtaacctgcagatatacacattcccatgcactgacataagttgaaaacacatagtactttctagatggaatttttttctttaggcctcaatgaaatacatttcccctatataattccaacagcagtttctctagttctaaattttaccaattcccaaaaaacctaagttttgctgaaatctgatttgaccaatttatcaaatatgtcattttaattacctagctggaagttagagacatgaaatgtactagaggagcccttttccaatg

SEQ ID NO: 44 - DNA, 234 bp, Artificial Sequence
  Synthetic
ggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgct

SEQ ID NO: 45 - DNA, 524 bp, Artificial Sequence
  Synthetic
gaagttgcaacattcgtttgataggaattccagaaaaggagagttatgagaatagggcagaggacataattaaagaaataattgatgaaaactttgcagaactaaagaaaggttcaagtcttgagattgtcagtgcttgtcgagtacctagtaaaattgatgaaaagagactgactcctagacacatcttggtgaaattttggaattctagtgataaagagaaaataataagggcttctagagagagaagagaaattacctaccaaggaacaagaatcaggttgacagcagacttatcactggacacactggatgctagaagtaaatggagcaatgtcttcaaagttctgctggaaaaaggctttaatcctagaatcctatatccagccaaaatggcatttgattttaggggcaaaacaaaggtatttcttagtattgaagaatttagagattatgttttgcatatgcccaccttgagagaattactggggaataatataccttagcacgccagggtgactaca

SEQ ID NO: 46 - DNA, 470 bp, Artificial Sequence
  Synthetic
gaatactcataattcttatctctataatcaaaagtataatttactgtagaaaaataaagagatgcttgttctgaaagtaagatcagtgaactgcttttcagtctcaatctttgagaattgtaaattcatcaaataattgcttacatagtaaaaatttaaggtattagaaaacctgcataacaaatagtattatatattaaatattttgatatgtaaagctctacacaaagctaaatatagtgtaataatgtttacactaataagcaaatatgttaatcttctcatttttttactgtcatataatcttagtgatatgcctattaatagttttaaataaataaattggctcatctggctttttgaaaattttgaaattcttacagatgttgattaggtatatctacaaattaatttcaattttaaaatgatgatataaaaataaatataagtatttttcttgtgtatgtata

SEQ ID NO: 47 - DNA, 540 bp, Artificial Sequence
  Synthetic
gaggacaatatccatgactgctcaaaacttaaaagtcctttgggggtcaaatggcatacggcagtcacctatgtgaacagctgcttgtttgtggccgtgctggtgattctgatcggatgttacatagccatatccaggtacatccacaaatccagcaggcaattcataagtcagtcaagccgaaagcgaaaacataaccagagcatcagggttgttgtggctgtgttttttacctgctttctaccatatcacttgtgcagaattccttttacttttagtcacttagacaggcttttagatgaatctgcacaaaaaatcctatattactgcaaagaaattacacttttcttgtctgcgtgtaatgtttgcctggatccaataatttactttttcatgtgtaggtcattttcaagaaggctgttcaaaaaatcaaatatcagaaccaggagtgaaagcatcagatcactgcaaagtgtgagaagatcggaagttcgcatatattatgattacactgatgtgtaggccttttattgtttgttg

SEQ ID NO: 48 - DNA, 453 bp, Artificial Sequence
  Synthetic
aggaaaaagttgccctcaagcctgacctggaaccgttcctcaccttcatcctcaccatgccctgctatctccagctgctgagcagctgaggtgctgcagccccttcccttccagcccacaagtgtgtgcatattgagctcctgctgtggttaagcactgcaacagactctaccagagatgcaaagagaagcgagagaggcaccttgttctccaagaacttactgtccaatcatgctgtggtgcattcctttgaagatcatgaagaatagccaaacttgtctttgagggtgaatttgacactttaaaataatcagaagtcactgagacccaaagctagtgaataattcaagtgactaagctagatacttgaggatgatagagcaacactaaactaaagcaatgagtctgctttttttaatgatttatacaatgactccaaaggcaatcccaaag

SEQ ID NO: 49 - DNA, 506 bp, Artificial Sequence
  Synthetic
taatacccaactgactaactaaacaaatatcaacttgtaatactcaatgaatttttttgccatttacatttgaccgttggctttagtgaatgtccatatttaattttttaaggcaccattacacagtttatcctacatttatcacatttcttaaagtgttaagattctatggctcatttctatgtatttttcttactttacaaaataacctgaaacagtatagattttgtaacacttaatttgagcagcttttttattacattgaattatataaagtgcatgttaccttagaaaaattagtatttgctgctttactcttttgcaaaacatttgctgtaatgaatggatttgtatttccaatatgtatcttgactgcattttgtaatatttactgctttattcctaattctgctttaaagtactgaactgggcatgaaacattaaaatattaatccagaaactgtataaactggatgttgcttaaaatctgtatcactgccatgttg

SEQ ID NO: 50 - DNA, 445 bp, Artificial Sequence
  Synthetic
gattgagaaccaccagtttagctagtcaatatgaggatggtggtttattctcagaagaaaaagatatgtaagtcttttagctcctaagagtgaagcaaaagcaagacttcaacttcaacctatcttaatgttttaaatgttagggacaataagttgaaatagttagaggagcttcttttcagaaccccagatgagagccaatgtcagataaagtaagcatagcaatgtagcaggaactacaatagaagacattttcactggaattacaaagcagaattaaaattatattgtagaaggaaacaccaagaaaagaatttccagggaaaatcctctttgcaggtattaattcttataattttttgtcttttggattatctgtttactgtctcatctgaactgatcccaggtgaacggtttattgcctagatttgtactcagaggaatt

SEQ ID NO: 51 - DNA, 438 bp, Artificial Sequence
  Synthetic
tacgaggttcctagagctctgctgacccttggccgaaactctaaaatgtacctattaaagataaatgcttctaccaaagtaaaactctgtgagttgtttcagggcagaatgtaccagccagtcagcgttgtttaacaaaataatcagatttttgcctagcactcggttttggtggagctgacgattttgagggctgaggctggttaggtagctggaatgtgcctatgtgaccagctcacttgcagacaccctgccggaagcagagcttaatcttcctaggactgaggtcttagcacatgtactggtggagtttccagaccaccagtatgaataaaagcttgttctgtgtgacccagcaagtggaaggacaaagaactgtgagcctcagatctttggacctttccaatgcgtctctttctcctgttattgctgcaatgt

SEQ ID NO: 52 - DNA, 476 bp, Artificial Sequence
  Synthetic
cacactactgtattttggatggatcaaacctccttaattttaatttctaatcctaaagtaaagagatgcaattgggggccttccatgtagaaagtggggtcaggaggccaagaaagggaatatgaatgtatatccaagtcactcaggaacttttatgcaggtgctagaaactttatgtcaaagtggccacaagattgtttaataggagacgaacgaatgtaactccatgtttactgctaaaaaccaaagctttgtgtaaaatcttgaatttatggggcgggagggtaggaaagcctgtacctgtctgtttttttcctgatccttttccctcattcctgaactgcaggagactgagcccctttgggctttggtgaccccatcactggggtgtgtttatttgatggttgattttgctgtactgggtacttcctttcccattttctaatcattttttaacacaagctgactcttccctt

SEQ ID NO: 53 - DNA, 454 bp, Artificial Sequence
  Synthetic
gattgttaattcctcagttataccagattttataaaatatttgagaatagatggctaacaagaggttagaaatacttttccttaattttaatccacagtatgttacatgcattctaccactacattttggtgctatttaaggtgtgcaattttctataggtgacttttgcaattcagggaagatttgggcatattaaatgaaagaatatctaattgggggaggtgtgaagggaaagaaattcttttcaaaagctgaccacaaagagtagttaaaagtttttgtcactatcttcacaagtgtgtaaagcacagatttcaacagagtgcttggcatattgtagggtgctcaatggtggtttttattattattactcagattccacagtggcaagaaacatcattctacataatggaaaacatttacatcaaatcccacttactttaatgcgaactt

SEQ ID NO: 54 - DNA, 441 bp, Artificial Sequence
  Synthetic
gatttgattacagtggctttgactataatgtgggagccaatttttgcctcagtcttcatttttatatttaccttgttattctcaggcatttttttcttctatgtgagagttaaaatcattctgtaatttccccccaaaatcatattggtattctagttggcaatgtcttacatttatgttaagtttgagggaattggtagttcaagtataagttaattaaggccattttatttctaagtgaacagacttgaaactccagagctactgaagtaaaagttagaatcatttgcattttcattcagataggagataattttgtaaattttgatgctattattttaactctattagcttaagtaatgtcataatagaaaacacaagcatttgaccaaatgagatccattcagcgactaactggcaaggcactcaaaacatgtcatt

SEQ ID NO: 55 - DNA, 220 bp, Artificial Sequence
  Synthetic
gacttgggctcatatgacataaggatgatcatctctggcacaacagctcacttttcttccaaggataagttgcaagaggtgaaactattttttgaatctcttgaggctcaaggatcacatctggatatttttcaaactgttctggaaacgataaccaaaaatataaaatggctggagaagaatcttccgactctgaggacttggctaatggttaatactt

SEQ ID NO: 56 - DNA, 561 bp, Artificial Sequence
  Synthetic
gagagaaaacaatatagccccctacccttttcccaatcctttgccctcaaatcagtgacccaagggagggggggatttaaagggaaggagtgggcaaaacacataaaatgaatttattatatctaagctctgtagcaggattcatgtcgttctttgacagttctttctctttcctgtatatgcaataacaaggttttaaaaaaaaaaaaaaaaagtgagactattagacaaagtatttatgtaattatttgataactcttgtaaataggtggaatatgaatgcttggaaaattaaactttaatttattgacattgtacatagctctgtgtaaatagaattgcaactgtcaggttttgtgttcttgttttcctttagttgggtttatttccaggtcacagaattgctgttaacactagaaaacacacttcctgcaccaacaccaataccctttcaaaagagttgtctgcaacatttttgttttcttttttaatgtccaaaagtgggggaaagtgctatttcctattttcaccaaaattggggaaggagtgccactttccagc

SEQ ID NO: 57 - DNA, 485 bp, Artificial Sequence
  Synthetic
ttattctcattatacctgcttcatatgggtatattactattaaaacagaataccatagagtaattgcattatttgaaaattctctcattttacaatgcacttcaccaatgaaacagctaatttccattttgaaaattaaaagaaaacagcacagagaagttaaatgcggtgtagcaaagttatggggtctgcttgagggcactaacctcaacagattattcctcctctccttagaataaccatgaaaatacaaatttacttagcacatttttgctttttaagtagctggttcattttctgaatttctcacattcagagttccagtcattattgttacatcatgtttgcagaaaccttgtcttatttagtgtctatttgcatataaccctgaaaacattattatttgaaaacttttctatatctcaaattaatatacattttcataacctacctttgtattaagacttgcaattttatcaatctat

SEQ ID NO: 58 - DNA, 414 bp, Artificial Sequence
  Synthetic
ctcgttgcaatcgcctttgcagtcaaccacttctggtgccaggaggagccggagcctgcacacatgatcctgaccgtcggaaacaaggcagatggagtcctggtgggaacagatggaaggtactcttcgatggcggccagtttcaggtccagtgagcatgagaatgcctatgagaatgtgcccgaggaggaaggcaaggtccgcagcaccccgatgtaaccttctctgtggctccaaccccaagactcccaggcacatgggatggatgtccagtgctaccacccaagccccctccttctttgtgtggaatctgcaatagtgggctgactccctccagccccatgccggccctacccgcccttgaagtatagccagccaaggttggagctcagaccgtgtctaggttggggctcg

SEQ ID NO: 59 - DNA, 251 bp, Artificial Sequence
  Synthetic
cttcgctggtggggatgctggagatgcctttgatggctttgattttggcgatgatcctagtgacaagtttttcacatcccataatggcatgcagttcagtacctgggacaatgacaatgataagtttgaaggcaactgtgctgaacaggatggatctggttggtggatgaacaagtgtcacgctggccatctcaatggagtttattaccaaggtggcacttactcaaaagcatctactcctaatggttatg

SEQ ID NO: 60 - DNA, 331 bp, Artificial Sequence
  Synthetic
atcaacacatttagcttggcgttttattcattccatcacccttactgtaactactatggcaactcaaataattgtcatgggactatgtccatcttaatgggtctggatggcatggtgctcctcttaagtgtgctggaattctgcattgctgtgtccctctctgcctttggatgtaaagtgctctgttgtacccctggtggggttgtgttaattctgccatcacattctcacatggcagaaacagcatctcccacaccacttaatgaggtttgaggccacccaaagatcaacagacaaatgctccagaaatctatgctgactgtgacacaag

SEQ ID NO: 61 - DNA, 509 bp, Artificial Sequence
  Synthetic
ggaaaggaatcatagcccatgccaccccacaatgccagataaaccaagtgaaattcaagtttcagtcttccaaccgagtttggaaaaaagaccggactaccattattgggaagttctgcactgcccttttgcctgtcaatgacagagaaaagatggtctgcttacctgaaccagtgaacttacaagcaagtgtgactgtttcctgtgacctgaagatagcctgtgtgtagtttaacctgggcaggacacatctccctgcattttttttttttttttttgagagagaggtgtgatgagggatgtgtgtgtgcagcttattgtagaccattactactaaggagaaaagcaaagctctttcttattttcctcataatcagctaccctggaggggagggagaactcattttacagaacttggtttcctttgccgatcttatgtacatacccattttagctttcccatgcatacttaactgcacttgctttatctccttgggcattcgtactta

SEQ ID NO: 62 - DNA, 325 bp, Artificial Sequence
  Synthetic
aaacatgtaatactccagcagggatatgaagcctctgaattgtagaacctgcatttatttgtgactttgaactaaagacatcccccatgtcccaaaggtggaatacaaccagaggtctcatctctgaactttcttgcgtactgattacatgagtctttggagtcggggatggaggaggttctgcccctgtgaggtgttatacatgaccatcaaagtcctacgtcaagctagctttgcagtggcagtaccgtagccaatgagatttatccgagacgcgattattgctaattggaaattttcccaataccccaccgtgatgacttga

SEQ ID NO: 63 - DNA, 519 bp, Artificial Sequence
  Synthetic
gcacgtatgcaatcttttattcctcttgttttatggataagagatttgttcaggtcaaaagttgcaaaactgttctgaagcttaatattagagacccaagtaagattgtttttagtcttgcttgaggaatgcacactccatttaaaaaattgtgcgctctgtagatttggattttacagcagcaattacttggctaaattagagtactgcaatcttgtaagtagaaaacagccaccatgacaataaacaaaaacacttaaaaatatttttagcttgttttacctacatctagtttcatttcatcttgctttgctttgttgactctgctaccgctgatgtttgccttgtcaatatcagaataggggcatcagtgtcccgtgaaatacattttgttgtgctatgagcctctgtaatcaaatgcttgtactcagaagagagtgtaaaatcagcatttctatgtaacatatatctctaattatctgtgtaattttataacatactttgacaaccccagtgagt

SEQ ID NO: 64 - DNA, 315 bp, Artificial Sequence
  Synthetic
ggaattcttctcttacttcaataaaatgggttttaacataactttaaattcagttaaatatacaatattgaatacctatagttgactttgggatggggactttttcaagtcattaagagtgtttgtttaaggtgatctcattgatggtagttctcagccgtctcaaaaactgcaagctaatcagtcagacattctttaatgaccccaattttttcactttaattgttaccatgttttctatttttactgatttttgctaaagcatgtaagagtgaatttattatagcagtaatcttgtgtttctcctgatgtgca

SEQ ID NO: 65 - DNA, 153 bp, Artificial Sequence
  Synthetic
acgccaagacagacacgctgcagatcgaggactttctggaggagacgctggggccgcccgacttccccagcctggcgcctcgttacagggagtccaacaccgccggcaacgacgttttccacaagttctccgcgttcatcaagaacccggtgc

SEQ ID NO: 66 - DNA, 366 bp, Artificial Sequence
  Synthetic
tcgctgtgagtaccttcaccagaaattgtcccacattaaaggtctcatcctggagtttgaggaaaagaacaggggcagctgaagttatcaagggaatttttgagcctctgcttagtgaaacacaaaggaacaaagcagctataaactaaatagaatgcaactatctgcttttcttatgctgaccactggagtccatggtggcaagtagagagctgctctaggttcttgaggtttggttttcattattaatttttagggtatgggcactgtgcaaagactccatagctgtgcctaggagtctaggaaaagtgacagaggcttggcttttttacctttagttcagccaagtcattttcaagtcctgag

SEQ ID NO: 67 - DNA, 551 bp, Artificial Sequence
  Synthetic
gccgccctgtaggctggggatgggctgctgtgtgaatgttgacgttcgtttcatggagaaaggggaggtgaaagattgaagagcaggttcctgtcaatgttctgagttcgagctggaggtgtagattgaatagtctacatggtctgtgagtgtgtgagatgaacccttccatcctttgacacctggttgtatgtgtaggctaagaaggaaggaccctcctgtcagtgtgcaaagctgtaatctcatggactagaagagagggggccaaggggatggacaggagaagtcatgcagaatctaagcaggaatgcagatagaacacatctaggctcttttccccaggagagtgatgatggagcatatagatctggctcaaattcagcctccatcacttaccagtcaggaaccctggcgatatcactttaactttctgaacctcagagtcttcacctataagacggggaaaataataccaccctttcaagattgttgagataaataagtgatataaaacatgtaaagcttagttctggccacagtgtagctactca

SEQ ID NO: 68 - DNA, 152 bp, Artificial Sequence
  Synthetic
cgaggccaagtcttactttgaaaagtcaaaggagcagctgacacccctgatcaagaaggctggaacggaactggttaacttcttgagctatttcgtggaacttggaacacagcctgccacccagtgaagtgtccagaccattgtcttccaac

SEQ ID NO: 69 - DNA, 194 bp, Artificial Sequence
  Synthetic
gcacagacaccaaggacagagacgctggctaggccgccctccccactgttaccaacatgaagctgctcgcagcaactgtgctactcctcaccatctgcagccttgaaggagctttggttcggagacaggcaaaggagccatgtgtggagagcctggtttctcagtacttccagaccgtgactgactatggcaag

SEQ ID NO: 70 - DNA, 434 bp, Artificial Sequence
  Synthetic
agctgaccaaaatcctgcagggccacagtcggcactgttatttgcttcttttaatcagcaaaggcctcaagtcttaaaataaaaggggagaagaacaaactagctgtcaagtcaaggactggctttcaccttgccctggtgtctttttccagatttcaatatattctctgatggcctgacaggcctattaagtagatgtgatattttcttccaagatgacctccattctcggcagacctaagagttgcctctgagttagctctttggaatcgtgaacacaggtgtgctatattgtccttgtcctaactgtcacttgccatggcctgaatgttggcttaactgaatattgtatgaaaagacatgcctccatatgtgcctttctgttagctctctttgactcaagctgtggggctcctctatacatgctatacatg

SEQ ID NO: 71 - DNA, 465 bp, Artificial Sequence
  Synthetic
atattggtcattgatcttcgttcatgaattagtctacagaaaaaaaatgttctgtaaaattagtctgttgaaaatgttttccaaacaatgttactttgaaaattgagtttatgtttgacctaaatgggctaaaattacattagataaactaaaattctgtccgtgtaactataaattttgtgaatgcattttcctggtgtttgaaaaagaagggggggagaattccaggtgccttaatataaagtttgaagcttcatccaccaaagttaaatagagctatttaaaaatgcactttatttgtactctgtgtggcttttgttttagaattttgttcaaattatagcagaatttaggcaaaaataaaacagacatgtatttttgtttgctgaatggatgaaaccattgcattcttgtacactgatttgaaatgctgtaaatatgtcccaatttgtattgattctcttt

SEQ ID NO: 72 - DNA, 528 bp, Artificial Sequence
  Synthetic
taagcaatgaacccatctcgaaggaggagtttgtaatacacaccatatttcctcctaatggaatgaatctgtacaaaaacaatcacagtgaaagcagtagtaaccgaagctacagagacagtggtattccagactcagatcactcacactcatgatgctgaaggactcacagcagacttgtgttttgggttttttaaacctaagggaggtgatggtaggaaccctgttctactgcaaaacactggaaaaagagactgaaaaaaagcaatgtactgtacatttgccatataatttatatttaagaactttttattaaaagtttcaaatttcaggttactgctgcgattgatgtagtggagatgcctgaacacaattctatattttagtattttttagtaatttgtactgtattttccttgcaaatattggagttataaaccatttactttgtgttctactgagtaagatgacttgttgactgtgaaagtgaattttcttgctgtgtcgaacaatcaggactgcattcat

SEQ ID NO: 73 - DNA, 534 bp, Artificial Sequence
  Synthetic
agcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgt

SEQ ID NO: 74 - DNA, 424 bp, Artificial Sequence
  Synthetic
actgggacgtgtgcgcagacatggtgggcaccttcacagacaccgaggaccctgccaagttcaagatgaagtactggggcgtagcctcctttctgcagaaaggaaatgatgaccactggatcgtcgacacagactacgacacgtatgccgtacagtactcctgccgcctcctgaacctcgatggcacctgtgctgacagctactccttcgtgttttcccgggaccccaacggcctgcccccagaagcgcagaagattgtaaggcagcggcaggaggagctgtgcctggccaggcagtacaggctgatcgtccacaacggttactgcgatggcagatcagaaagaaaccttttgtagcaatatcaagaatctagtttcatctgagaacttctgattagctctcagtcttcagctctatttatctt

SEQ ID NO: 75 - DNA, 489 bp, Artificial Sequence
  Synthetic
attaccttatcatgtcttagagcccgtctttatgtttaaaactaatttcttaaaataaagccttcagtaaatgttcattaccaacttgataaatgctactcataagagctggtttggggctatagcatatgcttttttttttttaattattacctgatttaaaaatctctgtaaaaacgtgtagtgtttcataaaatctgtaactcgcattttaatgatccgctattataagcttttaatagcatgaaaattgttaggctatataacattgccacttcaactctaaggaatatttttgagatatccctttggaagaccttgcttggaagagcctggacactaacaattctacaccaaattgtctcttcaaatacgtatggactggataactctgagaaacacatctagtataactgaataagcagagcatcaaattaaacagacagaaaccgaaagctctatataaatgctcagagttctttatgtatt

SEQ ID NO: 76 - DNA, 482 bp, Artificial Sequence
  Synthetic
ggcatgtgtgactgtttcagcgactgcggagtctgtctctgtggcacattttgtttcccgtgccttgggtgtcaagttgcagctgatatgaatgaatgctgtctgtgtggaacaagcgtcgcaatgaggactctctacaggacccgatatggcatccctggatctatttgtgatgactatatggcaactctttgctgtcctcattgtactctttgccaaatcaagagagatatcaacagaaggagagccatgcgtactttctaaaaactgatggtgaaaagctcttaccgaagcaacaaaattcagcagacacctcttcagcttgagttcttcaccatcttttgcaactgaaatatgatggatatgcttaagtacaactgatggcatgaaaaaaatcaaatttttgatttattataaatgaatgttgtccctgaacttagctaaatggtgcaacttagtttctccttgctttcatattatcg

SEQ ID NO: 77 - DNA, 491 bp, Artificial Sequence
  Synthetic
gaggaggaactgacgcagctacgccacgaactggagcggcagaacaatgaataccaagtgctgctgggcatcaaaacccacctggagaaggaaatcaccacgtaccgacggctcctggagggagagagtgaagggacacgggaagaatcaaagtcgagcatgaaagtgtctgcaactccaaagatcaaggccataacccaggagaccatcaacggaagattagttctttgtcaagtgaatgaaatccaaaagcacgcatgagaccaatgaaagtttccgcctgttgtaaagtctattttcccccaaggaaagtccttgcacagacaccagtgagtgagttctaaaagatacccttggaattatcagactcagaaacttttatttttttttttctgtaacagtctcaccagacttctcataatgctcttaatatattgcacttttctaatcaaagtgcgagtttatgagggtaaagctctactttcctactg

SEQ ID NO: 78 - DNA, 554 bp, Artificial Sequence
  Synthetic
gacatgacccgagtaactttctgctgatgaacaaatctgcaccccacttcagacctcggtgggcattcacaccaccccccatgccaccggctccactttccccttttattaatacattcacccagataatcattaaaattaacatgtgccaggtcttaggatgtgtcttggggtgggcacagtacccggtgactcttggggatatttatttattttccctgagcctatatcttcatctgtgaaatggggataaaaatacttgttgctgtcacaattattaccatctctccagctagcaaaattactaccagagccgttactacacacaaaggctattgaccgagcacataccatgtgccacacaccttgacaaaatcttttaatacagtttattatgtactattcaatctttacacaatgtcacgggaccagtattgtttacccaattttttataaggacactgaagcttagaggagtgaaatgttttgagtgttatttcagagagcaaatggcaaagactggatccaaacccatcttcctggacctgaagttc

SEQ ID NO: 79 - DNA, 498 bp, Artificial Sequence
  Synthetic
caccaagccgacctcagagttgttcatcttccttatgggacaaaaccggttgaccagaaaatgggcagagagagatgacctcggaagcatttccacagatggtgtcagggtttcaagaagtcttagggcttccaggggtcccctggaagctttagaatatttatgggtttttttttcaaatatcaattatatggtagattgaggattttttttctgtagctcaaaggtggagggagtttattagttaaccaaatatcgttgagaggaatttaaaatactgttactaccaaagatttttattaataaaggcttatattttggtaacacttctctatatttttactcacaggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccagga

SEQ ID NO: 80 - DNA, 473 bp, Artificial Sequence
  Synthetic
ctttgttttcctcctatcaaggcagctattttattaaatttttcacttagtctgagaaatagcagatagtctcatatttaggaaaactttccaaataaaataaatgttattctctgataaagagctaatacagaaatgttcaagttattttactttctggtaatgtcttcagtaaaatattttctttatctaaatattaacattctaagtctaccaaaaaaagttttaaactcaagcaggccaaaaccaatatgcttataagaaataatgaaaagttcatccatttctgataaagttctctatggcaaagtctttcaaatacgagataactgcaaaatattttccttttatactacagaaatgagaatctcatcaataaattagttcaagcataagatgaaaacagaatattctgtggtgccagtgcacactaccttcccacccatacacatccatgttcactgtaacaaact

SEQ ID NO: 81 - DNA, 482 bp, Artificial Sequence
  Synthetic
gaggcaaaggaaactgggtggggaggatcaattagagaggaggcacctgggatccaccttcttccttaggtcccctcctccatcagcaaaggagcacttctctaatcatgccctcccgaagactggctgggagaaggtttaaaaacaaaaaatccaggagtaagagccttaggtcagtttgaaattggagacaaactgtctggcaaagggtgcgagagggagcttgtgctcaggagtccagccgtccagcctcggggtgtaggtttctgaggtgtgccattggggcctcagccttctctggtgacagaggctcagctgtggccaccaacacacaaccacacacacacaaccacacacacaaatgggggcaaccacatccagtacaagcttttacaaatgttattagtgtccttttttatttctaatgccttgtcctcttaaaagttattttatttgttattattatttgttcttgactgtta

SEQ ID NO: 82 - DNA, 433 bp, Artificial Sequence
  Synthetic
gttcaaaatatttccaagcctgagtattgtctattggtatagatttttagaaatcaataattgattatttatttgcacttattacaatgcctgaaaaagtgcaccacatggatgttaagtagaaattcaagaaagtaagatgtcttcagcaactcagtaaaaccttacgccaccttttggtttgtaagaggttttttatacatttcaaacaggttgcacaaaagttaaaataatggggtcttttataaatccaaagtactgtgaaaacattttacatattttttaaatcttctgactaatgctaaaacgtaatctaattaaatttcatacagttactgcagtaagcattaggaagtgaatatgatatacaaaatagtttataaagactctatagtttctataatttattttactggcaaatgtcatgcaacaa

SEQ ID NO: 83 - DNA, 551 bp, Artificial Sequence
  Synthetic
gaagctgacacaccgtatgttgttagagtcttttatctggtcaggggaaacaaaatcttgacccagctgaacatgtcttcctgagtcagtgcctgaatctttattttttaaattgaatgttccttaaaggttaacatttctaaagcaatattaagaaagactttaaatgttattttggaagacttacgatgcatgtatacaaacgaatagcagataatgatgactagttcacacataaagtccttttaaggagaaaatctaaaatgaaaagtggataaacagaacatttataagtgatcagttaatgcctaagagtgaaagtagttctattgacattcctcaagatatttaatatcaactgcattatgtattatgtctgcttaaatcatttaaaaacggcaaagaattatatagactatgaggtaccttgctgtgtaggaggatgaaaggggagttgatagtctcataaaactaatttggcttcaagtttcatgaatctgtaactagaatttaattttcaccccaataatgttctatatagcctttgctaa

SEQ ID NO: 84 - DNA, 355 bp, Artificial Sequence
  Synthetic
ggagatgacttaagttggcagcagtaatcttcttttaggagcttgtaccacagtcttgcacataagtgcagatttgccccaagtaaagagaatttcctcaacactaacttcacggggataatcaccacgtaactacccttaaagcatatcactagccaaagaggggaatatctgttcttcttactgtgcctatattaagactagtacaaatgtggtgtgtcttccaactttcattgaaaatgccatatctataccatattttattcgagtcactgatgatgtaatgatatattttttcattattatagtagaatatttttatggcaagagatttgtggtcttgatcatacctatt

SEQ ID NO: 85 - DNA, 437 bp, Artificial Sequence
  Synthetic
ccctccctttggatgctggtgaatactgtgtgcctagaggaaatcgtaggcggttccgcgttaggcagcccatcctgcagtatagatgggatatgatgcataggcttggagaaccacaggcaaggatgagagaagagaatatggaaaggattggggaggaggtgagacagctgatggaaaagctgagggaaaagcagttgagtcatagtctgcgggcagtcagcactgacccccctcaccatgaccatcatgatgagttttgccttatgccctgaatcctgatggtttccctaaagttattacggaaacagacccctgctttcgaatttacatgttcatgatgtgcccttgttgtaaacctttacctgtcacttgtttacgtgggtctcctattaccagcttctaattgaatattgtgtttttgaaccagtctgtaa

SEQ ID NO: 86 - DNA, 549 bp, Artificial Sequence
  Synthetic
tcacctcggcgtactatcgtggtgcagtgggggccctcctggtgtttgacctaaccaagcaccagacctatgctgtggtggagcgatggctgaaggagctctatgaccatgctgaagccacgatcgtcgtcatgctcgtgggtaacaaaagtgacctcagccaggcccgggaagtgcccactgaggaggcccgaatgttcgctgaaaacaatggactgctcttcctggagacctcagccctggactctaccaatgttgagctagcctttgagactgtcctgaaagaaatctttgcgaaggtgtccaagcagagacagaacagcatccggaccaatgccatcactctgggcagtgccaggctggacaggagcctggcctggggagaagagggcctgttgcatcagcctctgaccttggccagcaccacctgcccccactggctttttgtgccccttgtccccacttcagccccaggacctttccttgccctttggttccagatatcagactgttccctgttcacagcaccctcagggtcttaaggtcttc

SEQ ID NO: 87 - DNA, 520 bp, Artificial Sequence
  Synthetic
aactttgtatagcccatgtacctaccttgtatagaaaaataattttaaaaatttgaatggaagggggtaaaggaagtcatgaagtttttttgcatttttatttaaatgaaggaattccaaataactcacctacagatttttagcacaaaaatagccattgtaaagtgttaaaatttacgataagtattctattggggaggaaaggtaactctgatctcagttacagtttttttttcctttttaatttcattattttgggtttttggtttttgcagtcctatttatctgcagtcgtattaagtcctattgctagaataggttactacaaaaaaggttatattctgaaagaaaaataactgacattatatataaccaattaatttaaagtattgccatttaaattacacactgagagcatgtcctatgcagacatagatttttctgttcatttatttttcttcattgcagtggattgatttgataaatagatgtgttgaattactacatttgctgtacatat

SEQ ID NO: 88 - DNA, 244 bp, Artificial Sequence
  Synthetic
ccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacga

SEQ ID NO: 89 - DNA, 296 bp, Artificial Sequence
  Synthetic
  misc_feature (41)..(41): n is a, c, g, or t
  misc_feature (45)..(45): n is a, c, g, or t
ccttaatattctgtgtcttttgcaattaaattgtcagtcancttncttcaaaaccttacagtccttcctaagttactcttcatgagatttcatccatttactaatactgtatttttggtggactaggcttgcctatgtgcttatgtgtagctttttactttttatggtgctgattaatggtgatcaaggtaggaaaagttgctgttctattttctgaactctttctatactttaagatactctatttttaaaacactatctgcaaactcaggacactttaacagggcagaatactc

SEQ ID NO: 90 - DNA, 353 bp, Artificial Sequence
  Synthetic
ggaaaggctccatggaagacatacatataggcatccttcttgatactgaaaactatcttctttgtttgaaaggaactattgctaaatgcagaacaagctcattgcagttacctattgtgcatctttttaaatacttgattatgtaaccataaatctgacagcatgtctcattggcttatctggtagcaaatctaggccccgtcagccaccctattgacattggtggctctgctaaacctcagggggacatgaaatcactgcctcttgggcatctggggacacatggtaatgctgtgccttgacagaagtatttgtttaaagaaatgtcaatgctgtcatttgtgaactctatc

SEQ ID NO: 91 - DNA, 319 bp, Artificial Sequence
  Synthetic
agtcgctccatttatcgcttgagatctccagccttaccgcggctcgaaatggaccccaactgctcctgcaccactggtgtctcctgcgcctgcaccggctcctgcaagtgcaaagagtgcaaatgcacctcctgcaagaagagctgctgctcctgctgccccgtgggctgtgccaagtgtgcccacggctgtgtctgcaaagggacgttggagaactgcagctgctgtgcctgatgtgggaacagctcttctcccagatgttaatagaacaagctgcacaacctggattttttttcaatacgatactgagccatttgct

SEQ ID NO: 92 - DNA, 452 bp, Artificial Sequence
  Synthetic
gatgaaatctcactgctaatgctcagagatcttttttcactgtaagaggtaacctttaacaatatgggtattacctttgtctcttcataccggttttatgacaaaggtctattgaatttatttgtttgtaagtttctactcccatcaaagcagctttctaagttattgccttggttattatggatgatagttatagcccttataatgccttaactaaggaagaaaagatgttattctgagtttgttttaatacatatatgaacatatagttttattcaattaaaccaaagaagaggtcagcagggagatactaacctttggaaatgattagctggctctgttttttggttaaataagagtctttaatcctttctccatcaagagttacttaccaagggcaggggaagggggatatagaggtcacaaggaaataaaaatcatctttcatcttt

SEQ ID NO: 93 - DNA, 481 bp, Artificial Sequence
  Synthetic
tctgatggtgcactactctgaggcctgtatggcccctttcatcaatatctattcaaatatacaggtgcatatatacttgttaaagctcttatataaaaaagccccaaaatattgaagttcatctgaaatgcaaggtgctttcatcaatgaaccttttcaaacttttctatgattgcagagaagctttttatatacccagcataacttggaaacaggtatctgacctattcttatttagttaacacaagtgtgattaatttgatttctttaattccttattgaatcttatgtgatatgattttctggatttacagaacattagcacatgtaccttgtgcctcccattcaagtgaagttataatttacactgagggtttcaaaattcgactagaagtggagatatattatttatttatgcactgtactgtatttttatattgctgtttaaaacttttaagctgtgcctcacttattaaagcac

SEQ ID NO: 94 - DNA, 465 bp, Artificial Sequence
  Synthetic
  misc_feature (388)..(388): n is a, c, g, or t
gtctactatagcccggagaccatcggcaggcgagtggctgtgggactcgggtcctgcctcgccatcctcatcctggcaatctgtgggctcaagctccagcgacgttggaagaggacacagagccagcaggggcttcaggagaattccagcggccagagcttctttgtgaggaataaaaaggttagaagggcccccctctctgaaggcccccactccctgggatgctacaatccaatgatggaagatggcattagctacaccaccctgcgctttcccgagatgaacataccacgaactggagatgcagagtcctcagagatgcagagacctcccccggactgcgatgacacggtcacttattcagcattgcacaagcgccaagtgggcnactatgagaacgtcattccagattttccagaagatgaggggattcattactcagagctgatccagtttggggtcggg

SEQ ID NO: 95 - DNA, 503 bp, Artificial Sequence
  Synthetic
gaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcctttgcatggagtgaccatagctccttctctcttacattgaatgtagagaatgtagccattgtagcagcttgtgttgtcacgcttcttcttttgagcaactttcttacactgaagaaaggcagaatgagtgcttcagaatgtgatttcctactaacctgttccttggataggctttttagtatagtatttttttttgtcattttctccatcagcaaccagggagactgcacctgatggaaaagatatatgactgcttcatgacattcctaaactatcttttttttattccacatctacgtttttggtggagtccctttttatcatccttaa

SEQ ID NO: 96 - DNA, 427 bp, Artificial Sequence
  Synthetic
gttttgctaactggctatctggaatacctgatcaagcataactatggcaaagataaagcagcaaccaagaaaccagttgtgaacataattactccgtctcatgtagaggagcgggggtgccagctaacaataacattttctgttccaaacgaagatgttttccaagaactagaaaaaagaggagtggtttgtgacaagcggaatccaaatggcattcgagtggctccagttcctctctataattctttccatgatgtttataaatttaccaatctgctcacttctatacttgactctgcagaaacaaaaaattagcagtgttttctagaacaacttaagcaaattatactgaaagctgctgtggttatttcagtattattcgatttttaattattgaaagtatgtcaccattgaccacatgtaacta

SEQ ID NO: 97 - DNA, 165 bp, Artificial Sequence
  Synthetic
tgcaccaagaagtatactccagaacaagtagctatggccaccgtaacagctctccaccgtactgttcctgcagctgttcctggcatctgctttttgtctggtggcatgagtgaagaggatgccactctcaacctcaatgctatacctactagggtccaatgcccg

SEQ ID NO: 98 - DNA, 257 bp, Artificial Sequence
  Synthetic
gctgtcaggattcttcaaggagaatgaatactgggaatcaagacaggactataccttatccataggcgcaggtgcacagggggaggccataaagatcaaacatgcatggatgggtcctcacgcagacacacccacagaaggacactagcctggcgcgcgtgcacacacacacacacacacacgagttcataatgtggtgatggccctaagttaagcaaaatgcttctgcacacaaaactctctggtttacttcaaat

SEQ ID NO: 99 - DNA, 463 bp, Artificial Sequence
  Synthetic
aggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgc

SEQ ID NO: 100 - DNA, 548 bp, Artificial Sequence
  Synthetic
tcaagtgggaagagcgctgttcaaggaccacctgagcgtgacctctgtggctgctacagcgtgttccagtgtcctgccgggctgtgccgagccatggaaccatggggagaccttcacttgcactgctgcccaccccgagttgaagaccccactaaccgccaacatcacaaaatccggaaacacattccggcccgaggtccacctgctgccgccgccgtcggaggagctggccctgaacgagctggtgacgctgacgtgcctggcacgtggcttcagcccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcgga

SEQ ID NO: 101 - DNA, 553 bp, Artificial Sequence
  Synthetic
  misc_feature (102)..(102): n is a, c, g, or t
  misc_feature (199)..(200): n is a, c, g, or t
  misc_feature (202)..(203): n is a, c, g, or t
  misc_feature (205)..(212): n is a, c, g, or t
cactactgctgcagctcacaaacacctctgcatattacatgtacctcctcctgctcctcaagagtgtggtctattttgccatcatcacctgctgtctgcttngaagaacggctttctgctgcaatggagagaaatcataacagacggtggcacaaggaggccatcttttcctcatcggttattgtccctagaagcgtcnncnnannnnnnnnttgggctttctttctgggtttgggccatttcagttctcatgtgtgtactattctatctattgtataatggttttcaaaccagtgggcacacagagaacctcactctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccctgaagcagtcttctttgctagttgaattatgtggtgtgtttttccgtaata

SEQ ID NO: 102 - DNA, 413 bp, Artificial Sequence
  Synthetic
gttggcttggtttcaataaggaacggggacacttacaaattgctgcttgtccaaatcaggatccactgcaaggaacaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgatt

SEQ ID NO: 103 - DNA, 353 bp, Artificial Sequence
  Synthetic
tctggctctcaggtgccacatgtgacatccagatgacccagtctccatcctccctgtctgcatctgtaggagacagagtcaccatcacttgccaggcgactcaggacattggcaactatttaaattggtatcagcacaaaccagggaaagcccctaacctcctgatctacgatgcatccaatttggaaacaggggtcccatcaaggttcagtggacgtggatctgggacacattttactttcaccatcagcagcctgcagcctgaagatattgcaacatattactgtcaacagtatggtaatctcccattcactttcggccctgggaccaaagtgcatatcaaacgaactgtg

SEQ ID NO: 104 - DNA, 466 bp, Artificial Sequence
  Synthetic
atatctatgaccactgttagcccattatattcattccaattagaagaaaagaaatgtgaatactatattccgtgttttgagtgacaagtttcgaaaaataaaaacactgtatttttaaaagggaaatgcacttaaatgaaaacagttattacaaaagttaagatttaaaaagaaaaagcaagagtttttattatgatgtaataccagtagaatatttaaaaggcacaccacatctgaataatcaatgtaaatattttctttcaaagttgtaagttttcatatcatgtgctgtaaagttttcctaaatgaggctttaacgtaaacactggtgacataaaccattcattgctacgttgcttattgtgtttttatgctgttttatacttttttatgagttatgatagcagcaattaagttgtttgtattttgcttaactaaaacaaaaatgcttttatcttgctataga

SEQ ID NO: 105 - DNA, 288 bp, Artificial Sequence
  Synthetic
tcctccctgtctgcatctgtaggagacagagtcaccatcacttgccaggcgagtcaggacattagtaattttttaacttggtatcagaagaagccagggaaagcccctaaagtcctgatctacgatgcatccaatttggaaacaggggtcccatcaaggttcagtggaagtggttctgggacagattttactttcaccatcgccagcctgcagcctgacgattttgcaacatattactgtcaacaatataatgatcaccccctcactttcggccctgggaccaaagtg

SEQ ID NO: 106 - DNA, 485 bp, Artificial Sequence
  Synthetic
aacatcatggacttattggctctatagatacagcccaggtaagatttaacattttaaaaaattctattctgtcatctctaatatgtttcccaattgtttcaggaccaggacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagctgggaaactgtctgtgataggtaatggctcctgtacctgcaatggcatccagagattgtcttttctgatgtctgagacatggaggagaagtcactttatagtgctgtttcttatgcatgtggtgtcctatttaca

SEQ ID NO: 107 - DNA, 242 bp, Artificial Sequence
  Synthetic
gacgcccctacagggccatagatttctcacatcaaggacctgcatttgttacctggcaccggtaccatttgttgtgtctggaaagagatctccagcgactcattggcaatgagtcttttgctttgccctactggaactttgccactgggaggaacgagtgtgatgtgtgtacagaccagctgtttggggcagcgagaccagacgatccgactctgattagtcggaactcaagattctccagc

SEQ ID NO: 108 - DNA, 162 bp, Artificial Sequence
  Synthetic
  misc_feature (114)..(114): n is a, c, g, or t
  misc_feature (119)..(119): n is a, c, g, or t
agtgggtctcagctattagtgggagtgcaggtaccacatactacgcagactccgtgaagggccggttcaccacctccagagacaattccaagaacacgctgtatctgcaaatgnacagnctgagagccgaggacacggccgtatattactgtgcgaaatccc

SEQ ID NO: 109 - DNA, 273 bp, Artificial Sequence
  Synthetic
agtcgggcccaggactggtgaagccttcggagaccctgtccctcacctgcactgtctctggtggctccatcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcga

SEQ ID NO: 110 - DNA, 259 bp, Artificial Sequence
  Synthetic
tcctccctgtctgcatctgtaggagacagagtcaccatcacttgccgggtgagtcagggcattagcagttatttaaattggtatcggcagaaaccagggaaagttcctaagctcctgatctatagtgcatccaatttgcaatctggagtcccatctcggttcagtggcagtggatctgggacagatttcactctcactatcagcagcctgcagcctgaagatgttgcaacttattacggtcaacggacttacaatgccc

SEQ ID NO: 111 - DNA, 435 bp, Artificial Sequence
  Synthetic
ataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgacaaaagggacacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctattctgtagtatttttatttggatatttctagtatggcacatgatatgagtcact

SEQ ID NO: 112 - DNA, 455 bp, Artificial Sequence
  Synthetic
ggtcatcgaccccttgacaagaagagagaagaggctcccagcctgatgctggaggctgtcttcacgctgacccagacctgggggtgttgtgtcctacaggatgtcagttgcaagaggctttgctacaacaggaaaggccaatcagaaatagtgttgatgagttaaataacaatgtggaagctgtttcccagacctcctcttcttcctttcagtacatgtatttgctgaaagacctgtggcaaaagaggcagaagcaagtaaaagataatgaaaatgtagtcaatgagtactcctcagaactggaaaagcaccaattatatatagatgagactgtgaatagtaatatcccaactaaccttcgtgtgcttcgttcaatcctggaaaacctgagaagcaaaatacaaaagttagaatctgatgtctcagctcaaatggaatattgtcgcaccccatgc

SEQ ID NO: 113 - DNA, 501 bp, Artificial Sequence
  Synthetic
  misc_feature (37)..(37): n is a, c, g, or t
  misc_feature (437)..(437): n is a, c, g, or t
  misc_feature (452)..(452): n is a, c, g, or t
ggacagttcctgtgatcagaggcaagatttgcccagngaacagaataaaggtgcttctttggatagctctcaatgttcgccctcccctgcctcccaggaagaccttgaatcagagatttcagaggattctgatcaggaagtggacattgagggcgataaaagctattttaatgctggatgatgaccactggcattggcatgttcagaaaactggatttaggaataatgttttgctacagaaaatcttcatagaagaactggaaggctatataagaaagggaatcaattctctggtattctggaaacctaaaaatatttggtgcactgctcaattaacaaacctacatggagaccttaattttgacttaacaaatagtttatgtactgctcttaggttgttttgataaagtgacattatagtgattaaattctttccnctttaaaaaaacagntagtggttttcactatttataaataggaccttcttgaacgacttttctg

SEQ ID NO: 114 - DNA, 532 bp, Artificial Sequence
  Synthetic
  misc_feature (108)..(108): n is a, c, g, or t
  misc_feature (111)..(111): n is a, c, g, or t
  misc_feature (121)..(123): n is a, c, g, or t
  misc_feature (142)..(142): n is a, c, g, or t
  misc_feature (171)..(172): n is a, c, g, or t
  misc_feature (174)..(174): n is a, c, g, or t
  misc_feature (176)..(176): n is a, c, g, or t
  misc_feature (187)..(188): n is a, c, g, or t
  misc_feature (431)..(431): n is a, c, g, or t
  misc_feature (433)..(434): n is a, c, g, or t
aaatgatacactactgctgcagctcacaaacacctctgcatattacatgtacctcctcctgctcctcaagagtgtggtctattttgccatcatcacctgctgtctgcntgnaagaacggcnnnctgctgcaatggagagaantcataacagacggtggcacaaggaggccnncntntcctcatcggnnattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtaganannctgcggcttctagccttgtccctctcttagtgttctttaatcagataactgcctggaagcctttcattttacacgccctgaagcagtcttctttgcta

SEQ ID NO: 115 - DNA, 286 bp, Artificial Sequence
  Synthetic
  misc_feature (201)..(201): n is a, c, g, or t
  misc_feature (226)..(226): n is a, c, g, or t
  misc_feature (255)..(255): n is a, c, g, or t
gaaactatatgtatctcgtggatctgccagtaccagccttccaaatgaaactctttcagagttagagacacctgggaaatactcacttacaccaccaaaccactggggccacccacatcgatacctgcagcatctttagtcaagttggaggagaaagacaacacttggtctaagacacggcagcaagacatccctgcatantgttccagataaaaatgaaagctgntcacacccacttgcctccccaatctgttnaaacagcttcgtgtctagtatgagctcagta

SEQ ID NO: 116 - DNA, 212 bp, Artificial Sequence
  Synthetic
  misc_feature (43)..(43): n is a, c, g, or t
  misc_feature (121)..(121): n is a, c, g, or t
tctcacctatacgtgcaagaaaggaacagttgcttgcggccancccctgttgtagaaaatgccaagacctttggaaagatgaaacctcgttatgaaatcaactccctgattagataccacntgcaaagatggtttcattcaacgtcaccttccaactatccggtgcttaggaaatggaagatgggctatacctaaaattacctgcatgaacc

SEQ ID NO: 117 - DNA, 472 bp, Artificial Sequence
  Synthetic
  misc_feature (77)..(79): n is a, c, g, or t
  misc_feature (81)..(81): n is a, c, g, or t
  misc_feature (83)..(84): n is a, c, g, or t
  misc_feature (87)..(99): n is a, c, g, or t
  misc_feature (103)..(104): n is a, c, g, or t
  misc_feature (107)..(109): n is a, c, g, or t
  misc_feature (113)..(117): n is a, c, g, or t
gcgagtcctgcggacagtgggtgggggtggagccggcagacgtggagcgcactggcatgggcattggccggccgccnnnangnntgnnnnnnnnnnnnngtgnnccnnnggcnnnnnccttcagcaacttcatcagctcttccagctggaccatcagctcccgcctgctctcctgcagggccgacatcctctgctccagttcatccttcctttgcctatccaagacgagaagcctatcagataaagagaccctcaaagatggatctaaggccaggtgaaatggatttcagacctgatcctgctacttaccagttatgagacgctgagcaagtcatttagcatccaatacctcataaaatggtgataatgtctgttttgcctacttcctggactgttctgagaataataggtaaaagtgctttggaaagtttagatgactatataaaggtattacttatattaccagagtgaa

SEQ ID NO: 118 - DNA, 466 bp, Artificial Sequence
  Synthetic
  misc_feature (39)..(39): n is a, c, g, or t
  misc_feature (41)..(41): n is a, c, g, or t
  misc_feature (59)..(59): n is a, c, g, or t
  misc_feature (65)..(67): n is a, c, g, or t
gatggacagtcatcaactggcggatatagcggtacatantnatccttagccaccagggncacaannntacagtagacaatacagacagagcttttgttgagctgtaactgagctatggaatagcttctttgatgtacctctttgccttaaattgctttttagttctaagattgtagaatgatcctttcaaattgtaatcttttctaacagagatattttaatatagttgctttcttaaaaaacaaaaaaactactgtcagtattaatactgagccagactggcatctacagatttcagatctatcattttattgattcttaagcttgtattaaaaactaggcaatatcatcatggatacataggagaagacacatttacaatcattcattgggccttttatctgtctatccatccatcatcatttgaaggcctaatatattgccaagtactcacatggtatgcatt

SEQ ID NO: 119 - DNA, 279 bp, Artificial Sequence
  Synthetic
  misc_feature (40)..(40): n is a, c, g, or t
ggaagaaagtccttgtcctttgagactcagaaggccccangctccagtatgccatcatgatgcctgctaaggcagccaccttggtgtacatgctcacagaggctctgttcatggagcagctgctgtttgaaaaattttgaaatgcaagatccacaactagatggaaggcactctagtctttgcagaaaaaaatgtacctgaatgtacattgcacaatgcctggcacaaagaaggaagaatataaatgatagttcgactcgtctgtggaagaacttacaa

SEQ ID NO: 120 - DNA, 184 bp, Artificial Sequence
  Synthetic
tgcttttcagggactatgcaatgaaaccttgacactgaagctgtacaacaatggctttacttcagtccaaggatatgctttcaatgggacaaagctggatgctgtttacctaaacaagaataaatacctgacagttattgacaaagatgcatttggaggagtatacagtggaccaagcttgctg

SEQ ID NO: 121 - DNA, 393 bp, Artificial Sequence
  Synthetic
agctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaagcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtg

SEQ ID NO: 122 - DNA, 438 bp, Artificial Sequence
  Synthetic
  misc_feature (27)..(28): n is a, c, g, or t
  misc_feature (82)..(82): n is a, c, g, or t
  misc_feature (298)..(301): n is a, c, g, or t
  misc_feature (304)..(308): n is a, c, g, or t
  misc_feature (314)..(317): n is a, c, g, or t
  misc_feature (327)..(327): n is a, c, g, or t
  misc_feature (329)..(329): n is a, c, g, or t
  misc_feature (353)..(355): n is a, c, g, or t
  misc_feature (360)..(360): n is a, c, g, or t
  misc_feature (362)..(362): n is a, c, g, or t
  misc_feature (378)..(378): n is a, c, g, or t
  misc_feature (385)..(385): n is a, c, g, or t
  misc_feature (407)..(407): n is a, c, g, or t
  misc_feature (409)..(409): n is a, c, g, or t
gctctgaggcctcctatgagctgacanngccaccctcggtgtcagtgtccccaggacagacggccaggatcacctgctctgnagatgcattgccaaagcaatatgcttattggtaccagcagaagccaggccaggcccctgtgctggtgatatataaagacagtgagaggccctcagggatccctgagcgattctctggctccagctcagggacaacagtcacgttgaccatcagtggagtccaggcagaagacgaggctgactattactgtcaatcagcagacagcagtggtacttnnnntgnnnnnttcggnnnngggaccaagntnaccgtcctaggtcagcccaaggcnnnccccncngtcactctgttcccgncctcctntgaggagctccaagccaacaangncacactagtgtgtctgatcagtgacttct

SEQ ID NO: 123 - DNA, 314 bp, Artificial Sequence
  Synthetic
  misc_feature (150)..(150): n is a, c, g, or t
  misc_feature (161)..(161): n is a, c, g, or t
  misc_feature (185)..(185): n is a, c, g, or t
ctactctggctccgaggtgccagatgtgacatccagatgacccagtctccatcctccctgtctgcatctgtaggagacagagtcaccatcacttgccgggcaagtcagagcattagcagctatttaaattggtatcagcagaaaccaggnaaagcccctangctcctgatctatgctgcatccantttgcaaagtggggtcccatcaaggttcagtggcagtggatctgggacagatttcactctcaccatcagcagtctgcaacctgaagattttgcaacttactactgtcaacagagttacagtacccctca

SEQ ID NO: 124 - DNA, 276 bp, Artificial Sequence
  Synthetic
  misc_feature (32)..(35): n is a, c, g, or t
  misc_feature (203)..(203): n is a, c, g, or t
  misc_feature (240)..(240): n is a, c, g, or t
gcataaattaatcagccccagagtgctttaannnnttctcttaaataccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttntaatcttatcttttggcttctgcagatggttgcaatngatccttaaccaataatggtcagtcctcatctctgt

SEQ ID NO: 125 - DNA, 290 bp, Artificial Sequence
  Synthetic
cttgctctgttgcagattggcagatgccgcctccctatgtggtgctggacttgccgcaggagaccctggaggaggagacccccggcgccaacctgtggcccaccaccatcaccttcctcaccctcttcctgctgagcctgttctatagcacagcactgaccgtgaccagcgtccggggcccatctggcaacagggagggcccccagtactgagcgggagccggcaaggcacagggaggaagtgtggaggaacctcttggagaagccagctatgcttgccagaactcagcc

SEQ ID NO: 126 - DNA, 552 bp, Artificial Sequence
  Synthetic
  misc_feature (35)..(35): n is a, c, g, or t
  misc_feature (45)..(45): n is a, c, g, or t
  misc_feature (50)..(51): n is a, c, g, or t
  misc_feature (58)..(59): n is a, c, g, or t
  misc_feature (63)..(63): n is a, c, g, or t
  misc_feature (74)..(76): n is a, c, g, or t
  misc_feature (85)..(86): n is a, c, g, or t
  misc_feature (91)..(91): n is a, c, g, or t
  misc_feature (95)..(95): n is a, c, g, or t
  misc_feature (100)..(100): n is a, c, g, or t
  misc_feature (105)..(105): n is a, c, g, or t
  misc_feature (109)..(109): n is a, c, g, or t
  misc_feature (124)..(124): n is a, c, g, or t
  misc_feature (129)..(130): n is a, c, g, or t
  misc_feature (152)..(152): n is a, c, g, or t
  misc_feature (160)..(160): n is a, c, g, or t
  misc_feature (174)..(174): n is a, c, g, or t
  misc_feature (190)..(190): n is a, c, g, or t
  misc_feature (201)..(201): n is a, c, g, or t
  misc_feature (212)..(212): n is a, c, g, or t
  misc_feature (222)..(222): n is a, c, g, or t
  misc_feature (236)..(236): n is a, c, g, or t
tctccaaatcaaaccacagtatatgttgtaacaantatctatgancactnntagcccnntatnttcattccaannngaagaaaannaatgntgantactntattnccgngttttgagtgacaantttcnnaaaataaaaacactgtattttnaaaagggnaaatgcacttaaangaaaacagttattacnaaaagttaagnatttaaaaagnaaaagcaagnagtttttattatgnatgtaataccagtagaatatttaaaaggcacaccacatctgaataatcaatgtaaatattttctttcaaagttgtaagttttcatatcatgtgctgtaaagttttcctaaatgaggctttaacgtaaacactggtgacataaaccattcattgctacgttgcttattgtgtttttatgctgttttatacttttttatgagttatgatagcagcaattaagttgtttgtattttgcttaactaaaacaaaaatgcttttatcttgctatagaataaacacatttcagtaaaaactgtggactgtattttgttgcaac

SEQ ID NO: 127 - DNA, 540 bp, Artificial Sequence
  Synthetic
  misc_feature (148)..(148): n is a, c, g, or t
  misc_feature (197)..(197): n is a, c, g, or t
  misc_feature (299)..(299): n is a, c, g, or t
  misc_feature (320)..(320): n is a, c, g, or t
  misc_feature (331)..(331): n is a, c, g, or t
  misc_feature (335)..(335): n is a, c, g, or t
  misc_feature (402)..(402): n is a, c, g, or t
  misc_feature (411)..(411): n is a, c, g, or t
  misc_feature (417)..(417): n is a, c, g, or t
  misc_feature (426)..(426): n is a, c, g, or t
  misc_feature (450)..(452): n is a, c, g, or t
  misc_feature (455)..(455): n is a, c, g, or t
  misc_feature (460)..(460): n is a, c, g, or t
  misc_feature (465)..(465): n is a, c, g, or t
  misc_feature (486)..(486): n is a, c, g, or t
gctgtgttgagagagctgcgttgcgtttgtttacagaccacgcaaggagttcatcccaaaatgatcagtaatctgcaagtgttcgccataggcccacagtgctccaaggtggaagtggtagcctccctgaagaacgggaaggaaattngtcttgatccagaagccccttttctaaagaaagtcatccagaaaatttnggacggtggaaacaaggaaaactgattaagagaaatgagcacgcatggaaaagtttcccagtcttcagcagagaagttttctggaggtctctgaacccaggnaagacaagaaggaaagatttngtgtgttgttnattngtttttccagtagttagctttcttcctggattcctcactttgaagagtgtgaggaaaacctatgttngccgcttangctttncagctcagntaatgaagtgtttagcatagtacnnntgntattngctgntattttatctgctatgctatngaagttttggcaattgactatagtgtgagccaggaatcactggctgttaatctt

SEQ ID NO: 128 - DNA, 440 bp, Artificial Sequence
  Synthetic
  misc_feature (185)..(185): n is a, c, g, or t
  misc_feature (217)..(217): n is a, c, g, or t
  misc_feature (234)..(234): n is a, c, g, or t
  misc_feature (266)..(266): n is a, c, g, or t
  misc_feature (309)..(309): n is a, c, g, or t
  misc_feature (365)..(365): n is a, c, g, or t
cactgaattttctcttaggctgaccatatattttattaaagtagtcttgaattcaggagtggaaaaaaaaaagttatgttttagacctcaatgtaagctgaaaggaaataaaggaacttctatatttccatttctcaggctgattttccttcccctgttctcttatctgtatcatgacatgagangtgaacctcagaaatgtaaaaacaaaaagtcntagaataaagcattccnttgaattttaaatgaacatgcttttagcagcntggatacgaagcagactattttcaacaaattgaatgtttcccnacttgtgtgagagccaaacaagtgccatacagaaagggacctcttcaatgttaccnaaagctttgtggcagcatttaagtgtggtggctccatagaagtcactatagcatggacgagccacttggcttgct

SEQ ID NO: 129 - DNA, 551 bp, Artificial Sequence
  Synthetic
  misc_feature (158)..(158): n is a, c, g, or t
  misc_feature (516)..(516): n is a, c, g, or t
agtcagtgttgtcttaatatccttgataatgctgtaaagtttatttttacaaatatttctgtttaagctatttcacctttgtttggaaatccttcccttttaaagagaaaatgtgacacttgtgaaaaggcttgtaggaaagctcctccctttttttnctttaaacctttaaatgacaaacctaggtaattaatggttgtgaatttctatttttgctttgtttttaatgaacatttgtctttcagaataggattctgtgataatatttaaatggcaaaaacaaaacataattttgtgcaattaacaaagctactgcaagaaaaataaaacatttcttggtaaaaacgtatgtatttatatattatatatttatatataatatatattatatatttagcattgctgagctttttagatgcctattgtgtatcttttaaaggttttgaccattttgttatgagtaattacatatatattacattcactatattaaaattgtacttttttactatgtgnctcattggttcatagtctttattttgtcctttgaa

SEQ ID NO: 130 - DNA, 514 bp, Artificial Sequence
  Synthetic
  misc_feature (41)..(41): n is a, c, g, or t
  misc_feature (43)..(43): n is a, c, g, or t
  misc_feature (68)..(68): n is a, c, g, or t
  misc_feature (91)..(91): n is a, c, g, or t
  misc_feature (97)..(97): n is a, c, g, or t
  misc_feature (103)..(103): n is a, c, g, or t
  misc_feature (109)..(109): n is a, c, g, or t
  misc_feature (133)..(133): n is a, c, g, or t
  misc_feature (138)..(138): n is a, c, g, or t
  misc_feature (150)..(150): n is a, c, g, or t
  misc_feature (158)..(158): n is a, c, g, or t
  misc_feature (170)..(170): n is a, c, g, or t
  misc_feature (316)..(316): n is a, c, g, or t
  misc_feature (411)..(411): n is a, c, g, or t
  misc_feature (481)..(481): n is a, c, g, or t
gaaacgtttgcaacatgatcaaggtgttagttctccaccananaagttgtattcttcttttgccaccncaaaccatcacagagtctttaantgcaantcaatnggtcantgctagtcaaagctatgttcttanaaaanccccagacagcntcagagcntcagaaaatccntgtggagtggctgctctgtaccgtgggcatccggcagccaggaagtgagacaacataattataactttgttttatgatgctgcatcatttgtactgtttaggtcgacgtgaggacatcatcttatttagaattttccgtttggcantctcttttgggtgggagttatgctgggggttgtaaataatgacaaggctgagatttttatgatgtttaaattgggcacaatgattttgaccttantccccaaacttcttttcttttctactgtttaacatacacaggctatttatacacgtccccagctcccatntgaaacctgtgactcaggtttatgaatggtgtt

SEQ ID NO: 131 - DNA, 481 bp, Artificial Sequence
  Synthetic
gttgggaaccactattatttctattttgtagatgagaaaatgaagataaacatcaaagcatagattaagtaattttccaaagggtcaaaattcaaaattgaaaccaaggtttcagtgttgcccattgtcctgttctgacttatatgatgcggtacacagagccatccaagtaagtgatggctcagcagtggaatactctgggaattaggctgaaccacatgaaagagtgctttatagggcaaaaacagttgaatatcagtgatttcacatggttcaacctaatagttcaactcatcctttccattggagaatatgatggatctaccttctgtgaactttatagtgaagaatctgctattacatttccaatttgtcaacatgctgagctttaataggacttatcttcttatgacaacatttattggtgtgtccccttgcctagcccaacagaagaattcagcagccgtaagtctaggacagg

SEQ ID NO: 132 - DNA, 526 bp, Artificial Sequence
  Synthetic
  misc_feature (41)..(41): n is a, c, g, or t
  misc_feature (54)..(62): n is a, c, g, or t
  misc_feature (76)..(76): n is a, c, g, or t
  misc_feature (82)..(82): n is a, c, g, or t
  misc_feature (96)..(96): n is a, c, g, or t
  misc_feature (105)..(106): n is a, c, g, or t
  misc_feature (120)..(120): n is a, c, g, or t
  misc_feature (123)..(123): n is a, c, g, or t
  misc_feature (239)..(239): n is a, c, g, or t
  misc_feature (500)..(500): n is a, c, g, or t
gttgtaacaatatctatgaccactgttagcccattatattncattccaattagnnnnnnnnnaatgtgaatactanattccngtgttttgagtgancaagtttcnnaaaataaaaacacngtntttttaaaagggaaatgcacttaaatgaaaacagttattacaaaagttaagatttaaaaagaaaaagcaagagtttttattatgatgtaataccagtagaatatttaaaaggcacnccacatctgaataatcaatgtaaatattttctttcaaagttgtaagttttcatatcatgtgctgtaaagttttcctaaatgaggctttaacgtaaacactggtgacataaaccattcattgctacgttgcttattgtgtttttatgctgttttatacttttttatgagttatgatagcagcaattaagttgtttgtattttgcttaactaaaacaaaaatgcttttatcttgctatagaataaacacatttcagtaaaaantgtggactgtattttgatgcaacaac

SEQ ID NO: 133 - DNA, 436 bp, Artificial Sequence
  Synthetic
gtgattcaaacttctgtgtactgggtgatgcacccattgtgattgtggaagatagaattcaatttgaactcaggttgtttatgaggggaaaaaaacagttgcatagagtatagctctgtagtggaatatgtcttctgtataactaggctgttaacctatgattgtaaagtagctgtaagaatttcccagtgaaataaaaaaaaattttaagtgttctcggggatgcatagattcatcattttctccaccttaaaaatgcgggcatttaagtctgtccattatctatatagtcctgtcttgtctattgtatatataatctatatgattaaagaaaatatgcataatcagacaagcgtaatgcataatcagacaagcttgaatattgtttttgcaccagacgaacagtgaggaaattcggagctatacatatgtgcag

SEQ ID NO: 134 - DNA, 528 bp, Artificial Sequence
  Synthetic
  misc_feature (217)..(217): n is a, c, g, or t
  misc_feature (422)..(422): n is a, c, g, or t
  misc_feature (454)..(454): n is a, c, g, or t
  misc_feature (487)..(487): n is a, c, g, or t
gagtctgagcatgagctgcagccagggccactgcgaggggggctgggccagtgcaccttccagggctccgtccagtagtttcccctgccttaatgtgacatgaggcccattcttctctctttgaagagagcagtcaacattcttagtagtgggtttctgttctattggatgactttgagatttgtctttgtttccttttggaattgttcaaatgttncttttaatgggtggttgaatgaacttcagcattcaaatttatgaatgacagtagtcacacatagtgctgtttatatagtttaggagtaagagtcttgttttttattcagattgggaaatccattccattttgtgaattgggacatagttacagcagtggaataagtattcatttagaaatgtgaatgagcagtaaaactgatganataaagaaattaaaagatatttaattcttgcnttatactcagtctattcggtaaaattttttttnaaaaatgtgcatacctggatttccttggcttctttgagaat

SEQ ID NO: 135 - DNA, 554 bp, Artificial Sequence
  Synthetic
  misc_feature (63)..(63): n is a, c, g, or t
  misc_feature (185)..(185): n is a, c, g, or t
  misc_feature (187)..(187): n is a, c, g, or t
  misc_feature (189)..(189): n is a, c, g, or t
  misc_feature (195)..(195): n is a, c, g, or t
  misc_feature (199)..(199): n is a, c, g, or t
  misc_feature (204)..(204): n is a, c, g, or t
  misc_feature (206)..(206): n is a, c, g, or t
  misc_feature (211)..(211): n is a, c, g, or t
  misc_feature (219)..(219): n is a, c, g, or t
  misc_feature (223)..(223): n is a, c, g, or t
  misc_feature (237)..(237): n is a, c, g, or t
  misc_feature (246)..(246): n is a, c, g, or t
  misc_feature (265)..(265): n is a, c, g, or t
  misc_feature (269)..(269): n is a, c, g, or t
  misc_feature (275)..(275): n is a, c, g, or t
  misc_feature (278)..(279): n is a, c, g, or t
  misc_feature (407)..(407): n is a, c, g, or t
tgagtgggctttgagagagggggaagagtgagtctgagcacgagttgcagccagggccagtgnggagggggtttgggccagtgcaccttccggggccccatcccttagtttccactgcctcctgtgacgtgaggcccattcttcactctttgaagcgagcagtcagcattcttagtagtgggttncngntctgtnggangactntngagantattcttngttncctgttggagttgntcaaatgtnccttttaacggatggttgnatgngcgtcngcnnccaggtttatgaatgacagtagtcacacatagtgctgtttatatagtttaggagtaagagtcttgttttttattcagattgggaaatccattccattttgtgaattgtgacataataatagcagtggnaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttct

SEQ ID NO: 136 - DNA, 415 bp, Artificial Sequence
  Synthetic
gagtgagtctcagcacatgttgcagccagggccagtgggagggggtctgggccagtgcaccttccagggccccatccattagcttccactgcctcgtgtgatatgaggcccattcctgcctctttgaagagagcagtcagcattcttagcagtgagtttctgttctgttggatgactttgagatttatctttgtttcctgttggaattgttcaaatgttccttttaacaaatggttggatgaacttcagcatccaagtttatgaatgacagtagtcacacatagtgctgtttatatagtttaggggtaagagtcctgttttttattcagattgggaaatccattccattttgtgagttgtcacataataacagcagtggaatatgtatttgcctatattgtgaacgaattagcag

SEQ ID NO: 137 - DNA, 492 bp, Artificial Sequence
  Synthetic
ttgtcaaggggctttgcattcaaactgcttttccagggctatactcagaagaaagataaaagtgtgatctaagaaaaagtgatggttttaggaaagtgaaaatatttttgtttttgtatttgaagaagaatgatgcattttgacaagaaatcatatatgtatggatatattttaataagtatttgagtacagactttgaggtttcatcaatataaataaaagagcagaaaaatatgtcttggttttcatttgcttaccaaaaaaacaacaacaaaaaaagttgtcctttgagaacttcacctgctcctatgtgggtacctgagtcaaaattgtcatttttgttctgtgaaaaataaatttccttcttgtaccatttctgtttagttttactaaaatctgtaaatactgtatttttctgtttattccaaatttgatgaaactgacaatccaatttgaaagtttgtgtcgacgtctgtctagcttaaatgaatg

SEQ ID NO: 138 - DNA, 530 bp, Artificial Sequence
  Synthetic
  misc_feature (67)..(67): n is a, c, g, or t
  misc_feature (258)..(258): n is a, c, g, or t
  misc_feature (274)..(274): n is a, c, g, or t
  misc_feature (293)..(294): n is a, c, g, or t
  misc_feature (417)..(417): n is a, c, g, or t
  misc_feature (422)..(422): n is a, c, g, or t
  misc_feature (467)..(467): n is a, c, g, or t
  misc_feature (486)..(486): n is a, c, g, or t
gacagtgatcaggatagctgccgactaagcattgactcacagagcagcagcagcggaccccgaaagnaccttcgcacggatgccttcagccagcaccacctcgagccgctcgagtgcccatttgagcggcagcactacccagaggcctatgcctcccccagccacaccaaaggcgagcagggcctctacccgctgcccttgctcaacagcaccctggacgacgggaaggccaccctgaccccttccaacacgccactngggcgcaacctctcgnctcaccagacctaccccgnngtggcagggcgagagatggtggggcccacgctgcccggatacccaccccacatccccaccagcggacagggcagctatgcctcctctgccatcgcaggcatggtggcaggaagtgaatactcntggcnatgcctatggccacaccccctactcctcctacagcgaggcctggngcttccccaactccagctntgctgagttccccatattattacagttccacatcaaggccgagt

SEQ ID NO: 139 - DNA, 482 bp, Artificial Sequence
  Synthetic
gcagctgagctagacatgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaagagaggctggttccagaggatgcatctggctcaccgggtgttccgaaaccaaagaagaaacttcgccttatcagctt

SEQ ID NO: 140 - DNA, 492 bp, Artificial Sequence
  Synthetic
  misc_feature (83)..(83): n is a, c, g, or t
  misc_feature (177)..(177): n is a, c, g, or t
  misc_feature (284)..(284): n is a, c, g, or t
  misc_feature (335)..(335): n is a, c, g, or t
  misc_feature (348)..(348): n is a, c, g, or t
  misc_feature (388)..(390): n is a, c, g, or t
  misc_feature (444)..(445): n is a, c, g, or t
agaaggtccttaacacagtcgtgaacaaacacaaggacctgaagacacacgggttcacactggagtcctgccgtagcatgatngcgctcatggatacagatggctctggaaagctcaacctgcaggagttccaccacctctggaacaagattaaggcctggcagaaaattttcaaanactatgacacagaccagtccggcaccatcaacagctacgagatgcgaaatgcagtcaacgacgcaggattccacctcaacaaccagctctatgacatcattaccatngcggtacgcagacaaacacatgaacatcgactttgacagtttcatctgctngcttcgttaggcntggagggcatgttcagagcttttcatgcatttgacaaggnnngagatggtatcatcaagctcaacgttctggagtggctgcagctcaccatgtatnnctgaaccaggctggcctcatccaaagccatgcaggatcactcaggat

SEQ ID NO: 141 - DNA, 531 bp, Artificial Sequence
  Synthetic
tcataaaactacttgtcagcatcacagttacttttgtcttatgctttactccctttcatgtgatgttgctgattcgctgcattttagagcatgctgtgaacttcgaagaccacagcaattctgggaagcgaacttacacaatgtatagaatcacggttgcattaacaagtttaaattgtgttgctgatccaattctgtactgttttgtaaccgaaacaggaagatatgatatgtggaatatattaaaattctgcactgggaggtgtaatacatcacaaagacaaagaaaacgcatactttctgtgtctacaaaagatactatggaattagaggtccttgagtagaaccaaggatgttttgaagggaagggaagtttaagttatgcattattatatcatcaagattacattttgaaaaggaaatctagcatgtgaggggactaagtgttctcagagtgatgttttaatccagtccaataaaaatatcttaaaactgcattgtacagctccctccctgcgttttattaaatga

SEQ ID NO: 142 - DNA, 440 bp, Artificial Sequence
  Synthetic
  misc_feature (335)..(335): n is a, c, g, or t
  misc_feature (339)..(339): n is a, c, g, or t
  misc_feature (342)..(346): n is a, c, g, or t
gaaccagtgcttctataactccagttacctgaatgtccagcgggagaatgggaccgtctccagatacgagggaggccgagaacatgttgctcacctgctgttccttagggacaccaagaccttgatgtttggttcctacctggacgatgagaagaactgggggctgtctttctatgctgacaagccagagacgaccaaggagcaactgggagagttctacgaagctctcgactgcttgtgcattcccaggtcagatgtcatgtacaccgactggaaaaaggataagtgtgagccactggagaagcagcacgagaaggagaggaaacaggaggagngggnatnnnnncccggtccgctcttgtgtctatactgacactttagggtcgctttcttttgtaacacgttatgtagacctacagtgtttcacttctagtaccaat

SEQ ID NO: 143 - DNA, 288 bp, Artificial Sequence
  Synthetic
  misc_feature (132)..(132): n is a, c, g, or t
  misc_feature (202)..(202): n is a, c, g, or t
ggtcacagctggatctgcttgttgcatttccagctgtgcagcacgtctcagagattcttgaagaatgaagacatttctgctctcagctccgggggtgaggtgtgcctggcctctgcctccaccctcctcctnttcaccaggtgcatgcatgccctctctgagtctggactttgcttcccctccaggagggaccaccctcccngactggcctgggatatctttacaagcaggcactgtatttttttattcgccatctgatccccatgcctagcagagtgctggcactta

SEQ ID NO: 144 - DNA, 523 bp, Artificial Sequence
  Synthetic
  misc_feature (41)..(41): n is a, c, g, or t
  misc_feature (440)..(440): n is a, c, g, or t
  misc_feature (442)..(442): n is a, c, g, or t
  misc_feature (485)..(486): n is a, c, g, or t
  misc_feature (488)..(491): n is a, c, g, or t
  misc_feature (493)..(498): n is a, c, g, or t
gacaacagccctggaggggaacagagtgagagagatgtttngctctggtacagcctgtgttgtttgcccagtttctgatatactgtacaaaggcgagacaatacacattccaactatggagaatggtcctaagctggcaagccgcatcttgagcaaattaactgatatccagtatggaagagaagagagcgactggacaattgtgctatcctgaatggaaaatagaggatacaatggaaaatagaggataccaactgtatgctactgggacagactgttgcatttgaattgtgatagatttctttggctacctgtgcataatgtagtttgtagtatcaatgtgttacaagagtgattgtttcttcatgccagagaaaatgaattgcaatcatcaaatggtgtttcataacttggtagtagtaacttaccttaccttaccnanaaaaatattaatgtaagccatataacatgggattttcctcaannannnnannnnnnccttttgtacttcactcagatacta

SEQ ID NO: 145 - DNA, 393 bp, Artificial Sequence
  Synthetic
  misc_feature (133)..(133): n is a, c, g, or t
  misc_feature (204)..(204): n is a, c, g, or t
  misc_feature (259)..(259): n is a, c, g, or t
ctcaccgaggcgctcaaaggcatggacaagatgttcttgaacaacaccaccactaacaggcacacgtctggggaaggcccaggtagtaaaactggcgacaaggaggagaaacacaggaaatgaaaaatttttnaaaaaagaaggaaaaatgttttaaatacaaaaggaaaaacagacaaaaatttaattttagctttaaaatantggattggctttggaagaattatattaggtagaatacacatacaatcaaaatttnaaaaaaaaaagctaaataacttaaaaaaaaactgaggcgtataacggagcaacaatatcggttctcagtgtctatttcaagatacatttggagacaaccgtccggattttccacttcggttctttcgagtttag

SEQ ID NO: 146 - DNA, 472 bp, Artificial Sequence
  Synthetic
  misc_feature (87)..(87): n is a, c, g, or t
  misc_feature (110)..(110): n is a, c, g, or t
actcatcatactttggtgtgcactagtgcaattccaagataatatcactggtatgagtcagtgaagtctacatcctctgatagctgnttcaaatgtgcattttgtggaanttttgagaaaaatagagcaaaatcaacatgactggtggtgagagaccacacattttatgagagtttggaattattgtagacatgcccaaaacttatccttgggccataattatgaaaactcatgatcaagatatatgtgtatacatacatgtatctggtttgtcaggctacaaggtaggctgcaaaattaaatctagacattcttttaatgccaccacacgtgttccgcttctctcttttaaagtatttataaaaatataaattgtacattttgtaaaatattatgtttgatttctctacttgtcatatcactaaataaacacgattttattgctgcgtgactctgtattactttggtgcat

SEQ ID NO: 147 - DNA, 369 bp, Artificial Sequence
  Synthetic
  misc_feature (121)..(121): n is a, c, g, or t
gctgggccagtgcatctaacagccctgtgcagcagcttcccttgcctcgtgtaacatgaggcccattcttcactctgtttgaagaaaatagtcagtgttcttagtagtgggtttctatttngttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaata

SEQ ID NO: 148 - DNA, 245 bp, Artificial Sequence
  Synthetic
ggtgaggggatgacccctggagatgaagggaagaggtgaagccttagcaaaaatgcctcctcaccactccccaggagaatttttataaaaagcataatcactgattccttcactgacataatgtaggaagcctctgaggagaaaaacaaagggagaaacatagagaacggttgctactggcagaagcataagatctttgtacaatattgctggccctggttcacctgtttactgttatcacaata

SEQ ID NO: 149 - DNA, 261 bp, Artificial Sequence
  Synthetic
gaatgaagaaaagtcgcctcaacgacaaacaaaagcaccgactagatttccttcagctgatgattgactcccagaattcgaaagaaactgagtcccacaaagctctgtctgatctggagctcgcagcccagtcaataatcttcatttttgctggctatgaaaccaccagcagtgttctttccttcactttatatgaactggccactcaccctgatgtccagcagaaactgcaaaaggagattgatgcagttttgcccaata

SEQ ID NO: 150 - DNA, 524 bp, Artificial Sequence
  Synthetic
gtgccagacttctgagaagcacctgccagcaacagcttccttctttgagcttagtccatccctcatgaaaaatgactgaccactgctgggcagcaggagggatgatgaccaactaattcccaaaccccagtctcattggtaccagccttggggaaccacctacacttgagccacaattggttttgaagtgcatttacaagtttctggcatcactaccactactgattaaacaagaataagagaacattttatcatcatctgctttattcacataaatgaagttgtgatgaataaatctgcttttatgcagacacaaggaattaagtggcttcgtcattgtccttctacctcaaagataatttattccaaaagctaagataaatggaagactcttgaacttgtgaactgatgtgaaatgcagaatctcttttgagtctttgctgtttggaagattgaaaaatattgttcagcatgggtgaccaccagaaagtaatcttaagccatctagatgtcacaattgaaac

SEQ ID NO: 151 - DNA, 454 bp, Artificial Sequence
  Synthetic
gaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgta

SEQ ID NO: 152 - DNA, 399 bp, Artificial Sequence
  Synthetic
tttgatggggacagcccacgtctgatagggtgggacatggggcagcagttagagggttgtgctctttctagtgtgggatagtttgcaagatgatatgttgtagccacttctgcagtgacggggacagaaggagtcagtgttcttaactacagtggtagcagcagaaaagaagggtaagcagggttctagaaatttgtgttatgttttctccccactgtatttatttctttggttagtggtgcaagaaattctgttttcctgtagcaaattaataaagcgttcaaacataaggaattacgacaacagcttgtagatgccagacttcaacaaacagcacagctgataaaagaagctgatgaaagacatcagagagagagagagtttgtaagttctacttct

SEQ ID NO: 153 - DNA, 567 bp, Artificial Sequence
  Synthetic
  misc_feature (33)..(33): n is a, c, g, or t
  misc_feature (38)..(38): n is a, c, g, or t
  misc_feature (49)..(49): n is a, c, g, or t
  misc_feature (354)..(354): n is a, c, g, or t
  misc_feature (542)..(542): n is a, c, g, or t
gactttgatttctcagaggcacccatgtttacncagcntttggttaacncctatgccatagctggttacaatgccaccctaaactgcagtgtgagaggaaatcctaagcctaaaataacctggatgaaaaacaaagttgctattgtggatgatccaagatacaggatgttcagcaaccagggagtctgtaccctggaaattcgcaagcccagcccctatgatggaggcacttactgctgcaaagcagtcaatgaccttgggacagtggagattgaatgcaaactggaggtgaaaggtgggctctccttctgcagactcctcttgcaaggcgtacctccaaacataattgattcntatctgcgagacttacactcaagcaatcctgaggaatactgagggagggcctggctactgtctctctgcactctgctgctttgaaatctggttgaaatgagaaaaagcattttctgttttcccaccaggcccccaagtgtggtctttttctttcctcctaatgttgaagagaaaaaaaaaaaaaaaangtttgcccagattgcttaattaaaa

SEQ ID NO: 154 - DNA, 516 bp, Artificial Sequence
  Synthetic
  misc_feature (116)..(116): n is a, c, g, or t
agcagcagaatcagagtacgagatgcttgacactgtgtccttcttccatccaggattggggagtcagaggactgtgcaggaatcgtgtccttcctgtgctctccagatgccagctncgtcaacggggagaacattgcggtggcaggctactccactcggctctgagaggagtgggggcggctgcgtagctgtggtcccaggcccaggagcctgagggggtgtctaggtgatcatttggatctggaggcagagtctgccattctgccagactagcaatttgggggcttactcatgctaggcttgaggaagaagaaaaacgcttcggcattctccttaggacttatctgcttgtagatttggctgatccaattaacatgtggggttcttggtgtgggtctggggagctgaaggattttatggagctggtgctttggaggaatcttaagggaaaggagtagaagctcaggcctttgaaggatttcagctcctcctctctgtaatttgtgctttaagcat

SEQ ID NO: 155 - DNA, 129 bp, Artificial Sequence
  Synthetic
ctgcactttccgtagaccttaaaatctcagaggtagggctgccaccaaggtgaagatgggaacgcagatgatccatgagtttgccctggtttcactggcccaagtggtttgtgctaaccacgtctgtct

SEQ ID NO: 156 - DNA, 551 bp, Artificial Sequence
  Synthetic
  misc_feature (122)..(122): n is a, c, g, or t
  misc_feature (156)..(156): n is a, c, g, or t
acgccgactttaccaaccaagatattgtatgtgtgtgacattcagctaacattgatctaggcacttagtttgctaccacattgttcccttcattgattgaaactgtaaataacataacactnttaaggcagctaagcaaatattttaataagccantgaaaggcaagatgccagagaaaatctgtatattcagctatttggagaactcgtgttttccacaaattaaactggagatgtcatttgaaattttcttcccttaaacatgctgtcacaacatggattccttctcatggatgtctttctaggcttataaatatatggtgtgattgctataattttgtgaaattttattcagcaattaatagtgatttcagcaatatgtactaagattccaaggcagaaataaatgtataaaggatttgagcctgtatgtgtaagaagaaactctctcttcagtcatatttcctaaattcagtgtaagtacctcgctgatttagcactggagttattccttgaatgtgtaaataatgatgttctattctgacctaatg

SEQ ID NO: 157 - DNA, 539 bp, Artificial Sequence
  Synthetic
  misc_feature (27)..(27): n is a, c, g, or t
  misc_feature (40)..(40): n is a, c, g, or t
  misc_feature (49)..(49): n is a, c, g, or t
  misc_feature (64)..(64): n is a, c, g, or t
  misc_feature (84)..(84): n is a, c, g, or t
  misc_feature (90)..(91): n is a, c, g, or t
atgaacgacctggtgtccgagtaccancagtaccaggacnccacggccnacgaacaaggggagntcgaggaggaggagggcgangacganncgtagatgcccccgcgagacgggttagggaaagcggaggaggaaagcgagggggtggggggcttcccgggacgataacctggcagtggaaggaaagaagcatggtctactttaggtgtgcgctgggtctctggtgctcttcactgttgcctgtcacttttttttccttttttgtaatattgatgacatcaatgtaacatttgagatatttctgaattactgttgtaatggctaaaatcacataaacgtttgtgtcggaatggtgtcctctctttctcttcctttttctctttattaacgatttaaatgtaactttctgaacacattgcattgaattcttcctttaacaaaaagcaaaggcgtaggtaaaagctcaaatgaatttattctttcggtatggtaaaattgaaccaatcacagttaagatgagagatcaacctgagttttaa

SEQ ID NO: 158 - DNA, 295 bp, Artificial Sequence
  Synthetic
gtatttggtcccagttgggtacattttaaaatcctgattttggagacttaaaaccaggttaatggctaagaatgggtaacatgactcttgttggattgttattttttgtttgcaatggggaatttataagaagcatcaagtctctttcttaccaaagtcttgttaggtggtttatagttcttttggctaacaaatcattttggaaataaagattttttactacaaaaatgaaatttgtttggacttccacttgagacagtaaagagagtattagacacccagtaaaaactgccat

SEQ ID NO: 159 - DNA, 486 bp, Artificial Sequence
  Synthetic
  misc_feature (103)..(103): n is a, c, g, or t
acaggaaattgctacttaccgccgccttctggaaggagaagacgtaaaaactacagaatatcagttaagcaccctggaagagagagatataaagaaaaccagnaagattaagacagtcgtgcaagaagtagtggatggcaaggtcgtgtcatctgaagtcaaagaggtggaagaaaatatctaaatagctaccagaaggagatgctgctgaggttttgaaagaaatttggctataatcttatctttgctccctgcaagaaatcagccataagaaagcactattaatactctgcagtgattagaaggggtggggtggcgggaatcctatttatcagactctgtaattgaatataaatgttttactcagaggagctgcaaattgcctgcaaaaatgaaatccagtgagcactagaatatttaaaacatcattactgccatctttatcatgaagcacatcaattacaagctgtagaccacctaatatca

SEQ ID NO: 160 - DNA, 344 bp, Artificial Sequence
  Synthetic
gctgctgccgcggggaagcaggaactggccaagtacttcttggcagagctgctgtctgaacccaaccagacggagaatgatgccctggaacctgaagatctgtcccaggctgctgagcaggatgaaatgaggcttgagctgcagagatctgctaactcaaacccggctatggcaccccgagaacgcaaagctggctgcaagaatttcttctggaagactttcacatcctgttagctttcttaactagtattgtccatatcagacctctgatccctcgcccccacaccccatctctcttccctaatcctccaagtcttcagcgagacccttgcattagaaactga

SEQ ID NO: 161 - DNA, 482 bp, Artificial Sequence
  Synthetic
  misc_feature (117)..(117): n is a, c, g, or t
  misc_feature (126)..(126): n is a, c, g, or t
  misc_feature (128)..(128): n is a, c, g, or t
atacacccaggctatgcattgaagagttttccactgtatacatttttatccagatgaaggtatttttatattttgacaataggaaacagtgaccattttcagagtaatcaaatctgnaacaaatgnancatcttttagccaccaccaccctgttgcaattaagacaaccgtgggggaacacaccactttttactgttgaaaccaacacaacgttgaaatccaggcttatacgcagactccgattcctagagaactaaatttggctttagtgtgacgggatttgattaagcacttagtatagtcttttgaacacggaaatcctgttgtacttaaagctagcggacccgtgaacaactttgtcaggttcacgtcctataacggttaaaaaacacacacacacatacacaaaccgtttctatgagagattgatgaactttgtttaaaattttaaaaaaaggaacacgttctgtaaacgagtcgct

SEQ ID NO: 162 - DNA, 482 bp, Artificial Sequence
  Synthetic
  misc_feature (68)..(68): n is a, c, g, or t
  misc_feature (70)..(71): n is a, c, g, or t
gtagcaaactcatactggatcatttcagttaccttgaactaatagcacataatggttttttgttgttntnntttttaatgtagcccttacctggatatacatagtctgcaatcaccaaagtataatatcttgtaaggctatattttttaaagcatattttttcttgagcattaaattatcctaaatggtaatatattgtggataagtctgggcttattggacataatacatatttgggttggtactggttgaatccttcagttaactgctttgttgctttttgcaagattttttatcttaaacatgtcaggcatcttaagtcacctttatactgttttgttcctctgagtttctttcagtatgttatacaaatgccagacataacatgtagcagccatacttgcatggaaactgactacacatacataatactgcattttattgtaaggttttcacattaatacagcaattaccctgactaa

SEQ ID NO: 163 - DNA, 218 bp, Artificial Sequence
  Synthetic
taagatattgctcttctcttacctgcctagaggcagcgggatggactacatgacctcctggagtcccagccagttctgggagtctgttaagtccgggatgtgtgggagctttttaaggactgatcattggctctgaggacacttcaactagttagccttctatcttgaggtatataaactgtgaaaaagggtttctattctctctgaaagcacatgtc

SEQ ID NO: 164 - DNA, 465 bp, Artificial Sequence
  Synthetic
caatatctgacaccactttggactcaagagactcagtaacgtattatcctgtttatttagcttggttttagctgtgttctctctggataacccacttgatgttaggaacattacttctctgcttattccatattaatactgtgttaggtattttaagaagcaagttattaaataagaaaagtcaaagtattaattcttaccttctattatcctatattagcttcaatacatccaaaccaaatggctgttaggtagatttatttttatataagcatgtttattttgatcagatgttttaacttggatttgaaaaaatacatttatgagatgttttataagatgtgtaaatatagaactgtatttattactatagtaaaggttcagtaacattaaggaccatgataatgataataaaccttgtacagtggcatattctttgatttatattgtgtttctctgcccatt

SEQ ID NO: 165 - DNA, 237 bp, Artificial Sequence
  Synthetic
  misc_feature (27)..(27): n is a, c, g, or t
  misc_feature (46)..(46): n is a, c, g, or t
  misc_feature (136)..(136): n is a, c, g, or t
  misc_feature (186)..(186): n is a, c, g, or t
actattctctggcccgatttatcatancccttttctcctccaaatntttctcctctcaccttttctctgggacttaagctgctatatcccctcagagctcacaaatgcctttacattctttccctgacctcctgantttttttttcttttctcaaatgttacctacaaagacatgcctggggtaanccacccggctacctaattcctcagtaacctccatctaaaatctccaaggaa

SEQ ID NO: 166 - DNA, 145 bp, Artificial Sequence
  Synthetic
  misc_feature (61)..(67): n is a, c, g, or t
  misc_feature (70)..(70): n is a, c, g, or t
catgctgtcaccctgaatcataatcgctcctttgcacctctaaaaagatgtcccttacccnnnnnnnggngggctcctgagcctctgcgtaaggctgaacgtctcactgactgagctagtcttcttgttgctcgggtgcatttga

SEQ ID NO: 167 - DNA, 202 bp, Artificial Sequence
  Synthetic
actccttacagtctctagaattaaatgtactcatttagacaacatattaaatgcatattttagccactttagagaaacctcataggcacagagtttccaagattaattttaagaatatcttcacgaacttgaccctcctactccacattgcaacatttccatcagacagcatttcaattccagtattatgtatattgcaaat

SEQ ID NO: 168 - DNA, 440 bp, Artificial Sequence
  Synthetic
gaatgttttctctcaaatttcctctgacccatgggaatgaaaatacttaaatactgtaaagcatatctattaataattcccccaattttttaaactaacaaaatggaatgttaactgaatggaattaaacatagcaattgtcaagccatcaaaattatatatcgaaccacttaggcatgttacggggtaaagtcctcacccctgcatttgagaaatattttattatttttattttaaggcaaaacagtgtttactgtgatgggcagaacagccttttgtatctaggagtccatttgagttctttccaatatttccatatcgttcataaatgtgtctgggggcttccttgtttggaaaatatggatatcgctttgttttcctttgaaagtaattatatcttaagaacatagtatcatgaatggagtagacagggaggctgc

SEQ ID NO: 169 - DNA, 529 bp, Artificial Sequence
  Synthetic
gactgtctcatgtatctgcaagggccgaggaaattaatgacccaaggaggctatgatatggtccaaaaacttttcctggattttttccgtaggcggctgagccagaggccaactgcagaggaactggaacagaggaacattttgaaacctcggaatgaacaagaggaacaggaggagaagagagagatcaagaggaggctaacccgaaagctcagtcaaaggcccacggtggaagagcttcgggaaagaaagatcctcatccgcttcagtgactacgtggaggtggctgacgctcaggactatgaccgcagggcagataagccgtggacccgcctcaccgctgcagacaaagctgccatccgaaaggagctcaatgaattcaaaagcactgagatggaagttcatgaattgagtagacacttaacaaggtttcaccgaccttaacagtcgaattcctcttgagtgctatgctgtcttcaaaacataaatttataagaaccataagtgctggtatttattcacttcccca

SEQ ID NO: 170 - DNA, 431 bp, Artificial Sequence
  Synthetic
  misc_feature (178)..(178): n is a, c, g, or t
gcacacagagatttgagaaccattgttctgaatgctgcttccatttgacaaagtgccgtgataatttttgaaaagagaagcaaacaatggtgtctcttttatgttcagcttataatgaaatctgtttgttgacttattaggactttgaattatttctttattaaccctctgagttttngtatgtattattattaaagaaaaatgcaatcaggattttaaacatgtaaatacaaattttgtataacttttgatgacttcagtgaaattttcaggtagtctgagtaatagattgttttgccacttagaatagcatttgccacttagtattttaaaaaataattgttggagtatttattgtcagttttgttcacttgttatctaatacaaaattataaagccttcagagggtttggaccacatctctttggaaa

SEQ ID NO: 171 - DNA, 402 bp, Artificial Sequence
  Synthetic
  misc_feature (247)..(247): n is a, c, g, or t
gtctgcttcttgtaaaaacctgaacccagaaacaacacaatccgttgcaaacccaaaggacccaagtactttccaatctcagtcactctaggactctgcactgaatggctgacctgacctgatgtccattcatcccaccctctcacagttcggacttttctcccctctctttctaagagacctgaactgggcagactgcaaaataaaatctcggtgttctatttatttattgtcttcctgtaagacnttcgggtcaaggcagaggcaggaaactaactggtgtgagtcaaatgccccctgagtgactgcccccagcccaggccagaagacctcccttcaggtgccgggcgcaggaactgtgtgtgtcctacacaatggtgctattctgtgtcaaacacctct

SEQ ID NO: 172 - DNA, 529 bp, Artificial Sequence
  Synthetic
atcatccttccttagactgatgctttgactgaatcatcactagctatggcattaaaaggcctctcttctcatctggtgccaaaggttccgttgcagctttttacaaccatccggtgtggtttggaggatttgttttttttttttcccaacagaaaagaacagccattagaagaaggctcccattttctgatgttccgccccactgtgaagagtgtgctcgttttaaattcatgttgattcttgtaagcactggactgtcttcatcaagtatttccctacagaactcctcaagaaaacagagatcatttggctagagattgtctgagtgactccaagctactcactgtattggacgggagtagtaatttattttaaagataaagtgactaagtggggaaatttataaagctaaatattatatattttatttttcatacatgtttgaagtgcaaatctgtggatattccatttgtaggaccaagtcgacatgcccatcctgacattgtatgctacgagaactcttctgatg

SEQ ID NO: 173 - DNA, 511 bp, Artificial Sequence
  Synthetic
gtcagatgtttgagcagtggcctacaaatcagtaattttcggatgggagagtttctttacattgccgtggcatcttaaaagctatcttcatgtaaattgactgtactaggcctactggggatcagagttcccaagaaaggaaaccttttcttgtatctggattcaaatttatttccaatgtttcaagcgggaaacatgactctttattgtctgtaaatctaacattattacttttcctcttagaagaatattgtattgttagatgtttgttgagctggtaacatcgttgcaaccactgcaatatcttcgttagtaatctgtataatactttgtatacaagtactggtaagattgttattaaatgtagcttcagtcattaaattactatagcaaagtagtacttcttctgtaatatttacaatgtattaagcccacagtatattttatttcaatgtaattaaactgttaacttattcaaagagaaaacatctcatcatgtctattgtccaaa

SEQ ID NO: 174 - DNA, 450 bp, Artificial Sequence
  Synthetic
  misc_feature (34)..(34): n is a, c, g, or t
gctcgacgtatttcaaacatttcaaaatgctttnatctatgtttatcacattttaataccacagcacttataatgatgtcactacatatagaagctcaaagttaagggatttgctgaagactgtaaagttaatggaagaattgagacaaaaatccagtgtagctggccacttatccagggctttttctacttcatcacaaggaatgttttgaaagtgtctgctttttttatccttaaaattcacctgtcagggaggcattaaaaatttggaaatgtatgccagcaaaatgtgagctctgtattttttggcattcttatgtttgggtttaataagattaagaaaatgatactgggaattttctttttcctgaaactttgaatcaccctagtaagtcaaagtactaaaaaatgtactagatcattaagacttatgtgctcttactgattgaa

SEQ ID NO: 175 - DNA, 277 bp, Artificial Sequence
  Synthetic
taagctagttttctgaggtgttttcacacgtctttttatagttacttcatcttagatttttgaagggatatgacttcctactaaggatttagtttaccacaacaattctgactacaataagacattttgaggaggatatttggctactgtaaacatggctggtggaaaatcacgattgtggcttgatgtggcaagccgaaaccacttggctctggaaatctaagttcatactggtttaattaagctctctcctgacaacccccagaattaaatgaac

SEQ ID NO: 176 - DNA, 530 bp, Artificial Sequence
  Synthetic
aagtgtgcataatttcatttaacgttaaagaaatagatccaattcctttcttgcaaccaaaaataaataaaatacgttgcctcaatataaggtttgggctattctgtgtttctatagaagcaatctgtttttggtaaaatgtacttttaaggatccagtcatctgaagtattttatgtagagttagagatttcacaatattgactatacatatatttaaaatataaattatccagctgatgtttgaatttgtcttactttcctggccacctcgttgtcctattttataagctggggagttaactagcttaacaaaagatgcttagcttttgtaaaagaacaagtgtttcattttacaaagacactccaaatgatagttacttgattttctcgagacctttaactatggtgatgaataacaggacttgctttcaagccttaataaatgtaaaatgccttttaatgaagatacagctgagtgttttcctcatgaatctgaaccaattaccaatttgtgttccagtcttgatt

SEQ ID NO: 177 - DNA, 543 bp, Artificial Sequence
  Synthetic
tatactcactcaaggcagtgcaagatcttgaagtactttttagcagttaagtaatattgaattgtattgaatagtttacatagtttattctagtctttgaaaattactgaacatggacaatgtgcatgtcattgacatctgccttagaacttctgggacaatcctgattcgagagattctatcccattatttacatataccaaaaatactttgttaatttaatgtgttggcttcccaactcctgaacacgacacaattttattattagattttgtatggtgattttaggctatgaaaacatgatcattatatgtatatagatacatttttatttgttacaaatgtttgagcagctcactagcccacccctcctctattttgggtaagagaatttactaccttttttaactatgtagttgagagcaacatgtattttgttatttttagaatggtcagtatattgctataaaattttaaatgagactatgaaagttaaagtattctgattctggttaaattaacgaatatggttccaggccctgt

SEQ ID NO: 178 - DNA, 507 bp, Artificial Sequence
  Synthetic
atattcctacatcaagttactactgagagtaaatttattttgagttttatcccgtaagttctgttttgattttttttaaaaaacaaacccttttagtcactttaatcagaattttaaatgttcatgttacataccaaattataatatctaatggagcaatttgtcttttgctatattctccaagattatctcttaagaccatatgccccctgttttaatgtttcttacatcttgtttttactcatttctgactggacaaagttcttccaaacaattctgagaaacaaaaacacacacgcagaattaacaattcttttccctgtgcttcttatgtaagaatcctcctgtggcctctgcttgtacagaactgggaaacaacacttggttagtctcttttaagttacaaaaagccaattgatgtttcttattctttttaaattttaaatattttgttataaatactcacaggataccttatttccctagctatcatctcctgacttaatg

SEQ ID NO: 179 - DNA, 559 bp, Artificial Sequence
  Synthetic
  misc_feature (36)..(36): n is a, c, g, or t
  misc_feature (51)..(51): n is a, c, g, or t
  misc_feature (56)..(56): n is a, c, g, or t
acttctcagcaaataaatctcccttaagtaggaaanctagatttcatattngcttnctttgaattaacagcaactttccacaggtaaatctgttcttgcaaagatgtgagcagaatagttaaaaataatatttttatgtttcatggttctaaatggaagccataaatgcagtaaatactatctgttgtttaactactttaatcgtcattttttacattttcaagtttattaggttaagaaaaacagggcagccttggaaggcagctactacagaaaactgcagttttgcgttaaagataaagtagtattttcagctccctgaaaaaccattcctgctgaaactgctgtagaaattgtgaagctgcatgagtggagagtattgaatctgtggttatagtagttttctcaggtttgtttatcttgatgtttgatgcactgtgttttatagttattaaaattgagtaatattatttctatgcagtgttatgtgtcattggccttttgtgaatgtgcatgttttaaactgcaaattttaaacattttgtcctctaattgttat

SEQ ID NO: 180 - DNA, 147 bp, Artificial Sequence
  Synthetic
gacaggagggtgtccacatatgttaacatcagttggatctcctatagaagtttctgctgctctctttccttctccctgagctggtaactgcaatgccaacttcctgggcctttctgactagtatcacacttctaataaaatccacaa

SEQ ID NO: 181 - DNA, 504 bp, Artificial Sequence
  Synthetic
gaattcacacggtactcagaggcactgctggggaagtttgttggtctttattagataaatttccagagacctgtccataatacccaacagaacatgactgtttctttgaggaaagggttataatgtctgtggtgtacaagtcgtttttggtataacttctttcctgctgctgctgcttcccggcaaacatagttttcctatttcaggcagagtgcggtatattccaggaaacactgtttcctactcacttagcttacttctttgttgaatgcctcactaatggcaagtttcaagatgttttgggtgacaatgcacacatgctgggcaaaagggtgatggccagtggctggcagctgggccagcagaagctaggacatctgtgagttgtcattctcatctatccatgtccactggcctgccagcatccgccagtgccttgccagtgtgcacggtcccacactgtggcccctgagtcccctaatgtacacgctgcagccagaatgc

SEQ ID NO: 182 - DNA, 514 bp, Artificial Sequence
  Synthetic
  misc_feature (397)..(397): n is a, c, g, or t
gatcttttattaagcatactttcacagaataacttgtttaaactatattcatataaaaaagttaaacacgctttttttcctgcctaaaacacaaatacaactgccagtatgtatttttaatggaaccctattttataatggtacgttactgaatgtgtttcatatgcgtgaccgttaagatattatcatttaggtgaaggtttcaactcaaaaccacccaacccggtggttaacgatttaatacatataaccaaaccggcagcgtttagagttgggatatacatttaaacattttcctggttaaggttcccaagagagtgtaaaggttttagcagaaagcaaaatatcttgcatctttatggaagtttaaagcatgtttgcaaatattgcagcccantgaaagaatttgcatgtacaggaaagttgtggatggagacggtttgtggaattttaagtgctcattgtagtaaacttttgctttgtagatttgaaggtacagacttatacaggcaag

SEQ ID NO: 183 - DNA, 545 bp, Artificial Sequence
  Synthetic
gtgtcttaatggcatgtcagataatttgtttttccagagaagagagctcaaagaggaattgggaaaaataaattgaactctggaatcttctctctcaagtcctaaaatgaacaaacagatatgattgtgtttgagggaaatatgtccctagcaggaaaagaattcaaagaggttcaaagaatatgtcacttactcctacttgctataggaataaccttgctgataagaaaaaaagggacaatattggagaaactacctcttgtttaattgatctgtccaactctgagatcacttggtaactggtttcatgtgtatccaaaaatcagcatttggatttaagctttctgaatttggtagtttaagaaacagatttagtttttcagtggttttaactcatgtgaaataatgattttccaccagctctgatgcaaagagatataattttaatgaacgatttatccagcagtttgttccaggggttgcctctccttatctacggggattactttgtacatgcagataagttttcgcaaacctatttccat

SEQ ID NO: 184 - DNA, 521 bp, Artificial Sequence
  Synthetic
  misc_feature (82)..(82): n is a, c, g, or t
  misc_feature (176)..(176): n is a, c, g, or t
agaatcttctacctcataacttccttccaaaggcagctcagaagattagaaccagacttactaaccaattccaccccccacnaacccccttctactgcctactttaaaaaaattaatagttttctatggaactgatctaagattagaaaaattaattttctttaatttcattatgnacttttatttacatgactctaagactataagaaaatctgatggcagtgacaaagtgctagcatttattgttatctaataaagaccttggagcatatgtgcaacttatgagtgtatcagttgttgcatgtaatttttgcctttgtttaagcctggaacttgtaagaaaatgaaaatttaatttttttttctaggacgagctatagaaaagctattgagagtatctagttaatcagtgcagtagttggaaaccttgctggtgtatgtgatgtgcttctgtgcttttgaatgactttatcatctagtctttgtctatttttcctttgatgttcaagtcctagtctata

SEQ ID NO: 185 - DNA, 295 bp, Artificial Sequence
  Synthetic
  misc_feature (114)..(114): n is a, c, g, or t
  misc_feature (224)..(224): n is a, c, g, or t
accaatgaggttcctgaggtcacagtgttttccaagtctcccgtgacactgggtcagcccaacaccctcatctgtcttgtggacaacatctttcctcctgtggtcaacatcacntggctgagcaatgggcactcagtcacagaaggtgtttctgagaccagcttcctctccaagagtgatcattccttcttcaagatcagttacctcaccttcctcccttctgntgatgagatttatgactgcaaggtggagcactggggcctggatgagcctcttctgaaacactgggagcctg

SEQ ID NO: 186 - DNA, 414 bp, Artificial Sequence
  Synthetic
  misc_feature (157)..(157): n is a, c, g, or t
gtatcaaaatcttccaattatcatgctcacctgaaagaggtatgctctcttaggaatacagtttctagcattaaacaaataaacaaggggagaaaataaaactcaaggagtgaaaatcaggaggtgtaataaaatgttcctcgcattcccccccgcntttttttttttttttgactttgccttggagagccagagcttccgcattttctttactattctttttaaaaaaagtttcactgtgtagagaacatatatgcataaacataggtcaattatatgtctccattagaaaaataataattggaaaacatgttctagaactagttacaaaaataatttaaggtgaaatctctaatatttataaaagtagcaaaataaatgcataattaaaatatatttggacataacagactt

SEQ ID NO: 187 - DNA, 498 bp, Artificial Sequence
  Synthetic
tcctccttgttctactcatatatatctatcttatatagtttactattttacttctagagatagtacataaaggtggtatgtgtgtgtatgctactacaaaaaagttgttaactaaattaacattgggaaatcttatattccatatattagcatttagtccaatgtctttttaagcttatttaattaaaaaatttccagtgagcttatcatgctgtctttacatggggttttcaattttgcatgctcgattattccctgtacaatatttaaaatttattgcttgatacttttgacaacaaattaggttttgtacaattgaacttaaataaatgtcattaaaataaataaatgcaatatgtattaatattcattgtataaaaatagaagaatacaaacatatttgttaaatatttacatatgaaatttaatatagctatttttatggaatttttcattgatatgaaaaatatgatattgcatatgcatagttcccatgtt

SEQ ID NO: 188 - DNA, 404 bp, Artificial Sequence
  Synthetic
  misc_feature (374)..(374): n is a, c, g, or t
caagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagntgccgcaccagcccgaacaccattgaggga

SEQ ID NO: 189 - DNA, 419 bp, Artificial Sequence
  Synthetic
  misc_feature (249)..(249): n is a, c, g, or t
acagtgttctctaatgttacagatgagatgcgcattgccaaagaggagatttttggaccagtgcagcaaatcatgaagtttaaatctttagatgacgtgatcaaaagagcaaacaatactttctatggcttatcagcaggagtgtttaccaaagacattgataaagccataacaatctcctctgctctgcaggcaggaacagtgtgggtgaattgctatggcgtggtaagtgcccagtgcccctttggntgggattcaagatgtctggaaatggaagagaactgggagagtacggtttccatgaatatacagaggtcaaaacagtcacagtgaaaatctctcagaagaactcataaagaaaatacaagagtggagagaagctcttcaatagctaagcatctccttacagtcactaatat

SEQ ID NO: 190 - DNA, 466 bp, Artificial Sequence
  Synthetic
taagcattttagggtgggagggactattaagtaattttaagtgggtggggttatttagaatgttagaataatattatgtattagatatcgctataagtggacatgcgtacttacttgtaaccctttaccctataattgctatccttaaagatttcaaataaactcggagggaactgcagggagaccaacttatttagagcgaattggacatggataaaaaccccagtgggagaaagttcaaaggtgattagattaataatttaatagaggatgagtgacctctgataaattactgctagaatgaacttgtcaatgatggatggtaaattttcatggaagttataaaagtgataaataaaaacccttgcttttacccctgtcagtagccctcctcctaccactgaaccccattgcccctacccctccttctaactttattgctgtattctcttcactctatatttct

SEQ ID NO: 191 - DNA, 526 bp, Artificial Sequence
  Synthetic
gtgcctcctacgatgagtttcaggagagtaagtgtttcccttgtccagctgaaggatgccccaaaatggggcactatgctgaccaatttaaggggaaaacaagtgctgtggaacaaacctttttcctgaacacaggagagagtggtaactttactagttggagatataaggtatcagtcacactttctggaaaagagaaagtgaatgggtacatcaggattgctttgtatggaagtaatgaaaactcgaaacaatatgagattttcaaaggatccctcaaaccagatgcaagtcacacgtgtgctattgatgtggattttaatgttggaaaaatacagaaagttaaattcctctggaacaaacgtgggataaatctatctgagcccaaactgggggcttcccaaatcacagtgcaaagtggtgaagatgggactgagtataatttttgtagcagcgacactgtggaagaaaacgtcttgcaatctctttacccttgttaaaaacgtggtgcggctattgcggtaat

SEQ ID NO: 192 - DNA, 496 bp, Artificial Sequence
  Synthetic
tgtcaggagttctctattcctcccaactctgatcaagatcttaatgaatccctgccacctccacctgcagaactgttataaatattacaacttgctttttagctgatcttccatcctcaaatgactcttttttctttatatgttaacatatataaaatggcaactgatagtcaattttgatttttattcaggaactatctgaaatctgctcagagcctatgtgcatagatgaaacttttttttaaaaaaagttatttaacagtaatctatttactaattatagtacctatctttaaagtatagtacattttacatatgtaaatggtatgtttcaataatttaagaactctgaaacaatctacatatacttattacccagtacagttttttttcccctgaaaagctgtgtataaaattatggtgaataaacttttatgtttccatttcaaagaccagggtggagaggaataagagactaagtatatgcttcaagttt

SEQ ID NO: 193 - DNA, 548 bp, Artificial Sequence
  Synthetic
aactggaagtctgaagcgagccctgcacaatgccgaatgccagaagactgtcaccatctccaagccctgtggcaaactgaccaagcccaaacctcaagcagaatctaagaagaagaaaaaggaaggcaagaaacaggagaagatgctggattaaaagatgtcacctgtggaacataaaaaggacatcagcaaacaggatcagttaactattgcatttatatgtaccgtaggctttgtattcaaaaattatctatagctaagtacacaataagcaaaaacaaaaagaaaagaaaatttttgtagtagcgttttttaaatgtatactatagtaccagtaggggcttataataaaggactgtaatcttatttaggaagttgacttatagtacatgataaatgatagacaattgaggtaagttttttgaaattatgtgacattttacattaaattttttttacattttttgggcagcaatttaaatgttatgactatgtaaactacttctcttgttaggtaatttttttcacctagatttttttcccaattg

SEQ ID NO: 194 - DNA, 546 bp, Artificial Sequence
  Synthetic
tgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttat

SEQ ID NO: 195 - DNA, 387 bp, Artificial Sequence
  Synthetic
tcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgt

SEQ ID NO: 196 - DNA, 213 bp, Artificial Sequence
  Synthetic
attccagtctacttgagttagcataatacagaagtcccctctactttaacttttacaaaaaagtaacctgaactaatctgatgttaaccaatgtatttatttctgtggttctgtttccttgttccaatttgacaaaacccactgttcttgtattgtattgcccagggggagctatcactgtacttgtagagtggtgctgctttaattcataaa

SEQ ID NO: 197 - DNA, 532 bp, Artificial Sequence
  Synthetic
caggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatagagggtgttgcacgacatctagcag

SEQ ID NO: 198 - DNA, 263 bp, Artificial Sequence
  Synthetic
tcctccctgtctgcatctgtaggagacagagtcaccatcacttgccgggcaagtcagggcattagaaatgatttagcctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccttg

SEQ ID NO: 199 - DNA, 284 bp, Artificial Sequence
  Synthetic
aattgtgttgacgcagtctccaggcaccctgtctttgtctccaggggaaagagccaccctctcctgcagggccagtcagagtattagcagcagtttcttagcctggtaccagcagaaagttggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaatatggtagctcgc

SEQ ID NO: 200 - DNA, 384 bp, Artificial Sequence
  Synthetic
gttcatcaccatggactggacctggaggttcctctttgtggtggcagcagctacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagtgtcgatatttggagtggttca

SEQ ID NO: 201 - DNA, 476 bp, Artificial Sequence
  Synthetic
gctttttttgttgtcagctatcttaagaatcattaaatacacctgctttgggtaaaactctttgcaagcagtaattaacactagtaacagtgaaagcacaagatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctg

SEQ ID NO: 202 - DNA, 216 bp, Artificial Sequence
  Synthetic
gtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaa

SEQ ID NO: 203 - DNA, 344 bp, Artificial Sequence
  Synthetic
gttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaac

SEQ ID NO: 204 - DNA, 132 bp, Artificial Sequence
  Synthetic
ttcagtcccagccagaagaatctctgaaagcatattgtgaatgtatacaatgtagtacaaacaatctctgtgatgattaacagtatgtcaccacttcattttttaaaaaggatcacgtctaatgcccatttt

SEQ ID NO: 205 - DNA, 383 bp, Artificial Sequence
  Synthetic
tccagtgcttctagtaggagggctgaaagggagcaacttttcctccaatcctggaaattcgacacaattagatttgaactgctggaaatacaacacatgttaaatcttaagtacaagggggaaaaaataaatcagttattgaaacataaaaatgaataccaaggacctgatcaaatttcacacagcagtttccttgcaacactttcagctccccatgctccagaatacccacccaagaaaataataggctttaaaacaatatcggctcctcatccaaagaacaactgctgattgaaacacctcattagctgagtgtagagaagtgcatcttatgaaacagtcttagcagtggtaggttgggaaggagatagctgcaaccaaaa

SEQ ID NO: 206 - DNA, 211 bp, Artificial Sequence
  Synthetic
gaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaagg

SEQ ID NO: 207 - DNA, 416 bp, Artificial Sequence
  Synthetic
gagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtc

SEQ ID NO: 208 - DNA, 493 bp, Artificial Sequence
  Synthetic
gtgattcaaatgggttttccacgctagggcggggcacagattggagagggctctgtgctgacatggctctggactctaaagaccaaacttcactctgggcacactctgccagcaaagaggactcgcttgtaaataccaggatttttttttttttttgaagggaggacgggagctggggagaggaaagagtcttcaacataacccacttgtcactgacacaaaggaagtgccccctccccggcaccctctggccgcctaggctcagcggcgaccgccctccgcgaaaatagtttgtttaatgtgaacttgtagctgtaaaacgctgtcaaaagttggactaaatgcctagtttttagtaatctgtacattttgttgtaaaaagaaaaaccactcccagtccccagcccttcacattttttatgggcattgacaaatctgtgtatattatttggcagtttggtatttgcggcgtcagtctttttctgttgtaact

SEQ ID NO: 209 - DNA, 505 bp, Artificial Sequence
  Synthetic
gaaaccagccttaaacctgatttataaaccttatcaaatccgaatattagatcctttcattatcagaacagcagcttatgaactgcttcattttccaaaagtgtttcccaaaaatcagaaacctaaacacccaacaacaggaattattgccatcacattggcgttttacatatgtcacgaagttcacctagctggttttaaatacaacttttctgacctcaagagtcctttgcactactatgggaatgccaccatgtctttgatgaataagaacgcgtatcacaatgtgactgcagagcagctctttttgaaggacattatagaaaaaaacctcgtaatcaacttgactcaagattgactctacagactcagaagatgatgctaacagtgttagttttatttttgtactgcaatttttagtttaaaatatgttggatgcactcgtcaaataattatgtatactgtctgttgctgcctggtgattcataaccaccagcttaatttc

SEQ ID NO: 210 - DNA, 243 bp, Artificial Sequence
  Synthetic
atatcaagtcttgactccctacttcccgtcattcctcacaggacagaagcagagtgggtggtggttatgtttgacagaaggcattaggttgacaacttgtcatgattttgacggtaagccaccatgattgtgttctctgcctctggttgaccttacaaaaaccattggaactgtgactttgaaaggtgctcttgctaagcttatatgtgcctgttaatgaaagtgcctgaaagaccttcctta

SEQ ID NO: 211 - DNA, 162 bp, Artificial Sequence
  Synthetic
gccccagatctaagcggtgcggtaatctgagtacttgcatgctgggcacatacacgcaggacttcaacaagtttcacacgttcccccaaactgcaattggggttggagcacctggaaagaaaagggatatgtccagcgacttggagagagaccatcgccctc

SEQ ID NO: 212 - DNA, 382 bp, Artificial Sequence
  Synthetic
tcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtt

SEQ ID NO: 213 - DNA, 550 bp, Artificial Sequence
  Synthetic
gaaagtgtcctatttgtaggagtacaatcaagggtacagttcgtacatttctttcatgaagaagaaccaaaacatcatctaaactttagaattaatttattaaatgtattataactttaacttttatcctaatttggtttccttaaaatttttatttatttacaactcaaaaaacattgttttgtgtaacatatttatatatgtatctaaaccatatgaacatatattttttagaaactaagagaatgataggcttttgttcttatgaacgaaaaagaggtagcactacaaacacaatattcaatcaaaatttcagcattattgaaattgtaagtgaagtaaaacttaagatatttgagttaacctttaagaattttaaatattttggcattgtactaatacctggttttttttttgttttgtttttttgtacagacagggcagcatactgagaccctgcctttaaaaacaaacagaacaaaaacaaaacaccagggacacatttctctgtcttttttgatcagtgtcctatacatcgaaggtgtgcata

SEQ ID NO: 214 - DNA, 537 bp, Artificial Sequence
  Synthetic
gagtctgagcacgagttgcagccagggccagtgggagggggtctgggccagtgcaccttccaaggccccatccattagtttccactgcctcgtgtgacatgaggcccattcttcactctttgaagagagcagtcagtattgttagtagtgagtttctgttctattggatgactttgagatttatctttgtttcctgttggaattgttcaaatgttccttttaacggatggttgaatgaacttcagcatccaagtttatgaatgacagtagtcacacatagtgctgtttatatagtttaggagtaagagtgttgttttttattcagattgggaaatccattccattttgtgaattgtgacaaataacagcagtggaaaaagtatgtgcttagaattgtgaaagaattagcagtaaaatacatgagataaagacctcaagaagttaaaagatacttaattcttgccttatacctcactctattctgtaaatttgaaaaaaaagcatggatacctggatatccttggcttctttgagaat

SEQ ID NO: 215 - DNA, 255 bp, Artificial Sequence
  Synthetic
gttcctgaaatcctgagtgttgcctgccagtcgccatgagaacttcctaccttctgctgtttactctctgcttacttttgtctgagatggcctcaggtggtaactttctcacaggccttggccacagatctgatcattacaattgcgtcagcagtggagggcaatgtctctattctgcctgcccgatctttaccaaaattcaaggcacctgttacagagggaaggccaagtgctgcaagtgagctgggagtgacc

SEQ ID NO: 216 - DNA, 244 bp, Artificial Sequence
  Synthetic
gcttccactggaaaatccagtagttctgaacagctttcactttgctgctgactgctgcacctcctacatctcacaaagcatcccgtgttcactcatgaaaagttattttgaaacgagcagcgagtgctccaagccaggtgtcatattcctcaccaagaaggggaggcaagtctgtgccaaacccagtggtccgggagttcaggattgcatgaaaaagctgaagccctactcaatataataataa

SEQ ID NO: 217 - DNA, 499 bp, Artificial Sequence
  Synthetic
gaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcctttgcatggagtgaccatagctccttctctcttacattgaatgtagagaatgtagccattgtagcagcttgtgttgtcacgcttcttcttttgagcaactttcttacactgaagaaaggcagaatgagtgcttcagaatgtgatttcctactaacctgttccttggataggctttttagtatagtattttttttttgtcattttctccatcagcaaccagggagactgcacctgatggaaaagatatatgactgcttcatgacattcctaaactatcttttttttattccacatttacgtttttggtggagtcccttttgcatcat

SEQ ID NO: 218 - DNA, 445 bp, Artificial Sequence
  Synthetic
gcataggagataaaacccccactgagatgctctcatgcctcagctggacccaccgtgtagacacacgacatgcaagagttgcagcggctgctccaactcactgctcaccctctctgtgagcagaaagaaccctactgacatgcatggttaacttcctcatcagaactctgcccttcccttctgttcttttgtgctttcaaataactaacacgaacttccagaaaattaacatttgaacttagctgtaattctaaactgacctttccccgtactaacgtttggtttccccgtgtggcatgcttttctgagccttcctactttaaagcatggaacatgcaggtgatttgggaagtgtagaaagacctgagaaaacgagcctgtttcagaggaacatcgtcacaacgaatacttctggaagcttaacaaaactaaccctgctgtcctt

SEQ ID NO: 219 - DNA, 311 bp, Artificial Sequence
  Synthetic
aatgaatgttctcctgggcagcgttgtgatctttgccaccttcgtgactttatgcaatgcatcatgctatttcatacctaatgagggagttccaggagattcaaccaggatgtttctacacctgtgggttatgacaaagacaactgccaaagaatcttcaagaaggaggactgcaagtatatcgtggtggagaagaaggacccaaaaaagacctgttctgtcagtgaatggataatctaatgtgcttctagtaggcacagggctcccaggccaggcctcattctccctggcctctaatagtcaatgattgt

SEQ ID NO: 220 - DNA, 536 bp, Artificial Sequence
  Synthetic
aacctcactcgactgacattttttttcttgattcctttgttcatcatgattgggtgctacttggtcattattcataatctccttcacggcaggacgtctaagctgaaacccaaagtcaaggagaagtccataaggatcatcatcacgctgctggtgcaggtgctcgtctgctttatgcccttccacatctgtttcgctttcctgatgctgggaacgggggagaacagttacaatccctggggagcctttaccaccttcctcatgaacctcagcacgtgtctggatgtgattctctactacatcgtttcaaaacaatttcaggctcgagtcattagtgtcatgctataccgtaattaccttcgaagcatgcgcagaaaaagtttccgatctggtagtctacggtcactaagcaatataaacagtgaaatgttatgaataataaggttctttcatttcaatcccatcaaaattcacttcactaactactctggcgtcaatggatattctgtataatactatcaagtcccttttctctt

SEQ ID NO: 221 - DNA, 512 bp, Artificial Sequence
  Synthetic
  misc_feature (443)..(443): n is a, c, g, or t
acagcaagcctatgtagttcaattaatatataaggaaaaggaaggtctttcttcatgatacaagcattataaagtttttactgtagtagtcaattaatggatatttccttgttaataaaattttgtgtcataatttacaaattagttctttaaaaattgttgttatatgaattgtgtttctagcatgaatgttctatagagtactctaaataacttgaatttatagacaaatgctactcacagtacaatcaattgtattataccatgagaaaatcaaaaaggtgttcttcagagacattttatctataaaattttcctactattatgttcattaacaaacttctttatcacatgtatcttctacgtgtaaaacatttctgatgattttttaacaaaaaatatatgaatttcttcatttgctcttgcatctacattgctataanggatataaaatgtggtttctatattttgagatgttttttccttacaatgtgaactcatcgtgatcttgg

SEQ ID NO: 222 - DNA, 555 bp, Artificial Sequence
  Synthetic
ttaattagttgtgtactctggcctctgtcatatcttcacaatggtgctcatttcatggggtattatccattcagtcatcgtaggtgatttgaaggtcttgatttgttttagaatgatgcacatttcatgtattccagtttgtttattacttatttggggttgcatcagaaatgtctggagaataattctttgattatgactgttttttaaactaggaaaattggacattaagcatcacaaatgatattaaaaattggctagttgaatctattgggattttctacaagtattctgcctttgcagaaacagatttggtgaatttgaatctcaatttgagtaatctgatcgttctttctagctaatggaaaatgattttacttagcaatgttatcttggtgtgttaagagttaggtttaacataaaggttattttctcctgatatagatcacataacagaatgcaccagtcatcagctattcagttggtaagcttccaggaaaaaggacaggcagaaagagtttgagacctgaatagctcccagatttcagtcttttc

SEQ ID NO: 223 - DNA, 446 bp, Artificial Sequence
  Synthetic
gccaagcggaccagagctgtgcagcccctcaggctacctagcaacaaggcccaggtgaagccagggcagacatgcagtgtggccggctgggggcagacggcccccctgggaaaacattcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagcca

SEQ ID NO: 224 - DNA, 399 bp, Artificial Sequence
  Synthetic
ttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctct

SEQ ID NO: 225 - DNA, 493 bp, Artificial Sequence
  Synthetic
agacagaatccatctcgttgctctgtttcccttagtaatgttgaggcaagaagatttttcaacaaggagtttctaagtaaacccaaagcatagttcatgtactggaaatggcagcagtttctgatgctgaggcagtttgcaatcccatgacaactggatttaaaagtacagtacagatagtcgtactgatcatgagagactggctgatactcaaagttgcagttacttagctgcatgagaataatactattataagttaggtgacaaatgatgttgattatgtaaggatatacttagctacattttcagtcagtatgaacttcctgatacaaatgtagggatatatactgtatttttaaacatttctcaccaactttcttatgtgtgttctttttaaaaattttttttcttttaaaatatttaacagttcaatctcaataagacctcgcattatgtatgaatgttattcactgactagatttattcataccat

SEQ ID NO: 226 - DNA, 485 bp, Artificial Sequence
  Synthetic
ggagctgctggttgcaattgttctctgtgttcgagacaaaccagcctattttgcttatagattatatagtgcaattcatgactttggtttccataataaaactgtaatcaggattctcattgccagaagtgaaatagacctgctgaccataaggaaacgatacaaagagcgatatggaaaatccctatttcatgatatcagaaattttgcttcagggcattataagaaagcactgcttgccatctgtgctggtgatgctgaggactactaaaatgaagaggacttggagtactgtgcactcctctttctagacacttccaaatagagattttctcacaaatttgtactgttcatggcactattaacaaaactatacaatcatatttcctcttctatccttgaaattattctaagccaaagaaaactatgaatgaaagtatatgatactgaatttgcctactatcctgaatttgcctactatctaa

SEQ ID NO: 227 - DNA, 386 bp, Artificial Sequence
  Synthetic
gtagcatgttgtcagtggccaaggctacacagaaggctccctgctgcccggagcaggtacatccaccagagcaaagggaaccacttttattttgcatgagtttggtaactgattactctcccctcaaagaaaagacattcaggtgtttctcaacgacatcttctgtccagcaagctcggtttgaatacgtcacttaccagtgccattgcaggacccaaattcacagttcataaaagatgtgaccactacatgtaaaaatagcattctacttgatcttacagtatgtatgtatgtatgtatggagacatatgtgtgtgtgtggatgtctacatggttaatggaaagcactgtgctctgaagtggatcagtctcaagtgtctggtaac

SEQ ID NO: 228 - DNA, 280 bp, Artificial Sequence
  Synthetic
ccatccaggtttaaccctgcaagctgtttccacatcagaactcccttcaaacacaaagattgctgtgaaaacgaaaatgtgtagacgcactctcaaaaactgcacggacatacaaaatcaatcttttcctttgatcttgtggctaaaccagcatttctgtgtttgagagatttcctgttaggtgcttcgtctgaaagtgaactctcataattcaaattgtataaaataaagctacatttctaagagcttggtgtagggcaattggaataatgtcctgtta

SEQ ID NO: 229 - DNA, 496 bp, Artificial Sequence
  Synthetic
gaggttccccgtgtgtaggtagatctggtctttagaggcagatagataggtcagtgcaaatactctggtccatgggccatatgaaaaggctaagcttcactgtaaaataataactgggaattctggattgtgtatgggtgttggtgaacttggttttaattagtgaactgctgagagacagagctattctccatgtactggcaagacctgatttctgagcatttaatatggatgccgtgggagtacaaaagtggagtgtggcctgagtaatgcattatgggtggtttaccatttcttgaggtaaaagcatcacatgaacttgtaaaggaatttaaaaatcctactttcataataagttgcataggtttaataatttttaattatatggcttgagtttaaattgtaataggcgtaactaattttaactctataatgtgttcattctggaataatcctaaacatatgaattatgtttgcatgttcacttccaagagcc

SEQ ID NO: 230 - DNA, 454 bp, Artificial Sequence
  Synthetic
gtagctctgatgagaatggggtcccagatggctcaggctgtgacctccctgggcaccaccctccccaggctgggtgtggaggagttggggccccctgccttcaggaggcttgtagtttagaagggaagtaggcattaccatagacgactcctagaggacagtgctatgtaaaaatgtgtgtctataaatgtttatcatgcatgtattctagagctcattcatttattcaacaaacatttggtgagcacctatttcggttcgagaaacttcatttatctcctataattggcaaacttaaaaatgcagcagaaacttacattccaaccttagagactcatagtgagcacaaggaaagttttgccctgagattcatggttatggctgggtaccaccaaatagaagaatggcttaggggagtgccccttcactgagatgtgtttctttgttgaacttt

SEQ ID NO: 231 - DNA, 294 bp, Artificial Sequence
  Synthetic
cccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaacctatgaccgtgcagagggagccccgagtccgagtcaagcattgtga

SEQ ID NO: 232 - DNA, 565 bp, Artificial Sequence
  Synthetic
gtttagtagcctcaattctccattaattaaaagtgtgggctgggcgtgggggctcatgcctgtaatcccagcactttgggaggccgaggtgggcagatcacctgaggtcaggagttcaagaccagcctggccaacatggtgaaaccccgtctctacaaaaatacaaaaattagccaggcgtgatggcaggtgcctgtaatcctagctacttggcaggctaacgcaggagaatcacttgaccgggagacagaggttgcagtgagctgagatcgtacctattgcactccatcctggatgaaagagccagactctgtctcaaaacaaacaaaaaagcgtggggacttctggggacagacaaggtgcctgttatatatttactcagtctttgccctgaatggtctcagcttgagaccatttcaaactggagagaagcaagccagccaatagaatggggtgatttacagggatttctgtttactgtcaaaatatttctcatctgcactatgtttccatttgtggtcctgaaggaaattcttataactcaacatttgtctggtcttataag

SEQ ID NO: 233 - DNA, 497 bp, Artificial Sequence
  Synthetic
tggcctcgtcaatgctgtgttatgaacaatcttaaagaacctctcaacctggaggcttgtaaactaggcgtgcctggtttttatcacaatcagggctgctatgaactgatctctggtccaatgaaccgacacgcctggggggttgcctggtttggatttgccattctctgctggactttttgggttctcctgggtaccatgttctactggagcagaattgaatattaagcataaagtgttgccaccatacctccttccccgagtgactctggatttggtgctggaaccagctctctcctaatattccacgtttgtgccccacactaacgtgtgtgtcttacattgccaagtcagatggtacggacttcctttaggatctcaggcttctgcagttctcatgactcctacttttcatcctagtctagcattctgcaacatttatatagactgttgaaaggagaatttgaaaaatgcataataactacttccatccctgc

SEQ ID NO: 234 - DNA, 438 bp, Artificial Sequence
  Synthetic
  misc_feature (56)..(56): n is a, c, g, or t
  misc_feature (229)..(229): n is a, c, g, or t
  misc_feature (406)..(406): n is a, c, g, or t
agccctgttgatcactggacataaagcttcttttccccaataattcttctttactnaaaatagtcaggatctttatctacagatgtactctccaggttacctgtgatgatagccccctaatgtcctgctagaaaagtctccaagcagagatgacattacttctgaatgctcataaaccacaccatgaaataaaagctctttgttgttttaagattgtgaagtgtcgttnatgggtccccacagatggtccctgctggactcacctggaatctctccacagccatacccactcatcactatcattgagacctgcacatcttaatagaaatattataaacatcgaaaatcatgacttacctagaagttcgcttgtaactaatgaaattaaacaaatgtgttgcctttntgtcatgtgtttctctcctgtgacatttcaaa

SEQ ID NO: 235 - DNA, 531 bp, Artificial Sequence
  Synthetic
aaggcaagttccgttatcggcgcgtggctgaaggcacccaggtgcttgagttgcccttcaaaggtgatgacatcaccatggtcctcatcttgcccaagcctgagaagagcctggccaaggtggagaaggaactcaccccagaggtgctgcaggagtggctggatgaattggaggagatgatgctggtggtccacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacatgcctttcctggtttttataagagaagttcctctgaacactattatcttcatgggcagggtagccaacccttgtgtta

SEQ ID NO: 236 - DNA, 556 bp, Artificial Sequence
  Synthetic
gtgtggctgatctgaagtcagtcggaatttgtaaacagggtagcaaacaagatatttttcttccatgtatacaataatttttttaaaaagtgcaatttgcgttgcagcaatcagtgttaaatcatttgcataagatttaacagcattttttataatgaatgtaaacattttaacttaatggtacttaaaataatttaaaagaaaaatgttaacttagacattcttatgcttcttttacaactacatcccattttatatttccaattgttaaagaaaaatatttcaagaacaaatcttctctcaggaaaattgcctttctctatttgttaagaatttttatacaagaacaccaatataccccctttattttactgtggaatatgtgctggaaaaattgcaacaacactttactacctaacggatagcatttgtaaatactctaggtatctgtaaacactctgatgaagtctgtatagtgtgactaacccacaggcaggttggtttacattaatttttttttttgaatgggatgtcctatggaaacctatttcaccag

SEQ ID NO: 237 - DNA, 543 bp, Artificial Sequence
  Synthetic
gtgttgtcactcactgtgtattaccagtatttacttgctttcttgatttcaccaaaaccaaatttaatttaaaggaccacattaatttttcaaagggaaagagacaattaattgtacataatgtatacacacacaaaaaaaaaaatacctgtagaaatattattccagcatagcaggaaaacaaacaaaagtattggactgtcggaggtgagcctgtgcgtctgtaaccctttgtgactcctgagcgtgcgctgtcttctaggttaactcacgaagtacattctctgtcttactgatactgtaggttcacccatttttttttaatttcctcgcaaataacaagacccacagaagtgactctagctacttaatggttctgttcttttatatgcagcaaacacaccgtccatttctgaagaggcttcggcctgaaggcattttccaatgatgttagtgcacaaaacgctttaaattagactggaactgccagaatcaaatgtaaatgaggaatttctcgtacccctactgcatggtatcgatttt

SEQ ID NO: 238 - DNA, 382 bp, Artificial Sequence
  Synthetic
gtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttg

SEQ ID NO: 239 - DNA, 520 bp, Artificial Sequence
  Synthetic
ttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgt

SEQ ID NO: 240 - DNA, 286 bp, Artificial Sequence
  Synthetic
ctcctgattctcatgaaataccctgagatcgaagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaagggcacagtcgtagtgccaactctggactctgttttgtatgacaaccaagaatttcctgatccag

SEQ ID NO: 241 - DNA, 246 bp, Artificial Sequence
  Synthetic
agttcaagtacagtgactatttcaagccattttccacaggaaaacgagtgtgtgctggagaaggcctggctcgcatggagttgtttcttttgttgtgtgccattttgcagcattttaatttgaagcctctcgttgacccaaaggatatcgacctcagccctatacatattgggtttggctgtatcccaccacgttacaaactctgtgtcattccccgctcatgagtgtgtggaggacaccctgaac

SEQ ID NO: 242 - DNA, 528 bp, Artificial Sequence
  Synthetic
gagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgag

SEQ ID NO: 243 - DNA, 520 bp, Artificial Sequence
  Synthetic
ggaagcgatttgcgatgttcacctccacgatatttgctgtggttggattcttgggagctggatactcgtttatcatctcagccatttcaatcaacaagggtcctaaatgcctcatggccaatagtacatggggctaccccttccacgacggggattatctcaatgatgaggccttatggaacaagtgccgagagcctctcaatgtggttccctggaatctgaccctcttctccatcctgctggtcgtaggaggaatccagatggttctctgcgccatccaggtggtcaatggcctcctggggaccctctgtggggactgccagtgttgtggctgctgtgggggagatggacccgtttaaacctccgagatgagctgctcagactctacagcatgacgactacaatttcttttcataaaacttcttctcttcttggaattattaattcctatctgcttcctagctgataaagcttagaaaaggcagttattccttctttccaaccagctttgctcgagtta

SEQ ID NO: 244 - DNA, 492 bp, Artificial Sequence
  Synthetic
ggcataggaacagttttctcttcatatattatgattatgaataataggaaagttgtattaattcagtatttgtcattatgcagtattttaatacctacataagtctattccattattatattttgcgcttggcatatttattcctttatgcttttaatcactaacatattttacttaagagtataaaactatgctaaataaattgtattgtatatggaatatgcttgccattatgaagaacagctgggtataccattatttcaagtaataaatctgaattctattagtttaaaaattgtaaaattcaagttaaatgacgtgtatgatatataagctgaacacattttctatagcccttaatttagttgtgttaatttttcaaggtgatgtatcaacagctttttttttttgcatttgttttttcaatgtgtttacattgtatgaattgagctttttgcccacagattcttgatttgtagttgtttggcagga

SEQ ID NO: 245 - DNA, 529 bp, Artificial Sequence
  Synthetic
gaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagctaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactgatgtgtttgataattagtttagtttgtggcttcatggaa

SEQ ID NO: 246 - DNA, 499 bp, Artificial Sequence
  Synthetic
  misc_feature (319)..(319): n is a, c, g, or t
ctactgacctgtgctttctggtgtggagtccagggctgctaggaaaaggaatgggcagacacaggtgtatgccaatgtttctgaaatgggtataatttcgtcctctccttcggaacactggctgtctctgaagacttctcgctcagtttcagtgaggacacacacaaagacgtgggtgaccatgttgtttgtggggtgcagagatgggaggggtggggcccaccctggaagagtggacagtgacacaaggtggacactctctacagatcactgaggataagctggagccacaatgcatgaggcacacacacagcaaggntgacgctgtaaacatagcccacgctgtcctgggggcactgggaagcctagataaggccgtgagcagaaagaaggggaggatcctcctatgttgttgaaggagggactagggggagaaactgaaagctgattaattacaggaggtttgttcaggtcccccaaaccaccgtcagatttgatg

SEQ ID NO: 247 - DNA, 528 bp, Artificial Sequence
  Synthetic
gtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcagggggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaa

SEQ ID NO: 248 - DNA, 284 bp, Artificial Sequence
  Synthetic
ctgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtc

SEQ ID NO: 249 - DNA, 543 bp, Artificial Sequence
  Synthetic
atatcttttcttcctctaaagagtgtacaaggtggggtatgccaaggtatcaaaacaatatatgtgagtgtaatttaaactgtggaatatcaactgtactatggacgtgtttgtatcatttagatgtcattttaaatatttacattttagcaagacttttaaaaaggactcatttcatttcaaagtgcaaattgtttgccaggcttctggcaaatggttctttcaactgtgaacttatagtgtacatatctgtatatttataaatattatatatattcatacatccttcagtttaaaggtacattgtacagtctgtagttaggaggtatagcctatagcttatgttaaatggttgaaatggttctttttatagaaagtcaaacacagatgttacaggattttgtgtttggtttgtcatttttttattttttattttgactattgcatgagtaattaattccagatcttttgtattcacttctgtattttatgtttggttgaggggtgcttttagttgtgtggcatttgtattcattgatcttt

SEQ ID NO: 250 - DNA, 532 bp, Artificial Sequence
  Synthetic
aaatgatacactactgctgcagctcacaaacacctctgcatattacatgtacctcctcctgctcctcaagagtgtggtctattttgccatcatcacctgctgtctgcttagaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataacggttttcaaaccagtgggcacacagagaacctcactctgtaataacaatgaggaatagccacggcgatctccagcaccaatctctccatgttttccacagctcctccagccaacccaaatagcgcctgctatagtgtagacatcctgcggcttctagccttgtccctctcttagtgttctttaatcagataactgcctggaagcctttcattttacacgccctgaagcagtcttctttgcta

SEQ ID NO: 251 - DNA, 476 bp, Artificial Sequence
  Synthetic
gaaaaaccagccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagattttgattgaaagtcctagggtgattctatttctgctgtgatttatctgctgaaagctcagctggggttgtgcaagctagggacccattcctgtgtaatacaatgtctgcaccaatgct

SEQ ID NO: 252 - DNA, 432 bp, Artificial Sequence
  Synthetic
tagtctaattgaatcccttaaactcagggagcatttataaatggcaaatgcttatgaaactaagatttgtaatatttctctctttttagagaaatttgccaatttactttgttatttttccccaaaaagaatgggatgatcgtgtatttatttttttacttcctcagctgtagacaggtccttttcgatggtacatatttctttgcctttataatcttttatacagtgtcttacagagaaaagacataagcaaagactatgaggaatatttgcaagacatagaatagtgttggaaaatgtgcaatatgtgatgtggcaaatctctattaggaaatattctgtaatcttcagacctagaataatactagtcttataataggtttgtgactttcctaaatcaattctattacgtgcaatacttcaatacttcat

SEQ ID NO: 253 - DNA, 571 bp, Artificial Sequence
  Synthetic
agagagacacagctgcagaggccacctggattgcgcctaatgtgtttgagcatcacttaggagaagtcttctatttatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtaagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaagatactgccttgtttaatgttaattatgcagtgtttccctc

SEQ ID NO: 254 - DNA, 499 bp, Artificial Sequence
  Synthetic
agagattcattgcagctcagcatggctcagaccagctcatacttcatgctgatctcctgcctgatgtttctgtctcagagccaaggccaagaggcccagacagagttgccccaggcccggatcagctgcccagaaggcaccaatgcctatcgctcctactgctactactttaatgaagaccgcgagacctgggttgatgcagatctctattgccagaacatgaattcgggcaacctggtgtctgtgctcacccaggccgagggtgcctttgtggcctcactgattaaggagagtggcactgatgacttcaatgtctggattggcctccatgaccccaaaaagaaccgccgctggcactggagcagtgggtccctggtctcctacaagtcctggggcattggagccccaagcagtgttaatcctggctactgtgtgagcctgacctcaagcacaggattccagaaatggaaggatgtgccttgtgaagacaagttctcct

SEQ ID NO: 255 - DNA, 336 bp, Artificial Sequence
  Synthetic
gcaacaacagattctcttggctcatatttcttttctttctcatcttgatgatgatcgtcgtcatcaagaatttaatgattaaaatagcatgcctttctctctttctcttaataagcccacatataaatgtactttttcttccagaaaaattctccttgaggaaaaatgtccaaaataagatgaatcacttaataccgtatcttctaaatttgaaatataattctgtttgtgacctgttttaaatgaaccaaaccaaatcatactttttctttgaatttagcaacctagaaacacacatttctttgaatttaggtgatacctaaatccttcttatgt

SEQ ID NO: 256 - DNA, 410 bp, Artificial Sequence
  Synthetic
tggtttgggctccacattgcaatgttcaatgccacgtgctgctgacaccgaccggagtactagccagcacaaaaggcagggtagcctgaattgctttctgctctttacatttcttttaaaataagcatttagtgctcagtccctactgagtactctttctctcccctcctctgaatttaattctttcaacttgcaatttgcaagggttacacatttcactgtgatgtatattgtgttgcaaaaaaaaaaaagtgtctttgtttaaaattacttggtttgtgaatccatcttgctttccccattggaactagtcattaacccatctctgaactggtagaaaaacatctgaagagctagtctatcagcatctgacaggtgaattggatggttctcagaaccatttcacccag

SEQ ID NO: 257 - DNA, 475 bp, Artificial Sequence
  Synthetic
gagagctcgctttgagtgactgggttttgtgattgcctctgaagcctatgtatgccatggaggcactaacaaactctgaggtttccgaaatcagaagcgaaaaaatcagtgaataaaccatcatcttgccactaccccctcctgaagccacagcagggtttcaggttccaatcagaactgttggcaaggtgacatttccatgcataaatgcgatccacagaaggtcctggtggtatttgtaactttttgcaaggcatttttttatatatatttttgtgcacatttttttttacgtttctttagaaaacaaatgtatttcaaaatatatttatagtcgaacaattcatatatttgaagtggagccatatgaatgtcagtagtttatacttctctattatctcaaactactggcaatttgtaaagaaatatatatgatatataaatgtgattgcagcttttcaatgttagccacagt

SEQ ID NO: 258 - DNA, 425 bp, Artificial Sequence
  Synthetic
tcatcaacaatgagctttcccatttcttagaggaaatcaaagagcaggaggttgtggacaaagtcatggaaacactggacaatgatggagacggcgaatgtgacttccaggaattcatggcctttgttgccatggttactactgcctgccacgagttctttgaacatgagtgagattagaaagcagccaaacctttcctgtaacagagacggtcatgcaagaaagcagacagcaagggcttgcagcctagtaggagctgagctttccagccgtgttgtagctaattaggaagcttgatttgctttgtgattgaaaaattgaaaacctctttccaaaggctgttttaacggcctgcatcattctttctgctatattaggcctgtgtgtaagctgactggccccagggactcttgttaacagtaact

SEQ ID NO: 259 - DNA, 548 bp, Artificial Sequence
  Synthetic
ccatacgccgtgaaatgtccacttttgcttctgtcggaactcattgctgaaggacagtacttggttgggtcagatttatttgtttgattttcatatcccgtgaaagtttttaattcatatttttccttatagggaaaaatgcaaaaaagaaacaataaagaaagaaatattaactactgtagaactgattttacaaattaatatttgtgctttgaaaaaaagtttctatttagttatttaagaagaatgagaaggccaatagttttagattattttatctggtatggtgctaatattttatttgaaaaaagttactgcaacttaacttaaaattgctaacgttttttcttcttttaaaaatacaattattgtatattaattatagcaatgtgattttgtaggttattttatatttgagttgtgattgaaagtatgttgtatatggtattgtgagatgatttgtacttggaagcattcacaaagtagcaccaaataaattacactttattctttaatgtcattgtcaatctacttttaaccaatattca

SEQ ID NO: 260 - DNA, 555 bp, Artificial Sequence
  Synthetic
gcagatttcttgcttcatatgacaaagcctcaattactaattgtaaaaactgaactattcccagaatcatgttcaaaaaatctgtaatttttgctgatcgaaagtgcttcattgactaaacagtattagtttgtggctataaatgattatttagatgatgactgaaaatgtgtataaagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaacacctgtgttaatgctttgttaatgcttttcccactctcatttgttaatgctttcccactctcgggaaggatttgcattttgagctttatctctaaatgtgacatgca

SEQ ID NO: 261 - DNA, 526 bp, Artificial Sequence
  Synthetic
actggatgcgttaataacccatgttttaccttttgaaaaaataaatgaaggatttgacctgcttcactctgggaaaagtatccgtaccgtcctgacgttttgaggcaatagagatgccttcccctgtagcagtcttcagcctcctctaccctacaagatctggagcaacagctaggaaatatcattaattcagctcttcagagatgttatcaataaattacacatgggggctttccaaagaaatggaaattgatgggaaattatttttcaggaaaatttaaaattcaagtgagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagt

SEQ ID NO: 262 - DNA, 540 bp, Artificial Sequence
  Synthetic
gaattgcaaaactgacatcccatttcacagcaatagtgacctttatttaaattgttgtgttatagtttatgcttcttaaatcatttttcaacctaaacagccaatttctaagcagacaggaaaactaaataataagttaattaatataacaaagatgcaggttcctgctcattccagtaatgtctttgaaagcaaaactaatatttattttctagattatccctgtgaataattgagaactttttggagtcaagtatgaataaaggtgtggcagaatataataatctggactattttctataggataattgctgggttataaaatcttaggtttgcttatgcccagtagctcctgcggaggcttaataataggcaattttgaatttgttcaaacctgtaatggcttgtaaacaaagatgaccatcagctgtttctcacatctatagtgacaataaagcgggaagtataagatttaataggaggggttaaggttcatgagaaccatggaaagatgtggtctgagatgggtgctgcaaagat

SEQ ID NO: 263 - DNA, 181 bp, Artificial Sequence
  Synthetic
tgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttct

SEQ ID NO: 264 - DNA, 253 bp, Artificial Sequence
  Synthetic
gtgtctggagacacagaccgtgaccttggcgcagcggtgtgcatcagaggcgtgtgctgagaagggtggtgttaagctgtggaaaatgactcaagagcaataaatcagtgccaaagcttccctgcgcatctgaatagacacaagtgaagcccgtgtgcgcacagtgcagaggccgagcctgtatttccaggtagacgtggactttattgactgtgaattcatttacctgtaacttctgacatttcactctgtg

SEQ ID NO: 265 - DNA, 495 bp, Artificial Sequence
  Synthetic
tcgtcttcctggttgtctacaaggtgtacaagtatgaccgcgcctgccccgatgggttcgtcctcaagaacacccagtgcatcccagaaggcttggagagctactacgcggagcaagactccagtgcccgggagaaattttacacagtcataaaccactacaacctggccaagcagagcatcacgcgctccgtatcgccctggatgtcagttctgtcagaagagaagctgtccgagcaggagactgaagcggctgagaagtcagcttagcgggatgggcaagttccttacaatgtgtcacttgcaaataacaaagggactttgagggacatttcattaaatataattactgatactttagaggttactcatttacggtgcaattgcttctgtttgctaatgctgctttgcaaataaaacttgctgccgaccacccacgggcataaaatcaagtgcatttcagcattgcctaaagagctctgacaccacttttcat

SEQ ID NO: 266 - DNA, 365 bp, Artificial Sequence
  Synthetic
gtatcgtcttcctcaacaagtgcgagacctgggtgtccaacctgcgctacaaccacatgctgcggaagaagaacctgctgcttcagtacaacagcggggaggacctggccgtcaacatcatcttccccgagaagatcgacatgaccaccttcagcaaggaggccggcgacgaggagatctaagcagcgttcccacagccccctctagattcttggagttccgcagagcttactatacgcggtctgtcctaatctttgtggtgttcgctatctcttgtgtcaaatctggtgaacgctacgcttacatatattgtctttgtgctgctgtgtgacaaacgcaatgcaaaaacaatcctctttctctct

SEQ ID NO: 267 - DNA, 491 bp, Artificial Sequence
  Synthetic
aaccaatttgtgttgttctgattcaataattggtttctgggtggccaattcagaagaagagtgtacatgctcaacagtctccaggaccatcagtatactgcatttcatgtgcaccaaatattttgaaagacatttataaataattggcttatgactcatatttctctatgaataccttcatacagcaggtataactcttttctttatgggcttaaatattttgtcactgatcctgcaaatggacatcattttagcacactagcggtttatattttaaggaccttcattctctgttctgcacctcttctggaaattgagtaaattttgctttttttttttactcagttgcaacttacgcttggcatcttcagaatgcttttctagcattaagagatgtaaatgataaaggaattattgtatgaaatattacaaagcgtagactatgcattgttattcattataatattttttgctgtcataatcgcctcata

SEQ ID NO: 268 - DNA, 364 bp, Artificial Sequence
  Synthetic
tggatatttatcttgtacccgctgctaaacacactgcaggagggactctgaaacctcaagctgtctacttacatcttttatctgtgtctgtgtatcatgaaaatgtctattcaaaatatcaaaacctttcaaatatcacgcagcttatattcagtttacataaaggccccaaataccatgtcagatctttttggtaaaagagttaatgaactatgagaattgggattacatcatgtattttgcctcatgtatttttatcacacttataggccaagtgtgataaataaacttacagacactgaattaatttcccctgctactttgaaaccagaaaataatgactggccattcgttacatctgtct

SEQ ID NO: 269 - DNA, 342 bp, Artificial Sequence
  Synthetic
aaaaaaggaaatcgctgcagaatgaaggaatatcccttgaggtgacccagccaacctgtggccagaaggagggttgtaccttgaaaagaccactgaaagcattttggggtgtcaagtaagggtgggcagaggaggtagaaaatcaattcaattgtcgcatcattcatggttctttaatattgatgctcagtgcattggccttagaatatcccagcctctcttctggtttggtgagtgctgtgtaagtaagcatggtagaattgtttggagacatatatagtgatccttggtcactggtgtttcaaacattctggaaagtcacatcgatcaagaatatttttt

SEQ ID NO: 270 - DNA, 423 bp, Artificial Sequence
  Synthetic
  misc_feature (369)..(370): n is a, c, g, or t
atagtaccagtaggggcttataataaaggactgtaatcttatttaggaagttgacttatagtacatgataaatgatagacaattgaggtaagttttttgaaattatgtgacattttacattaaattttttttacattttttgggcagcaatttaaatgttatgactatgtaaactacttctcttgttaggtaatttttttcacctagatttttttcccaattgagaaaaatatatactaaacaaaatagcaataaaacataatcactctatttgaagaaaatatcttgttttctgccaatagattttttaaaatgtagtcagcaaaatgggggtggggaagcagagcatgtcctagttcaatgttgacnntttttttttttttaaagaaaagcattaagacataaaattctttcactttggca

SEQ ID NO: 271 - DNA, 227 bp, Artificial Sequence
  Synthetic
taatgtatctacatacctacacctatctatatataagctcatggtagaaaaccatagctaagtagcatcgcagacttaagcgtacaaagtgatcttgttcacaagtaatctgttgacagtgccaataaatgataaaaaaaaaattaacatgtcacaatgtaacggatgaccatatgcacaattccatgaattaaatctgtttcctgtgttagtcagtattcttaaat

SEQ ID NO: 272 - DNA, 472 bp, Artificial Sequence
  Synthetic
  misc_feature (127)..(127): n is a, c, g, or t
ctacctcactagtagttcacgtgatgtctgacagatgttgagtttcattgtgtttgtgtgttcaaatttttaaatattctgagatactcttgtgaggtcactctaatgccctgggtgccttggcacngttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggtatggtggtgtgtgcctgtagtcctatctactcaagggtggggcaggaggatcacttgagcccaggaatttgaggccacagtgaattaggattgcacc

SEQ ID NO: 273 - DNA, 473 bp, Artificial Sequence
  Synthetic
tcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcact

SEQ ID NO: 274 - DNA, 269 bp, Artificial Sequence
  Synthetic
gctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcacttaggaacgtaatcgtgtcccctatc

SEQ ID NO: 275 - DNA, 519 bp, Artificial Sequence
  Synthetic
ttggctcttggtggaattgaattcatcttgtgtcttattcaagtaataaatggagtgcttggaggcatatgtggcttttgctgctctcaccaacagcaatatgactgctaaaagaaccaacccaggacagagccacaatcttcctctatttcattgtaatttatatatttcacttgtattcatttgtaaaactttgtattagtgtaacatactccccacagtctacttttacaaacgcctgtaaagactggcatcttcacaggatgtcagtgtttaaatttagtaaacttcttttttgtttgtttatttgtgtaacatactccccacagtctacttttacaaacgcctgtaaagactggcatcttcacaggatgtcagtgtttaaatttagtaaacttcttttttgtttgtttatttgtttttgtttttttttaaggaatgaggaaacaaaccaccctctgggggtagtttacagactgagtgacagtactcagtatatctgagataaactctataatg

SEQ ID NO: 276 - DNA, 259 bp, Artificial Sequence
  Synthetic
caccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccacc

SEQ ID NO: 277 - DNA, 382 bp, Artificial Sequence
  Synthetic
gaagacttactgttggccatagttaattgtgtgaggaacacgccggcctttttagccgaaagactgcatcgagccttgaagggtattggaactgatgagtttactctgaaccgaataatggtgtccagatcagaaattgaccttttggacattcgaacagagttcaagaagcattatggctattccctatattcagcaattaaatcggatacttctggagactatgaaatcacactcttaaaaatctgtggtggagatgactgaaccaagaagataatctccaaaggtccacgatgggcttttccaacagctccaccttacttcttctcatactatttaagagaacaagcaaatataaacagcaacttgtgttcctaacagg

SEQ ID NO: 278 - DNA, 262 bp, Artificial Sequence
  Synthetic
ggatcgcagtcatccagagatgtgacctcctccagccgccaaatccgcaccaaggtcatggatgtgcacgatggcaaggtggtgtccacccacgagcaggtccttcgcaccaagaactgaggctgcccagccccgctcaggcctaggaggccccccgtgtggacacagatcccactggaagatcccctctcctgcccaagcacttcacagctggaccctgcttcaccctcaccccctcctggcaatcaatacagcttcatta

SEQ ID NO: 279 - DNA, 542 bp, Artificial Sequence
  Synthetic
tgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagccca

SEQ ID NO: 280 - DNA, 293 bp, Artificial Sequence
  Synthetic
tgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaa

SEQ ID NO: 281 - DNA, 539 bp, Artificial Sequence
  Synthetic
  misc_feature (76)..(76): n is a, c, g, or t
gtgctacagatactacatttcaaagagttggcattttccctttggccactcaagcagcatttgatgtatctaaagnaacaaagtcattgtttattttttaaaaaattatatgcagttgtacaagatactacattccattgaaatgttggctatgtcctaaccaggcaaccagataacaaaaacattttgagtcttttatctaggtagttctaattattcagctacttagtttaacaaaggaaaatatcctgacttctctcatttcatttgtagacttttcattgtataggcacaaccaaagagtcagactggtttaaaactccagaaggaaaaaaagtatcccacacagtggatgttgtttctaagaatgctacaaaatcctgacatctcagacatctcaatgttaaaggaagaaaaaaaataccttttcatttcaaagaactaatatactttgatattgtgtaaaccttactcaagtttattgtcaagctttaactgcctttttagaactttttaaaatttcgagcccacaaatctat

SEQ ID NO: 282 - DNA, 408 bp, Artificial Sequence
  Synthetic
gaaatgcttggatccttaaggagtttacgaaatctgctgttttatcaaagtgaaaaaaaattgcttattactcttcattttacactaaagcttaatgtcactaagtttcatgtctgtacagattatttaaatcatggaaatgaaaaaaatgttctctgcttgctaccaaaggacaaactcttggaaatgaacactttctgctttccttcctccaaagaattaataggcaacagtgggagaaaaaaaaggcataatggcaaatccttcaagcagggataaaagtcgatcttcaaacattaacttaagcagaccaaaaattctgatgaccgcatctagattatttttttataaaaatgattttcactatagctatgttacgctaagctactgtccaatctcttgtgatgt

SEQ ID NO: 283 - DNA, 390 bp, Artificial Sequence
  Synthetic
ggtgggtgtcaccggaaccggattgagaacaggtttccagatgaagctacttgtatgggcttctgcgcaccaaagaaaattccatcattttgctacagtccaaaagatgagggactgtgctctgccaatgtgactcgctattattttaatccaagatacagaacctgtgatgctttcacctatactggctgtggagggaatgacaataactttgttagcagggaggattgcaaacgtgcatgtgcaaaagctttgaaaaagaaaaagaagatgccaaagcttcgctttgccagtagaatccggaaaattcggaagaagcaattttaaacattcttaatatgtcatcttgtttgtctttatggcttatttgcctttatggttgtatctgaa

SEQ ID NO: 284 - DNA, 393 bp, Artificial Sequence
  Synthetic
  misc_feature (191)..(191): n is a, c, g, or t
  misc_feature (248)..(248): n is a, c, g, or t
gtgagactgaattcttgcaatgcataagatataaaagcaaatatgactcactcatttcttggggtcgtattcctgatttcagaagaggatcataactgaaacaacataagacaatataatcatgtgcttttaacatatttgagaataaaaaggactagcaaataaaactcattttgcatttaaaagttggnattatattttagtcccaagaagacaaagtcgcagattaacaacactttaaaaatatnactcccattttattgtgttagattgcagtacaatgaatgaataaggggatttatttaaaagatcttaactttcttgactgatttaaaaagctaatcaatttattaaagccagaaatgttctcctctgggaaagcaatttctatct

SEQ ID NO: 285 - DNA, 162 bp, Artificial Sequence
  Synthetic
gataagtactttagcttctttctgcagccatttcatctgcgccaacactttggaacctaatactgtgtaaggctttacaatatacggattggctttttgtgacccagattgattggttgccacatgttatgtttgttgaagtggttctcatgcaaaaatatt

SEQ ID NO: 286 - DNA, 406 bp, Artificial Sequence
  Synthetic
tcccaacacccaacaacaattaactgttaacatcttgatctgtgcactagtctgtgattattagggtgttagtgataagtatgcataaatgtcaaagatgggaagaaagatgaaaaacaagaaatagttgtgtggttgttgtgggattatggttattttgtttcggtttccttgaaaggtcatcattctagtgttttggtagtccacctttactacatatatttccattatatatgaaatgtgttcattatagaaactttgaagttacagaaatgtagaagagaaactcacccatgttttcaccatccaaagagtgtggttaacatcttgatatattttcttcatcttgtttctgtgcacaggtttttggtttgttaatatggttgtggtcattctatctgtaata

SEQ ID NO: 287 - DNA, 503 bp, Artificial Sequence
  Synthetic
ttacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttatttgggggtaatattgtaagttttttagaagacaattttcataacttgataaattatagttttgtttgttagaaaagtagctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaat

SEQ ID NO: 288 - DNA, 348 bp, Artificial Sequence
  Synthetic
catcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagttagagccgatatcactggaagatattcaaatcgtctctatgcttacgaacctgcagatacagctctgttgcttgacaacatgaagaaagctctcaagttgctgaagactgaattgtaaagaaaaaaaatctccaagcccttctgtctgtcaggccttgagact

SEQ ID NO: 289 - DNA, 491 bp, Artificial Sequence
  Synthetic
gaaatatcactgacactccagactaaagcagagtctaggtttctgcaattttgttacagtaatttgtaaatagctttagtaaactcaccttgcatggtagattaataagatgacttactgtacatgaattacacaataatgagatctggtggctatttccacattttgaaaaggattcagttatttactgacagtggtgagcatcctttttaaaataatgttctgatacttaaacattagagagcagtatctttaaatgaattattaacactttggaatacttacattttctgttatttttgattgcctgataaccagtttcaatgatgaaaatgaaaacaagtgctgaagatgaaatggaagagaaccgttttaatctggattttgttttgtcacacctggaaaatactttgcaaatatgttctaaattgaaaacaatttttttatgatcacatggttcactaccaaatgaccctcaaataagccagatg

SEQ ID NO: 290 - DNA, 366 bp, Artificial Sequence
  Synthetic
  misc_feature (40)..(40): n is a, c, g, or t
  misc_feature (198)..(198): n is a, c, g, or t
  misc_feature (339)..(339): n is a, c, g, or t
aatgcctaaaaatgcctatttgggattttttttttttttnaaattaagagaagctctcttctgtgtagaacagttgttccaaaatagcttagtgttttgttttcctgttgcatgacagatttaactattctttccagcagtggaggtgctgtcagagtccagtgttctagaagaggcagtgtctaaagcctaattttncttttctaattctggtagctattaccaggaatttttgaaagttttgtttaagtagtctaatattttttatgtaaagagcattaaattttgctatgtataaatttttgtaacctaacagtgaatcaatattttctatcagtnccaagggcttcctgtagttctattcaa

SEQ ID NO: 291 - DNA, 498 bp, Artificial Sequence
  Synthetic
tgtttcctcatagctagttctcaagctgcatgtaagattttaacgggaagagaaaataggcctggacctgaaggtctcaaatatgttgagaagaaagtatgaactataaggaacttgagatgtagatttattttgcaggaaatacgaggaaaataggaaagaagtgtttgccgcatcaagtgtaaagaatgatactgaggatttttacattttatgaaatgaaataatggcatttacaaaatgaaaaatgtagtttcacaactaagttttgttaacagagactgcatgctttgcttatagttcttaattttggttttgacattcatttaattttttccatgttaaatatgtagtttaattatttactcaaaataaacattgttcatgcttttaggcctttgggggaattgatttttatccacaggtagaaaatggtctttgcacacactacacttatttcaaatatacaatgtgctcccgaactttcgcattagtctt

SEQ ID NO: 292 - DNA, 552 bp, Artificial Sequence
  Synthetic
atgtcatccgtatttcaaccggagtaaattgctagatttctgcaagtcgaaagatattgttctggttgcctatagtgctctgggatctcaacgagacaaacgatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtttttgagttccagttgactgcagaggacatgaaagccatagatggcctagacagaaatctccactattttaacagtgatagttttgctagccaccctaattatccatattcagatgaatattaacatggagggctttgcctgatgtctaccagaagccctgtgtgtggatggtgacgcagaggacgtctctatgccggtgactggacatatcacctctacttaaatccgtcctgtttagcgacttcagtcaactacagctgagtccataggccagaaag

SEQ ID NO: 293 - DNA, 318 bp, Artificial Sequence
  Synthetic
tgctcaccatcagatcaacctctgattttacatcatgatgtaatcaccactggagcttcactttgttactaaattattaatttcttgcctccagtgttctatctctgaggctgagcattataagaaaatgacctctgctccttttcattgcagaaaattgccaggggcttatttcagaacaacttccacttactttccactggctctcaaactctctaacttataagtgttgtgaacccccacccaggcagtatccatgaaagcacaagtgactagtcctatgatgtacaaagcctgtatctctgtgatgatttctgt

SEQ ID NO: 294 - DNA, 534 bp, Artificial Sequence
  Synthetic
gaagataaccggctcattcacttcctcccagaagacgcgtggtagcgagtaggcacaggcgtgcacctgctcccgaattactcaccgagacacacgggctgagcagacggcccctgtgatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaac

SEQ ID NO: 295 - DNA, 347 bp, Artificial Sequence
  Synthetic
gagctggcgctcaaggatgctcgtgccaagcaggaggagctggaagccgccctgcagcgggccaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggac

SEQ ID NO: 296 - DNA, 225 bp, Artificial Sequence
  Synthetic
aatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaat

SEQ ID NO: 297 - DNA, 578 bp, Artificial Sequence
  Synthetic
cccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacc

SEQ ID NO: 298 - DNA, 594 bp, Artificial Sequence
  Synthetic
  misc_feature (166)..(166): n is a, c, g, or t
taaccatgacaatcaacgaggacatggcgctggaggagcctctatacttaccttctgggatgctaggctgtacaaaatggcagttggatttatgcttgctcatccttatggatttacacgagtaatgtcaagctaccgttggccaagatattttgaaaatggaaangatgttaatgattgggttgggccaccaaatgataatggagtaactaaagaagttactattaatccagacactacttgtggcaatgactgggtctgtgaacatcgatggcgccaaataaggaacatggttaatttccgcaatgtagtggatggccagccttttacaaactggtatgataatgggagcaaccaagtggcttttgggagaggaaacagaggattcattgttttcaacaatgatgactggacattttctttaactttgcaaactggtcttcctgctggcacatactgtgatgtcatttctggagataaaattaatggcaactgcacaggcattaaaatctacgtttctgatgatggcaaagctcatttttctattagtaactctgctgaagatccatttattgcaattcatgctgaatctaa

SEQ ID NO: 299 - DNA, 535 bp, Artificial Sequence
  Synthetic
atgcaacagcgaaagacattgcccctactttaacactctatgtggggaaaaagcagcttgtagagattgagaaggtggttctacaccctaactaccaccaggtagatattgggatcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgta

SEQ ID NO: 300 - DNA, 531 bp, Artificial Sequence
  Synthetic
ccgagccgagcttactgtgagtgtggagatgttatcccaccatgtaaagtcgcctgcgcaggggagggctgcccatctccccaacccagtcacagagagataggaaacggcatttgagtgggtgtccagggccccgtagagagacatttaagatggtgtatgacagagcattggccttgaccaaatgttaaatcctctgtgtgtatttcataagttattacaggtataaaagtgatgacctatcatgaggaaatgaaagtggctgatttgctggtaggattttgtacagtttagagaagcgattatttattgtgaaactgttctccactccaactcctttatgtggatctgttcaaagtagtcactgtatatacgtatagagaggtagataggtaggtagattttaaattgcattctgaatacaaactcatactccttagagcttgaattacatttttaaaatgcatatgtgctgtttggcaccgtggcaagatggtatcagagagaaacccatcaattgctcaaatactc

SEQ ID NO: 301 - DNA, 468 bp, Artificial Sequence
  Synthetic
agggcaagtgttcccaaattgacgccacctaataatcatcaccacaccgggagcagatctgaaggcacactttgatttttttaaggataagaaccacagaacactgggtagtagctaatgaaattgagaagggaaatcttagcatgcctccaaaaattcacatccaatgcatactttgttcaaatttaaggttactcaggcattgatcttttcagtgttttttcacttagctatgtggattagctagaatgcacaccaaaaagatacttgagctgtatatatatatgtgtgtgtgtgtgtgtgtgtgtgtgtgtgtgcatgtatgtgcacatgtgtctgtgtgatatttggtatgtgtatttgtatgtactgttattcaaaatatatttaatacctttggaaaatcttgggcaagatgacctactagttttccttgaaaaaaagttgctttgttattaatattgtgct

SEQ ID NO: 302 - DNA, 563 bp, Artificial Sequence
  Synthetic
gaaatctgtggaccgaagcatacaaatggtggtatcttgtctgtttaatccagagaagagactgataaattccgttgttactcaagatgactgcttcaagggtaaaagagtgcatcgctttagaagaagtttggcagtatttaaatctgttggatcctctcagctatctagtttcatgggaagttgctggttttgaatattaagctaaaagttttccactattacagaaattctgaattttggtaaatcacactgaaactttctgtataacttgtattattagactctctagttttatcttaacactgaaactgttcttcattagatgtttatttagaacctggttctgtgtttaatatatagtttaaagtaacaaataatcgagactgaaagaatgttaagatttatctgcaaggatttttaaaaaattgaaacttgcattttaagtgtttaaaagcaaatactgactttcaaaaaagtttttaaaacctgatttgaaagctaacaattttgatagtctgaacacaagcatttcacttctccaagaagtacctgtgaacagt

SEQ ID NO: 303 - DNA, 380 bp, Artificial Sequence
  Synthetic
tttcccctgtgcatgtgcgaaggaaaatcatttcagatgacagtgtttaaccatggtcaaaagaccattctgtcctatccttcttagaagcttcgaactcaaaatcatggaaaggttttaagatttgaggttggttttagggttgctagatttagcaagtaaaaataaggatggccccgttaaattttaacttaaaattaacaagttttttgttaattttttgttttttgtctcagcatcagtatatcccatgcaatatttgaggtgtgctcatactaaaattatttgtgtatctgaaattcaaattaaactgggtgtctttttcttttcatctggcaaccctactaagatcataaacccttggaaatctgtgtgtgtgc

SEQ ID NO: 304 - DNA, 434 bp, Artificial Sequence
  Synthetic
gactctttttcctctactgagattcatctgtgtgaaatatgagttggcgaggaagatcgacctattattggcctagaccaaggcgctatgtacagcctcctgaaatgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctcatagccaggaacagggtcacccacagactgggtgtgagtgtgaagatggtcctgatgggcaggagatggacccgccaaatccagaggaggtgaaaacgcctgaagaaggtgaaaagcaatcacagtgttaaaagaaggcacgttgaaatgatgcaggctgctcctattttgga

SEQ ID NO: 305 - DNA, 544 bp, Artificial Sequence
  Synthetic
tcctctgttttgtgtgtcgtgaatccaaggaccacaaatcccataatgtcagcttgatcgaagaagctgcccagaattatcaggggcagattcaagagcagatccaagtcttgcagcaaaaggagaaggagacagtacaagtgaaggcacaaggtgtacacagggtcgatgtcttcacggaccaggtagaacatgagaagcaaaggatcctcacagaatttgaactcctgcatcaagtcctagaggaggagaagaatttcctgctatcacggatttactggctgggtcatgagggaacggaagcggggaaacactatgttgaaattccactgatgcccacagttgaacgatctcaagaagctcgttgttacccctgaagaccaatggcagaactagccacccaggcagctgctggaggtatacaaagtcgtcttgtggcagaagtgaagagtttcagtttctcaacccaacccctgttcctctggaactggagaaaaactcagtgaaggcaaaatcaagacacgactccatcacagggagcctg

SEQ ID NO: 306 - DNA, 466 bp, Artificial Sequence
  Synthetic
gaggcagtgctgtgtggttcctgccgtccggactctttttcctctactgagattcatctgtgtgaaatatgagttggcgaggaagatcgacctattattggcctagaccaaggcgctatgtacagcctcctgaagtgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctgatagccaggaacagggtcacccacagactgggtgtgagtgtgaagatggtcctgatgggcaggaggtggacccgccaaatccagaggaggtgaaaacgcctgaagaaggtgaaaagcaatcacagtgttaaaagaagacacgttgaaatgatgcaggctgctcctatgttggaaa

SEQ ID NO: 307 - DNA, 417 bp, Artificial Sequence
  Synthetic
gtgcaggagaaggacttgcccgcatggagctatttttatttctaaccacaattttacagaactttaacctgaaatctgttgatgatttaaagaacctcaatactactgcagttaccaaagggattgtttctctgccaccctcataccagatctgcttcatccctgtctgaagaatgctagcccatctggctgctgatctgctatcacctgcaactctttttttatcaaggacattcccactattatgtcttctctgacctctcatcaaatcttcccattcactcaatatcccataagcatccaaactccattaaggagagttgttcaggtcactgcacaaatatatctgcaattattcatactctgtaacacttgtattaattgctgcatatgctaatacttttctaatgctgactt

SEQ ID NO: 308 - DNA, 502 bp, Artificial Sequence
  Synthetic
gagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggggaattttattcataaatgtccacagaaacagtaaatgttctcgcttcagaacttaattcatctaatccctcctgtttgtctcaaattataggataactttgaaactttctgaattaacgttatttaaaaggaaatgtagatgttattttagtctctatcttcaggttattatcacttaaaaacctgcgaaagctgtcaacttttgtggttgtagcaagtattaataaatatttataaatcctctaatgtaagtctagctacctatccaatac

SEQ ID NO: 309 - DNA, 516 bp, Artificial Sequence
  Synthetic
gaagcaattgctcatgttggccaaacatggtgcaccgagtgatttccatctctggtaaagttacacttttatttcctgtatgttgtacaatcaaaacacactactacctcttaagtcccagtatacctcatttttcatactgaaaaaaaaagcttgtggccaatggaacagtaagaacatcataaaatttttatatatatagtttatttttgtgggagataaattttataggactgttctttgctgttgttggtcgcagctaaataagactggacatttaacttttctaccatttctgcaagttaggtatgtttgccaggagaaaagtatcaagacgtttaactgcagttgactttctccctgttcctttgagtgtcttctaactttattctttgttctttatgtagaattgctgtctatgattgtactttgaatcgcttgacttgttgaaaatatttctctagtgtattatcactgtctgttctgcacaataaacataacagcctctgtgatccc

SEQ ID NO: 310 - DNA, 388 bp, Artificial Sequence
  Synthetic
ttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagaggcctttcttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtt

SEQ ID NO: 311 - DNA, 537 bp, Artificial Sequence
  Synthetic
gagaacacggtggcagagacggagtgccgctatgccctgcagctgcagcagatccagggactcatcagcagcatcgaggcccagctgagcgagctccgcagtgagatggagtgccagaaccaagagtacaagatgctgctggacatcaagacacgtctggagcaggagatcgccacctaccgcagcctgctcgagggccaggacgccaagaagcgtcagcccccgtagcacctctgttaccacgacttctagtgcctctgttaccaccacctctaatgcctctggtcgccgcacttctgatgtccgtaggccttaaatctgcctggcgtcccctccctctgtcttcagcacccagaggaggagagagccggcagttccctgcaggagagaggaggggctgctggacccaaggctcagtccctctgctctcaggaccccctgtcctgactctctcctgatggtgggccctctgtgctcttctcttccggtcggatctctctcctctctgacctggatacgctttggtttctcaacttc

SEQ ID NO: 312 - DNA, 252 bp, Artificial Sequence
  Synthetic
ttctctgaccacaacttcgcaggatctaacaagattgctgagatcctatgtaaggacctgtggcgcaatcccctgcaatactacaagaggatgaagccacctgaagagggaacagagacgtcaggggactcccagttgttgagttgaatatgatggagcatcagattttacctaatacagcagaactcctaaaaagttacagccatatgcaggacggcagtactcagcatggtcttatgcacaggaactaaa

SEQ ID NO: 313 - DNA, 447 bp, Artificial Sequence
  Synthetic
ggacatgtgtttaaagtggctttctggcccttctttcaatggcttcttccctaaaacgtggagactctaagttaatgtcgttactatgggccatattactaatgcccactggggtctatgatttctcaaaattttcattcggaatccgaaggatacagtctttaaactttagaattcccaagaaggctttattacacctcagaaattgaaagcaccatgactttgtccattaaaaaattatccatagtttttttagtgcttttaacattccgacatacatcattctgtgattaaatctccagatctctgtaaatgatacctacattctaaagagttaattctaattattccgatatgaccttaaggaaaagtaaaggaataaatttttgtctttgttgaagtatttaatagagtaaggtaaagaagatattaagtccctttcaaaat

SEQ ID NO: 314 - DNA, 505 bp, Artificial Sequence
  Synthetic
  misc_feature (281)..(281): n is a, c, g, or t
agaacagcagctttctagggacagctggaaagggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtngggggattgaaatgcaagcaattagtggatcactgttagggtaagggaatgtatgtacacatctattttttatacttttttttttaaaaaagaatgtcagttgttatttattcaaattatctcacattatgtgttcaacatttttatgctgaagtttcccttagacattttatgtcttgcttgtagggcataatgccttgtttaatgtccattctgcagcgttt

SEQ ID NO: 315 - DNA, 270 bp, Artificial Sequence
  Synthetic
ggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtc

SEQ ID NO: 316 - DNA, 478 bp, Artificial Sequence
  Synthetic
caaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgacaaatatagcagggtaatgtaagatgataacgttagagaaactgaaactgggtgagggctatctaggaattctctgtactatcttaccaaattttc

SEQ ID NO: 317 - DNA, 470 bp, Artificial Sequence
  Synthetic
aaagcattcagcaaatggcaccctaaaatatgccgctggtttgccactgacttcaaaatgaggtcacttgggcacagcacatgcagggaggggcttttctctgagcctccttcatctgcccaaagacaaatcctcaaaaggaaattcaatcatcatgaatcacaaccccaagtatctcatcagccagggaagagtaagtgggatcacagggaaggatgttggcagcgacaccatcccatacaggctcttacctcttctcctgagggctgctccagacaacatttattacccagaagaccttttgtctgaaaaccagccaagctttattcaggacacacttcttgccttcactttcccacttccgtggccacctccatgcagaagccctaagcccacattctttcaatagctcacggtggtgcatgagtgtccatcatctgactcttctcggagtctcatattttgtggga

SEQ ID NO: 318 - DNA, 264 bp, Artificial Sequence
  Synthetic
tgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctcatagccaggaacagggtcacccacagactgggtgtgagtgtgaagatggtcctgatgggcaggagatggacccgccaaatccagaggaggtgaaaacgcctgaagaag

SEQ ID NO: 319 - DNA, 548 bp, Artificial Sequence
  Synthetic
agtctgctgaagaggcattgcacagatcaaacaaggatggatcatttcttattcggaaaagctctggccatgattccaaacaaccatatacactagttgtattctttaataagcgagtatataatattcctgtgcgatttattgaagcaacaaaacaatatgccttgggcagaaagaaaaatggtgaagagtactttggaagtgttgctgaaatcatcaggaatcatcaacatagtcctttggttcttattgacagtcagaataacacaaaagattccaccagactgaagtatgcagttaaagtttcataaagggggaaaaaaaagatcaataccattgcttcagacactttcccaaagtttctccttttgagaaaaagtcccaaaacttcatattttggattatgaatcatccagtaataaaatggaagatggagtcagctattgaagtggtcatccatttctttttaagaagctcatgtggacttgttctattgcctgacctgatgaactgttaatatctggtgaggttgagttatcatgctacta

SEQ ID NO: 320 - DNA, 505 bp, Artificial Sequence
  Synthetic
actcgcacaccaaccttatatttggacttcaaattggacccaggagccaaacattcccaacctatccctaaagggtggacaagcttcatttacacgatatctggagatgtgtatattgggcccgatgatgcacaacaaaaaatagaacctcatcacacagcagtgcttggagaaggtgacagtgtccaggtggagaacaaggatcccaagagaagccactttgtcttaattgctggggagccattaagagaaccagttatccaacatggtccatttgtgatgaacaccaatgaagagatttctcaagctattcttgatttcagaaacgcaaaaaatgggtttgaaagggccaaaacctggaaatcaaagattgggaactagtggaaagcggaagagcaggtcttgatgtgtcctagaattttgccatttctgagattgagccattgaaggcattccatttctaaagcttatttagccggtgcttctaaagaattccacactaacg

SEQ ID NO: 321 - DNA, 234 bp, Artificial Sequence
  Synthetic
gtacctgtctataaggagtcctgcttatcacaatgaatgttctcctgggcagcgttgtgatctttgccaccttcgtgactttatgcaatgcatcatgctatttcatacctaatgagggagttccaggagattcaaccaggaaatgcatggatctcaaaggaaacaaacacccaataaactcggagtggcagactgacaactgtgagacatgcacttgctacgaaacagaaattt

SEQ ID NO: 322 - DNA, 561 bp, Artificial Sequence
  Synthetic
ggatgtgatagcattcctgctggcctgcgtggcaactgtgatatttatcatcacaaaattttgcctgttttgtttccgaaagcttgccaaaacaggaaagaagaagaaaagagattagttatatcaaaagcctgaagtggaatgactgaaagatgggactcctcctttatttcagcatggagggttttaaatggaggatttcctttttcctgtgacaaaacatcttttcacaacttaccttgttaagacaaaatttattttccagggatttaatacgtactttagttggaattattctatgtcaatgatttttaagctatgaaaaatacaatggggggaaggatagcatttggagatatacctaatgttaaatgacgagttactggatgcagcacgcaacatggcacatgtgtatacatatgtagctaacccttcgttgtgcacatgtaccctaaaacttaaagtataatttaaaaaaagcaaaaaaaaaaaataccaactcttttttttaaaccaggaaggaaaatgtgaacatggaaacaacttctagtattggatctg

SEQ ID NO: 323 - DNA, 515 bp, Artificial Sequence
  Synthetic
agcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttcttt

SEQ ID NO: 324 - DNA, 567 bp, Artificial Sequence
  Synthetic
gtgtgtagtcttacgatgtttagtaacctcaataagtttatttcttaaatctagtaatttgtagattcctctggattttgtatatgcatagtcatgtaagctgaaaatatggcaatacttgcttcttcccaattgctttaccttttttcttaccttattgcactggttagcaaccccaatacagagaccaccagagcaggtatagactcctgaaagacaatataatgaagtgctccagtcaggcctatctaaactggattcacagctctgtcacttaattgctacatgatctagagccagttactttgtgtttcagccatgtatttgcagctgagagaaaataatcattcttatttcatgaaaattgtggggatgatgaaataagttaacacctttaaagtgtgtagtaaagtatcaggatactatattttaggtcttaatacacacagttatgccgctagatacatgctttttaatgagataatgtgatattatacataacacatatcgatttttaaaaattaaatcaaccttgctttgatggaataaactccatttagtcacaaa

SEQ ID NO: 325 - DNA, 374 bp, Artificial Sequence
  Synthetic
gacccagacatattgtactctttctaaaaataataataataatgctaacagaaagaagagaaccgttcgtttgcaatctacagctagtagagactttgaggaagaattcaacagtgtgtcttcagcagtgttcagagccaagcaagaagttgaagttgcctagaccagaggacataagtatcatgtctcctttaactagcataccccgaagtggagaagggtgcagcaggctcaaaggcataagtcattccaatcagccaactaagttgtccttttctggtttcgtgttcaccatggaacattttgattatagttaatccttctatcttgaatcttctagagagttgctgaccaactgacgtatgtttcccttt

SEQ ID NO: 326 - DNA, 466 bp, Artificial Sequence
  Synthetic
gaggcagtgctgtgtggttcctgccgtccggactctttttcctctactgagattcatctgtgtgaaatatgagttggcgaggaagatcgacctattattggcctagaccaaggcgctatgtacagcctcctgaaatgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctgatagccaggaacagggtcacccacagactgggtgtgagtgtgaagatggtcctgatgggcaggagatggacccgccaaatccagaggaggtgaaaacgcctgaagaaggtgaaaagcaatcacagtgttaaaagaaggcacgttgaaatgatgcaggctgctcctatgttggaaa

SEQ ID NO: 327 - DNA, 514 bp, Artificial Sequence
  Synthetic
gaatattttccacaagatgctgcaatgtgagttatcacttcatttatcttaaagaaagactaaactggttgtcagttacatctgacagaaaaaaaaaaaaaatcactgtgtaaccaggttaagtggtaaaataatccagggcgtcagtcaaaggcattttgctgactttaatattgattatatttttaacagggaatttaaggaaaatattacctgggaattaaaaaatatatatatattaaaacaagaattttcctttgcctctgtctagcttaaacctactacctcaagctgcttaagttccttaagtattgtttgtaatcaccaataaataagtgcatttgtaattcatcagtcattattagcttttattaaaagaagattacgttttacaatgtaactataatctcttgaatttggtatcttattaatgagttttaaagatgtaaaacctaaccttttttaaagctccattgtcttatgtttttagaggcttttccgtaaacatatatct

SEQ ID NO: 328 - DNA, 393 bp, Artificial Sequence
  Synthetic
tgattaccttaatgagttgggacctcgctttaaaagattagcatgcatgtttggttctgcagtgcagtcaaataattagggctttttaccatcaaaatttttaaaagtgctaatgtgtattcgaacccaatggtagtcttaaagagttttgtgccctggctctatggcggggaaagccctagtctatggagttttctgatttccctggagtaaatactccatggttattttaagctacctacatgctgtcattgaacagagatgtggggagaaatgtaaacaatcagctcacaggcatcaatacaaccagatttgaagtaaaataatttaggaagatattaaaagtagatgagaggacacaagatgtagtcgatccttatgcgattatatcat

SEQ ID NO: 329 - DNA, 515 bp, Artificial Sequence
  Synthetic
gacattcttttgtaccttgttcaactttagagttctaagaaagcttatttattagcgatataaccttgctttgcaaacagaatgcaagcgttaactttggtcttctgtattttggactaaatactaattgactagagtgctgtaaacttgctgtaacatttatggcaattgcaagttgccctgctaggcagttgtaatctggcattaacttattttctatatccagtttaatatgaatctggtgttgatgcaatgcctcagtgatgcattagatctctaataaagtctgtatatacatgtacactttgatcctgctggaaaattttatcagcaaacacattgtctaatctttcaaaacagatttaaggaaaggactgaaagtacagactgaacagtgtggttctttgaaaggtttggttttttaatttttattctaaaattcaaccttttttttgtcgatttaaccatttccattttgaactgctatttgtattgtgctttttacttgagtcgtc

SEQ ID NO: 330 - DNA, 513 bp, Artificial Sequence
  Synthetic
  misc_feature (215)..(215): n is a, c, g, or t
aatcacccaaggatggatatcaggagaatatctctggaaaatacatacaaactgtttattcaacttctgataggtctgtcattgaaagagatatgtgcacttactgccgaaaacccttgggtgtagaaactaaaatgattttagatgaattacaaatttgctgccattctacttgctttaagtgtgaaatatgcaagcagcctttggaaaatctncaagcgggtgatagtatttggatttatagacagacaatacactgtgaaccttgctactctaaaattatggcaaagtggattccataactctggcacaaggaaatcaagatgaaaagcactcattaaggaattaaagttacaagttttatcttaataatatgtaatctagaaaagctttcacattgaagatcaactcttgtacaaaattaacaattctgttattgcataagtaatctaattgtcttcaataaggtcacacacataaaaagagccatctggtctctggctagagttagca

SEQ ID NO: 331 - DNA, 385 bp, Artificial Sequence
  Synthetic
tagaagtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctccggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatctt

SEQ ID NO: 332 - DNA, 434 bp, Artificial Sequence
  Synthetic
  misc_feature (129)..(129): n is a, c, g, or t
  misc_feature (377)..(377): n is a, c, g, or t
  misc_feature (379)..(380): n is a, c, g, or t
caaatgctgagcaccagccacgtgcacagcagttgcttttactagtcttagctctacgatttaaatccatgtgtccaagggggaaaacatattatatttgtaaccaaaaactactagtttaccagaggnctgaagggagataaagaggagttggttaatgggtacaaaaatccagttagatgaaaggaataatatagatagtgttcagtagcagaatagaatgaacataaactattagtttaaattatgtgaaattccttctatttgatcatattttacaagaaaaaacatcaattttatatagtccaacttaatacctagccttatgagttgtataaggtaaggttacctacctgagaagctgattaacattggtngnncaatcttattcattagagaacatggtgcttagggtctgagaccttttgaaaggt

SEQ ID NO: 333 - DNA, 469 bp, Artificial Sequence
  Synthetic
aaacagacccaattcatcaactggaaagcaattctatgctggtgctatgcaattatgctctgtgtttcatatgttgctttggcttattttttttcttaaaggaatgtgttgttcatgaaaaactgatagaagcaacagaagaattcgcaggaagaaaaatcatagtgttaatgaattattgagggcgaaaaaaaggtgttttcttctttgactacggagtccaaatccacttaaattctgttttcctgaaaagaggtacagcataagaaatagctctttattgatgttttaaaagcagcaacttggtggtgtactactggaactaatgactgcaaagtgttaaacgactgaaatatacaaacagtctcttagttactcatttccatcttctcttcaactttcacatcagtcttccggaatcaagatcaacatatcaggtggtcattgcctttctccattgtctagtaga

SEQ ID NO: 334 - DNA, 456 bp, Artificial Sequence
  Synthetic
ttatctgcgaatctgcttgcttcatcccgttcagaagacagctttcatgcagttttacaagacagcaaaagattggtggctgttcagtttctatttctgcttgccattggccatcactgcatttttttatacactaatgacctgtgaaatgttgagaaagaaaagtggcatgcagattgctttaaatgatcacctaaagcagagacgggaagtggccaaaaccgtcttttgcctggtccttgtctttgccctctgctggcttccccttcacctcagcaggattctgaagctcactctttataatcagaatgatcccaatagatgtgaacttttgagctttctgttggtattggactatattggtatcaacatggcttcactgaattcctgcattaacccaattgctctgtatttggtgagcaaaagattcaaaaactgctttaaggctgggccaca

SEQ ID NO: 335 - DNA, 520 bp, Artificial Sequence
  Synthetic
agggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccc

SEQ ID NO: 336 - DNA, 438 bp, Artificial Sequence
  Synthetic
gcttccgggaaggtgtctcaagtgggtgggcagacttctgacgaagccctgagcatgctgtctgaaggttctgatgccagcacaattgaaattcacactgcaagtgaatcctgcaacaaaaatgagggtgaccctgctctcccaacccatggagacctatgaaggggatgtgctgggggtccagaccccatattcctcagactcaacaattcttgttctttagaactgtgttctcaccttcccaacactgcactgccgaagtgtagcggcccccaaaccttgctctcatcaccagctagagcttcttcccgaagggcctttaggataggagaaagggttcatgcacacacgtgtgagaatggaagagccccctccagaccactctacagctgctctagccttagttgccactaggaagttttctgaggctggctgtaa

SEQ ID NO: 337 - DNA, 519 bp, Artificial Sequence
  Synthetic
gaagacactaccaacatgatattacgtattgatctgaccacacacaatgttactctagaagaaccaatagaaatcaactggtcatgaagatcaccatcaattttagttgtcaatgggaaaaaacaccaggatttaagtttcacagcacttacaattttccctcttcacttggttcttgtactctacaaaatatagctttcataacatcgaaaagttattttgtagcgtacatcaatgataatgctaattttattatagtaatgtgacttggattcaattttaaggcatatttaacaaaatttgaatagccctatttatccttgttaagtatcagctacaattgtaaactagttactaaacatgtatgtaaatagctaagatataatttaaacgtgatttttaaattaaataaaatttttatgtaattatatatactatatttttctcaatgtttagcagatttaagatatgtaacaacaattatttgaagatttaattacttcttagtatgtgcattta

SEQ ID NO: 338 - DNA, 495 bp, Artificial Sequence
  Synthetic
atggccatggctcagaaaccttatacctagctcctggaggtggggacgattggatctatgatttgggcatcaaatattcgtttacatcaaacccacctgtagagaagcttttgccgctgtctctaaaatagcttggcatgtcattaggaatgtttaatgcccctgattttatcattctgcttccgtattttaatttactgattccagcaagaccaaatcattgtatcagattatttttaagttttatccgtagttttgataaaagattttcctattccttggttctgtcagagaacctaataagtgctactttgccattaaggcagactagggttcatgtctttttaccctttaaaaaaaaattgtaaaagtctagttacctactttttctttgattttcgacgtttgactagccatctcaagcaactttcgacgtttgactagccatctcaagcaagtttaatcaaagatcatctcacgctgatcattggatcc

SEQ ID NO: 339 - DNA, 482 bp, Artificial Sequence
  Synthetic
atttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactg

SEQ ID NO: 340 - DNA, 436 bp, Artificial Sequence
  Synthetic
gactctttttcctctactgagattcatctgtgtgaaatatgagttggcgaggaagatcgacctattattggcctagaccaaggcgctatgtacagcctcctgaaatgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctcatagccaggaacagggtcacccacagactgggtgtgagtgtgaagatggtcctgatgggcaggagatggacccgccaaatccagaggaggtgaaaacgcctgaagaaggtgaaaagcaatcacagtgttaaaagaaggcacgttgaaatgatgcaggctgctcctatgttggaaa

SEQ ID NO: 341 - DNA, 459 bp, Artificial Sequence
  Synthetic
tgagcctggggttctggtgttagaatatttttaagtaggctttactgagagaaactaaatattggcatacgttatcagcaacttcccctgttcaatagtatgggaaaaataagatgactgggaaaaagacacacccacaccgtagaacatatattaatctactggcgaatgggaaaggagaccattttcttagaaagcaaataaacttgatttttttaaatctaaaatttacattaatgagtgcaaaataacacataaaatgaaaattcacacatcacatttttctggaaaacagacggattttacttctggagacatggcatacggttactgacttatgagctaccaaaactaaattctttctctgctattaactggctagaagacattcatctatttttcaaatgttctttcaaaacatttttataagtaatgtttgtatctatttcatgctttact

SEQ ID NO: 342 - DNA, 432 bp, Artificial Sequence
  Synthetic
ctacgatgagtttgagtactgctgggacacctttgtgtaccgccagggatgtcccttccagccctgggatggactagaggagcacagccaagccctgagtgggaggctgcgggccattctccagaatcagggaaactgaaggatgggcctcagtctctaaggaaggcagagacctgggttgagcagcagaataaaagatcttcttccaagaaatgcaaacgagccgttcaccaccatctccagctgctcacagacaccagcaaagcaatgtgctcctgatcaagtagattttttaaaaatcagagtcaattaattttaattgaaaatttctcttatgttccaagtgtacaagagtaagattatgctcaatattcccagaatagttttcaatgtattaatgaagtgattaattggctccatatttagactaat

SEQ ID NO: 343 - DNA, 379 bp, Artificial Sequence
  Synthetic
gaagaggccatcaagcacatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgc

SEQ ID NO: 344 - DNA, 557 bp, Artificial Sequence
  Synthetic
ttccctgctggaggatcccaagattaaggagattgctgcaaagcacaaaaaaaccgcagcccaggttctgatccgtttccatatccagaggaatgtgattgtcatccccaagtctgtgacaccagcacgcattgttgagaacattcaggtctttgactttaaattgagtgatgaggagatggcaaccatactcagcttcaacagaaactggagggcctgtaacgtgttgcaatcctctcatttggaagactatcccttcaatgcagaatattgaggttgaatctcctggtgagattatacaggagattctctttcttcgctgaagtgtgactacctccactcatgtcccattttagccaagcttatttaagatcacagtgaacttagtcctgttatagacgagaatcgaggtgctgttttagacatttatttctgtatgttcaactaggatcagaatatcacagaaaagcatggcttgaataaggaaatgacaattttttccacttatctgatcagaacaaatgtttattaagcatcagaaactctgccaacactga

SEQ ID NO: 345 - DNA, 342 bp, Artificial Sequence
  Synthetic
tggtgtcaggggtggtcctatgcccaagctggctgaccggaagctgtgtgcggaccaggagtgcagccaccctatctccatggctgtggcccttcaggactacatggcccccgactgccgattcctgaccattcaccggggccaagtggtgtatgtcttctccaagctgaagggccgtgggcggctcttctggggaggcagcgttcagggagattactatggagatctggctgctcgcctgggctatttccccagtagcattgtccgagaggaccagaccctgaaacctggcaaagtcgatgtgaagacagacaaatgggatttctactgccagtgagctca

SEQ ID NO: 346 - DNA, 438 bp, Artificial Sequence
  Synthetic
taattacgtctgaggctggaagctgggaaacccaataaatgaactcctttagtttattacaacaagaagacgttgtgatacaagagattcctttcttcttgtgacaaaacatctttcaaaacttaccttgtcaagtcaaaatttgttttagtacctgtttaaccattagaaatatttcatgtcaaggaggaaaacattagggaaaacaaaaatgatataaagccatatgaggttatattgaaatgtattgagcttatattgaaatttattgttccaattcacaggttacatgaaaaaaaatttactaagcttaactacatgtcacacattgtacatggaaacaagaacattaagaagtccgactgacagtatcagtactgttttgcaaatactcagcatactttggatccatttcatgcaggattgtgttgttttaac

SEQ ID NO: 347 - DNA, 329 bp, Artificial Sequence
  Synthetic
aaagcccaacatcccatggctgtttctcacagatcccaaattggccatggaagtttattttggcccttgtagtccctaccagtttaggctggtgggcccagggcagtggccaggagccagaaatgccatgctgacccagtgggaccggtcgttgaaacccatgcagacacgagtggtcgggagacttcagaagccttgcttctttttccattggctgaagctctttgcaattcctattctgttaatcgctgttttccttgtgttgacctaatcatcattttctctaggatttctgaaagttactgacaatacccagacaggggctttgc

SEQ ID NO: 348 - DNA, 531 bp, Artificial Sequence
  Synthetic
ggacattcgattggatggaaaccctattaatctcagcaaaactccacaagcatacatgtgtctacctcgtctgcctgttgggagccttgtctaatttcagataatggttagcattacgatggctactataaataaaccattcttactgctctcttccaaaacaaaactcagcatgatactttgagattgtgttctgagagatgatatgactacataaaatacaattaaaaatgttataatataatgaaaatgtagtaatttaagaaaacaccagatgagttaggaataaacctataacatttacaaaaagagcaaaactaagtgatagaaaatatttcacacatgttcttatagatcatgtatcacttgcaagttttaggagttcatatcctatatcatttcaaattaagtacataataaagtaaaattttgaaatgaacactttaggtatttttgccaagatttagatgtttttaattaaacttttctcttcctttttttttcactaaggcatgtttattcccctaatcc

SEQ ID NO: 349 - DNA, 512 bp, Artificial Sequence
  Synthetic
tgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatca

SEQ ID NO: 350 - DNA, 542 bp, Artificial Sequence
  Synthetic
gtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgt

SEQ ID NO: 351 - DNA, 556 bp, Artificial Sequence
  Synthetic
ctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaa

SEQ ID NO: 352 - DNA, 275 bp, Artificial Sequence
  Synthetic
  misc_feature (70)..(70): n is a, c, g, or t
  misc_feature (170)..(170): n is a, c, g, or t
tgattgcattcaacttcgttacttgagcttcagcaacttaagaacttctgaagttcttaaagttctgaanttcttaaagcccatggatcctttctcagaaaaataactgtaaatctttctggacagccatgactgtagcaaggcttgatagcagaagtttggtggttcanaattatacaactaatcccaggtgattttatcaattccagtgttaccatctcctgagttttggtttgtaatcttttgtccctcccacccccacagaagattttaag

SEQ ID NO: 353 - DNA, 243 bp, Artificial Sequence
  Synthetic
gtaacttgtacacggctcatcgagaaaaagaaaagacaacaagaggattacctgctttacaagcaaatgaagcaactgaaaaaccccttggaaatagtcagcatacctgataatcatggacatattgatccctctctgagactcatctgggatttggctttccttggaagctcttacgtgatgtgggaaatgacaacacaggtgtcacactactacttggcacagctcactagtgtgaggcag

SEQ ID NO: 354 - DNA, 489 bp, Artificial Sequence
  Synthetic
aaagcccttcatctaatatttgttgctattgccaatttttcaatgaaatgacctaaaaacaacaaaaaaaaataacctatacggtagttgctttagggggtggggggatgctatctgttagtgcttaaaagggggtaaatgcttgccgctttagaggtggatggtgctcataaaaggccccagtcgggggtatttaaaaaggactgaacagaaatccttagctagtagaatggcagcacgctgtaaaattattactgtattgtgtactggctataagatgtagacacctttcagtaagccaatcatttgtaaccattctagcagtgtcatattaggttaataaggctgctgtgttttaaagggcatttttatttgggttttggtgaaattctttaatttgttgattatattcacataaaatcagcattcattgacacatagctctaatgacatatgtatgaaaaaccatacactggatgacctagtcga

SEQ ID NO: 355 - DNA, 396 bp, Artificial Sequence
  Synthetic
gcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttact

SEQ ID NO: 356 - DNA, 375 bp, Artificial Sequence
  Synthetic
agggtaacttccagtgtcacaatgagcagttctgtaagtgggtgcctctcagcacatttctatgaatatattatgtagataggctgtattgattttggtagcattgacaccttcttaggcaattagttgaagaaaactgcaaaatattttcttatgtaatagctgtatagagcaatagcaatcaaagcatgagaaggcactaacgctgggatgaaagatgagattcagaggtgactgagaatcatgtgagtgatggctgtatattttgtgtaaaatatatgtgtgaaaatgaactaagagtgagttactcagcactctcaagaattatgcagattctgcatttttcttatgccgtgtgcctaaaaacctacttga

SEQ ID NO: 357 - DNA, 511 bp, Artificial Sequence
  Synthetic
tctcggttggagcgggtgggtgggattgtggcgttgtggtctttgcattgggggaggggggagggaccggatgggcggggggagggggagggggaggggtgggcggccgaaagccaactgtttgtactgaatggcaagaatgttctagtaaatgtgtaccaaaatgtgaattactttgtacgattacagtctccacgtcgacctaacccaatattattggtattaatgtgctttttttgtataaagtgcaaacatttcgtcccaaagtctaagtactttagtgcagtaaaatgttgtttcatgtcctgtcaagaattcgtatagtacgagcctggatctgcgtgtcaaactgttccatttgtttatgtaaagtgatattaaaaaagatataaactataactgtccgttacttttggcaaaagatacaaccacataatgtatataattcctagtttccatatttatccgcatgtaaagggccggtttatccatgttacagctcttcaatatt

SEQ ID NO: 358 - DNA, 455 bp, Artificial Sequence
  Synthetic
gtgagctgggagtcctcatcaggtattgctgaattttggatcaatgggacacctttggtgaaaaagggtctgcgacagggttactttgtggaagctcagcccaagattgtcctggggcaggaacaggattcctatgggggcaagtttgataggagccagtcctttgtgggagagattggggatttgtacatgtgggactctgtgctgcccccagaaaatatcctgtctgcctatcagggtacccctctccctgccaatatcctggactggcaggctctgaactatgaaatcagaggatatgtcatcatcaaacccttggtgtgggtctgaggtcttgactcaacgagagcacttgaaaatgaaatgactgtctaagagatctggtcaaagcaactggatactagatcttacatctgcagtctttcttctttgaatttcctatctgtatgtctgcc

SEQ ID NO: 359 - DNA, 560 bp, Artificial Sequence
  Synthetic
gtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggccacgagcttgttctttgttctttgtcacagggactgaaaacctctcctcatgttctgctttcgattcgttaagagagcaacattttacccacaca

SEQ ID NO: 360 - DNA, 559 bp, Artificial Sequence
  Synthetic
gtgtgctcccgtgagatgctgaatctcctttgcagcaatgccttatttataatttgtggatttgacagtaagaactttaacacgagtcgcttggatgtgtatctatcacataatccagcaggaacttctgttcaaaacatgttccattggacccaggctgttaagtctgggaaattccaagcttatgactggggaagcccagttcagaataggatgcactatgatcagtcccaacctccctactacaatgtgacagccatgaatgtaccaattgcagtgtggaacggtggcaaggacctgttggctgacccccaagatgttggccttttgcttccaaaactccccaatcttatttaccacaaggagattcctttttacaatcacttggactttatctgggcaatggatgcccctcaagaagtttacaatgacattgtttctatgatatcagaagataaaaagtagttctggatttaaagaattatccgtttgtttttccaaaatactttattctctcatacatagtattttcataatgtttgacatgcagtgcttcttt

SEQ ID NO: 361 - DNA, 489 bp, Artificial Sequence
  Synthetic
gccagggcattggctcatgtctgtaatcccagcactttgggaggtcgggggggtgggggaggatcacggggtcaggagatcgagaccatcctggccaacatgatgaaaccctatctctactaaaaatacaaaaattagccgggcatggtggtgcacgcctatagtcccagctactcggggggctgaggtaggagaatcgtttgaactcaggaggcagaggttgcaatgagccaagatcgcgccactgcactccagcctgggtgacagagcgagaccgtctcaaaaagaaagaagtgactaggttcagagaaccagggttcaaagcccagggatgcaaaggttgcagtgagttgagtcatgggatcccagacttttttaaatgtttgcaatgtttcccgtttacagaatgctacaagaataatgtacgtactacctaaaaggatgtctaaatgtttgttaataaaaataagaaatagctacagtgacaga

SEQ ID NO: 362 - DNA, 526 bp, Artificial Sequence
  Synthetic
gattagtaaagcacatgttccctcttggaagatgactctgctaaatgtttgcagtcttgtaaataatttgaacagcccagctgaggaaacaggagaagttcatgaagaggagcttgttgcaagaaggaaacttcctactgctttagatggctttagcttggaagcaatgttgacaatataccagctccacaaaatctgtcacagcagggcttttcaacactgggagttaatccaggaagatattcttgatactggaaatgacaaaaatggaaaggaagaagtcataaagagaaaaattccttatattctgaaacggcagctgtatgagaataaacccagaagaccctacatactcaaaagagattcttactattactgagagaataaatcatttatttacatgtgattgtgattcatcatcccttaattaaatatcaaattatatttgtgtgaaaatgtgacaaacacacttatctgtctcttctacaattgtggtttattgaatgtgtttttctgcactaataga

SEQ ID NO: 363 - DNA, 498 bp, Artificial Sequence
  Synthetic
gtggaccttagaatacagttttgagtagagttgatcaaaatcaattaaaatagtctctttaaaaggaaagaaaacatctttaaggggaggaaccagagtgctgaaggaatggaagtccatctgcgtgtgtgcagggagactgggtaggaaagaggaagcaaatagaagagagaggttgaaaaacaaaatgggttacttgattggtgattaggtggtggtagagaagcaagtaaaaaggctaaatggaagggcaagtttccatcatctatagaaagctatataagacaagaactcccctttttttcccaaaggcattataaaaagaatgaagcctccttagaaaaaaaattatacctcaatgtccccaacaagattgcttaataaattgtgtttcctccaagctattcaattcttttaactgttgtagaagacaaaatgttcacaatatatttagttgtaaaccaagtgatcaaactacatattgtaaagcccattttt

SEQ ID NO: 364 - DNA, 404 bp, Artificial Sequence
  Synthetic
gccttcaagtggccgtttctggggcctcgacagtgcatcgcctcggagactgactcgctgcccatgatcgtcagcatcaaggagggaggcaggaccaggccccaggtggtcagcctgcccaacatgagggtgcagaagtgcagctgtgcctcggatggtgcgctcgtgccaaggaggctccagccataggcgcctagtgtagccatcgagggacttgacttgtgtgtgtttctgaagtgttcgagggtaccaggagagctggcgatgactgaactgctgatggacaaatgctctgtgctctctagtgagccctgaatttgcttcctctgacaagttacctcacctaatttttgcttctcaggaatgagaatctttggccactggagagcccttgctcagttttc

SEQ ID NO: 365 - DNA, 337 bp, Artificial Sequence
  Synthetic
actgactggacgcacgtggaaaggagctatttttggaggctttaagagtaaagaatctgtcccgaaacttgtggctgactttatggctaagaagttttcactggatgcattaataacaaatattttaccttttgaaaaaataaatgaaggatttgacctgcttcgctctggaaagagtatccgtaccgtcctgacgttttgaaacaatacagatgccttcccttgtagcagttttcagcctcctctaccctacatgatctggagcaacagctaggaaatatcattaattctgctcttcagagatgttaaaaataaattacacgtgggagctttccaa

SEQ ID NO: 366 - DNA, 331 bp, Artificial Sequence
  Synthetic
gagacgtggtaagtgcggtgcagttttcaactgacctctggacgcagaacttcagccatgaaggtaacaggcatctttcttctcagtgccttggccctgttgagtctatctggtaacactggagctgactccctgggaagagaggccaaatgttacaatgaacttaatggatgcaccaagatatatgaccctgtctgtgggactgatggaaatacttatcccaatgaatgcgtgttatgttttgaaggtcggaaacgccagacttctatcctcattcaaaaatctgggccttgctgagaaccaaggttttgaaatcccatcaggtcaccgc

SEQ ID NO: 367 - DNA, 365 bp, Artificial Sequence
  Synthetic
ggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgaaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctagggatctgtgccag

SEQ ID NO: 368 - DNA, 513 bp, Artificial Sequence
  Synthetic
gtctgagccagagttgtagccaggccttgcactactgccatagccaatcaatctcccaaagccaagtttacctgctgttctcacccccaatgaggtcttaggcagattctttactttgtaattcaaaaggcctgttaacctttgttcttgttatgcatgaataacttgttgactttttttttttctctttttcaactagtgtttcaacaggtttatttagattcagaatgtaaatttacaaatgatatagatcaccctgttattgctgtttttcagggacagtagaaagtgttttgttttttgagtgaaacaacttattaataaaaatccttaaatcacttttgtaatccaggacaagaaaatgtggcattagagtagaaatatctttggaaatgtgaaagaccccatagtgaaatatttgggatcagaagccagaggtgtaaaagtggtcaattcttggtttacttcatttaatctttcttttcataaagatacatacctggatttgtttat

SEQ ID NO: 369 - DNA, 361 bp, Artificial Sequence
  Synthetic
gagctctgccacaaacatggctatccaccggtcccagccatggtttcaccacaaaatttctagagatgaggctcagcgattgattattcagcaaggacttgtggatggagttttcttggtacgggatagtcagagtaaccccaaaactttcgtactgtcaatgagtcatggacaaaaaataaagcactttcaaattataccagtagaagatgacggtgaaatgttccacacactggatgatggccacacaagatttacagatctaatacagctggtggagttctatcaactcaataagggcgttcttccttgcaagttgaaacattattgtgctaggattgctctctagacaagccagaag

SEQ ID NO: 370 - DNA, 522 bp, Artificial Sequence
  Synthetic
aaataggcactcacaatgacaaccagagccagtttcttgtctttttatacattttgtcatcccagagactcggtatttgcttactgtgtttcaagtagaggaaatcgtggtcttgaactattctgtaccacagcaaacaatctatgttgctttactatcaactgctgtaatcgtttataaaacttacctagctccttcccttcttctatcatagctttaaacattagaattcataggcaaatcagttaaaacattaggatcataggcaaatcagttaccttgcagaaagagctttgtatgacagacattgtcttattttatttctgtaaaatattagctgtatgaatatgatttaattaacaagaaaacatttcttcctgattgacaacagtgttagcaaggtgcaaagcgaaactggttgctcaagttgatagaaaacaaaattctgaatatcttcaaattaattcggtaaaaacacattattttttcatatgtgatgtattcatgcagaacaactatctt

SEQ ID NO: 371 - DNA, 513 bp, Artificial Sequence
  Synthetic
atattagttaccctggtgtgctgtattctctaaaacctttaaatgtttgcatgcagccattcgtcaaatgtcaaatattctctctttggctggaatgacaaaaactcaaataaatgtatgattaggaggacatcataacctatgaatgatggaagtccaaaatgatggtaactgacagtagtgttaatgccttatgtttagtcaaactctcatttaggtgacagcctggtgactccagaatggagccagtcatgctaaatgccatatactcacactgaaacatgaggaagcaggtagatcccagaacagacaaaactttcctaaaaacatgagagtccaggctgtctgagtcagcacagtaagaaagtcctttctgctttaactcttagaaaaaagtaatatgaagtattctgaaattaaccaatcagtttatttaaatcaatttatttatattcttctgttcctggattcccattttacaaaacccactgttctactgttgtattgcccagt

SEQ ID NO: 372 - DNA, 470 bp, Artificial Sequence
  Synthetic
aacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcatt

SEQ ID NO: 373 - DNA, 498 bp, Artificial Sequence
  Synthetic
aatatagttaaacctttgccgccagtctatcttacttttactcgggagagttcatgtgaaattaagctgaaatggagcatacctttgggacctattccagcaaggtgttttgattatgaaattgagatcagagaagatgatactaccttggtgactgctacagttgaaaatgaaacatacaccttgaaaacaacaaatgaaacccgacaattatgctttgtagtaagaagcaaagtgaatatttattgctcagatgacggaatttggagtgagtggagtgataaacaatgctgggaaggtgaagacctatcgaagaaaactttgctacgtttctggctaccatttggtttcatcttaatattagttatatttgtaaccggtctgcttttgcgtaagccaaacacctacccaaaaatgattccagaatttttctgtgatacatgaagactttccatatcaagagacatggtattgactcaacagtttccagtcatggcc

SEQ ID NO: 374 - DNA, 528 bp, Artificial Sequence
  Synthetic
ccgtcccacgttacatcaaattcagttttatataggccatatataatatatatttataatgtataatttttatgtatttttcaaaactacaaactggaatccaactataaagtgtttaagaatctacacagaatattcaaattatagaacatgttttttccctttgccccataatcagtatttgccaaattacatgcaattccttaaaaactaaatcacattggtaaaaggcctacagctttgtacttacattgtgccaaaggctgaggaaatgttttctttcgaatttttatgtgtattgtaaaatgttctaccgtactttagtagtttgaagttttcaagtgcataactatttttgaccagcagaaggcgatacgcttcagtattttatgcaattttttttcacttcgaagggaaagtgtattataaaaaaagattttttttttttaaaacatgctactcttaattttcatgttggtgatgaaattcccagtggtgtttcttaaggttctatcttgtgccatgatg

SEQ ID NO: 375 - DNA, 554 bp, Artificial Sequence
  Synthetic
aaactcacacttaaaacacatgccagttgggaaggtctgaaaactcagtgcataataggaacacttgagactaatgaaagagagagttgagaccaatctttatttgtactggccaaatactgaataaacagttgaaggaaagacattggaaaaagcttttgaggataatgttactagactttatgccatggtgctttcagtttaatgctgtgtctctgtcagataaactctcaaataattaaaaaggactgtattgttgaacagagggacaattgttttacttttctttggttaattttgttttggccagagatgaattttacattggaagaataacaaaataagatttgttgtccattgttcattgttattggtatgtaccttattacaaaaaaaatgatgaaaacatatttatactacaaggtgacttaacaactataaatgtagtttatgtgttataatcgaatgtcacgtttttgagaagatagtcatataagttatattgcaaaagggatttgtattaatttaagactatttttgtaaagctctactgt

SEQ ID NO: 376 - DNA, 494 bp, Artificial Sequence
  Synthetic
taacaatccatgatgctgttttgcatattttgatgaagaaagattacagtacttcaaagtttaatcccagtcaggaaaaagatggaaaaattgattttaccataaatacaaatggaggattacgtaatcgggtatatgaggtgccagttgaaacaaaattctaatcaacatataattcagaaggatcttcatctgactatgacataaaaacaactttatacccagaaagttattgataagttcatacattgtacgaagagtatttttgacagaatatgtttcaaactttggaacaagatggttctagcatggcatatttttcacatatctagtatgaaattatataagtattctaaattttatatcttgtagctttatcaaagggtgaaaattattttgttcatacatatttttgtagcactgacagatttccatcctagtcactaccttcatgcataggtttagcagtatagtggcgccactgttttgaatct

SEQ ID NO: 377 - DNA, 529 bp, Artificial Sequence
  Synthetic
ctttctatattgttatcagtccaggaaacaggtaaacagatgtaattagagacattggctctttgtttaggcctaatctttctttttacttttttttttcttttttctttttttttaaagatcatgaatttgtgacttagttctgccctttggagaacaaaagaaagcagtcttccatcaaatcaccttaaaatgcacggctaaactattcagagttaacactccagaattgttaaattacaagtactatgctttaatgcttctttcatcttactagtatggcctataaaaaaaataataccacttgatgggtgaaggctttggcaatagaaagaagaatagaattcaggttttatgttattcctctgtgttcacttcgccttgctcttgaaagtgcagtatttttctacatcatgtcgagaatgattcaatgtaaatatttttcattttatcatgtatatcctatacacacatctccttcatcatcatatatgaagtttattttgagaagtctacattgcttacattt

SEQ ID NO: 378 - DNA, 456 bp, Artificial Sequence
  Synthetic
aattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattat

SEQ ID NO: 379 - DNA, 503 bp, Artificial Sequence
  Synthetic
actctagtgggaccttgactggagaaactccacgatcttcctgaagaagagaaatggatgtttccaaattccacaataagcaatatgtggtgataatgaaaagaatgattcagtcttgacggtgaatggaagacacttacctaacaagtactgctcatttacactcaaattaatcttgaagtagtcttaaaatgtgtaagaagttaaaacttgagaagcaaaaaatgcctgcaaaaagaagatcattttgtatacagagaaccggatgaatataagcaatgaagatgaacatttattgatcttctacatacaagacttcaccataaggccaggagcagtgctcacgccttgtaatcccagcactttgggaggccaaggtgggcggatcaccttgaggtcaggagttcaagaccagcctgaccaacatggtgaaaccctgtctctactaaatattagcggggtgtggtggcgggcacctgtagtcgcagcctttcgggaggctg

SEQ ID NO: 380 - DNA, 405 bp, Artificial Sequence
  Synthetic
ctgagcatactcaacaaaacccatgcatttcataaactaatagaagttgaggattgttgaatctatttcacttattttggctgtggtttccatctgaaagtagaggttgtatacaccatatactgttcttcattttattaatatttttctccttgacctctcataaatttactttacacaattcttaccctgtacatatgtaaacataagtgtacgattcttaaccatggagtagaggtactagaatgcttacggccatctctttgtacaggaactgcattgactttcagtaaacataaagccacaactcctacatgatgttatgtaccatatgatctgttttgtatcttaaatttgatttacatatattatttatttctggtaactcactcagtttatgctgtg

SEQ ID NO: 381 - DNA, 441 bp, Artificial Sequence
  Synthetic
atagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagat

SEQ ID NO: 382 - DNA, 400 bp, Artificial Sequence
  Synthetic
attttattgacttcgtggccagggaaaccacatgttccaaggaaagtaatgaagagttgaccgaaagctgtgagaccaaaaaacttggccaaagcctagattgcaacgctgaagtttatgtggtaccctgggagaaaaaaatttaccctactgtcaactgtcaaccactgggaatgatctcactgatgaaaaggcctccaggtttttcacctttccgatcatcacgaataggggaaataaaagaagaaacaactagtcacctaaggtcctgcgagtacaagggtcgacccccaaaggcaggggcagagccagcatctgagagggaggtctcttgaccaatgggcagaatcttcactccaggcacatagccccaaccacctctgccagcaaccttgagagg

SEQ ID NO: 383 - DNA, 346 bp, Artificial Sequence
  Synthetic
catcgctctcaagaccgaagacatcatcacagcgattcgccacttgagagagagaggcctggagttcttatctgttccctccacgtactacaaacaactgcgggagaagctgaagacggccaagatcaaggtgaaggagaacattgatgccctggaggagctgaaaatcctggtggactacgacgagaaaggctacctcctgcagatcttcaccaaaccggtgcaggaccggcccacgctcttcctggaagtcatccagcgccacaaccaccagggttttggagccggcaacttcaactcactgttcaaggctttcgaggaggagcagaacctgcggggtaacctc

SEQ ID NO: 384 - DNA, 385 bp, Artificial Sequence
  Synthetic
tgccacgcacgactgaacacagacagcagccgcctcgccatgaagctgctgatggtcctcatgctggcggccctcctcctgcactgctatgcagattctggctgcaaactcctggaggacatggttgaaaagaccatcaattccgacatatctatacctgaatacaaagagcttcttcaagagttcatagacagtgatgccgctgcagaggctatggggaaattcaagcagtgtttcctcaaccagtcacatagaactctgaaaaactttggactgatgatgcatacagtgtacgacagcatttggtgtaatatgaagagtaattaactttacccaaggcgtttggctcagagggctacagactatggccagaactcatctgttg

SEQ ID NO: 385 - DNA, 511 bp, Artificial Sequence
  Synthetic
tccacacacggccaggcctgtttatctacactgctgcccactcctctctccagctccacatgctgtacctggatcattctgaagcaaattccgagcattacatcattttgtccataaatatttctaacatccttaaatatacaatcggaattcaagcatctcccattgtcccacaaatgtttggctgtttttgtagttggattgtttgtattaggattcaagcaaggcccatatattgcatttatttgaaatgtctgtaagtctctttccatctacagagtttagcacatttgaacgttgctggttgaaatcccgaggtgtcatttgacatggttctctgaacttatctttcctataaaatggtagttagatctggaggtctgattttgtggcaaaaatacttcctaggtggtgctgggtacttcttgttgcatcctgtcaggaggcagataatgctggtgcctctctattggtaatgttaagactgctgggtgggtttggagttcttggc

SEQ ID NO: 386 - DNA, 270 bp, Artificial Sequence
  Synthetic
gcaacactcaagctgagaggtccataataggcatgatcgacatgtttcacaaatacaccagacgtgatgacaagattgacaagccaagcctgctgacgatgatgaaggagaacttccccaacttccttagtgcctgtgacaaaaagggcacaaattacctcgccgacgtctttgagaaaaaggacaagaatgaggataagaagattgatttttctgagtttctgtccttgctgggagacatagccacagactaccacaagcagagccatg

SEQ ID NO: 387 - DNA, 558 bp, Artificial Sequence
  Synthetic
gatagcatcgtcaaccctgatggctttgctggattcccctgtgcctcttacaacgtcttcactgcaaacaagtgtttcccttgtccaagtggaggctgcccacagatgggtcactatgctgatagatatcctgggaaaacaaatgatgtgggccagaaattttatctagacactggtgatgccagtaattttgcacgttggaggtataaggtatctgtcacactgtctggaaaaaaggttacaggacacatactagtttctttgttcggaaataaaggaaactctaagcagtatgaaattttcaagggcactctcaaaccagatagtactcattccaatgaatttgactcagatgtggatgttggggacttgcagatggttaaatttatttggtataacaatgtgatcaacccaactttacctagagtgggagcatccaagattatagtggagacaaatgttggaaaacagttcaacttctgtagtccagaaaccgtcagggaggaagttctgctcaccctcacaccgtgttaggagactactgttatttgaccaatg

SEQ ID NO: 388 - DNA, 95 bp, Artificial Sequence
  Synthetic
gactccaaggaagctcagatgcctgcagtcattaggttttagcgtcccatgagccttggtatcaagaggccacaagagtgggaccccaggggctc

SEQ ID NO: 389 - DNA, 471 bp, Artificial Sequence
  Synthetic
gaacctaggagagtcaacatctggaggattttagtctttcttacacatatgtgtgattttaaacgaatattctcagaccacaggaaactcttcatccccctgttgtttaccagtaacagtatatcacagacctttccaaatgtttgtatatgtaatcagatgtacatttatattgaaaaacaaatgagatggacttaaagagcacatcctgataaatactttctctctcacctgtactatatttctattagactaaagttatgtgattttttttttacattttttcagatgactagcaattttgatagtttataagataatgcaaagaactttctctgacaaactaactgcagtaacagaaacctttcttttcagttactctttttcaagaatgaaagattattatacaaaaaattgtatactacttgatggaaccaactttgtacatcttggccatgtcactggtcattg

SEQ ID NO: 390 - DNA, 220 bp, Artificial Sequence
  Synthetic
gaagagggagctctattgccaccatgagtttctccggcaagtaccaactgcagagccaggaaaactttgaagccttcatgaaggcaatcggtctgccggaagagctcatccagaaggggaaggatatcaagggggtgtcggaaatcgtgcagaatgggaagcacttcaagttcaccatcaccgctgggtccaaagtgatccaaaacgaattcacggtggg

SEQ ID NO: 391 - DNA, 508 bp, Artificial Sequence
  Synthetic
gatcttaaagccacggagaagcctctcatcttatggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctggggatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttga

SEQ ID NO: 392 - DNA, 330 bp, Artificial Sequence
  Synthetic
tggcctcagaatgcattcttacacatctatcctagacatttataagcactctaatggataacaatccaagaataaatgattgtaaaagatgatgccgaagagttgatgtcaatctttttttcctaagaaaaaaagtccgcgagtattaaatatttagatcaatgtttataaaatgattactttgtatatctcattattcctattttggaataaaaactgaccttctttaatcatatacttgtcttttgtaaatagcagcttttgtgtcattctccccactttattagttaatttaaattggaaaaaaccctcaaactaatattcttgtct

SEQ ID NO: 393 - DNA, 361 bp, Artificial Sequence
  Synthetic
tcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtg

SEQ ID NO: 394 - DNA, 275 bp, Artificial Sequence
  Synthetic
gaaatactcagcttaggcatttttactttaacccctaaattgattttgtaaatgccacaaatgcatagaattgttaccaacctccaaagggctctttaaaatcatatttttttattcatttgaggatgtcttataaagactgaaggcaaaggtcagaatgcttacgggtgttatttttataagttgttgaattccttaatttagaaaagctcattattttttgcacactcacaatattctctctcagaaatcaatggcatttgaaccaccaaaaa

SEQ ID NO: 395 - DNA, 471 bp, Artificial Sequence
  Synthetic
gaaaaccagagagtcccattttaagaaattagatatttaatatagcattgtgtgttctattttagtaacagcagaatctcttgacattacacaactcagtgaaacaacatcatttaagccaaaatatctcccaactgactgatagactctgagcactaatatcatagtgctgtgatgatggacaattacatagtaccgataacagccatgcactgtgcaaagcatgcccttctgcacaggagagcaaggcacttgcagtagtgatctatgccagcaaaacatcattttgagacaaacatttttgtggcagatgtttttcctaaaaagtactatatcatccaagaaatatttgagtaaaatcccttgttcttttgggtgacattaactgacatttgctttttttcaagacctaatagaaaataagaaagcccataatgtatttagaaacaggaatcctcagagcaattctct

SEQ ID NO: 396 - DNA, 523 bp, Artificial Sequence
  Synthetic
agtggcacattacatatcggatctaagagattaataccattagaagttacacagttttagttgtttggagatagttttggtttgtacagaacaaaataatatgtagcagcttcattgctattggaaaaatcagttattggaatttccacttaaatggctatacaacaatataactggtagttctataataaaaatgagcatatgttctgttgtgaagagctaaatgcaataaagtttctgtatggttgtttgattctatcaacaattgaaagtgttgtatatgacccacatttacctagtttgtgtcaaattatagttacagtgagttgtttgcttaaattatagattcctttaaggacatgccttgttcataaaatcactggattatattgcagcatattttacatttgaatacaaggataatgggttttatcaaaacaaaatgatgtacagattttttttcaagtttttatagttgctttatgccagagtggtttaccccattcacaaaatttcttatgca

SEQ ID NO: 397 - DNA, 530 bp, Artificial Sequence
  Synthetic
tttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttg

SEQ ID NO: 398 - DNA, 242 bp, Artificial Sequence
  Synthetic
tggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttca

SEQ ID NO: 399 - DNA, 554 bp, Artificial Sequence
  Synthetic
tgctatgccttgtttttgtcaccaaaatcctggacagatgcagatctggcctgccagaagcggccctctggaaacctggtgtctgtgctcagtggggctgagggatccttcgtgtcctccctggtgaagagcattggtaacagctactcatacgtctggattgggctccatgaccccacacagggcaccgagcccaatggagaaggttgggagtggagtagcagtgatgtgatgaattactttgcatgggagagaaatccctccaccatctcaagccccggccactgtgcgagcctgtcgagaagcacagcatttctgaggtggaaagattataactgtaatgtgaggttaccctatgtctgcaagttcactgactagtgcaggagggaagtcagcagcctgtgtttggtgtgcaactcatcatgggcatgagaccagtgtgaggactcaccctggaagagaatattcgcttaattcccccaacctgaccacctcattcttatctttcttctgtttcttcctccccgctgtcatttcagtctcttcattttgtc

SEQ ID NO: 400 - DNA, 514 bp, Artificial Sequence
  Synthetic
gtctacttcatgccaatgagctactcctcaacaggggctggttttgccatttgaggaatgacagccactatgttgtgtacacaagagagctggatggcatcgacagaatctttatcgtggttctgaattttggagaatcaacactgttaaatctacataatatgatttcgggccttcccgctaaaataagaataaggttaagtaccaattctgccgacaaaggcagtaaagttgatacaagtggcatttttctggacaagggagagggactcatctttgaacacaacacgaagaatctccttcatcgccaaacagctttcagagatagatgctttgtttccaatcgagcatgctattccagtgtactgaacatactgtatacctcgtgttaggcacctttatgaagagatgaagacactggcatttcagtgggattgtaagcatttgtaatagcttcatgtacagcatgctgcttggtgaacaatcattaattcttcgatatttctgtagcttg

SEQ ID NO: 401 - DNA, 555 bp, Artificial Sequence
  Synthetic
caccacctacctatgatgccgtggtacagatggagtaccttgacatggtggtgaatgaaacactcagattattcccagttgctattagacttgagaggacttgcaagaaagatgttgaaatcaatggggtattcattcccaaagggtcaatggtggtgattccaacttatgctcttcaccatgacccaaagtactggacagagcctgaggagttccgccctgaaaggttcagtaagaagaaggacagcatagatccttacatatacacaccctttggaactggacccagaaactgcattggcatgaggtttgctctcatgaacatgaaacttgctctaatcagagtccttcagaacttctccttcaaaccttgtaaagaaacacagatccccttgaaattagacacgcaaggacttcttcaaccagaaaaacccattgttctaaaggtggattcaagagatggaaccctaagtggagaatgagttattctaaggacttctactttggtcttcaagaaagctgtgccccagaacaccagagatttcaacttagtca

SEQ ID NO: 402 - DNA, 476 bp, Artificial Sequence
  Synthetic
ggaacaggccgtgcgagcactttcagctggctggacacagtcacagtcacgcaggatgggctgtccatgatgatcaacaggaagaacatggtggtctcctttggagatggggttaccttcgtggtcgtcctacaccaggtgtggaagaaacatcctgtccaccgtgactttctaggcttctacgtggtggacagtcaccggatgtcagcacagacgcatgggctgctggggcaattcttccaaccctttgactttaaagtgtctgacatccggccaggctctgaccccacaaagccagatgccacattggtggtgaagaaccatcagctgattgtcaccaggggctcccagaaagactacagaaaggatgccagcatcggcacgaaggttgtctgctggttcgtccacaacaacggagaagggctgattgatggtgtccacactgactacattgtccccaacctgttttgagtaga

SEQ ID NO: 403 - DNA, 480 bp, Artificial Sequence
  Synthetic
  misc_feature (332)..(332): n is a, c, g, or t
  misc_feature (335)..(335): n is a, c, g, or t
  misc_feature (342)..(342): n is a, c, g, or t
  misc_feature (394)..(394): n is a, c, g, or t
gatttagtggtggactttaacctcttaaataaatgttagtatatcagattgtgtccttgaaaaatattttacttgtatgaatcatgacaacgtctaaatctttactattcttctggcaaaagcatcagtaagaaagaaggcgaaaaagagaagtatagcctttatgtcagaaaaacattctttttagctgcttactttctcatgaaaagtaaagatgtttacagtgtatgccaagttttcagtttctgtataacaacaggtagaggttctaatcatattgaaaattgtgttataatggtctgagccatgttgctaggaaacaataggttccnaantttgtantcctgctctcctgtgctgaaaagtgactggatactgtacaggttcatgttcntctggctgcagttaaatggtcttttgcattttgctctggctttcaggccagaagcatgcatttttctacaagagcatcacaacaac

SEQ ID NO: 404 - DNA, 494 bp, Artificial Sequence
  Synthetic
atcagtaatctgtcgcatttcatcaagattaattaaaatttgggacctgcttcattcaagcttcatatatgctttgcagagaactcataaaggagcatataaggctaaatgtaaaacacaagactgtcattagaattgaattattgggcttaatataaatcgtaacctatgaagtttattttctattttagttaactatgattccaattactactttgttattgtacctaagtaaattttctttaggtcagaagcccattaaaatagttacaagcattgaacttctttagtattatattaatataaaaacatttttgtatgttttattgtaatcataaatactgctgtataaggtaataaaactctgcacctaatccccataacttccagtatcattttccaattaattatcaagtctgttttgggaaacactttgaggacatttatgatgcagcagatgttgactaaaggcttggttggtagatattcaggaa

SEQ ID NO: 405 - DNA, 425 bp, Artificial Sequence
  Synthetic
caaacgtattggcaggcgaaccccttccgtgctgtggccgagcctggcatccaactcaaggctgtgaagtcttccacaggccccggggaacagctgcggaacgctctgtggcatacaggagacacagagtcccaggtgcggctgctgtggaaggacccgcgaaacgtgggttggaaggacaagaagtcctatcgttggttcctgcagcaccggccccaagtgggctacatcagggtgcgattctatgagggccctgagctggtggccgacagcaacgtggtcttggacacaaccatgcggggtggccgcctgggggtcttctgcttctcccaggagaacatcatctgggccaacctgcgttaccgctgcaatgacaccatcccagaggactatgagacccatcagctgcggcaagcctagggacc

SEQ ID NO: 406 - DNA, 410 bp, Artificial Sequence
  Synthetic
gaacatgctctgacatcggtgcatccgcgaactcgatattcagctggctgggatgctaaatttttcttcatccctctatcttatttacctacatcactggcagactacattttgactagatcttggcccaaaccagcccaggcagtctaaagaaaactgggttggtgcttcttggaatgaaggcaaaaatctgaaattgttagtgtctcagtaatcctgatttagaacccaggctttttgtaacaatgtgttttcttgcctaaattcatttatctggcatcatcagagtactaacatgtttatatttcagatatccaaagcttaccactttaggtgatgaatctttactattttagcccttttttgatgagactatttgtctaaagtgaatcatttgttcttgccttatt

SEQ ID NO: 407 - DNA, 350 bp, Artificial Sequence
  Synthetic
ttagtctcaggttcaaggctacaacaaaaatcaccatctttgtcaaactttggagagggaaaatcttcactttcttaagcaacaatggatattgcctgtgtttgccactgtgtttccctgcctctcaattcgctgaaaaaggaactacctatccttacatttcacctactaatgtctcttctaacatcttagaggtccatggagaaggcatatggagaacatgttttatactgctctataaatagtattccaatcactgtgcttaatttaaatagcattctcttatcatttatcagccttttatgtattttccaagtaaaatattaacatattacttcattggtcttctt

SEQ ID NO: 408 - DNA, 403 bp, Artificial Sequence
  Synthetic
aaaatgttgcgttctcagtccaaaaagaagtggaaaagaatctgaagtcatgcttggacaatgttaatgttgtgtccgtagacactgccagaacactattcaaccaagtgatggaaaaggagtttgaagacggcatcattaactggggaagaattgtaaccatatttgcatttgaaggtattctcatcaagaaacttctacgacagcaaattgccccggatgtggatacctataaggagatttcatattttgttgcggagttcataatgaataacacaggagaatggataaggcaaaacggaggctgggaaaatggctttgtaaagaagtttgaacctaaatctggctggatgacttttctagaagttacaggaaagatctgtgaaatgctatctctcctgaa

SEQ ID NO: 409 - DNA, 418 bp, Artificial Sequence
  Synthetic
ggaactctaaaccttgtgatgactactaacaaatgtaaaattatgagtgattaagaaaacattgctttgtggttatcactttaagttttgacacctagattatagtcttagtaatagcatccactggaaaaggtgaaaatgttttattcagcatttaacttacatttgtactttagagtatttttgtataaaatccatagatttattttacatttagagtatttacactatgataaagttgtaaataattttctaagacagtttttatatagtctacagttgtcctgatttcttattgaatttgttagactagttctcttgtcttgtgatctgtgtacaattttagtcactaagactttcctccaagaactaagccaacttgatgtgaaaagcacggctgtatataatggtgatgtca

SEQ ID NO: 410 - DNA, 326 bp, Artificial Sequence
  Synthetic
ttcgacactgcctcaactggaaaaacattcccaggtttcttctcacctatgttaggagagtttgtcagtgagactgagtctaggggctcagaatctggcatcttcacaaatacaaaggaatccagttctcatcaccctgggatagctgaattcccttcccgtggtaaatcttcaagttacagcaaacaatttactagtagcacgagttacaacagaggagactccacatttgaaagcaagagctataaaatggcagatgaggccggaagtgaagccgatcatgaaggaacacatagcaccaagagaggccatgctaaatctcgccc

SEQ ID NO: 411 - DNA, 498 bp, Artificial Sequence
  Synthetic
tcactgaatctaaccatagctgacctttgtagtagaatttacaacttataattacaatgcacaatttataattacaatatgtatttatgtcttttgctatggagcaaatccaggaaggcaagagaaacattctttcctaaatataaatgaaaatctatcctttaaactcttccactagacgttgtaatgcacacttatttttttcccaaggagtaaccaatttctttctaaaacacatttaaaattttaaaactatttatgaatattaaaaaaagacataattcacacattaataaacaatctcccaagtattgatttaacttcatttttctaataatcataaactatattctgtgacatgctaattattattaaatgtaagtcgttagttcgaaagcctctcactaagtatgatctatgctatattcaaaattcaacccatttactttggtcaatatttgatctaagttgcatctttaatcctggtggtcttgcctt

SEQ ID NO: 412 - DNA, 537 bp, Artificial Sequence
  Synthetic
gtattctgtggattctatatttcatattgagatcagcattcaaaatagttctatttctatctgcaaatagtttcaaatgagtttaaaaaaataacatctgaaaagaaatgctaatgtaatcatttatcttatctagcaagaagattctaaaacattctttaacatacatctaagtcagtttcacatatttgtagctagaatatcctatactggttatagttgatatgtaacagttggtgattttagatttctttgattgtgaaacagggagctatgagagatgtgtccatgtgaaatttacagttactgcctaggagttaatgatcgttctgggtcagcttgaatgtccccattctataaattcaacacttattttctgaattcataaaaataaccaaaaaatgtgagctataatgtttccctcaagaacaaacagaaacgagatttgccaaaaactaaaattcaacaaatgatgttgagtgggagattggctttgcctttagcgtgtaaatggaagcactgccattagactgaatt

SEQ ID NO: 413 - DNA, 575 bp, Artificial Sequence
  Synthetic
gaaagtctatgcttaggccagtcagtacacccaattttaaaaaataacatattcttgctttcacaaatatagttgaacaagatttccctaaaaattccaccaggattaatctctaaaattctagtctctgatttgcaaatgcacatttgtcactgaataatggaattatgtataacaagccaaacattcttattttagacaaccatagaactgtcccacaaaatatttctaagcttatttctaactattaggaggaatgtgcttttccatctaaaatactcaccaaaatatagttaattgtggctttatgaagttaacagtctcattacagatttagtttaccaatcaacagcatgtctactgcttggatccatacaaaactatcggttcaagttgatgtgacaagggaagggagcaccagatgacacataaatctgtctgattctatgcctgtatttccaacaaacttactgtcagagaatatgacctaaatccattttctaaactgttttcatgtgttgcaaattattctagtcaactgctgttttatgtcatactctgtgtaatctctgatt

SEQ ID NO: 414 - DNA, 344 bp, Artificial Sequence
  Synthetic
ctcatgcgaaacattgtcagggtggtcgtcctggacaaagtcacagacctgctgctgttctttgggaagctgctggtggtcggaggcgtgggggtcctgtccttcttttttttctccggtcgcatcccggggctgggtaaagactttaagagcccccacctcaactattactggctgcccatcatgacctccatcctgggggcctatgtcatcgccagcggcttcttcagcgttttcggcatgtgtgtggacacgctcttcctctgcttcctggaagacctggagcggaacaacggctccctggaccggccctactacatgtccaagagccttctaaagattct

SEQ ID NO: 415 - DNA, 397 bp, Artificial Sequence
  Synthetic
ggattcaaaaggtgtcacagtccacttaattagtcaaattagcaatggctaaacagtatcaagtactgcagaatttatcactgaaatggataagaggaaatagtttagtcacaggtttttacagtccagcaagggccaaagaggtatagtatacaagttaatagtatttgtgttgagcaacatggggctagtgggatcacagaaatctggaaaaaaaaaaaaaaaggctttggcttatcaagcctagtgtaaatttctgcatctcacacgactttagtttggccaggtatttatctgccaaaacaaggacaaatcttgttgtattaacagcagggtcacttctcattttctttgctgacttacctttttactgaccgttgtgaatttctgtctcaaa

SEQ ID NO: 416 - DNA, 474 bp, Artificial Sequence
  Synthetic
gttctggcatcatttacgtagtttacgctaccaatctgaattcgaggcccatatgagaggagcttagaaacgaccaagaagagaggcttgttggaatcaattctgcacaatagcccatgctgtaagctcatagaagtcactgtaactgtagtgtgtctgctgttacctagagggtctcacctccccactcttcacagcaaacctgagcagcgcgtcctaagcacctcccgctccggtgaccccatccttgcacacctgactctgtcactcaagcctttctccaccaggcccctcatctgaataccaagcacagaaatgagtggtgtgactaattccttacctctcccaaggagggtacacaactagcaccattcttgatgtccagggaagaagccacctcaagacatatgaggggtgccctgggctaatgttagggcttaattttctcaaagcctgacctttcaaatccatgat

SEQ ID NO: 417 - DNA, 155 bp, Artificial Sequence
  Synthetic
gcaacggaagagtcctgggcggaaggaggcttctgtatgcttgtgaaaaagaacaatctgtgccaacggaaggttcttcaacaactttgctgcaaaacatgtacatttcaaggctgagcagccatcttagatttctttgttcctgtagacttata

SEQ ID NO: 418 - DNA, 441 bp, Artificial Sequence
  Synthetic
agcggcgggactgagctgttgagttagagccaacatgagtgagcgacaaggtgctgggccaaccaatggaaaagacaagacatctggtgaaaatgatggacagaagaaagttcaagaagaatttgacattgacatggatgcaccagagacagaacgtgcagcggtggccattcagtctcagttcagaaaattccagaagaagaaggctgggtctcagtcctagtgggagaaccccctcctagtccacctgaaagcaccaaattcaaccatcatctgtcaagaaattaaaagaacaacaccctagagagaagtcatccacacacaatccacacacgcatagcaaacctccaatgcatgtacagaaacctgtgatatttatacccttgtaggaaggtatagacaatggaattgtgagtagcttaatctctatgtttctctcca

SEQ ID NO: 419 - DNA, 505 bp, Artificial Sequence
  Synthetic
agcccagtttcactgccatatactcttcaaggactttctgaagcctcacttatgagatgcctgaagccaggccatggctataaacaattacatggctctaaaaagttttgccctttttaaggaaggcactaaaaagagctgtcctggtatctagacccatcttctttttgaaatcagcatactcaatgttactatctgcttttggttataatgtgtttttaattatctaaagtatgaagcattttctggggttatgatggccttacctttattaggaagtatggttttattttgatagtagcttcctcctctggtggtgttaatcatttcatttttacccttactgtttgagtttctctcacattactgtatatactttgcctttccataatcactcagtgattgcaatttgcacaagtttttttaaattatgggaatcaagatttaatcctagagatttggtgtacaattcaggctttggatgtttctttagcagttttgtgat

SEQ ID NO: 420 - DNA, 428 bp, Artificial Sequence
  Synthetic
gtacagcaaacagatgcgtctacatccatacattactgtgtttggctgaattccactctaatatgatgctccattatgcaccatactgtgatgacctttctactccgaaacctgctggagcctgcccttggccgtggggtgtcagccaatcactgcttgttccacttgttgtacattttatttttgagtctttttctttctcatatacagaaaaatagtatgaaaataaaataaatgtatgaaacagtattaatgcagaaatgtgctactaatggatgtctgagtcaccagaaattccattcttaaagaggcggttagcacctattagacgtaacagtgatgtcttttaaaaaatccaaaagcatattgcaacaataagtttgagactttgtgtgaacaaagggaaattcagcctcttatgtctttgt

SEQ ID NO: 421 - DNA, 470 bp, Artificial Sequence
  Synthetic
aacaaagactcttcttgcgtctctgcttcaggtaacttcaacatctccgctgatgagcctataactgtgacacctcctgactcacaatcatatatctccgtcaattactctgtgagaatcaatgaaacatatttcaccaatgtcactgtgctaaatggttctgtcttcctcagtgtgatggagaaagcccagaaaatgaatgatactatatttggtttcacaatggaggagcgctcatgggggccctatatcacctgtattcagggcctatgtgccaacaataatgacagaacctactgggaacttctgagtggaggcgaaccactgagccaaggagctggtagttacgttgtccgcaatggagaaaacttggaggttcgctggagcaaatactaataagcccaaactttcctcagctgcataaaatccatttgcagtggagttccatgtttattgtccttatgccttct

SEQ ID NO: 422 - DNA, 514 bp, Artificial Sequence
  Synthetic
tggatctagcattggcacagaccccaacagaaattttgatgctggttggtgtgaaattggagcctctcgaaacccctgtgatgaaacttactgtggacctgccgcagagtctgaaaaggagaccaaggccctggctgatttcatccgcaacaaactctcttccatcaaggcatatctgacaatccactcgtactcccaaatgatgatctacccttactcatatgcttacaaactcggtgagaacaatgctgagttgaatgccctggctaaagctactgtgaaagaacttgcctcactgcacggcaccaagtacacatatggcccgggagctacaacaatctatcctgctgctgggggctctgacgactgggcttatgaccaaggaatcagatattccttcacctttgaacttcgagatacaggcagatatggctttctccttccagaatcccagatccgggctacctgcgaggagaccttcctggcaatcaagtatgttgccagctacgtcctg

SEQ ID NO: 423 - DNA, 489 bp, Artificial Sequence
  Synthetic
agtatttcccatttatcgcagaccttttttaggaagcaagcttaatggctgataattttaaattctctctcttgcaggaaggactatgaaaagctagaattgagtgtttaaagttcaacatgttatttgtaatagatgtttgatagattttctgctactttgctgctatggttttctccaagagctacataatttagtttcatataaagtatcatcagtgtagaacctaattcaattcaaagctgtgtgtttggaagactatcttactatttcacaacagcctgacaacatttctatagccaaaaatagctaaatacctcaatcagtctcagaatgtcattttggtactttggtggccacataagccattattcactagtatgactagttgtgtctggcagtttatatttaactctctttatgtctgtggattttttccttcaaagtttaataaatttattttcttggattcctgataatgtgcttctg

SEQ ID NO: 424 - DNA, 536 bp, Artificial Sequence
  Synthetic
aatcccgagagtccggagggctgtgctaccccaagaagaggaaggatcagggggtgggcaactggtaactgaagtcaccaagaaagaagattcctgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtggcggcctgcaatctccccatagtccggggcccctgccgagccttcatccagctctgggcatttgatgctgtcaaggggaagtgcgtcctcttcccctacgggggctgccagggcaacgggaacaagttctactcagagaaggagtgcagagagtactgcggtgtccctggtgatggtgatgaggagctgctgcgcttctccaactgacaactggccggtctgcaagtcagaggatggccagtgtctgtcccggggtcctgtggcaggcagcgccaagcaa

SEQ ID NO: 425 - DNA, 489 bp, Artificial Sequence
  Synthetic
ttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaa

SEQ ID NO: 426 - DNA, 426 bp, Artificial Sequence
  Synthetic
tttgcccaaactcacccagtgagtgtgagcatttaagaagcatcctctgccaagaccaaaaggaaagaagaaaaagggccaaaagccaaaatgaaactgatggtacttgttttcaccattgggctaactttgctgctaggagttcaagccatgcctgcaaatcgcctctcttgctacagaaagatactaaaagatcacaactgtcacaaccttccggaaggagtagctgacctgacacagattgatgtcaatgtccaggatcatttctgggatgggaagggatgtgagatgatctgttactgcaacttcagcgaattgctctgctgcccaaaagacgttttctttggaccaaagatctctttcgtgattccttgcaacaatcaatgagaatcttcatgtattctggagaacaccattcctgatttc

SEQ ID NO: 427 - DNA, 571 bp, Artificial Sequence
  Synthetic
gtttcttcattgatcaaccaggtttgggttacacaaatcaattgtgggggaaaaatcaaataaaacaattgcttattatattttccaaaggactgagcatttatcttttattcacgaagatatcatatgaggatgataatgatctttaacagattttttagagatagaatttataaagaggctgatactaagaatactacaatcaaaattgaagctagagaatgtaaaaatagaaagtaaatagttctaagaatattctggcataaattatttttatttagccaataaaatagcctccaaatgtatatctcagacaccatagagctgctaacaatgagaatcaaggaagatgcttgcacttagatttcgtttgttgtatttcagtagttctggatgtcctttgttaaaattggaaaatggaaaaatgtctcgacagaaatgtcaatctggtgattctgtgaactgtaaaatgttcacttttaaaaataaagttgtaaacaagttactcatataagttggtattacagtagcaaaaacagaaaaccatgtgatccatcctgtattttgattg

SEQ ID NO: 428 - DNA, 514 bp, Artificial Sequence
  Synthetic
cagactgttcagtgtttgtcaagcttctggtctaatatgtactcgaaagactttccgcttacaatttgtagaaacacaaatatcgttttccatacagcagtgcctatatagtgactgattttaactttcaatgtccatctttcaaaggaagtaacaccaaggtacaatgttaaaggaatattcactttacctagcagggaaaaatacacaaaaactgcagatacttcatatagcccattttaacttgtataaactgtgtgacttgtggcgtcttataaataatgcactgtaaagattactgaatagttgtgtcatgttaatgtgcctaatttcatgtatcttgtaatcatgattgagcctcagaatcatttggagaaactatattttaaagaacaagacatacttcaatgtattatacagataaagtattacatgtgtttgattttaaaagggcggacattttattaaaatcaatattgtttttgctttttctgaggagtctctttcagtttca

SEQ ID NO: 429 - DNA, 507 bp, Artificial Sequence
  Synthetic
ggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacatgtttaattaaatttcacaatataaagttctacagttaattatgtgcatattaaaacaatggcctggttcaatttctttctttccttaataaatttaagttttttccccccaaaattatcagtgctctgcttttagtcacgtgtattttcattaccactcgtaaaaaggtatcttttttaaatgaattaaatattgaaacactgtacaccatagtttaca

SEQ ID NO: 430 - DNA, 548 bp, Artificial Sequence
  Synthetic
ggaatctgtcaagagcaggtatttcatacggttaaaaaaatgaacatgcagactccttttcaatatgggtttatatatataagtattttttgtgtattatgactacgttaggagtttaatattgtcaaggacagtacaactgcaaagggatgctgtatagcagcacatcagaagtcggaaggaactgacacattctctcagagctcaaggtcttaaagagcttgagttaaatctaggtacagttacaggcatgtatagacttaaatggatgcaatggaagctaactaaaataaggcttagttgtcctttctatttaaataccccaagttgtcttcttacttcctctcccctctcccattttgcactgtgtgtcgatgcaatcttcgctagcacaaaatattgtcgctaatagtcatttctgttttcccattgtaaatgctgttgagctttattctattttatgttactttgttaatgaaatttaggaaagcagttgtttctttaaatttattgtgatattctatatctagcggcctttatatgcaaat

SEQ ID NO: 431 - DNA, 521 bp, Artificial Sequence
  Synthetic
gagagcacaacatcgaagtcctggaggggaatgaacagttcatcaatgcggccaagatcatccgccaccccaaatacaacagccggactctggacaatgacatcctgctgatcaagctctcctcacctgccgtcatcaattcccgcgtgtccgccatctctctgcccactgcccctccagctgctggcaccgagtccctcatctccggctggggcaacactctgagttctggtgccgactacccagacgagctgcagtgcctggatgctcctgtgctgagccaggctgagtgtgaagcctcctaccctggaaagattaccaacaacatgttctgtgtgggcttcctcgagggaggcaaggattcctgccagggtgattctggtggccctgtggtctccaatggagagctccaaggaattgtctcctggggctatggctgtgcccagaagaacaggcctggagtctacaccaaggtctacaactatgtggactggattaaggacaccatagctgccaacagc

SEQ ID NO: 432 - DNA, 412 bp, Artificial Sequence
  Synthetic
gtcaaaccatgactcgcacatggcaaaagaacgggcccacagtacagcctcacattcttcttccaattctgaagatacagagatgtgatgaaaacaagtaatagctttggctgtttatttgatagctgtttctgggtatttaataggaatcctttctcaaggaatgagttgtgacctgtttactgtctctttagaagaaaaactccactggaaaccattcaccatgtgtgactgtcttctgttatcatttgtcttacaggcggctattgcagacggctaatttatgcttaacttaggaagagataaggcaagagctagatttttttcatgtgatcttttccaagcttcaacttaacttaactacatttctctgtatgatgatgtctcttacttctacaggttccttgagcac

SEQ ID NO: 433 - DNA, 417 bp, Artificial Sequence
  Synthetic
ggagtttgttgacctcatgaacagcaaagaatccaagtttaccttcaagatgaatccaggtgatgtgattacttttgataactggcgcttacttcatggccgacgtagctatgaagcaggaactgagatatcccgccatctagaaggagcttatgctgactgggatgtggtcatgtcaaggcttcgtatcttaaggcagagggtggagaatggaaactgaagtcacctgtagataattttaataagattccaatgaccatattttgtgagatatggcacattattcacagaccatgatctttgtgatttacatataatttccttaacaatgaacatgtaacttctctcacaagagtactctttactttgtaatcatatacaatgtcaactttttagatgtttcaccactcttttgca

SEQ ID NO: 434 - DNA, 538 bp, Artificial Sequence
  Synthetic
atcttcctcgcagaattcacagaattttgcaacttataaggaaggttacaacgtatatggcatcgaaagtgttaaaatttaggggatgaccttgaatgatgccatgaggaacaaggcaaggctgtcaattacaggaagtactggagaaaatggacgtgttatgactccagaatttcccaaagcagtgcatgctgtcccttacgtgagtcctggcatgggaatgaatgtcagtgtgactgatctctcgtgattgataagaaccttttgagtgccttacacaatggttttcttgtgtgtttattgtcaaagtggtgagaggcatccagtatcttgaagacttttctttcagccaagaattcttaaatatgtggagttcatcttgaattgtaaggaatgattaattaaaacacaacatctttttctactcgagttacagacaaagcgtggtggacatgcacagctaacatggaagtactataatttacctgaagtctttgtacagacaacaaacctgtttctgcagccactattgttagtc

SEQ ID NO: 435 - DNA, 330 bp, Artificial Sequence
  Synthetic
taaaatggggatcgcctcccaacagcagatttcgacattacctgagagtcttgattttaggcttgttttttgtaaacccatgtgtttgtagagattttaggcgtcttcggatatcttctcacctatgttccctggctaagaagtcagaggtagccaatgtttccttaaattcatttttaaacttaccattggtgcatatgttccagatggcagatgctgtcaataatctcaccattgatgacctttgtgtatgtagttcttgcatcctatactggataagcctgttttaacctgctatgatgggtgcttccattgcttcataatcttcat

SEQ ID NO: 436 - DNA, 529 bp, Artificial Sequence
  Synthetic
actgcaagttcctttatgtagctcgaaatgccaaagactgtatggtttcctactaccatttccaaaggatgaaccacatgcttcctgaccctggtacctgggaagagtattttgaaaccttcatcaatggaaaagtggtttggggttcctggtttgaccacgtgaaaggatggtgggagatgaaagacagacaccagattctcttcctcttctatgaggacataaagagggacccaaagcatgaaattcggaaggtgatgcagttcatgggaaagaaggtggatgaaacagtgctagataaaattgtccaggagacgtcatttgagaaaatgaaagaaaatcccatgacaaatcgttctacagtttccaaatctatcttggaccagtcaatttcctccttcatgagaaaaggaactgtgggggattggaaaaaccacttcactgttgcccagaatgagaggtttgatgaaatctatagaagaaagatggaaggaacctccataaacttctgcatggaactctgagcaag

SEQ ID NO: 437 - DNA, 479 bp, Artificial Sequence
  Synthetic
tttcttccctccagtgactaatgaagaactctttttaacctcagaccaacttggctacagctatgccatcgatctgccagtttcagttgaagaaactccaggttggcccacaactctcttagtagtcatgggaacactggtggctttggttggtctttttgtgctgttggcttttcttcaatatagaagacttcgaaaaggatatacacccctaatggagacacatttaagcagcaagagatacacagaagaagcctagggtgctcatgccttacctaagagaagaggctggccaagccacagttctgacgctgacaataaaggaactaatcctcactgttccttcttgagttgaagatctttgacataggttcttctatagtgatgatgatctcattcagaagatgcttagctgtagtttccgctttgcttgcttgtttaacaaacccaactaaagtgcttgaggctacctctacctt

SEQ ID NO: 438 - DNA, 462 bp, Artificial Sequence
  Synthetic
gattatacttagctgcttttatctgtagtgtgggtgactttttataactattatgttttataatgtcttcctgagagaatttcctaattttgtagttcatataagagaaatacaattttctagcattttctgaccttacaataaatcaattatgaatgcattttaacaaatattaaggtaggataaaccaacagaataaaaagttggtatttacttacattgaatatcatgcaaataaactcagaaaatgtcaccaaatactgtgggctatatttctcaatgatagggggaagaattcaaccttggagaaaatgaccagatcactttactgcttatatcttcagtattagaaaaactgcatgtatgattttaatagaaatgctttttataaagctaacattcagaaatgggataaaatgcaataacatctttcctttgtgcattaaattcccttctaattct

SEQ ID NO: 439 - DNA, 394 bp, Artificial Sequence
  Synthetic
  misc_feature (58)..(58): n is a, c, g, or t
  misc_feature (95)..(95): n is a, c, g, or t
  misc_feature (99)..(99): n is a, c, g, or t
  misc_feature (119)..(119): n is a, c, g, or t
  misc_feature (123)..(123): n is a, c, g, or t
  misc_feature (130)..(130): n is a, c, g, or t
  misc_feature (151)..(151): n is a, c, g, or t
  misc_feature (155)..(155): n is a, c, g, or t
  misc_feature (161)..(161): n is a, c, g, or t
  misc_feature (212)..(212): n is a, c, g, or t
ttcctgaggcacatcctaacgcaagtttgaccatgtatgtttgcaccccttttccccnaaccctgaccttcccatgggccttttccaggattccnaccnggcagatcagttttagtganacanatccgcntgcagatggcccctccaaccntttntgttgntgtttccatggcccagcattttccacccttaaccctgtgttcaggcacttnttcccccaggaagccttccctgcccaccccatttatgaattgagccaggtttggtccgtggtgtcccccgcacccagcaggggacaggcaatcaggagggcccagtaaaggctgagatgaagtggactgagtagaactggaggacaagagttgacgtgagttcctgggagtttccagagatg

SEQ ID NO: 440 - DNA, 497 bp, Artificial Sequence
  Synthetic
aaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagccatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaact

SEQ ID NO: 441 - DNA, 526 bp, Artificial Sequence
  Synthetic
aataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtcgataat

SEQ ID NO: 442 - DNA, 266 bp, Artificial Sequence
  Synthetic
gcatgagtcatgttcacacttgaagtctagtagtgcactgtaataattcattttttaaaagattatttaatgcccatttcaaaatacagtagtttacacagctacagaaacaatttggggcaagttttaaaacactgaaacagtaatagttattggtgtcacataaaactgatttgttttttacagccaaacctctgtcagtcagaggcattcattagttttatacatgtaatttgaaaatcactaaacctcgttttctcagcagc

SEQ ID NO: 443 - DNA, 388 bp, Artificial Sequence
  Synthetic
gcacccagccttttaccatgtgggtttctttagtgtcttaaaagcgtccataagccaccattctgtggaaccaaggccccctccacgcaaacaccctccctcctggggacctctggagcctcagccagaagtaccattaggtttaattttaatttgttttgctggagaaacatcaggtttgtaggagactgagttgttagcaggtgtgcttagctcttgatagtgaacgtgtaccttgggaactggctcacccacctgctaatagcaccatcgtcactattaagcagacatttcagttggtagaatccatgtagaagtcatggacttttctgggaaatgacttttctgggaaatgacagtttctttgacatattttctttgcccactt

SEQ ID NO: 444 - DNA, 544 bp, Artificial Sequence
  Synthetic
ggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattcttta

SEQ ID NO: 445 - DNA, 472 bp, Artificial Sequence
  Synthetic
atttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtata

SEQ ID NO: 446 - DNA, 532 bp, Artificial Sequence
  Synthetic
attgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaatt

SEQ ID NO: 447 - DNA, 488 bp, Artificial Sequence
  Synthetic
gggactcatccgaggtcactatgaggcaaagcaaggtgggttcctgccagggggagggagtctacacagcacaatgaccccccatggacctgatgctgactgctttgagaaggccagcaaggtcaagctggcacctgagaggattgccgatggcaccatggcatttatgtttgaatcatctttaagtctggcggtcacaaagtggggactcaaggcctccaggtgtttggatgagaactaccacaagtgctgggagccactcaagagccacttcactcccaactccaggaacccagcagaacctaattgagactggaacattgctaccataattaagagtagatttgtgaagattcttcttcagaatctcatgctttctggtagtattggaggagggggttggttaaaatgaaaattcacttttcatagtcaagtaactcagaacttttatggaaacgcatttgcaaagttctatggctgtcacctta

SEQ ID NO: 448 - DNA, 487 bp, Artificial Sequence
  Synthetic
gatacaattacctgcacgacacatggaaattggactaaattaccagaatgcagggaagtaaaatgcccattcccatcaagaccagacaatggatttgtgaactatcctgcaaaaccaacactttattacaaggataaagccacatttggctgccatgatggatattctctggatggcccggaagaaatagaatgtaccaaactgggaaactggtctgccatgccaagttgtaaagcatcttgtaaattacctgtgaaaaaagccactgtggtgtaccaaggagagagagtaaagattcaggaaaaatttaagaatggaatgctacatggtgataaagtttctttcttctgcaaaaataaggaaaagaagtgtagctatacagaggatgctcagtgtatagatggcactatcgaagtccccaaatgcttcaaggaacacagttctctggctttttggaaaactgatgcatccgatgtaaagccatgct

SEQ ID NO: 449 - DNA, 528 bp, Artificial Sequence
  Synthetic
atatgtttttcacagcccgttacaaattggcaatgtttggttaatgtttgtattacttggaaatcgctacagcttggactatttttttctaaatttttagcattagtccatttctgctgctaacaattgaatccagaaatctactttctccatcttccactgttagtgccagtgagcaatactgttgtgcaacaaaaatgtcactttatctcagtgtgaatgagtagtctaaattccctttctaccattgatttaaatatatatattggtaagagagactgcccatgtgtttagaatagaattttttaaatgaaatgatcaacaggtggaatttgaaatatattcttctacaaaagagatttctttcccttttatattttgatgattgttttcttaagattaagatatgttcttgctcttttataagattatttaaattatgtttccctctgattttttttcaccattgtatttactaagttattggatttacatgaaatctggcactttagggtgttctttttctca

SEQ ID NO: 450 - DNA, 514 bp, Artificial Sequence
  Synthetic
gtctggcacaccatggatgacaatgaagaaaatttggatgaatcaaccattgacaatctaaacaaaatcctacaagtctttgtgttggaatatcttcatttgtaatactctgatttagtttaggataattggttctagaattgaattcaaaagtcaaggcatcatttaaaataatctgatttcagacaaatgctgtgtggaaacatctatcctatagatcatcctattcttatgtgtctttggttatcagatcaattacagaataattgtgttgtgatattgtgtcctaaattgctcattaatttttatttacagattgaaaaagaggcaccgtgtaaagaaaatggcaaaataaatatctttccaaggatcatcatcacgatagctaaacagtacttaaatagcggttggaactaggtagcctttcgaattttatgattttttcatatgtggaaatctattacatgtaatacaaaacaaacatgtagtttgaaggcggtcagatttctttgag

SEQ ID NO: 451 - DNA, 468 bp, Artificial Sequence
  Synthetic
gatttacaaaggtcctcccattgcaaagcagtgtttgtcctaatttatatattgtttttctagttcattttgtgtttccaacttttcatgtaaaattttaattatttttgaatgtgtggatgtgagactgaggtgccttttggtactgaaattctttttccatgtacctgaagtgttacttttgtgatataggaaatccttgtatatatactttattggtccctaggcttcctattttgttaccttgctttctctatggcatccaccattttgattgttctacttttatgatatgttttcataagtggttaagcaagtattctcgttacttttgctcttaaatccctattcattacagcaatgttggtggtcaaagaaaatgataaacaacttgaatgttcaatggtcctgaaatacataacaacattttagtacattgtaaagtagaatcctctgttcataatgaac

SEQ ID NO: 452 - DNA, 335 bp, Artificial Sequence
  Synthetic
gcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgacca

SEQ ID NO: 453 - DNA, 459 bp, Artificial Sequence
  Synthetic
aagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaa

SEQ ID NO: 454 - DNA, 524 bp, Artificial Sequence
  Synthetic
acactaattggtttcctctagggtgatattcgtcattactctgtctcttcaatccatccagctaaatggaataggtgatgacttggatgtgactcctacttggcttctatcaccaacagaaattataccatatagtgaaaggcaattttctaaataatttcattactaatatgaactgtgaagttgtcattttttcatttgtccttttctgctatcaccttcctcttgtcagaatgaatatagacacatgtatctaagtgggaccaaagaaaaaatagcgaactttcaccaaagttttcatgaaaacccaaaagctttaaaagttactatcaagaaattgaaaggaaacccacagaataggataaaatatttgtaaatcatatttgataaaagtcttgtaaccagatacataaagagctcttacaactcaataaaaggcaagtaatttaaaaataggcaaaagaattgctggatggtatggtagttctatttttagtttttaccctaactactctgacttgatc

SEQ ID NO: 455 - DNA, 530 bp, Artificial Sequence
  Synthetic
tacagagtattcctctcttcacaccaggaccagccactgttgcagcatgagttcccagcagcagaagcagccctgcatcccaccccctcagcttcagcagcagcaggtgaaacagccttgccagcctccacctcaggaaccatgcatccccaaaaccaaggagccctgccaccccaaggtgcctgagccctgccaccccaaagtgcctgagccctgccagcccaagcttccagagccatgccaccccaaggtgcctgagccctgcccttcaatagtcactccagcaccagcccagcagaagaccaagcagaagtaatgtggtccacagccatgcccttgaggagccggccaccagatgctgaatcccctatcccattctgtgtatgagtcccatttgccttgcaattagcattctgtctcccccaaaaaagaatgtgctatgaagctttctttcctacacactctgagtctctgaatgaagctgaaggtcttagtaccagagctagttttcagctgctcagaattcatct

SEQ ID NO: 456 - DNA, 520 bp, Artificial Sequence
  Synthetic
ttttctactcagcagatgctgtgtgttttgatgttgacagcacggtcatcagtgaagaaggaatcggatgctttcattggatttggaggaaatgtgatcaggcaacaagtcaaggataacgccaaatggtatatcactgattttgtagagctgctgggagaaccggaagaataacatccattgtcatacagctccaaacaacttcagatgaatttttacaagttacacagattgatactgtttgcttacaattgcctattacaacttgctataaaaagttggtacagatgatctgcactgtcaagtaaactacagttaggaatcctcaaagattggtttgtttgtttttaactgtagttccagtattatatgatcactatcgatttcctggagagttttgtaatctgaattctttatgtatattcctagctatatttcatacaaagtgttttaagagtggagagtcaattaaacacctttactcttaggaatatagattcggcagccttcagtgaatatt

SEQ ID NO: 457 - DNA, 486 bp, Artificial Sequence
  Synthetic
gagacacgttacagaacctatgttcaggttgcgggtgagctgccctctccaaatccagccagagatgcacattcctcggccagtctcagccaacagtaccaaaagtgatttttgagtgtgccagggtaaaggcttccagttcagcctcagttattttagacaatctcgccatctttaatttcttagcttcctgttctaataaatgcacggctttacctttcctgtcagaaataaaccaaggctctaaaagatgatttcccttctgtaactccctagagccacaggttctcattccttttcccattatacttctcacaattcagtttctatgagtttgatcacctgatttttttaacaaaatatttctaacgggaatgggtgggagtgctggtgaaaagagatgaaatgtggttgtatgagccaatcatatttgtgattttttaaaaaaagtttaaaaggaaatatctgttctgaaaccccacttaa

SEQ ID NO: 458 - DNA, 448 bp, Artificial Sequence
  Synthetic
agtggttttatatcgcatcggcctttcgaaacgaggagtacaataagtcggttcaggagatccaagcaaccttcttttacttcacccccaacaagacagaggacacgatctttctcagagagtaccagacccgacaggaccagtgcatctataacaccacctacctgaatgtccagcgggaaaatgggaccatctccagatacgtgggaggccaagagcatttcgctcacttgctgatcctcagggacaccaagacctacatgcttgcttttgacgtgaacgatgagaagaactgggggctgtctgtctatgctgacaagccagagacgaccaaggagcaactgggagagttctacgaagctctcgactgcttgcgcattcccaagtcagatgtcgtgtacaccgattggaaaaaggataagtgtgagccactggagaagcagcacga

SEQ ID NO: 459 - DNA, 376 bp, Artificial Sequence
  Synthetic
acccgacaggaccagtgcatctataacaccacctacctgaatgtccagcgggaaaatgggaccatctccagatacgtgggaggccaagagcatttcgctcacttgctgatcctcagggacaccaagacctacatgcttgcttttgacgtgaacgatgagaagaactgggggctgtctgtctatgctgacaagccagagacgaccaaggagcaactgggagagttctacgaagctctcgactgcttgcgcattcccaagtcagatgtcgtgtacaccgattggaaaaaggataagtgtgagccactggagaagcagcacgagaaggagaggaaacaggaggagggggaatcctagcaggacacagccttggatcagg

SEQ ID NO: 460 - DNA, 227 bp, Artificial Sequence
  Synthetic
ggttatgacccttacttttggtgatgtggttgctgttcgccactatgagaaggcataaaaatgttcctggtcggggcttggaagagctcttcagtttttctgtttcctcaagtctcagtgctatcctattacaacatggctgatcattaattagaaggttatccttggtgtggaggtggaaaatggtgatttaaaaacttgttactccaagcaacttgcccaatttt

SEQ ID NO: 461 - DNA, 187 bp, Artificial Sequence
  Synthetic
gtgaccaaaccaacggtaattatcagtcaagaaggagacaaagtggtcatcaggactctcagcacattcaagaacacggagattagtttccagctgggagaagagtttgatgaaaccactgcagatgatagaaactgtaagtctgttgttagcctggatggagacaaacttgttcacatacagaaat

SEQ ID NO: 462 - DNA, 335 bp, Artificial Sequence
  Synthetic
agacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattttagacacttctgcagggatctgcctgcatcctgacggggtgccgtccccagcacggtgattagtcccagagctcggctgccacctccaccggacacctcagacacgcttctgcagctgtgcctcggctcacaacacagattgactgctctgactttgactac

SEQ ID NO: 463 - DNA, 410 bp, Artificial Sequence
  Synthetic
taagcagacccggcattggcaatgtagctgtaattttctgacaaaatttaagacaaaattgtcaacttgaaactaaaacatgccaaggttttgatatacttgtcttaagatattaatgaaacaattttgaacactgataggaaggtccacatccacaaagtttctcttgagttttgttatgtgttttgctgtgtttgattttcagtgattgtctggtatatttacagtcctcaaacatggttatttctgtcagtgacttaacattcggttttaccagccagcagtattcttcagtaaataaagaatggaattgctgaatgtaatcattgaacctcgagtcactgtaaaagttcagtaattgcttattgtattagttttagatgctggcaccgcatgtgctctgtttattc

SEQ ID NO: 464 - DNA, 471 bp, Artificial Sequence
  Synthetic
gtcatgcagccaatccaaacggcagatactactggggtggacagtacacctgggacatggcaaagcatggcacagatgatggtgtagtatggatgaattggaaggggtcatggtactcaatgaggaagatgagtatgaagatcaggcccttcttcccacagcaatagtccccaatacgtagatttttgctcttctgtatgtgacaacatttttgtacattatgttattggaattttctttcatacattatattcctctaaaactctcaagcagacgtgagtgtgactttttgaaaaaagtataggataaattacattaaaatagcacatgattttcttttgttttcttcatttctcttgctcaccaagaagtaacaaaagtatagttttgacagagttggtgttcataatttcagttctagttgattgcgagaattttcaaataaggaagaggggtcttttatccttgtcg

SEQ ID NO: 465 - DNA, 364 bp, Artificial Sequence
  Synthetic
gccaaaatgatacctggaggcttatctgaggccaaacccgccactccagaaatccaggagattgttgataaggttaaaccacagcttgaagaaaaaacaaatgagacttatggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctga

SEQ ID NO: 466 - DNA, 499 bp, Artificial Sequence
  Synthetic
gactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgaggccacacccacggaactggcaaagctggttaacaagcgctcagactttgcctccaactgctgttccataaactcacctcctctttactgtgattcagagattgatgctgaattgaagaatatcctgtagtcctgaagcatgtttattaactttgaccagagttggagccacccaagggaatgatctctgatgacctaacctaagcaaaaccactgagcttctgggaagacaactaggatactttctactttttctagctac

SEQ ID NO: 467 - DNA, 536 bp, Artificial Sequence
  Synthetic
gtgtccctgctcgagggtgaagctgttgaggtcattcacaagctcctggacggctggtgggtcatcaggaaagacgacgtcacaggctactttccgtccatgtacctgcaaaagtcggggcaagacgtgtcccaggcccaacgccagatcaagcggggggcgccgccccgcaggtcgtccatccgcaacgcgcacagcatccatcagcggtcgcggaagcgcctcagccaggacgcctatcgccgcaacagcgtccgttttctgcagcagcgacgccgccaggcgcggccgggaccgcagagccccgggagcccgctcgaggaggagcggcagacgcagcgctctaaaccgcagccggcggtgcccccgcggccgagcgccgacctcatcctgaaccgctgcagcgagagcaccaagcggaagctggcgtctgccgtctgaggctggagcgcagtccccagctagcgtctcggcccttgccgccccgtgcctgtacatacgtgttctatagagcctggcgtctggacgccgagggc

SEQ ID NO: 468 - DNA, 515 bp, Artificial Sequence
  Synthetic
gaggtcccaaacagaattatcgaaatagcaaataaaactcccaagatcagtcaactttacaagcaagcatctggaacaatggtgtatgggttgtttatgttacaaaagaaaagcgtacacaagaagaacacaatttatgaaatacaggataatacaggatccatggatgtagtggggagtggaaaatggcacaatatcaagtgtgagaaaggagataaacttcgactcttctgccttcaactgagaacagttgaccgcaagctgaaactggtgtgtggaagtcacagcttcatcaaggtcatcaaggccaagaaaaacaaggaaggaccaatgaatgttaattgaaatatgaaagctgaaatgcaacaaacaacttccgcttaaaacaattaagttgttaataactgtgattttgtaaatttcagtaattcatttaaatgatgtttcagtagatatattctagcatattaagagcttttataactgagttatagattagtttgctttctggaata

SEQ ID NO: 469 - DNA, 441 bp, Artificial Sequence
  Synthetic
ggacaaagagcgctatgtctccctggtgatgcctgtggccctgttcaacctgattgacacttttcccttgagaaaagaagagatggtcctacaagccgaaatgagccagacctgtaacacctgacatgatggttcctctcttggcaattcctcttcattgtctacatagtgacatgcacacgggaaagccttaaaaatatccttgatgtacagattttatttgtaattttaaaagtctattttattatgagctttctttgcacttaaaaattagcatgctgctttttgtacttggaagtgtttcaaaaaattatatgaccatatttactctttctaacctttctttactccatcatggctggttgatttgtagagaaattagaacccataaccatacacaggctatcaacatgttattcaatgtgacacctaactcttttc

SEQ ID NO: 470 - DNA, 371 bp, Artificial Sequence
  Synthetic
aaatctgttctaagacatatgatcaacagatgagaactggtggttaatatgtgacagtgagattagtcatatcactaatatactaacaacagaatctaatcttcatttaaggcactgtagtgaattatctgagctagagttacctagcttaccatactatatctttggaatcatgaaaccttaagacttcagaatgattttgcaggttgtcttccattccagcctaacatccaatgcaggcaaggaaaataaaagatttccagtgacagaaaaatatattatctcaagtattttttaaaaatatatgaattctctctccaaatattaactaattattagattatattttgaaatgaacttgttggcccatc

SEQ ID NO: 471 - DNA, 191 bp, Artificial Sequence
  Synthetic
tggcccatctattacatctacagctgacccttgaacatgggggttaggggagctgacaattcgtgggtccgcaaaatcttaactacctaatagcctactattgaccataaaccttactgataacataaacagtaaattaacacatattttgcgtgttatatgtattatacactatattcctacaataaagt

SEQ ID NO: 472 - DNA, 252 bp, Artificial Sequence
  Synthetic
ttctctcgaccacttaaaacttcagacttcctgtcctgctggtatcatggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctgaagttctgctacaacctctagatctgcagcttgccacatcagcttaaa

SEQ ID NO: 473 - DNA, 406 bp, Artificial Sequence
  Synthetic
gataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagc

SEQ ID NO: 474 - DNA, 380 bp, Artificial Sequence
  Synthetic
tactgcttgcagtaattcaactggaaattaaaaaaaaaaaactagactccattgtgccttactaaatatgggaatgtctaacttaaatagctttgagatttcagctatgctagaggcttttattagaaagccatatttttttctgtaaaagttactaatatatctgtaacactattacagtattgctatttatattcattcagatataagatttgtacatattatcatcctataaagaaacggtatgacttaattttagaaagaaaattatattctgtttattatgacaaatgaaagagaaaatatatatttttaatggaaagtttgtagcatttttctaataggtactgccatatttttctgtgtggagtatttttata

SEQ ID NO: 475 - DNA, 377 bp, Artificial Sequence
  Synthetic
gagaacgggtacattcacccggtcaacctgacgtggccctttatggtggccgggaaacccgagagtgacctgaaagaagtggtgaccgcgagccgcttatgtggaaccaccgcgtcctgaccttggaggtgcgagtctgggaaaggcgcgctcccggggggagcgggccccgggaaggcgacccctgccctcagtgctctctgtctctgcttccccctcgcaatgctcctctctctgtcccaccccgcgagaacactttacaacgacgaggagattcgtttccaaaccagaggagatcaattgtacttacaaagattcccatctatttaactttattaacttctaccgtgaatgactctgcaagccttgctggtcca

SEQ ID NO: 476 - DNA, 444 bp, Artificial Sequence
  Synthetic
gaggcatttagagtatggacttttcttttctttttctttttctttttttctttttgagatggagtcacactctccaggctggagtgcagtggcacaatctcggctcactgcaatttgcgtctcccaagttcaagcgattctcctgctttagactatggatttctttaaggaatactggtttgcagttttgttttctggactatatcagcagatggtagacagtgtttatgtagatgtgttgttgtttttatcattggattttaacttggcccgagtgaaataatcagatttttgtcattcacactctcccccagttttggaataacttggaagtaaggttcattcccttaagacgatggattctgttgaactatggggtcccacactgcactattaattccacccactgtaagggcaaggacaccattccttctacatataaga

SEQ ID NO: 477 - DNA, 456 bp, Artificial Sequence
  Synthetic
gagacacaagctcataatgttccatgtataaaagtaataactttattgggtagagatattcttacaagatctagcacctctgccagtgcacagataggactgttttaaatgatttgggaacttttggttgcctgcagttgtgaacagagaacttctctacagagaaacaaaccactaaaagcaatatgaccgagttgagatgtggtttccaatgagcaattggtgaatttaagcaacctggatgtgcatatgtggaggctcccgtctcactgtttgatcaaacttcttttatgtagtcacgtagacttgattttttctgctgtgaaaatgaaaaaataaagcaatatgacaaaaagtttaaaaatgcataaaaaataggatttcctctaggctcctcgaagagatttttttaatatgatgcttgtcttactttcttagacacgttacatttcccct

SEQ ID NO: 478 - DNA, 500 bp, Artificial Sequence
  Synthetic
aagccatgtggcctaacttgatacaaaaataaaagtaattgtttggcaatctaaatttaaaacctgttagaactcaggacaggcgcttcaatgcgctttttaacaatatttaaggctgttttgatgagtgcgttgtgagaatcatcttaatgaattctttattgagtgtctaaaacatagtataatacacatggtattcttgccactggatagtcttcaataaaagtttaattgatttttttttgttggtctcttaagtaagtcttatttttaactaagcattgacagaatatcttaaaatggtaacctgggggtggcgggtgggtgctgtgtgcacggcagcctagccagtggggatcctgctgtttattataagtagttcacagactctgatggcattttggtaagctttccatctttaagaaattgaaccagcattctcttattaattctttaaactgtggaagtaatttccagttcttacactctgatacgcatcc

SEQ ID NO: 479 - DNA, 168 bp, Artificial Sequence
  Synthetic
tttccagaacatgaacgggtccgaatacttcgtgaagatccagtccttcctgggtggggcccccacggaggatttgaaggcgctcagtcagcagaatgtgagcatggacttggccacgttcatgaagctgcggacggatgcggtgctgccgttgactgtggctgaggt

SEQ ID NO: 480 - DNA, 547 bp, Artificial Sequence
  Synthetic
cttcgttcgcagagcttttcagattgtggaatgttggataaggaattatagacctctagtagctgaaatgcaagaccccaagaggaagttcagatcttaatataaattcactttcatttttgatagctgtcccatctggtcatgtggttggcactagactggtggcaggggcttctagctgactcgcacagggattctcacaatagccgatatcagaatttgtgttgaaggaacttgtctcttcatctaatatgatagcgggaaaaggagaggaaactactgcctttagaaaatataagtaaagtgattaaagtgctcacgttaccttgacacatagtttttcagtctatgggtttagttactttagatggcaagcatgtaacttatattaatagtaatttgtaaagttgggtggataagctatccctgttgccggttcatggattacttctctataaaaaatatatatttaccaaaaaattttgtgacattccttctcccatctcttccttgacatgcattgtaaataggttcttcttgttctgag

SEQ ID NO: 481 - DNA, 388 bp, Artificial Sequence
  Synthetic
gaagctggaatttgcccttctgtttctagtttttgatgagaatgaatcttggtacttagatgacaacatcaaaacatactctgatcaccccgagaaagtaaacaaagatgatgaggaattcatagaaagcaataaaatgcatgctattaatggaagaatgtttggaaacctacaaggcctcacaatgcacgtgggagatgaagtcaactggtatctgatgggaatgggcaatgaaatagacttacacactgtacattttcacggccatagcttccaatacaagcacaggggagtttatagttctgatgtctttgacattttccctggaacataccaaaccctagaaatgtttccaagaacacctggaatttggttactccactgccat

SEQ ID NO: 482 - DNA, 397 bp, Artificial Sequence
  Synthetic
ctgtaagccttcccgtaaatctcaggcttctggtgtatcggtatgggatgcagaactctggcaatgagatttcatggaactacactcttgagcaataccagaaaacttcattagctcaagaaaaagaaaaactgctgtatggattagcatcagtgaagaacgttactcttttgtcaaggtatttggatttgctcaaggacacgaaccttattaaaactcaggatgtgtttacagtcattcgatatatctcatataacagctatgggaagaacatggcctggaattggatacaactcaactgggactatctagtcaacagatatacactcaataacagaaaccttggccgaattgtcacaatagcagagccattcaacactgaactgcaactgtggca

SEQ ID NO: 483 - DNA, 508 bp, Artificial Sequence
  Synthetic
ctaagcactgtgtttatatgtcttgcaaagcctttaaattgttcctctttgtttatgaagaaagatactaatagagtacttaatatactcagggattccttctaagtgtacttcataagatattctttacaaactgaagataatgaaatagttattttaatacctttaaatatcattctttgtatcttaaatctgtgaagtagaacaatttggtcttagctttacattttcagtaccaaagaaacaccacttaatcttctctcttgattgatttttaaagtttaaataccagtacttgagggaccaatattaccatcttaatctttattttattattcagaattatacagacctaatatcattttattcacatatgtcttactactttaaatcctaccaaagtaacctgggcttaagttttacttgaggatcatatgaattagccaaaagaatggctgactgttgcctttactcttataaaatcaataagagaactctcctctttttt

SEQ ID NO: 484 - DNA, 393 bp, Artificial Sequence
  Synthetic
aaaggctggcatcttatggctcatcaaaggcggctgtgaccatgttctcatcagttatgagactggagctttccaagtggggaattaaagttgcttccatccaacctggaggcttcctaacaaatatcgcaggcaccagtgacaagtgggaaaagctggagaaggacattctggaccacctccccgctgaggtacaggaagactacggccaggactacatcttagcacagcggaatttcctcctattgatcaactcgttagccagcaaggacttctctccggtgctgcgggacatccagcatgctatcttggcgaagagcccttttgcctattacacgccagggaaaggcgcttacttgtggatctgccttgctcactatttgcctattggca

SEQ ID NO: 485 - DNA, 504 bp, Artificial Sequence
  Synthetic
ggaagtcttcataaagccgcagtagaacttgagctgaaaacccagatggtgttaactggccgccccactttccggcataactttttagaaaacagaaatgcccttgatggtggaaaaaagaaaacaaccacccccccactgcccaaaaaaaaaagccctgccctgttgctcgtgggtgctgtgtttactctcccgtgtgccttcgcgtccgggttgggagcttgctgtgtctaacctccaactgctgtgctgtctgctagggtcacctcctgtttgtgaaaggggaccttcttgttcgggggtgggaagtggcgaccgtgacctgagaaggaaagaaagatcctctgctgacccctggagcagctctcgagaactacctgttggtattgtccacaagctctcccgagcgccccatcttgtgccatgttttaagtcttcatggatgttctgcatgtcatggggactaaaactcacccaacagatctttccagaggtccatggtgg

SEQ ID NO: 486 - DNA, 484 bp, Artificial Sequence
  Synthetic
ggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaat

SEQ ID NO: 487 - DNA, 543 bp, Artificial Sequence
  Synthetic
tggccgagacagagtgccgctatgccacgcagctgcagcagatccaggggctcattggtggcctggaggcccagctgagtgagctccgatgcgagatggaggctcagaaccaggagtacaagatgctgcttgacataaagacacggctggagcaggagatcgctacttaccgcagcctgctcgagggccaggatgccaagatggctggcattggcatcagggaagcctcttcaggaggtggtggtagcagcagcaatttccacatcaatgtagaagagtcagtggatggacaggtggtttcttcccacaagagagaaatctaagtgtctattgcaggagaaacgtcccttgccactccccactctcatcaggccaagtggaggactggccagagggcctgcacatgcaaactccagtccctgccttcagagagctgaaaagggtccctcggtcttttatttcagggctttgcatgcgctctattccccctctgcctctccccaccttctttggagcaaggagatgcagctgtattgtgtaaca

SEQ ID NO: 488 - DNA, 537 bp, Artificial Sequence
  Synthetic
gcaggccatactggttccattgttctgtataatactgaataaataaatttacttttacatgatcgtataagtttctagataagataaacaaattctgtttaaatttttttaataaaaatcttaaaacactttttttctaacctagactgagaaattcatgtttacttttctaggtgtatgatactttgtaaagttgatactttcctaagaatttaacatgtcatatttttgaaatagatttaagtgtgcttcttattgctaaaaatactaaatgtcatgggtcatagtatctgatatcaatatcgttgataacatatccacaggtaacaccatgatgtaggcataaatggaaaacaaaaaccctactatttcaaatatattgtacttttttatttctgtaagccaactgtgtgccattttcactggacttttaaatctagactttagtgatgtctacattgtaaatgatcttttgtggatatttgtcacttggtttcagaaagttcacaaatgtagcaacagctcacatgactgagt

SEQ ID NO: 489 - DNA, 519 bp, Artificial Sequence
  Synthetic
gcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactagaattgaacattcaaaaacccctggaagagactctttaagacctcaaaccatttagaatgggcaatgtattttacgctgtgtta

SEQ ID NO: 490 - DNA, 509 bp, Artificial Sequence
  Synthetic
agttttctaagtacgtctgtagcatgatggtatagattttcttgtttcagtgctttgggacagattttatattatgtcaattgatcaggttaaaattttcagtgtgtagttggcagatattttcaaaattacaatgcatttatggtgtctgggggcaggggaacatcagaaaggttaaattgggcaaaaatgcgtaagtcacaagaatttggatggtgcagttaatgttgaagttacagcatttcagattttattgtcagatatttagatgtttgttacatttttaaaaattgctcttaatttttaaactctcaatacaatatattttgaccttaccattattccagagattcagtattaaaaaaaaaaaaaattacactgtggtagtggcatttaaacaatataatatattctaaacacaatgaaatagggaatataatgtatgaactttttgcattggcttgaagcaatataatatattgtaaacaaaacacagctcttacctaata

SEQ ID NO: 491 - DNA, 475 bp, Artificial Sequence
  Synthetic
gctgcctggggtggcaaggctgcaaacaaggaggcaacccaggaggcttttatgaagcgggccatggctaactgccaggcggccaaaggacagtatgttcacacgggttcttctggggctgcttccacccagtcgctcttcacagcctgctatacctactagggtccaatgcccgccagcctagctccagtgcttctagtaggagggctgaaagggagcaacttttcctccaatcctggaaattcgacacaattagatttgaactcgctggaaatacaacacatgttaaatcttaagtacaagggggaaaaaataaatcagttatttgaaacataaaaatgaataccaaggacctgatcaaatttcacacagcagtttccttgcaacactttcagctccccatgctccagaatacccacccaagaaaataataggctttaaaacaatatcggctcctcatccaaagaacaactgc

SEQ ID NO: 492 - DNA, 507 bp, Artificial Sequence
  Synthetic
agatggccctgggaacaatcctcaggaattgccctcaagaactcgcttctttgctttgagagtgccatggtcatgtcattctgaggtacataacacataaattagtttctatgagtgtataccatttaaagattttttcagtaaagggaatattacatgttgggaggaggagataagttatagggagctggatttcaaacggtggtccaagattcaaaaatcctattgatagtggccattttaatcattgccatcgtgtgcttgtttcatccagtgttatgcactttccacagttggtgttagtatagccagagggtttcattattatttctctttgctttctcaatgttaatttattgcatggtttattctttttctttacagctgaaattgctttaaatgatggttaaaattacaaattaaattgggaatttttatcaatgtgattgtaattaaaaatattttgatttaaataacaaaaataataccagattttaagccgcggaa

SEQ ID NO: 493 - DNA, 553 bp, Artificial Sequence
  Synthetic
  misc_feature (223)..(223): n is a, c, g, or t
gaatcttccctattagttcttcatgtggttcatggtactgatttagaaatccagtgtttgggggatttttttctctgggattcatgaatttagccctgttgtagcatgttaaaggtgacaaacagctggacaaatttttaaaaagtaaaataaaattttatctataattagtattattacatttagcttttcattgaaccgaaagaaaaaaagtgatattggnaccctggaaagattttgaaacttgagtggtttgataacccttctatgtattgtagggagaaaaaaaaaagtttattttattccactgtcctcccttaaaagcatcatttgagcaataaatgaatattgtctttaaaccaagggttagggaattttcctctctctctctctctcctctctctttctgttcaaagaacttcaaacatttgggaccacctggtattctgtattttcactggccatattggaagcagttctagttgcattgtattgagttgtgctggcagtagtttccatgcctgtcaatgtatcatagtcctttgttgcccag

SEQ ID NO: 494 - DNA, 549 bp, Artificial Sequence
  Synthetic
agtgtatgtgttgacatttctccccatctcttcccactctgttttctccccattatttgaataaagtgactgctgaagatgactttgaatccttatccacttaatttaatgtttaaagaaaaacctgtaatggaaagtgagactccttccctaatttcagtttagagcaacttgaagaagagtagacaaaaaataaaatgcacatagaaaaagagaaaaagggcacaaagggattggcccaatattgattcttttttataaaacctcctttggcttagaaggaatgactctagctacaataatacacagtatgtttaagcaggttcccttggttgttgcattaaatgtaatccacctttaggtattttagagcacagaacaacactgtgttgatctagtaggtttctatttttcctttctctttacaatgcacataatactttcctgtatttatatcataacgtgtatagtgtaaaatgtgaatgactttttttgtgaatgaaaatctaaaatctttgtaactttttatatctgcttttgtttcaccaa

SEQ ID NO: 495 - DNA, 465 bp, Artificial Sequence
  Synthetic
ttcctcaccctaaaactaagcgtgctgcttctgcaaaagatttttgtagatgagctgtgtgcctcagaattgctatttcaaattgccaaaaatttagagatgttttctacatatttctgctcttctgaacaacttctgctacccactaaataaaaacacagaaataattagacaattgtctattataacatgacaaccctattaatcatttggtcttctaaaatgggatcatgcccatttagattttccttactatcagtttatttttataacattaacttttactttgttatttattattttatataatggtgagtttttaaattattgctcactgcctatttaatgtagctaataaagttatagaagcagatgatctgttaatttcctatctaataaatgcctttaattgttctcataatgaagaataagtaggtaccctccatgcccttctgtaataaatat

SEQ ID NO: 496 - DNA, 417 bp, Artificial Sequence
  Synthetic
tggtgacacaaacagccttttcccaggtacttggtacctggagcgagtggacgagcagcatcgccgaaagtatgcccggcgtcccgtctaaaggtgttctgcagatccatggaaagcttcctgggaaacgtatgctagcagagcttctccccgtgaatcatatttttaagatcccactcttagctggtaaatgaatttgaatcgacatagtagccccataagcatcagccctgtagagtgaggagccatctctagcgggcccttcattcctctccatgctgcaatcactgtcctgggcttatggtgcctatggactaggggtcctttgtgaaagagcaagatggagcaatggagagaagacctcttcctgaatcactggactccagaaatgtgcatgcagatcagctgttgccttca

SEQ ID NO: 497 - DNA, 490 bp, Artificial Sequence
  Synthetic
tatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttt

SEQ ID NO: 498 - DNA, 523 bp, Artificial Sequence
  Synthetic
tttggtggttttattctatcggtataaaggcatcgatattttagatgcacccgtgtttgtaaaaatgtagagcacaatggaattatgctggaagtctcaaataatatttttttcctattttatactcatggaagagataagctaaagaggggacaataatgagaaatgttggtgtgcttttctaagcatttaaaacataattgccaattgaaaccctaaatatgtttacataccattaagatatgattcatgtaacaatgttaaattaattataatgggattgggtttgttatctgtggtagtatatatcctagtgttcctatagtgaaataagtagggttcagccaaagctttctttgttttgtaccttaaattgttcgattacgtcatcaaaagagatgaaaggtatgtagaacaggttcacgtgattacctttttcttttggcttggattaatattcatagtagaactttataaaacgtgtttgtattgtaggtggtgtttgtattatgcttatgactat

SEQ ID NO: 499 - DNA, 502 bp, Artificial Sequence
  Synthetic
tgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctagg

SEQ ID NO: 500 - DNA, 265 bp, Artificial Sequence
  Synthetic
tggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcc

SEQ ID NO: 501 - DNA, 510 bp, Artificial Sequence
  Synthetic
agatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaagcaagataattctatttgaagcatgctctgtaagttgcttcctaacatccttggactgagaaattatacttacttctggcataactaaa

SEQ ID NO: 502 - DNA, 551 bp, Artificial Sequence
  Synthetic
atggcttgatgtagcagtcatagcaagtttgtaaatagcatctatgttacactctcctagagtataaaatgtgaatgtttttgtagctaaattgtaattgaaactggctcattccagtttattgatttcacaataggggttaaattggcaaacattcatatttttacttcatttttaaaacaactgactgatagttctatattttcaaaatatttgaaaataaaaagtattcccaagtgattttaatttaaaaacaaattggctttgtctcattgatcagacaaaaagaaactagtattaagggaagcgcaaacacatttattttgtactgcagaaaaattgcttttttgtatcactttttgtgtaatggttagtaaatgtcatttaagtccttttatgtataaaactgccaaatgcttacctggtattttattagatgcagaaacagattggaaacagctaaattacaacttttacatatggctctgtcttattgtttcttcatactgtgtctgtatttaatctttttttatggaacctgttgcgcctat

SEQ ID NO: 503 - DNA, 386 bp, Artificial Sequence
  Synthetic
gaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacaca

SEQ ID NO: 504 - DNA, 245 bp, Artificial Sequence
  Synthetic
ttctctctgtgtggcaaacatttaaaaaaaaaaaaaaaaagcaggaaagatcccaagtcaaacagtgtggcttaaacattttttgtttcttggtgttgttatggcaagtttttggtaatgatgattcaatcattttgggaaattcttgcactgtatccaagttatttgatctggtgcgtgtggccctgtgggagtccactttcctctctctctctctctctgttccaagtgtgtgtgcaatgttc

SEQ ID NO: 505 - DNA, 433 bp, Artificial Sequence
  Synthetic
tatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaaca

SEQ ID NO: 506 - DNA, 358 bp, Artificial Sequence
  Synthetic
gtaaataaaacactcttccatatgatattcaacattttacaactgcagtattcacctaaagtagaaataatctgttacttattgtaaatactgccctagtgtctccatggaccaaatttatatttataattgtagatttttatattttactactgagtcaagttttctagttctgtgtaattgtttagtttaatgacgtagttcattagctggtcttactctaccagttttctgacattgtattgtgttacctaagtcattaactttgtttcagcatgtaattttaacttttgtggaaaatagaaataccttcattttgaaagaagtttttatgagaataacaccttaccaaacattg

SEQ ID NO: 507 - DNA, 300 bp, Artificial Sequence
  Synthetic
gaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatc

SEQ ID NO: 508 - DNA, 503 bp, Artificial Sequence
  Synthetic
tatcggctacatatgcagtctgtgaattatgtaacatactctatttcttgagggctgcaaattgctaagtgctcaaaatagagtaagttttaaattgaaaattacataagatttaatgcccttcaaatggtttcatttagccttgagaatggttttttgaaacttggccacactaaaatgtttttttttttttacgtagaatgtgggataaacttgatgaactccaagttcacagtgtcatttcttcagaactccccttcattgaatagtgatcatttattaaatgataaattgcactcgctgaaagagcacgtcatgaagcaccatggaatcaaagagaaagatataaattcgttcccacagccttcaagctgcagtgttttagattgcttcaaaaaatgaaaaagttttgcctttttcgatatagtgaccttctttgcatattaaaatgtttaccacaatgtcccatttctagttaagtcttcgcacttgaaagctaacat

SEQ ID NO: 509 - DNA, 545 bp, Artificial Sequence
  Synthetic
ggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgc

SEQ ID NO: 510 - DNA, 128 bp, Artificial Sequence
  Synthetic
cagcaacagcaaatcacgaccactgatagatgtctattcttgttggagacatgggatgattattttctgttctatttgtgcttagtccaattccttgcacatagtaggtacccaattcaattactatt

SEQ ID NO: 511 - DNA, 549 bp, Artificial Sequence
  Synthetic
aggttatgccatatcagagggaaattgagaaaaaaggtttggataaatacatcagacattatattggttggtctacgggactatcaggataacaaagctgatgtaattttaaagtacaatgcagatgaagctagaagcctgaaggcatatggcgagcttccagaacatgctaaaatcaatgaaacagacacatttggtcctggagatgatgatgaaatccagtttgacgatattggagatgatgatgaagacattgatgatatctaaattgaaccaagtgtttttacatgacaagttctctgaggatggttctacagttgggattttggccatcatcaaccaagaagagaaattcatttagtgtgtagtttctgaaagcaaactgatttattttcattgttttaaagtatttatttctttaaaagctgaggacactgaattaccttaagttaaatgttaatactttattgttttgatgtaatggaacttaaggataaaagaccataatatttgctgttaaaataaataaacgagtgcctttcctactgt

SEQ ID NO: 512 - DNA, 410 bp, Artificial Sequence
  Synthetic
ttatgttctaagtgcctccaagttcaaaacttattggaatgttgagagtgtggttacgaaatacgttaggaggacaaaaggaatgtgtaagtctttaatgccgatatcttcagaaaacctaagcaaacttacaggtcctgctgaaactgcccactctgcaagaagaaatcatgatatagctttccatgtggcagatctacatgtctagagaacactgtgctctattaccattatggataaagatgagatggtttctagagatggtttctactggctgccagaatctagagcaaagccatcccccctcctggttggtcacagaatgactgacaaagacatcgattgatatgcttctttgtgttatttccctcccaagtaaatgtttgtccttgggtccattttctatgctt

SEQ ID NO: 513 - DNA, 411 bp, Artificial Sequence
  Synthetic
ctttgcgggcacagagactgccacaaagtggagcggctacatggaaggggcagttgaggctggagaacgagcagctagggaggtcttaaatggtctcgggaaggtgaccgagaaagacatctgggtacaagaacctgaatcaaaggacgttccagcggtagaaatcacccacaccttctgggaaaggaacctgccctctgtttctggcctgctgaagatcattggattttccacatcagtaactgccctggggtttgtgctgtacaaatacaagctcctgccacggtcttgaagttctgttcttatgctctctgctcactggttttcaataccaccaagaggaaaatattgacaagtttaaaggctgtgtcattgggccatgtttaagtgtactggatttaactacctttg

SEQ ID NO: 514 - DNA, 318 bp, Artificial Sequence
  Synthetic
ttccacatcagtaactgccctggggtttgtgctgtacaaatacaagctcctgccacggtcttgaagttctgttcttatgctctctgctcactggttttcaataccaccaagaggaaaatattgacaagtttaaaggctgtgtcattgggccatgtttaagtgtactggatttaactacctttggcttaattccaatcattgttaaagtaaaaacaattcaaagaatcacctaattaatttcagtaagatcaagctccatcttatttgtcagtgtagatcaactcatgttaattgatagaataaagccttgtgatcact

SEQ ID NO: 515 - DNA, 396 bp, Artificial Sequence
  Synthetic
attcttctaattgctgtgtgtcccaggcagggagacggtttccagggaggggccggccctgtgtgcaggttccgatgttattagatgttacaagtttatatatatctatatatataatttattgagtttttacaagatgtatttgttgtagacttaacacttcttacgcaatgcttctagagttttatagcctggactgctacctttcaaagcttggagggaagccgtgaattcagttggttcgttctgtactgttactgggccctgagtctgggcagctgtcccttgcttgcctgcagggccatggctcagggtggtctcttcttggggcccagtgcatggtggccagaggtgtcacccaaaccggcaggtgcgattttgttaacccagcgacga

SEQ ID NO: 516 - DNA, 406 bp, Artificial Sequence
  Synthetic
gggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggagatgtcacagattcctgcagagccatggtcccaggcttcccaaaagtgtttgttggcaattattcccctaggctgagcctgctcatgtacc

SEQ ID NO: 517 - DNA, 507 bp, Artificial Sequence
  Synthetic
gaaaatgtaccttggtgccaccaacccattttgtgccacatgcaagttttgaataaggatgtatggaaaacaacgctgcatatacaggtaccatttaggaaataccgatgcctttgtgggggcagaatcacagacaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccgctgactctaatttatgaggatgccgaactctgatggcaataatatatgtattatgaaaatgaagttatgatttccgatgaccctaagtcc

SEQ ID NO: 518 - DNA, 527 bp, Artificial Sequence
  Synthetic
aacctacagcatattcttcacgcagagattttcatctattatactttatcaaagattggccatgttccacttggaaatggcatgcaaaagccatcatagagaaacctgcgtaactccatctgacaaattcaaaagagagagagagatcttgagagagaaatgctgttcgttcaaaagtggagttgttttaacagatgccaattacggtgtacagtttaacagagttttctgttgcattaggataaacattaattggagtgcagctaacatgagtatcatcagactagtatcaagtgttctaaaatgaaatatgagaagatcctgtcacaattcttagatctggtgtccagcatggatgaaacctttgagtttggtccctaaatttgcatgaaagcacaaggtaaatattcatttgcttcaggagtttcatgttggatctgtcattatcaaaagtgatcagcaatgaagaactggtcggacaaaatttaacgttgatgtaatggaattccagatgtaggcattccccc

SEQ ID NO: 519 - DNA, 494 bp, Artificial Sequence
  Synthetic
ggaacagttagttctcatctagaatgaaagttccatatatgcattggtgaatatatatgtatacacatacttacatacttatatgggtatctgtatagataatttgtattagagtattatatagcttcttagtagggtctcaagtaagttcattttttttatctgggctatatacagtcctcaaataaataatgtcttgattttatttcagcaggaataattttatttattttgcctatttataattaaagtatttttctttagtttgaaatgtgtattaaagttacatttttgagttacaagagtcttataactacttgaatttttagttaaaatgtcttaatgtaggttgtagtcactttagatggaaaattacctcacatctgttttcttcagtattacttaagattgtttatttagtggtagagagattttttttttcagcctagaggcagctattttaccatctggtatttatggtctaatttgtattt

SEQ ID NO: 520 - DNA, 444 bp, Artificial Sequence
  Synthetic
taatttctttacatactcaaaaccaagatagaaaaaggtgctatcgttcaacttcaaaacatgtttcctagtattaaggactttaatatagcaacagacaaaattattgttaacatggatgttacagctcaaaagatttataaaagattttaacctattttctcccttattatccactgctaatgtggatgtatgttcaaacaccttttagtattgatagcttacatatggccaaaggaatacagtttatagcaaaacatgggtatgctgtagctaactttataaaagtgtaatataacaatgtaaaaaattatatatctgggaggattttttggttgcctaaagtggctatagttactgattttttattatgtaagcaaaaccaataaaaatttaagtttttttaacaactaccttatttttcactgtacagacactaattca

SEQ ID NO: 521 - DNA, 467 bp, Artificial Sequence
  Synthetic
cggacattgccatcaacagctgaacagcctgcccaccatggaaggacccccaaccttcaacccgcctgtgccatatttcgggaggctgcaaggagggctcacagctcgaagaaccatcatcatcaagggctatgtgcctcccacaggcaagagctttgctatcaacttcaaggtgggctcctcaggggacatagctctgcacattaatccccgcatgggcaacggtaccgtggtccggaacagccttctgaatggctcgtggggatccgaggagaagaagatcacccacaacccatttggtcccggacagttctttgatctgtccattcgctgtggcttggatcgcttcaaggtttacgccaatggccagcacctctttgactttgcccatcgcctctcggccttccagagggtggacacattggaaatccagggtgatgtcaccttgtcctatgtccagatctaat

SEQ ID NO: 522 - DNA, 149 bp, Artificial Sequence
  Synthetic
aactgctttaagtcatgcttatgctgctggtgccagtcatttgaagaaaaacagtccttggaggaaaagcagtcgtgcttaaagttcaaagctaatgatcacggatatgacaacttccgttccagtaataaatacagctcatcttgaaa

SEQ ID NO: 523 - DNA, 379 bp, Artificial Sequence
  Synthetic
cctggtctgccacagatccatgatgtgcagttctctggagcaggcgctggctgtgctggtcactaccttccacaagtactcctgccaagagggcgacaagttcaagctgagtaagggggaaatgaaggaacttctgcacaaggagctgcccagctttgtgggggagaaagtggatgaggaggggctgaagaagctgatgggcagcctggatgagaacagtgaccagcaggtggacttccaggagtatgctgttttcctggcactcatcactgtcatgtgcaatgacttcttccagggctgcccagaccgaccctgaagcagaactcttgacttcctgccatggatctcttgggcccaggactgttgatgcctttgagtt

SEQ ID NO: 524 - DNA, 543 bp, Artificial Sequence
  Synthetic
ctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttattggttaaactcctttatggtgtgactgtgtcttattccatctatgagctttgtcagtgcgcgtagatgt

SEQ ID NO: 525 - DNA, 455 bp, Artificial Sequence
  Synthetic
tatgagccttcttgtttagtttcttttatctgctaagttgtaccttaattagagggcaatatatgtttcataaagaagagtctttataattttgtttgtcagatagtattttggaatttgtataataaggatgtttagaagccatataagtggctttttttaacagatagaatttgtatttttattgtactttaaaaagatttatgtaataggtatatatttagtggccatttattatcaatggtaacacaatggagtactaagatggtatttgcacatttaagatatgttactttaccaatttttaatggtaatcaactctgctactggcatgatgaaatagtacataactggtcattaattatgaacatttacttctccagtgcgtttttatgaagatctggttgaaaattgtatttctatgtaaactcaacgatatgtttggttttcctgaa

SEQ ID NO: 526 - DNA, 430 bp, Artificial Sequence
  Synthetic
gttattttcctatatctcacagtattaaaaaataaataattaaaaattaagaataaataaacgagttgacctcggtcacaaaagcagttttactatcgaatcaatcgctgttatttttttttaatgtaatttgtacatcttttttcaatctgtacatttgggctgtcttgtatgtttttatgctcctttttaaaaagcataatatgcctatagctgaaaaggaaacagggctgtttaagtcactgacttatgagaaagcaaagcactggtacagttatttaacaggcatacacaagcagggaaaagataatccatttagatctttaatgctttggaaatgcgtgtaacagtactgcaataatcacagctctgggaaaaacaacgaaactttcccttgtggagaggagggattttcctgctctatataagc

SEQ ID NO: 527 - DNA, 356 bp, Artificial Sequence
  Synthetic
tacccgcaggactggttccaagtcctcatcctgagaggtaacgggtcggaggcgcaccgcgtgccctgctcctgctacaacttgtcggcgaccaacgactccacaatcctagataaggtgatcttgccccagctcagcaggcttggacacctggcgcggtccagacacagtgcagacatctgcgctgtccctgcagagagccacatctaccgcgagggctgcgcgcagggcctccagaagtggctgcacaacaaccttatttccatagtgggcatttgcctgggcgtcggcctactcgagctcgggttcatgacgctctcgatattcctgtgcagaaacctggaccacgtctacaa

SEQ ID NO: 528 - DNA, 491 bp, Artificial Sequence
  Synthetic
aagcaaatagtgccctcagctactgcagaagaaaagtcccactgaggaaaagaaagtcttgtgatttttaaaggcaagttttcaagtgctctcatagttctatcctctaattccattaaatccatactaggagcgtcagtgagggttttcatagcttttggaaatactttggtctctgaactgtaattagcaagaagtaaaaacagaaacgtcaaacgtcaaatgtttgctttgttacctggaggactaaatgtagatgtctttagtatactttgtatgttcttaaatattggaagataattttgtgaatctgtagattttattttttcagtcttaccttacaaatttcttttctatgaataatagaggaactcacggcactctgccacttgttaatgaaaggaagtgcagaggatttagaaaagtacatgatccccagaccacaacaaaccaaaacataaactcatgtctgtgtcccatggtcatagtca

SEQ ID NO: 529 - DNA, 283 bp, Artificial Sequence
  Synthetic
tgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcaga

SEQ ID NO: 530 - DNA, 352 bp, Artificial Sequence
  Synthetic
tattggccttttgctaacacagtaagcatgtattttataaggcattcaataaatgcacaacgcccaaaggaaataaaatcctatctaatcctactctccactacacagaggtaatcactattagtattttggcatattattctccaggtgtttgcttatgcacttataaaatgatttgaacaaataaaactaggaacctgtatacatgtgtttcataacctgcctcctttgcttggccctttattgagataagttttcctgtcaagaaagcagaaaccatctcatttctaacagctgtgttatattccatagtatgcattactcaacaaactgttgtgctattggatactta

SEQ ID NO: 531 - DNA, 551 bp, Artificial Sequence
  Synthetic
cccggtgagcaaaaggttccctgtggggcccccgaagaatgatgataccccaaataggcagtactgggatgaagatctgttaatgaaagtgctggaatacctcaatcaagaaaaggcagaaaagggaagggagcatattgctaagagagcaatggaaaatatgtaagctgctttcattaattaccctactttcattcctcccaccccaagcaaatcccaacatttctcttcagtgtgttgacttctatcctgttaacactgtaatatctttaaatgatgtacaggcagatgaaaccaggtcactggggagtctgcttcatttcctctgagctgttatcttgtgtatggatatgtgtaaatgttatgactccttgataaaaaatttattatgtccattattcaagaaagatatctatgactgtgtttaatagtatatctaatggctgtggcattgttgatgctcacatatgataaaaaagtgtcctataattctattgaaagtttttaatatttattgaattattttgttactgtctgtagcgttttgtgga

SEQ ID NO: 532 - DNA, 390 bp, Artificial Sequence
  Synthetic
gaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgctttttaaaaaaaaaagaaaagaaaaagacagcatctcactatgttgccaaggctcatcttgagaagcaggcgggttgggtggg

SEQ ID NO: 533 - DNA, 512 bp, Artificial Sequence
  Synthetic
gagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacc

SEQ ID NO: 534 - DNA, 501 bp, Artificial Sequence
  Synthetic
gtgtgatgcttttgtaggtacctggaaacttgtctccagtgaaaactttgatgattatatgaaagaagtaggagtgggctttgccaccaggaaagtggctggcatggccaaacctaacatgatcatcagtgtgaatggggatgtgatcaccattaaatctgaaagtacctttaaaaatactgagatttccttcatactgggccaggaatttgacgaagtcactgcagatgacaggaaagtcaagagcaccataaccttagatgggggtgtcctggtacatgtgcagaaatgggatggaaaatcaaccaccataaagagaaaacgagaggatgataaactggtggtggaatgcgtcatgaaaggcgtcacttccacgagagtttatgagagagcataagccaagggacgttgacctggactgaagttcgcattgaactctacaacattctgtgggatatattgttcaaaaagatattgttgttttccctgatttagcaagca

SEQ ID NO: 535 - DNA, 505 bp, Artificial Sequence
  Synthetic
gtcaaggcattgtatgttgcttctgtggttattattctgtgatgcttagactacttgaacccataaacttggaagaatctttgagcaaattttctcagttgtctgtatgacttcagtatattcctgggaatgccataggattttttgtgcttgatacatggtatccagtttgcatagtatcacttctttgtaatccagttgctgttaagaatgatgtactttaaaggaaaagagaaaactgcatcacagtcccattctccagtgtccatgcaatgaattgctgagcatttaggaagcagcaccaagtctattacaggcatggtgtgaaacttgatgtttgacctgtgatcaaaattgaaccattgtacagtttggcttctgtttgcttcaaaatatgtagaattgtggttgatgattaatttgcgagactaactttgagagtgtaacagttttgaagaaaacattgaatgttttacaaatgaaggggcttcacggaatgttacaa

SEQ ID NO: 536 - DNA, 440 bp, Artificial Sequence
  Synthetic
  misc_feature (331)..(331): n is a, c, g, or t
ggactagagcaacatcgtgctgcccaaaggactaacctatgcaaactagttcacattttagtggatgtcgcagttaatgtgtaataagacattatttcccctgcataatgtacaacagcattgaaatgacacattaagcctagcatcacattgtatagtacagtcactcacaaacccttcaaggctaccctaatcattaacattaatatttgtttaaaagcaaatcaccgatttatctattgaaactacttaaatgacggcaaaccaggaatgacagatggctgtgtcagcaatggctttaatgtgttccctgcaagtggtctcctatgantagaactgcgttctcaaatgcactctcttcagggtcttaatattctgtgttttctctctgtatttgtaaaacattataacacattaatttcctatctctacacatttgg

SEQ ID NO: 537 - DNA, 372 bp, Artificial Sequence
  Synthetic
  misc_feature (57)..(57): n is a, c, g, or t
cagccatgtccatgggcctggagatcacgggcaccgcgctggccgtgctgggctggnctgggcaccatcgtgtgctgcgcgttgcccatgtggcgcgtgtcggccttcatcggcagcaacatcatcacgtcgcagaacatctgggagggcctgtggatgaactgcgtggtgcagagcaccggccagatgcagtgcaaggtgtacgactcgctgctggcactgccacaggaccttcaggcggcccgcgccctcatcgtggtggccatcctgctggccgccttcgggctgctagtggcgctggtgggcgcccagtgcaccaactgcgtgcaggacgacacggccaaggccaagatcaccatcgtggcaggcgtg

SEQ ID NO: 538 - DNA, 256 bp, Artificial Sequence
  Synthetic
cacaatacagggttgctatcagcactggatcttgttgaagtcaatcctcagttggccacctcagaggaagaggcgaagactacagctaacctggcagtagatgtgattgcttcaagctttggtcagacaagagaaggagggcatattgtctatgaccaacttcctactcccagttcaccagatgaatcagaaaatcaagcacgtgtgagaatttaggagacactgtgcactgacatgtttcacaacaggcattcca

SEQ ID NO: 539 - DNA, 444 bp, Artificial Sequence
  Synthetic
gagccctgattgatatgtatatagaaggtatagcagatttgggtgaaatgatcctccttctgcccgtatgtccacctgaggaaaaagatgccaagcttgccttgatcaaggagaaaataaaaaatcgctacttccctgcctttgaaaaagtcttaaagagccatggacaagactaccttgttggcaacaagctgagccgggctgacattcatctggtggaacttctctactacgtcgaggagcttgactccagtcttatctccagcttccctctgctgaaggccctgaaaaccagaatcagcaacctgcccacagtgaagaagtttctacagcctggcagcccaaggaagcctcccatggatgagaaatctttagaagaagcaaggaagattttcaggttttaataacgcagtcatggaggccaagaacttgcaataccaatgt

SEQ ID NO: 540 - DNA, 461 bp, Artificial Sequence
  Synthetic
gatttccaaatcagtcgttttctcaaaaaaatatcgtataagtgactcatcctgtctgctaactccagacctcccagcttgaagccaaatctttccatgtgagattgatatggatttcctagaagtactggaatgttgtcatatcttgccctattttaattctgctatagaaaacaattgccttcacttttaaggagtaatttgaatattaataactctggtctagattttcatataatgtattaaagacaaagtagtgaacatcaatgaacatctgatagagataaactgtaatcaggcataagcttgtttgtatgttctggcagtgactaatcagtaaatgatgtcggtttgcccagtatcacttatcttctgtatttttcctctgtcgtgtaaatagtataaccttttcatttatggacaattttttggactagtagccttcaatatacattctgctt

SEQ ID NO: 541 - DNA, 453 bp, Artificial Sequence
  Synthetic
  misc_feature (250)..(250): n is a, c, g, or t
aacactcacatacaagttcatactttaaaagaggaaagctacttaacaatgacaaatatttcacaataataatttttacttatataccatctttcaactgaacatttcagttcttccaagagcttcttagagtagtatattttgggggcagtcaaggaataaactacagtgtaaacatatcccagatgaaaactgctgtatggaaaaatgacagaaagtaactgattgacactgttgattcacagttcangcctcctatctgggaaagacatttctttcctctgctcactttaagaacttttaccgactccaaaaatctcaggaattaaacttttaacagttacagcaataaagaatagttagtactccaaaaatattatatttaagatgctcaacaagaaaaaaatgcaaatgtaatatttttttcaaattacttctttattgacttgtcca

SEQ ID NO: 542 - DNA, 509 bp, Artificial Sequence
  Synthetic
tgtgtaatacgccaaccagtcaagttgtgttttggccagagatttagatatgtccaatttcctggctcatttcattgtgctctatgggtacgtataaaaagcaagaattctgtttcctaggcaaacattgcaactcagggctaaagtcatccagtgaaacttttagagccagaagtaactttgtcccagtcctacaatgtgaaaagagtgaatagttgcctctttttagccattttcatggctggtacatattcgtacgcattacttttcagaatcaatacgcactttcagatattcttatttttattctcttaagtctttattaactttggagagagaaatgatgcatctttttattttaaatgaagtagatcaacatggtggaacaaaatgataaagaacagaaaacatttcaatatattactaataactttttccaatataaatcctaaaattcctataacatagtattttacagttttatgaagctttctattgtgacttttatg

SEQ ID NO: 543 - DNA, 483 bp, Artificial Sequence
  Synthetic
ttagtttctttgctctacgtgggcacctggcactaagggagtaccttattatcctacatcgcaaatttcaacagctacattatatttccttctgacacttggaaggtattgaaatttctagaaatgtatccttctcacaaagtagagaccaagagaaaaactcattgattgggtttctacttctttcaaggactcaggaaatttcactttgaactgaggccaagtgagctgttaagataacccacacttaaactaaaggctaagaatataggcttgatgggaaattgaaggtaggctgagtattgggaatccaaattgaattttgattctccttggcagtgaactactttgaagaagtggtcaatgggttgttgctgccatgagcatgtacaacctctggagctagaagctcctcaggaaagccagttctccaagttcttaacctgtggcactgaaaggaatgttgagttacctcttcatgtt

SEQ ID NO: 544 - DNA, 461 bp, Artificial Sequence
  Synthetic
tgttgcaaagaagtctgtcccccatttttcagatgaggataaggatccagagtaaagagaagatgctagacgaaaacccacattacctgttaggcctcagcatggcttatgtgcacgtgtaaatggagtccctgtgaatgacagcatgtttcttacatagataattatggatacaaagcagctgtatgtagatagtgtattgtcttcacaccgatgattctgctttttgctaaattagaataagagcttttttgtttcttgggtttttaaaatgtgaatctgcaatgatcataaaaattaaaatgtgaatgtcaacaataaaaagcaagactatgaaaggctcagatttcttgcagtttaaaatggtgtctgaggttgtactattttggccaagtctgtagaaagctgtcatttgattttgattatgtagttcatccagcccttgggcattgttatacacc

SEQ ID NO: 545 - DNA, 484 bp, Artificial Sequence
  Synthetic
tatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccgagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaa

SEQ ID NO: 546 - DNA, 493 bp, Artificial Sequence
  Synthetic
caaagcttttgagcgccacaggggaaagtgaaaaacaattccaggaagccataattgtgtttcaagaagagtttaaatgctgcggtttggtcaatggagctgctgattggggaaataattttcaacactatcctgaattatgtgcctgtctagataagcagagaccatgccaaagctataatggaaaacaagtttacaaagagacctgtatttctttcataaaagacttcttggcaaaaaatttgattatagttattggaatatcatttggactggcagttattgagatactgggtttggtgttttctatggtcctgtattgccagatcgggaacaaatgaatctgtggatgcatcaacctatcgtcagtcaaacccctttaaaatgttgctttggctttgtaaatttaaatatgtaagtgctatataagtcaggagcagctgtctttttaaaatgtctcggctagctagaccacagatatcttctagacata

SEQ ID NO: 547 - DNA, 318 bp, Artificial Sequence
  Synthetic
  misc_feature (230)..(230): n is a, c, g, or t
  misc_feature (258)..(258): n is a, c, g, or t
  misc_feature (264)..(264): n is a, c, g, or t
caaaaaagtgccctttttgtcactggtttctcctagcattcatgatttttttttcacacaatgaattaaaattgctaaaatcatggactggctttctggttggatttcaggtaagatgtgtttaaggccagagcttttctcagtatttgatttttttccccaatatttgattttttaaaaatatacacataggagctgcatttaaaacctgctggtttaaattctgtcanatttcacttctagccttttagtatggcnaatcanaatttacttttacttaagcatttgtaatttggagtatctggtactagctaagaa

SEQ ID NO: 548 - DNA, 444 bp, Artificial Sequence
  Synthetic
gaggcttttgacacagttattagttaaatcaaatgttcaaaaatacggagcagtgcctagtatctggagagcagcactaccatttattctttcatttatagttgggaaagtttttgacggtactaacaaagtggtcgcaggagattttggaacggctggtttaaatggcttcaggagacttcagttttttgtttagctacatgattgaatgcataataaatgctttgtgcttctgactatcaatacctaaagaaagtgcatcagtgaagagatgcaagactttcaactgactggcaaaaagcaagctttagcttgtcttataggatgcttagtttgccactacacttcagaccaatgggacagtcatagatggtgtgacagtgtttaaacgcaacaaaaggctacatttccatggggccagcactgtcatgagcctcactaagc

SEQ ID NO: 549 - DNA, 390 bp, Artificial Sequence
  Synthetic
cttttctcttctacattgacacacagaaatgtgtcaatgtaaagccaaaaccatcttctgtgtttatggccaatctattctcaaagttaaaagtaaaattgtttcagagtcacagttccctttatttcacataagcccaaactgatagacagtaacggtgtttagttttatactatatttgtgctatttaattctttctattttcacaattattaaattgtgtacactttcattacttttaaaaatgtagaaattcttcatgaacataactctgctgaatgtaaaagaaaattttttttcaaaaatgctgttaatgtatactactggtggttgattggttttattttatgtagcttgacaattcagtgacttaatatctattccatttgt

SEQ ID NO: 550 - DNA, 501 bp, Artificial Sequence
  Synthetic
ttcagtttttaatcgactggactgcagatgttttttcttttaacaaactggaattttcaaacagattatctgtatttaaatgtatagaccttgatatttttccaatactattttttaaaaaattgtatgatttacatatgaacctcagttctgaaattcattacatatctgtctcattctgccttttatactgtctaaaaaagcaaagttttaaagtgcaattttaaaactgtaaattacatctgaaggctatatatcctttaatcacattttatattttttcttcacaattctaacctttgaaaatattataactggatatttcttcaaacagatgtcctggatgatggtccataagaataatgaagaagtagttaaaaatgtatggacagtttttccggcaaaatttgtagcttatgtcttggctaaatagtcaaggggtaatatgggcctgttgtttagtgtctccttcctaaagagcacttttgtattgtaatttat

SEQ ID NO: 551 - DNA, 515 bp, Artificial Sequence
  Synthetic
gttttaattcaacccagccatgcaatgccaaataatagaattgctccctaccagctgaacagggaggagtctgtgcagtttctgacacttgttgttgaacatggctaaatacaatgggtatcgctgagactaagttgtagaaattaacaaatgtgctgcttggttaaaatggctacactcatctgactcattctttattctattttagttggtttgtatcttgcctaaggtgcgtagtccaactcttggtattaccctcctaatagtcatactagtagtcatactccctggtgtagtgtattctctaaaagctttaaatgtctgcatgcagccagccatcaaatagtgaatggtctctctttggctggaattacaaaactcagagaaatgtgtcatcaggagaacatcataacccatgaaggataaaagccccaaatggtggtaactgataatagcactaatgctttaagatttggtcacactctcacctaggtgagcgcattgagccagtggtg

SEQ ID NO: 552 - DNA, 494 bp, Artificial Sequence
  Synthetic
ctcccctcggagaagagctgttcaccacgagactggcacagttttctgagaaagactattcaaacagtctcaggaaatcaaatatcgaaagcactgacttctaagtaaaccacagcagttgaaagactccaaagaaatgtaagggaaactgccagcaacgcagcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactat

SEQ ID NO: 553 - DNA, 513 bp, Artificial Sequence
  Synthetic
ctggtaaacattccacgggacactgtcaacctctataccagctctggctgcctctgccctccacttaatgttaatgaggaatatatcatcatgggctatgaagatgaggaacgttccagattactcttggtggaaggctctatagctgagaagtggaaggatcgactcggtaaaaaagttaagcgctgggatatgaagcttcgtcatcttggactcagtaaaagtgattctagcaatagtgattccactcagagtcagaagtctggcaggaactcgaacccccggcaagcacgcaactaaatcccgaaatacaaaaagtaacacagtggacttcctattaagacttacttgcattgctggactagcaaaggaaaattgcactattgcacatcatattctattgtttactataaaaatcatgtgataactgattattacttctgtttctcttttggtttctgcttctctcttctctcaacccctttgtaatggtttgggggcagactcttaagt

SEQ ID NO: 554 - DNA, 313 bp, Artificial Sequence
  Synthetic
tgagcagtttcttcacaacaacgagaacattgaaaatgaggacctggtggcctgggtgacggtgggcttcctgcacatcccccactcagaggacattcccaacacagccacacctgggaactccgtgggcttcctgctccggccattcaacttcttcccagaggacccctccctggcatccagagacactgtgatcgtgtggcctcgggacaacggccccaactacgtccagcgctggatccctgaggacagggactgctcgatgcctcccccttttagctacaatgggacctatagacctgtgtgaccagcc

SEQ ID NO: 555 - DNA, 460 bp, Artificial Sequence
  Synthetic
taaggctccttcatgtggcgtattgggccatagcctataattggttagaacctcctattttaattggaattctggatctttcggactgaggccttctcaaactttactctaagtctccaagaatacagaaaatgcttttccgcggcacgaatcagactcatctacacagcagtatgaatgatgttttagaatgattccctcttgctattggaatgtggtccagacgtcaaccaggaacatgtaacttggagagggacgaagaaagggtctgataaacacagaggttttaaacagtccctaccattggcctgcatcatgacaaagttacaaattcaaggagatataaaatctagatcaattaattcttaataggctttatcgtttattgcttaatccctctctcccccttcttttttgtctcaagattatattataataatgttctctgggtaggtgttga

SEQ ID NO: 556 - DNA, 440 bp, Artificial Sequence
  Synthetic
gactcccgagggctagggctagagcagacccgggtaagtaaaggcagacccagggctcctctagcctcatacccgtgccctcacagagccatgccccggcacctctgccctgtgtctttcatacctctacatgtctgcttgagatatttcctcagcctgaaagtttccccaaccatctgccagagaactcctatgcatcccttagaaccctgctcagacaccattacttttgtgaacgcttctgccacatcttgtcttccccaaaattgatcactccgccttctcctgggctcccgtagcacactataacatctgctggagtgttgctgttgcaccatactttcttgtacatttgtgtctcccttcccaactagactgtaagtgccttgcggtcagggactgaatcttgcccgtttatgtatgctccatgtctagcccat

SEQ ID NO: 557 - DNA, 539 bp, Artificial Sequence
  Synthetic
gacacattttggtcttaatccatgtacacctttttatttctgtatttttccacttcactgtaaaaatagtatgtgtacataatgttttattggcatacgtctatggagaagtgcagaaacttcagaacatgtgtatgtattatttggactatggattcaggttttttgcatgtttatatctttcgttatggataaagtatttacaaaacagtgacatttgattcaattgttgagctgtagttagaatactcaatttttaatttttttaatttttttattttttattttctttttggtttggggagggagaaaagttcttagcacaaatgttttacataatttgtaccaaaaaaaaaaaaaaaggaaaggaaagaaaggggtggcctgacactggtggcactactaagtgtgtgtttttttaaaaaaaaaatggaaaaaaaaaagcctttaaactggagagacttctgacaacagctttgcctctgtattgtgtaccagaatataaatgatacacctctgaccccagcgttctgaataaa

SEQ ID NO: 558 - DNA, 540 bp, Artificial Sequence
  Synthetic
gttattcattctgtgatcatttattttaagcactcttaaagcaaaaaatgaatgtctaaaattgttttttgttgtacctgctttgactgatgctgagattcttcaggcttcctgcaattttctaagcaatttcttgctctatctctcaaaacttggtatttttcagagatttatataaatgtaaaaataataatttttatatttaattattaactacatttatgagtaactattattataggtaatcaatgaatattgaagtttcagcttaaaataaacagttgtgaaccaagatctataaagcgatatacagatgaaaatttgagactatttaaacttataaatcatattgatgaaaagatttaagcacaaactttagggtaaaaattgccattggacagttgtctagagatatatatacttgtggttttcaaattggactttcaaaattaaatctgtccctgagagtgtctctgataaaagggcaaatctgcacctatgtagctctgcatctcctgtcttttcaggtttgtcatcaga

SEQ ID NO: 559 - DNA, 298 bp, Artificial Sequence
  Synthetic
aaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaa

SEQ ID NO: 560 - DNA, 420 bp, Artificial Sequence
  Synthetic
atgtcagcattggcctctgtgtacaaaccaagagcttttacagatccagaatttattagtttaaaatgcaggtgaacttttttttgcgtttggtttacttgtctgtcaaatgtttccttaaacatgaaactgaataaggagaagagtatttttaacacttaaatttcttggcaaattttaaaacattttttagtctgtaatacactccacttgaagcacttaagtcttccttaaatgacttttcttaagtaatgatactgtgtgttttcccaaagcacttttaaaaaaatttttataaattactatctgttgaaaaggtgtccttttcctttcttctagtattttttttcttaccaaaattcactaatcttgaatgtttgtgatattaaatttcaaatgcagaatacttgactcatttaa

SEQ ID NO: 561 - DNA, 544 bp, Artificial Sequence
  Synthetic
gttgacaccacttagatttaaaggcagacagttttgctttagtacaatagtatacattttataatgatgaacttataatgattaagggacatttctataaaaatactacaatagttttatgcacaacttcccattaaaaatgagatttcttatttgtttgtctgtttttactctgggagtaatactttttaaattacctttacatatatagtcactggcatactgagaatatacaatgatcctggaaattgcagtaccaaaagcacacaacgattatagtaactataagatacaataaaccaaataaatgtgaaagtagattcatgaaaatgtattcctttaaaatattgttttcctacaggcctatttaacaagatgtttcattttctgtatattttgtagttaatataaatgttgctctaatcagattgcttaaaagcatttttattatatttatgttgttgaactaatatatgaaataagtaaatgtagctcccacaaggtaaacttcattggtaagattgcactgttctgattatgtaag

SEQ ID NO: 562 - DNA, 551 bp, Artificial Sequence
  Synthetic
tgacctagcattattttctggccccatttatcatatcccttttctcctccaaatgtttctcctctcacctcttctgtgggacttaaattgctatatctgctcagagctcacaaatgcctttgaattatttccctgacttcctgatttttttcttttctcaagtgttacctactaagagatgcctggagtaagccacccagctacctaattcctcagtaacctccatctataatctccatggaagcaacaaattccctttatgagatatatgtcaaatttttccatctttcatccagggctgactgaaaccgtggctaagaattgggagactctcttgtttcaagccaatttaacatcatttaccagatcatttgtcatgtccagtaacacagaagcaaccaactacagtatagcctgataacatgttgatttcttagctgacgttaatatttctttcttccttgtgttcccacccttggcattgccacccacccctcaattaaggcaacaatgaagttaatggataccctctgcctttggctcagaaatgt

SEQ ID NO: 563 - DNA, 557 bp, Artificial Sequence
  Synthetic
caaggaaccatatgtgctacccacaacacctcattcttacccagggtgcgctgcgtcctcatggtactgtaggcagctgaagaaccgccgttcccttgaaagggaacacctggcattctgtggtgtttcgtgctgtcttaaataatggtgcatttattatgttcaagttatttcaggattgccatatgtgcaaacaaatcatgcaatgcagccaaggaatatatgttgttgttgttgttttaaacccattttttttttagaattttcattaatactgtagttatacaccatatgcctcattttatcatagcctattgtgtatgaaagatgtttgtacaatgaattgatgtttagtttgctttagtcatttaaaaagatattgtaccaggatgtgctattaagagcacgtatccattattcttctcaacccaagaacctgtttcctggaccagtgaccaaacctcatatgtgaaatggccaaagcacatgcaggctcctggttgttcctctcaaacctgtgctgaccaaagattagtaaccagttatacccagtattt

SEQ ID NO: 564 - DNA, 320 bp, Artificial Sequence
  Synthetic
gtgaattttccatgaatgtttttaatattctcatctcaacattgtgatatatgctactaaaaaccttttcatatacatcttacctcatttcaagtgaattattttaatctttttctctctttccaaaaatttacaggaatgtttagtgtaattggatttcgctatcagttcccatccttaagttttgatattcaatatctgatagatacactgcatctttggtcatctaagatttgtttacaaatgtgcaaattatttagagcatagactttataagcattaaaaaaaactaatggaggtaaaacctaaatgcgatgtga

SEQ ID NO: 565 - DNA, 551 bp, Artificial Sequence
  Synthetic
tcctgccttcaccatgaagtccagcggcctcttccccttcctggtgctgcttgccctgggaactctggcaccttgggctgtggaaggctctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagcttgattcctgccatatggaggaggctctggagtcctgctctgtgtggtccaggtcctttccaccctgagacttggctccaccactgatatcctcctttggggaaaggcttggcacacagcaggctttcaagaagtgccagttg

SEQ ID NO: 566 - DNA, 451 bp, Artificial Sequence
  Synthetic
aacgatgtcacatgcgtgttcgaccccgtgcacaccatctcccacaccgtcatctcgctgcctaccttccgcgagttcacccgccctgaagagatcatcttcctccgggccatcacgccaccgcatcctgccagccaggctaacatcatcttcgacatcacggaagggaacctgcgggactcttttgacatcatcaagcgttacatggacggcatgaccgtgggtgtcgtgcgccaggtgcggcccatcgtgggcccatttcatgccgtcctgaagctggagatgaactatgtggtcgggggcgtggtttcccaccgaaatgttgtcaacgtccgcatcttcgtctctgagtactggttctgagggctggtctgccgcacagccgcaggtgcacctccaggccaaatcattgctgccagtgactgtggtctgtacttgtttataccctcag

SEQ ID NO: 567 - DNA, 436 bp, Artificial Sequence
  Synthetic
gctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaa

SEQ ID NO: 568 - DNA, 470 bp, Artificial Sequence
  Synthetic
gtgaacagcttggccttttttgggtgtcttgacaggccaagaagaacaaatgactcagaaccggattaacatgaaagttatccaggcgcagagttgaagaagcataagcaagcaagacaaaaacagagagaccgcaaggaggaagatctgtggtactgtcataaaaaacagtggagctctgtattagaaaagcccctcagaactgggaaggccaggtaactctagttacacagaaactggtactaaagtctatcaaactgattacacagactgtaagaattcaaagtcaactgacatctatgctacatatattatatagtttgtacttgactatgagccattaacttaaagcatatgtttcaaatagccattgctactattccttgtccggtgtaattttattttattgtttttactttggaagagatgaactgtgtatttaacttaagctattgctcttaaaaccaggg

SEQ ID NO: 569 - DNA, 544 bp, Artificial Sequence
  Synthetic
attcatccagacatattacagccagtttggggcccctgactgcaatgtgaaacctctcgctgctgctaggtttacaaacaagcccattgtcctgtgcctcctaatatcatttgtactgaagaccccatctggggacttgagactttggtcccagcccagactcctcagacttttctctcagttgggatgcttcactcgctgggggtgtttgtttgccctctcatttttcagtacttctacagaattttctctagagtcagtcattatgaaatgtacttccctccatcttaacctatcaactttctgcccctccttcaaggcccagtataaatgccacctcctccatgaagccttccctaattccaccccaaacccccaccttcaacaatatttcaacgcttctgcaatgatgaaaaagaaacatagttgtagtacttagcctacctagaccagcaagcattcatttttagctcgctcattttttaccatgttttccagtctgtttaacttctgcagtgccttcactacactgccttacataaac

SEQ ID NO: 570 - DNA, 529 bp, Artificial Sequence
  Synthetic
gagaggaccaaccagaattccctttggacatttgtgtttttttgtttttttattttgttttgttttttcttcttcttcttcttccttaaagacatttaagctaaaggcaactcgtacccaaatttccaagacacaaacatgacctatccaagcgcattacccacttgtggccaatcagtggccaggccaaccttggctaaatggagcagcgaaatcaacgagaaactggactttttaaaccctcttcagagcaagcgtggaggatgatggagaatcgtgtgatcagtgtgctaaatctctctgcctgtttggactttgtaattatttttttagcagtaattaaagaaaaaagtcctctgtgaggaatattctctattttaaatatttttagtatgtactgtgtatgattcattaccattttgaggggatttatacatatttttagataaaattaaatgctcttatttttccaacagctaaactactcttagttgaacagtgtgccctagcttttcttgcaaccagagta

SEQ ID NO: 571 - DNA, 168 bp, Artificial Sequence
  Synthetic
ggcatcatgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctgg

SEQ ID NO: 572 - DNA, 516 bp, Artificial Sequence
  Synthetic
aatgatggaatgttgactgtgtttggcacacaggacacggaccttcatggaagtccttgctctgcgtggcatctgtcagcttttcacctttcattcttattcttcacttttgctgctgagcctagctgtacaaacttgcactttcatttgctaatataaattcagttttattttaccattttagagactactaatgattaaatgtagaaggagagggtgcacatgtttttatgtggagtgtttaaaagataaatttataccactgtaatgtgcagcttttattaaaagagaaattggttgaactgctaggttgaatgagagacttcatctattggactattttttttaatccaggcatatggtctttagtaatggcttgtaatttgtgaaaacattaatttgggggttttccctgttttcagttgtccatgtacacatagtcattatattagaaaagaaagctgttcaacaaacttgtttaatttgtttaaatcaacatagcatgaaacaccaaat

SEQ ID NO: 573 - DNA, 508 bp, Artificial Sequence
  Synthetic
agccctggagaagacgaaagccaacatcaagtgggtgaaggagaacaaggaggtggtgctccagtggttcacagaaaacagcaaatagtccccagcccttgaagtcacccggccccgatgcaaggtgcccacatgtgtccatcccagcggctggtgcagggcctccattcctggagcccgaggcaccagtgtcctcccctcaaggacaaagtctccagcccacgttctctctgcctgtgagccagtctagttcctgatgacccaggctgcctgagcacctcccagcccctgcccctcatgccaaccccgccctaggcctggcatggcacctgtcgcccagtgccctggggctgatctcagggaagcccagctccagggccagatgagcagaagctctcgatggacaatgaacggccttgctgggggccgccctgtaccctctttcacctttccctaaagaccctaaatctgaggaatcaacagggcagcagatctgtatatttttttc

SEQ ID NO: 574 - DNA, 364 bp, Artificial Sequence
  Synthetic
gtacccagttaaattttcatttcagataaacaacaaataattttttagtataagtacattattgtttatctgaaagttttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtat

SEQ ID NO: 575 - DNA, 224 bp, Artificial Sequence
  Synthetic
gaagcgtttaggcatgtttaacatccagcactgtaagaagctgtccagctgggtactgctaatgaaatacctgggcaatgccaccgccatcttcttcctacctgatgaggggaaactacagcacctggaaaatgaactcacccacgatatcatcaccaagttcctggaaaatgaagacagaaggtctgccagcttacatttacccaaactgtccattactggaa

SEQ ID NO: 576 - DNA, 549 bp, Artificial Sequence
  Synthetic
  misc_feature (76)..(76): n is a, c, g, or t
aaactactaaccactgcaagctcttgtcaaattttagtttaattggcattgcttgttttttgaaactgaaattacntgagtttcattttttctttgaatttatagggtttagatttctgaaagcagcatgaatatatcacctaacatcctgacaataaattccatccgttgttttttttgtttgtttgttttttcttttcctttaagtaagctctttattcatcttatggtgcagcaattttaaaatttgaaatattttaaattgtttttgaactttttgtgtaaaatatatcagatctcaacattgttggtttcttttgtttttcattttgtacaactttcttgaatttagaaattacatctttgcagttctgttaggtgctctgtaattaacctgacttatatgtgaacaattttcatgagacagtcatttttaactaatgcagtgattctttctcactactatctgtattgtggaatgcacaaaattgtgtaggtgctgaatgctgtaaggagtttaggttgtatgaattctacaaccctataata

SEQ ID NO: 577 - DNA, 345 bp, Artificial Sequence
  Synthetic
gggttgagcgaattagggagattgctagtggaaattggagggaatttgttttgcatcatttgtctaggatctatgcaaatatagctccactaaaggaccatagggaagagccagccttgccttttcttatatgattttgtttacaaaattttactgggacttttaaatctagctatagagttgggaaaaaatatttccacttagatattttacatggttttgtttaaaattaccattacttgttttttaaaaacacatgaccacatatgtatatgtatatctacctaaacattgtatcatggtttcagtatgttattcatgtattactgggagatgctaccaaga

SEQ ID NO: 578 - DNA, 543 bp, Artificial Sequence
  Synthetic
gaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgattaaaaggtatactttagtagacatttataactcaaggataccttcttatttaatcttttcttatttttgtactttatcatgaatgcttttagtgtgtgcataatagctacagtgcatagttgtagacaaagtacattctggggaaacaacatttatatgtagcctttactgtttgatataccaaattaaaaaaaaattgtatctcattacttatactgggacaccat

SEQ ID NO: 579 - DNA, 444 bp, Artificial Sequence
  Synthetic
gaagccctggaaaatcgcctgagatacagatgaagattagaaatcgcgacacatttgtagtcattgtatcacggattacaatgaacgcagtgcagagccccaaagctcaggctattgttaaatcaataatgttgtgaagtaaaacaatcagtactgagaaacctggtttgccacagaacaaagacaagaagtatacactaacttgtataaatttatctaggaaaaaaatccttcagaattctaagatgaatttaccaggtgagaatgaataagctatgcaaggtattttgtaatatactgtggacacaacttgcttctgcctcatcctgccttagtgtgcaatctcatttgactatacgataaagtttgcacagtcttacttctgtagaacactggccataggaaatgctgtttttttgtactggactttaccttgatatatgt

SEQ ID NO: 580 - DNA, 300 bp, Artificial Sequence
  Synthetic
gttgttccaatgaatgtacagcacttccattaacttttgaaagcaacacagccttaaactcaatgcttttgctttatgacatgggaatgttctgtcatcaatggagtgtattcttgtaatagaattctttatatcgttctcaattctatagactttcaagcctatgtatgaatatgaaggggtttttttttttttgctttgttttctttttagattttgtacattccatctttataggtctgtttcatatgttttatgtatagaacactaagtcttgcactctctgacattgatactgat

SEQ ID NO: 581 - DNA, 472 bp, Artificial Sequence
  Synthetic
  misc_feature (149)..(149): n is a, c, g, or t
  misc_feature (153)..(153): n is a, c, g, or t
  misc_feature (428)..(428): n is a, c, g, or t
gctccccatttttataccaaaggtgctacatctatgtgatgggtggggtggggagggaatcactggtgctatagaaattgagatgcccccccaggccagcaaatgttcctttttgttcaaagtctatttttattccttgatattttttntttntttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttncccctaggggtgggaggaagcaaaagactctgtacctattttgt

SEQ ID NO: 582 - DNA, 324 bp, Artificial Sequence
  Synthetic
gtatgacaacccgggatcgtttgcaagtaactgaatccattgcgacattgtgaaggcttaaatgagtttagatgggaaatagcgttgttatcgccttgggtttaaattatttgatgagttccacttgtatcatggcctacccgaggagaagaggagtttgttaactgggcctatgtagtagcctcatttaccatcgtttgtattactgaccacatatgcttgtcactgggaaagaagcctgtttcagctgcctgaacgcagtttggatgtctttgaggacagacattgcccggaaactcagtctatttattcttcagcttgccc

SEQ ID NO: 583 - DNA, 445 bp, Artificial Sequence
  Synthetic
aagagctatgagctgccagatgggcaggttatcaccattggcaatgagcgcttccgctgccctgagaccctcttccagccttcctttattggcatggagtccgctggaattcatgagacaacctacaattccatcatgaagtgtgacattgacatccgtaaggacttatatgccaacaatgtcctctctgggggcaccaccatgtaccctggcattgctgacaggatgcagaaggagatcacagccctggcccccagcaccatgaagatcaagattattgctcccccagagcggaagtactcagtctggatcgggggctctatcctggcctctctctccaccttccagcagatgtggatcagcaagcctgagtatgatgaggcagggccctccattgtccacaggaagtgcttctaaagtcagaacaggttctccaaggatcccc

SEQ ID NO: 584 - DNA, 335 bp, Artificial Sequence
  Synthetic
taacacttggctcttggtacctgtgggttagcatcaagttctccccagggtagaattcaatcagagctccagtttgcatttggatgtgtaaattacagtaatcccatttcccaaacctaaaatctgtttttctcatcagactctgagtaactggttgctgtgtcataacttcatagatgcaggaggctcaggtgatctgtttgaggagagcaccctaggcagcctgcagggaataacatactggccgttctgacctgttgccagcagatacacaggacatggatgaaattcccgtttcctctagtttcttcctgtagtactcctcttttagatcc

SEQ ID NO: 585 - DNA, 444 bp, Artificial Sequence
  Synthetic
gacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtaatagtgacagcaaaggcccaggggagaggcattgccttctctgcccacagtctttccgtgtgattgtctttgaatctgaatcagccagtctcagatgccccaaagtttcggttcctatgagcccggggcatgatctgatccccaagacatg

SEQ ID NO: 586 - DNA, 522 bp, Artificial Sequence
  Synthetic
tcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgt

SEQ ID NO: 587 - DNA, 516 bp, Artificial Sequence
  Synthetic
cgctgctgtgcctcgatggcaaacggaagcctgtgactgaggctagaagctgccatcttgccatggccccgaatcatgccgtggtgtctcggatggataaggtggaacgcctgaaacaggtgctgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggctcccctgctgaaggtggggattgcccatccatctgcttacaattccctgctgtcgtcttagcaagaa

SEQ ID NO: 588 - DNA, 498 bp, Artificial Sequence
  Synthetic
gacaggtgaacatttccgcctggtctatgacaccaagggccgttttgctgttcaccgcatcacagtggaagaggcaaagtacaagttgtgcaaagtgaggaagattactgtgggagtgaagggaatccctcacctggtgactcatgatgctcgaaccatccgctacccagatcctgtcatcaaggtgaacgatactgtgcagattgatttagggactggcaagataatcaactttatcaaatttgatacaggcaatttgtgtatggtgattggtggagccaacctcggtcgtgttggtgtgatcaccaacagggaaagacatcctggttcttttgatgtggtgcatgtgaaggatgccaatggcaacagctttgccacgaggctttccaacatttttgtcattggcaatggcaataaaccttggatttccctgcccaggggaaagggcattcgacttactgttgctgaagagagagataagaggctggccaccaaaca

SEQ ID NO: 589 - DNA, 323 bp, Artificial Sequence
  Synthetic
agaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactat

SEQ ID NO: 590 - DNA, 562 bp, Artificial Sequence
  Synthetic
tcccagatacttcccattttggaagttttcaggacttggtctgatttcaggatactctgtcagatgggaagacatgaatgcacactagcctctccaggaatgcctcctccctgggcagaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggctcaaagttggggcaggctggctggcccgtcatgttcctcaaaagcacccttgacgtcaagtctccttcccctttccccactccctggctctcagaaggtattccttttgtgtacagtgtgtaaagtgtaaatcctttttctttataaactttagagtagcatgagagaattgtatcatttgaacaactaggcttcagcatatttatagcaatccatgttagtttttactttctgttgccacaaccctgttttatact

SEQ ID NO: 591 - DNA, 542 bp, Artificial Sequence
  Synthetic
gaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgatttgtgattgaaagctgcctttctatttacttgagtct

SEQ ID NO: 592 - DNA, 378 bp, Artificial Sequence
  Synthetic
gatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctggcggatgtgcgagctgatagtgagcggcagaatcaggagtaccagcggctcatggacatcaagtcgcggct

SEQ ID NO: 593 - DNA, 563 bp, Artificial Sequence
  Synthetic
  misc_feature (297)..(298): n is a, c, g, or t
tcaagtccaagttcaagtccaccatcgcggcgctggaggccaagattgcacagctggaggagcaggtcgagcaggaggccagagagaaacaggcggccaccaagtcgctgaagcagaaagacaagaagctgaaggaaatcttgctgcaggtggaggacgagcgcaagatggccgagcagtacaaggagcaggcagagaaaggcaatgccagggtcaagcagctcaagaggcagctggaggaggcagaggaggagtcccagcgcatcaacgccaaccgcaggaagctgcagcgggagnnggatgaggccacggagagcaacgaggccatgggccgcgaggtgaacgcactcaagagcaagctcagagggccccccccacaggaaacttcgcagtgatgcaccaggcgaggaaacgagacctctttcgttccttctagaaggtctggaggacgtagagttattgaaaatgcagatggttctgaggaggaaacggacactcgagacgcagacttcaatggaaccaaggccagtgaataagcaactttctacagttttgcaccacgg

SEQ ID NO: 594 - DNA, 481 bp, Artificial Sequence
  Synthetic
ggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaattttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtat

SEQ ID NO: 595 - DNA, 515 bp, Artificial Sequence
  Synthetic
taagaacttgatatatttcattctcttatatagtattcattttcctacctatattacacaaaaaagtatagaaaagatttaagtaattttgccatcctaggcttaaatgcaatattcctggtattatttacaatgcagaattttttgagtaattctagctttcaaaaattagtgaagttcttttactgtaattggtgacaatgtcacataatgaatgctattgaaaaggttaacagatacagctcggagttgtgagcactctactgcaagacttaaatagttcagtataaattgtcgtttttttcttgtgctgactaactataagcatgatcttgttaatgcatttttgatgggaagaaaaggtacatgtttacaaagaggttttatgaaaagaataaaaattgacttcttgcttgtacatataggagcaatactattatattatgtagtccgttaacactacttaaaagtttagggttttctcttggttgtagagtggcccagaattgcattct

SEQ ID NO: 596 - DNA, 552 bp, Artificial Sequence
  Synthetic
aacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagaatattcagattgtgtagatatgctatttaaataatttatcaggaaatactgcctgtagagttagtatttctatttttatataatgtttgcacactgaattgaagaattgttggttttttcttttttttgttttttttttttttttttttttttttgcttttgacctcccatttttactatttgccaatacctttttctaggaatgtgcttttttttgtacacatttttatccattttacattctaaagcagtgtaagttgtatattactgtttct

SEQ ID NO: 597 - DNA, 445 bp, Artificial Sequence
  Synthetic
atgggctcctgattctcatgaaataccctgagatcgaagagaagctccatgaagaaattgacagggtgattgggccaagccgaatccctgccatcaaggataggcaagagatgccctacatggatgctgtggtgcatgagattcagcggttcatcaccctcgtgccctccaacctgccccatgaagcaacccgagacaccattttcagaggatacctcatccccaagggcacagtcgtagtgccaactctggactctgttttgtatgacaaccaagaatttcctgatccagaaaagtttaagccagaacacttcctgaatgaaaatggaaagttcaagtacagtgactatttcaagccattttccacaggaaaacgagtgtgtgctggagaaggcctggctcgcatggagttgtttcttttgttgtgtgccattttgcagcattt

SEQ ID NO: 598 - DNA, 497 bp, Artificial Sequence
  Synthetic
cttctggggcagaacctctgctggtaccagacagccccagttcattagccgggactggcacggtgtgccagggcaagtggacgcagccatggctggccgcatctacatctcaggcatggcaccccgcccctccttgaccaagaaacaaaggtttaggcatcgcaaccgcaaaggctaccgttcacaacgaggccacagccgtggccgcaaccagaactcccgccggccatcccgcgccatgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcactactggctgggctgcccagctcctggccatctgtaggagtcagagcccacatggc

SEQ ID NO: 599 - DNA, 409 bp, Artificial Sequence
  Synthetic
ttaacttcattttgtatcttgcttaagtagcaggctcactaaaattagagaaagtccaataactctccccctttcccttgagaaatctttaagtttcgattctggagcaaaaactttcagcattaaatatttcagaggctccattcacagctttcagataaactggagtgttcagatggactgttttaataaaaatctttgagcaagtgagttatggcaagagaaactcagcctctttctgtataaacttaacagggaagggctggggtgtgaaaaagaagattgtatgaaaaccattggtaatttttattttttatttttgggactgcactatcctgttcacgaagacatgtgaacttggttcagtccaaatggggatttgtataaaccagtgctctccattagaaat

SEQ ID NO: 600 - DNA, 437 bp, Artificial Sequence
  Synthetic
acccgctctacaatatgagccttctgaagcatcaaagccttagaccagatgaagactccagccatgacctcatgctgcttcgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtac

SEQ ID NO: 601 - DNA, 423 bp, Artificial Sequence
  Synthetic
aataatttattcccacatctacatcagtgaaagctatctacctatcctgagtctatcttaaaggaaaaaaagaaaaaaaccttatctcttgcccttattttgaattttccactctttcattaatttgttttaagctcctgttggaaaaaaaggggtagtgcattttaaattgaccttcatacgcttttaaaataagacaaatctacttgataatgtacctttatttgatctcaagttgtataaaaccaataaatttgtgttactgcagtagtaatcttatgcacacggtgatttcatgttatatatgcaaagtaggcaactgttttcttagttacagaagtttcaagcttcacttttgtgcagtagaaacaaaagtaggctacagtctgtgccatgttgatgtacagtttctgaaattgtttt

SEQ ID NO: 602 - DNA, 395 bp, Artificial Sequence
  Synthetic
tgatagcgagtgattctcaccgtcctgaagtcaaactcaaaatggcacctgttcctcttgatgattctaacagacctgcttccttgactaaagacaggctgcttgctagtacacttgtccattctgtcaagaaggagcaagaattccgatccatctttgaccacattaagttgccacaggccagcaaaagcacttcagagtcatttattcaacacattgtgtccttggttcatcatgttaaagagcaatacttcaagtcagctgcaatgaccctaaacgagcggttcacttcgtatcagaaagccactgaagaacatagtactcggcaaaagagccctgaaatacacaggagaattgacatctcaccaagtaccctgaggaagcatacccgttta

SEQ ID NO: 603 - DNA, 539 bp, Artificial Sequence
  Synthetic
tggcgatggggcagagctgacggcagagttcctctacgacgaggtgcaccccaagcaacacgccttcaagcaggccttcgccaagcccaagggcccagggggcaagcggggccataagcgcctcatccgcggcccgggtgaaaatggggatgacagctaggaggctggagcagggccggccacgtgtggactgtggggctgcccaccttccgctccctgccaccatcctccttcctgggctccaggaaagtgtttctgggaggtcaggagggctggcagctgaacgcacttgcagcgtccgagggccaccgggctggcattttgtgacccttccctgttgctgtccctgcatctcgtctgtgtgcccagggtgtccggggaccctgcctggctggcttaagggggctgggtcaggggcctggcatgaacctggcctcccggggagctgagactagggtcccagcacagcccagaaacctttggccacaagaagtggggtcagtcagggctggggcaggggtcactgcagtttgggatgg

SEQ ID NO: 604 - DNA, 341 bp, Artificial Sequence
  Synthetic
attactaagccccaagagacatttcctgtgctagagtccaatatttgagtctctcgtgcaaatgagactattctttgtggtacaattccacctatcatatgtgaaaactgcagtaaaaataaacccagatgctaaatcattcctacaaaggtttgactgaaactgtggcagatgtctcatcttctttatatgttaagcagcatactcttctgatttttattgcaatcttttaccaagtggtgcacaaacttggtattgatgtctttattccattttgagtttagattgagaatatttttattttctgaaggcagagatatctactgtataattgcaccaaa

SEQ ID NO: 605 - DNA, 409 bp, Artificial Sequence
  Synthetic
aacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcaccggtcacccttaatatttattaagtgcctgagacacccggttaccttggccgtgaggacacgtggcctgcacccaggtgtggctgtcaggacaccagcctg

SEQ ID NO: 606 - DNA, 581 bp, Artificial Sequence
  Synthetic
  misc_feature (408)..(410): n is a, c, g, or t
cgccatcgtagtagaagcagggagcggaagaagtctcgatccaggagtagggagcggaagaaatcgagatccagaagcagagagaggaagaaatcgagatccagaagcagggaaagaaaacggcggatcaggtctcgttcccgctcaagatcaagacacaggcataggactagaagcaggagtaggacaaggagtaggagtcgagatagaaagaagagaattgaaaagccgagaagatttagcagaagtttaagccggactccaagtccacctcccttcagaggcagaaacacagcaatggatgcacaggaagctttagctagaaggttggaaagggcaaagaaattacaagaacagcgagaaaaggaaatggttgaaaaacaaaaacaacaagaaatagctgcagcnnntgcagctactggaggttctgttctcaatgttgctgccctgttggcatcaggaacacaagtaacacctcagatagccatggcagctcagatggcagccctgcaagctaaagctttggcagagacaggaatagctgttcctagctactataacccagccgctgttaatccaat

SEQ ID NO: 607 - DNA, 515 bp, Artificial Sequence
  Synthetic
gttgaggatctcttactctctaagccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtgggtactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctg

SEQ ID NO: 608 - DNA, 499 bp, Artificial Sequence
  Synthetic
cagctcatctcatgtcctgaagttggaatttaaataattcctatttgcaaattagaatgacagtgttggaatttggaggcagtagttgagcatattctctagtatatagctacacctttaataaaatgaaggaatgtcttcaatcatattttagtgggctatttataaatagtcttgaagtcaatttagtttatttatttaaaagataatgcatcctgaaagggatcatttatgaataacaatctgaagtcttttcataaaaaaaattaataaactttagttgtacatttagccagtgttatttgaagtatgtaacttttaaaatattaagtgtcttgtatgattagaatatgtgaatgagtaacttattttgtatcaggaatgttttggtactgtgttttcactcaaaccactgacttaacagatactgctgtgtataacatgtactaaatattacagttattgtgcataacagattgttcctcttatatttgtgtgt

SEQ ID NO: 609 - DNA, 383 bp, Artificial Sequence
  Synthetic
tattgcctaacttaagccatgacttttagatatgagatgacgggaagcaggacgaaatatcggcgtgtggctggagccttcccactggaggctgaaagtggcttgtggtattataatgttcagatttcaagaggaaggtgcaggtacacatgagttagagagctggtgagacagttgggaactctttgtgcttgtgatctactggactttttttttgcaggaagtgcattctctggtccttccctattttctgttctggatgtcagtgcagtgcactgctactgttttatccacttggccacagactttttctaacagctgcgtattatttctatatactaattgcattggcagcattgtgtctttgaccttgtatactagct

SEQ ID NO: 610 - DNA, 504 bp, Artificial Sequence
  Synthetic
ggccatgggtgcagaccgaggtatccacgtggaggtgcccccagcagaagcagaacgcttgggtcccctgcaggtggctcgggtcctggccaagctggcagagaaggagaaggtggacctggtgctgctgggcaaacaggccatcgatgatgactgtaaccagacagggcagatgacagctggatttcttgactggccacagggcacattcgcctcccaggtgacgctggagggggacaagttgaaagtggagcgggagatcgatgggggcctggagaccctgcgcctgaagctgccagctgtggtgacagctgacctgaggctcaacgagccccgctacgccacgctgcccaacatcatgaaagccaagaagaagaagatcgaggtgatcaagcctggggacctgggtgtggacctgacctccaagctctctgtgatcagtgtggaggacccgccccagcgcacggccggcgtcaaggtggagaccactgaggacctggtggc

SEQ ID NO: 611 - DNA, 433 bp, Artificial Sequence
  Synthetic
ctccctcacagtcttatagcacaatatacccaaatcagcccccccagtccgaggctgggcccaaggtatggtcggaggaggagctcctgcctgcggttttgtgtatgtgtgtatgtgtgtgcgtgtttgtgtgcgtgtttacctccacaggggacactctacactcagtgtaagatctgctgggaacagggccaccaggagtgcgtggatctcagtctctctgtctctctttctctccttttaattttggtgtatcaaatatttgattgacaaagtaagggccttgattaggaccaaattctcgtgtgttgctatggtctttatttaggacaacaattaacaatgcagtggcccattcttgtcactctacacatatgactatacgggacatatgtaatatataaatatatatataaaacattcccctctgtcc

SEQ ID NO: 612 - DNA, 505 bp, Artificial Sequence
  Synthetic
atatgggcaacgattatcttggaaattaaagagtcaaagcaaagaaatgaagggctggtaaaatgaattttgtaatatcctcaggatacttttatcttaaaagtatgttgttaaagattttgtaaattgtatttcaacaattttaaatgtgttgagcaagttgcagtgcaaacactgtcattatgtagagagtttatatgcacatgaataacctgtacctataaatcgtgcaataaccatatgcgactattttgccatggagaaatctgacagcattgcaaacaatagtattgtttgatgtagttaaccttaagttatttttcagtaatttcttcacaaatcaagattcaaacagctttaaacacttccaatgagataaaatatttactattatgcttattagaacaaaaggtgtttaaggatgaactaaatattttaattgagcatttatatggataatcatacattatgtaagcccatatgtatttacatccagagtcataat

SEQ ID NO: 613 - DNA, 315 bp, Artificial Sequence
  Synthetic
ttgggctgtgttggaatgggcctgcagcccaacaaacaagggaactaggaccgacagtgacttcaccagcttgctaggtcagaatgagagactggtgggtctgtctacctgtttcttctacaagatccctatttgactgtaaaagtagctaatactcacatgttctccaatcccaggtagccatggtagagttgggtagagttgagcagccgccccaggatccaaatgtggtgtctgaaatggaaagaactaaggcaaccaggaaggcactgatctgccttataagcacagtcatctgaaagtcaggcctgctgc

SEQ ID NO: 614 - DNA, 466 bp, Artificial Sequence
  Synthetic
gtccagcttgaacatctagaattcctagagaaacagaatgagcaggcgaaggagatgcagcagatggtgaaattggaagccgagatggaccgcagaccagcaacagtagtatgaaactccaaaatgcaaactgaagcagcaaacccacaaagcatcaaaagactcactcacaaaacttctgaacacaaactccatggatgaaagctgtttattttgtttcctttatgtgtaaacaagatgatatctgaaaccagagagacttggaatgtctgactgacttctatttaacagcttgagtattgcatttccttggccaaacaaaaatagctacaaatccacaaaaatttactattccagtaaggcagagtccaaccattgataatacaacttaaacatgtttgctataaaataccatcacaagtaaatgagcttggtgtgaacaactcttcctttgtgatgccttagg

SEQ ID NO: 615 - DNA, 532 bp, Artificial Sequence
  Synthetic
gtcttccaggggactctatcagaactggaccatttccaagtatataacgatgagccctctaatgctaggagtagcaaatggtcctaggaaggggactgaggattggggtgggggtggggtggaaaagaaagtacagaacaaaccctgtgtcactgtcccaagttaagctaagtgaacagaactatctcagcatcagaatgagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcagtcagttccaatgaggtggggatggagaagacaattgttgcttatgaaaaaaagctttagctgtctcctgttt

SEQ ID NO: 616 - DNA, 352 bp, Artificial Sequence; identical to SEQ ID NO: 530
  Synthetic

SEQ ID NO: 617 - DNA, 305 bp, Artificial Sequence
  Synthetic
ttcaacatgctccaaatataagacatttgtttgctgtacagaaagtatcacaaatggaatatatcagtacctctcaagctagtgtttctagctaaataaatgggtgtatataattttatggtggaaaagaactgtactgtctgttatgatttccttcaatgtgcataatgataaaataaataattttaatattcttttgtttccatggttacctgacctaaattagataaattgtagggctttagctttcttatttttgtcaaaagttggtgttgacatacattccctctaatttgaactggtat

SEQ ID NO: 618 - DNA, 422 bp, Artificial Sequence
  Synthetic
aggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggcagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcg

SEQ ID NO: 619 - DNA, 477 bp, Artificial Sequence
  Synthetic
tcagaagaggctcttggccctggaccccatgatggagcaggagattgaagagatccggcagaagtaccagtccaagcggcagcccatcctggatgccatagaggctaagaagagacggcaacaaaacttctgagcaaggccaggctgtgagggccccagctccacccaggctttgggtgaattctggatggcttgcctcatgtttgttagccagcacttctgctctgtcgtctctccacagcacctttgtgaactcaggaatgtgcgccagtgggaagggctctcttgacagtcagcgtgccatcttgatgtgtgtatgtacattggtcaggtatattatctcaaaggatttatattggcgcttttaactcagagttttaaaccccaggaacagagactcctagttgagtgatagctgggaaagttttacattgtctgtttttcttctcccaatagctttcaattgttctttctgga

SEQ ID NO: 620 - DNA, 533 bp, Artificial Sequence
  Synthetic
cacctggagaatctgccactgctgtaatgggcattaatttctgtgactcaacccaggcagccaacttccagctgtgcacccagacccgacagttctacgtctccattcagccacctgttggggagctgatggcccctgtgttcatgagtgaaaatgagtttaagaaggaacagggaaagctgatgggcatgaatgagatcacagagaaactcatgctgccagacacctgtcggagtgaccacattgtggtgcagaaagtgactgccactgccaacctgggtcgtgttccttgtgggacatctgatgagtacaggtttgcagggaggacactgactggtggaagcctcgttctgctgaccctggatgcccggccagctggagctgcccagctgactgtcaacagcgagaaaatggtgattggcaccatgctggtaaaggatgtgatacaggctctgacccagtgacttccaaatgctgtgacctgtttggctcccatctatacctccccatgacacctaggctgtcagtctctc

SEQ ID NO: 621 - DNA, 455 bp, Artificial Sequence
  Synthetic
catcctggacggcacggaccagatgtggctgcctgtggtgcgcacttggcgcttcaatgagcggcattacgggggcctcacaggcttcaacaaggcagaaacggccgccaagcacggggaggagcaggtaagatcttggaggcgctccttcgacatcccgccgcccccgatggacgagaagcacccctactacaactccattagcaaggagcgtcggtacgcaggcctgaagcccggggaactccccacctgcgagagcctcaaggacaccattgcccgggccctgcccttctggaacgaggagattgttccccagatcaaggccggcaagcgagtgctcattgcagcccacgggaacagcctgcggggcattgtcaagcacctggaagggatgtcagaccaggcgatcatggagctgaacctgcccacggggatccccattgtgtatgagctga

SEQ ID NO: 622 - DNA, 553 bp, Artificial Sequence
  Synthetic
ttcaacccattgattacgtccatcctagcaagctaaatggcatcccagctgctcctttctgtgcaaccaattaaagaacaatgagtgtgatgctccatgtctgaatttcgtccagcctctctctgaactgtgatctttgtcctcatgaactttcccttttgttcattgaactatatggactcttcatttcatattgatttactgtgcaatttacttttggacattgagaacttgaaattatttcctgatcccttccccttccactattaataattcatttctgtcaaactgtaagagtagactcattttttttttttttagtttttaacattggactgttatttcatttagagttctctatctctaaatatttatttagagaatgattttaaaagggaatgatatgcttgtttaaatgaaagagaaaagctgtagtaaactgtgttaattggtaatgactatttatcgtcgatactctgtagctgtgtaagttttgacaaatagtgtatctcgtggaatcagtggttagcattgccgctattatatttactca

SEQ ID NO: 623 - DNA, 402 bp, Artificial Sequence
  Synthetic
  misc_feature (92)..(92): n is a, c, g, or t
  misc_feature (94)..(99): n is a, c, g, or t
  misc_feature (155)..(155): n is a, c, g, or t
  misc_feature (180)..(180): n is a, c, g, or t
  misc_feature (211)..(211): n is a, c, g, or t
  misc_feature (345)..(345): n is a, c, g, or t
  misc_feature (371)..(371): n is a, c, g, or t
tgttgttgtccctattgctggtttattacactgtacagaccacaaaatgtaatattcttttgtataactactaaagaaaaatccttgtagancnnnnnnccttcaccatggctatctatacctgtacatgaaatgtgtttgtattgtgctgaagngcttaatgtcaacattacctgctgnttactctgaaaaaaggaatgaatggtagctntagaatttaggatattttatcaggttggcactttataaaatactccctgatttaaaaaattgtaagttatacacgttaatcatccacattctatcgacaatgtaccaacatcacaagctgttgcaaccacctgnctgttacttctctgagctgttaaaancctggaacttcaatttcaggggggcacaaat

SEQ ID NO: 624 - DNA, 487 bp, Artificial Sequence
  Synthetic
atatctccagccttcgttatgaacttttggaagacaagagccaagcaactgaggaattagccattctaattcataaacttagtgagaaactgaatcccagcatgctgagatgtgaatgatgcagcaacctggatttggctttgactatagcacaaatgtgggcaataatatttctaagtatgaaatacttgaaaaactatgatgtaaatttttagtattaactacctttatcatgtgaacctttaaaagttagctcttaatggttttattgttttatcacatgaaaatgcattttatttgtctgctttgacattacagtggcataccattgtgttgaaaagcccaatattactatattattgaaacttttattcattttagagtaaactccacatctttgcactacctgtttgcctccaagagactatcagttccttggggacagggaccatgtcttattcatctttgtgtctccagcatctagtac

SEQ ID NO: 625 - DNA, 528 bp, Artificial Sequence
  Synthetic
gaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttt

SEQ ID NO: 626 - DNA, 449 bp, Artificial Sequence
  Synthetic
actaaactctgaataagcactttttaaaaaactttctactcattttaatgattgtttaaaggtttctattttctctgatacttttttgaaatcagtaaacactgtgtattgttgtaaaatgtaaaggtcacttttcacatccttgactttttagatgtgctgctttgatatataggacattgatttgatttttattattaatgctttggttctgggttgtttcctaaaatatctgggtggcttaaaaaaaactctttaacttgtaataaacccttaactggcataggaaatggtatccagaatggaattttgctacatggggtctgggtgggggcaaagagacccagtcaattacatgtttggtaccaagaaaggaacctgtcagggcagtacaatgtgactttgaaaatatataccgtgggggtagttttaccctatatctataaaca

SEQ ID NO: 627 - DNA, 324 bp, Artificial Sequence
  Synthetic
gctcagggggcgttagggaagggatggtcgctcctgcggcctcctagatacctcgggagcgcaggccgcggccggcgggcctcagctcctgtggggagcgcctctagaatgtaatgggacgccccacccatttgccaggctggatccccactcgaacagggggccatgcagagactctgggctgcgcagcccccggcgccacggccaccccccggcctcagcgaggagcggtcggccatggccacccggggcagctgccctcagccaagcccagcgcaacaaaggaaaactacgaaccggctgtccaaggctgagcggtgactg

SEQ ID NO: 628 - DNA, 481 bp, Artificial Sequence
  Synthetic
gacgaatcaagcatccttgcggcacaccgcgcaacgtacagagattacgatttaatgaaggcaaatgagcccggcacttcctacatgagtgtgcaagataatggggaagattcagtgcgcgatgtcgaaggagaagattcagtgcgtggtgtcgaaggaaaggaagccaccgatgcttccgaccacagctgtgaggtggacgagcaagagagcagtgaggcctacatgagaaaaatcctcctccacaaacaggagaaaaagcggcagttcgaaatgagaagaaggcttcactacaacgaagaattgaacatcaaattagctagacaattaatgtggaaagagctacaaagtgaagataatgaaaacgaagaaacgccacaaggcacgaacgaagagaagactgctgcggaagaatcagaggaagctcctctgaccggtggactgcaaacccagtcatgcgacccttagaagatgcctgctt

SEQ ID NO: 629 - DNA, 544 bp, Artificial Sequence
  Synthetic
gagcccgaggcgtcctcgggctcggggccgccttccgggggcgcactggggcctcgggcgctgttcgccagccgcgtgcgcccgggccagcgcgtgtacgtggtggccgagcgtgacggggaccgccggctcctgcccgccgctgtgcacagcgtgaccctaagcgaggaggccgcgggcgcctacgcgccgctcacggcccagggcaccattctcatcaaccgggtgctggcctcgtgctacgcggtcatcgaggagcacagctgggcgcaccgggccttcgcgcccttccgcctggcgcacgcgctcctggctgcactggcgcccgcgcgcacggaccgcggcggggacagcggcggcggggaccgcgggggcggcggcggcagagtagccctaaccgctccaggtgctgccgacgctccgggtgcgggggccaccgcgggcatccactggtactcgcagctgctctaccaaataggcacctggctcctggacagcgaggccctgcacccgctgggcatggcggtcaagtccagctgaagcc

SEQ ID NO: 630 - DNA, 560 bp, Artificial Sequence
  Synthetic
ctgtgcagcagctgaccgacagcactcaaattaaaatggacattttggcgcaagttttacagattttattaaagtcgaagctattggtcttggaagatgaaaatgcaaatgttgatgaggtggaattgaagccagataccttaataaaattatatcttggttataaaaataagaaattaagggttaacatcaatgtgccaatgaaaaccgaacagaagcaggaacaagaaaccacacacaaaaacatcgaggaagaccgcaaactactgattcaggcggccatcgtgagaatcatgaagatgaggaaggttctgaaacaccagcagttacttggcgaggtcctcactcagctgtcctccaggttcaaacctcgagtccctgtgatcaagaaatgcattgacattctaattgagaaagaatatttggagcgagtggatggtgaaaaggacacctacagttacttggcttaacccttctggaagggtctgactgtgtgacccgcagcaaatagttcatgttggaaagaatgaaaacaacttcaagttcataggcagccagcc

SEQ ID NO: 631 - DNA, 432 bp, Artificial Sequence
  Synthetic
ctgcccttgtcactaatcaccaaggagtcattaacttcctccctctatgcccacccctgacacttcaccatttatcttaacacagaacatgaggttatgtgtgcaaattgagtcttttaggacagattctcagatgggatgatctggaggaggaggaaaaagatacacgcccttggggctgagcatgccccacactcttggatcttctctctgttccctggtgtcttcactaatgtcctcatgttggtgaagtgttgggggatggagggcgggtgagcagtcgggggacaaaaagtctatttttccttcacttttgtctcttcaatgtgtgttttcctcacactccttacttgactgctttcttcccccactttcatgtcccctctggattgtccagtgtaatgcatggcattgtggtgtctgtctaggaag

SEQ ID NO: 632 - DNA, 496 bp, Artificial Sequence
  Synthetic
ataggggtgtatgagctccagaggctgctcaacaggatggccatcaaattcaaaagcttcaagaccaagggctttggcctggatgcttgccgctgcatgatcaacctcatggataaagatggctctggcaagctggggcttctagagttcaagatcctgtggaaaaaactcaagaaatggatggacatcttcagagagtgtgaccaggaccattcaggcaccttgaactcctatgagatgcgcctggttattgagaaagcaggcatcaagctgaacaacaaggtaatgcaggtcctggtggccaggtatgcagatgatgacctgatcatagactttgacagcttcatcagctgtttcctgaggctaaagaccatgttcacattctttctaaccatggaccccaagaatactggccatatttgcttgagcctggaacagtggctgcagatgaccatgtggggatagaggcgctgtaggagcctggtcatctctacca

SEQ ID NO: 633 - DNA, 518 bp, Artificial Sequence
  Synthetic
gccccaactctttatcaagcaacattcttgttaactatatgtgaaacattttttcttctgaagattcttaaaaattgaatgtggctgaagttgaacatgggagcttattgctaatttagagataggaaactgaagcataaagaattaatgacttactttaattactggaattcttctgcaacatttgacaaaactaaccttgaataaggcccactgtaatacgtagctctcttaaatataacacttaggactagaagattagaaactaccaatcccaactacgtaataggaaaatgtaggatcaaaaggcccatgtatataagtactgaccactgggccataatgttgcttctcaggctatatgcagtcctttagtcagaagtcaataggcctatttattaatattttacagaccatattacctggattaccagggactatctttgctgcagagatcaagggttaagatctatgggaagatacttatttttctgaggtccttatgtcctgtcatataa

SEQ ID NO: 634 - DNA, 296 bp, Artificial Sequence
  Synthetic
tccctgtctaaaacacatcagtgtaaattcaggcttcagcacgccgtccagatccccaggataagagaccacttagccttccctttggtctgttctgtgactctctccccaaacacaaagaagcgtgtatgtagtcaccattccagaacctctcttttctaagatgcacaacacttatcaaagatatgtttagtttagactttatacccttagctttcccataagagctccctttgtggggaacttgccctcttctgcgaaataagcctcatccttaaagagaagtcaccggccgg

SEQ ID NO: 635 - DNA, 413 bp, Artificial Sequence
  Synthetic
  misc_feature (37)..(37): n is a, c, g, or t
  misc_feature (123)..(123): n is a, c, g, or t
ttccatggcactactctcattttgaagtttatgaacngtacgacagtgtattgtaaattccctttagtttgttacagtagccaattgaaaatgtatatcccaaaatttaaaaaaaaaagtacnagcagcaataaaattgcactagttttatctggtacatacaaaaacagccatttaaaatctgatgattctgtggaattggtgtccctgttagcaatggcagagaccagcctggagcctagatctggtgcttcttctgtgctgtggtttaccccaaacctttaggttgtttattcattcagattagatagactggagccataaagttaatttgcacctagctttttggagaatagccatgattaactgctattcgtggtgggggtgggggggaaccctatgatttactatgc

SEQ ID NO: 636 - DNA, 527 bp, Artificial Sequence
  Synthetic
  misc_feature (80)..(80): n is a, c, g, or t
  misc_feature (85)..(85): n is a, c, g, or t
  misc_feature (88)..(88): n is a, c, g, or t
tgttatctcttacctgccagaatgttctcaaaatgattagttccaaatactgaaactacttaatgaggcaattacttganttttnaantgatgacttcgaaggagtcatcactaggtgctttgtcctttttgtattctagttgcacccacctcttggattggatatagcaataacatttattggccgttgtgagctcttgatcccagtcattacccctgagaactaaaaatagatggttcttaattcaacttactgaaaatttccccaaacaatagcaaatctgacttttccctcttcagttgcctggtattaaggttggataaatgaagcatgcacagctacaggctttctacttaacttctgggtttgctattacaaatcctatttactctcatacccttctccttagtccttcatatttctctgcctctattcttctatactgcagatttttctcacctattgtacaaagaaattgcgatgtatattttcatgtaatttgattttggaattctgtcaccttatg

SEQ ID NO: 637 - DNA, 516 bp, Artificial Sequence
  Synthetic
cccaacttcgatggcctggagagtaatccatacagaagccggaagcagcgccaggagtgggaggtgaaggccctgctagagaaggtacctgcagagcttatttgtctggacccacgagccctggccgaggtggatgtcatctccctggagcagggaaagaaggagcagatagagaggctgggctatgacccgcaggctaaggctcccttccagccaaagccaaagcagaagggccgcagctccacggcaagcctggtgaagaggaagaggaaggtcatggatgaggaacacagggacaaggtccggcagagccttcagcagcagcatcataaggaggcgaaggccaagcccacgggggcccggccatctgccctggacagatttgtgcgctgagccagactccagggttgcctgggaacagtctctccccaagatcacctgtagggaaatgagtgttccctggaacaaggaggtgggggcagtgtggccccttccccaactgggggtggacagctg

SEQ ID NO: 638 - DNA, 424 bp, Artificial Sequence
  Synthetic
actcttgcccaaaactatacactgtatttctgttatagtaacaaaatagagttatgtagaattgtatggaactcaaatttaaaccatgctttcttgtagtactgattgaaacttacacgttttattctactcatagtgagcttattcttattttggattgattttccaaaaccacagcttcagcagcaacaatcagaatgtccaaatgctgtcctttcccttacagagaagaacaatggtaactaaaagctgcatataactagcaataactacattgaacggtgtgcattgttcatgattgttgtgttttaagacttgtatataaactgctttttccaaactctgtataacttttaaatggctggaactactcgtataaggactagactgtatttttgacatgctcctatttttgtaacttgaa

SEQ ID NO: 639 - DNA, 492 bp, Artificial Sequence
  Synthetic
ttcaggtgttactcagctgcatagttacgcagatgtaatgcacattattggcgtatctttaagttggattcaaatggccatttttctccaattttggtaaattggatatcttttttttacaaatacgaccattaacctcagttaaatttttgtttgttttcctgtttgatgctgtctatttgcattgagtgtaagtcatttgaactaatggtataactcctaaagcttctctgctccagttatttttattaaatatttttcacttggcttatttttaaaactgggaacataaagtgcctgtatcttgtaaaacttcatttgtttcttttggttcagagaagttcatttatgttcaaagacgtttattcatgttcaacaggaaagacaaagtgtacgtgaatgctcgctgtctgatagggttccagctccatatatatagaaagatcgggggtgggatgggatggagtgagccccatccagttagttggacta

SEQ ID NO: 640 - DNA, 496 bp, Artificial Sequence
  Synthetic
gaatcagcagtacacatgcccatcaccttcgctgtctgaatgactttgtagaagcccagatgacttactatgcacagtgttaccagtatatgttggacctccagaaacaactgggaagttttccatccaattatcttagtaacaacaatcagacttctgtgacacctgtaccatcagttttaccaaatgcgattggttcttctgccatggcttcaacaagtggcctagtaatcacctctccttccaacctcagtgaccttaaggagtgtagtggcagcagaaaggccagggttctctatgattatgatgcagcaaacagtactgaattatcacttctggcagatgaggtgatcactgtgttcagtgttgttggaatggattcagactggctaatgggggaaaggggaaaccagaagggcaaggtgccaattacctacttagaactgctcaattaagtaggtggactatggaaaggttgcccatcatgactttgtat

SEQ ID NO: 641 - DNA, 399 bp, Artificial Sequence; identical to SEQ ID NO: 224
  Synthetic

SEQ ID NO: 642 - DNA, 124 bp, Artificial Sequence
  Synthetic
gctgggacagaacttcttatagaaaagacagatccaaatatggccacagatgaacttccatgcaagctatggctgagtttcattgcttaaaatgctcctctgtttagaggggcctggtagaaat

SEQ ID NO: 643 - DNA, 499 bp, Artificial Sequence
  Synthetic
cagaataccaagaacgctgccggcaggaggtgcaagagcttctgaaggaccgtgagcctaaagagattgaatgggacgacctggcccagttgcccttcctgaccatgtgcctgaaggagagcctgcggttgcatcccccaatccctacattcgcccgcggctgcacccaggacgtggtgctcccagacagccgagtcatccccaaagggaatgtctgtaacatcaacatcttcgcaatccatcacaacccctcagtctggccagaccctgaggtctatgaccccttccgcttcgaccccgaaaacgcccagaagaggtcacctatggcttttattcctttctcggcggggcccaggaactgcatcgggcagaagttcgcgatggcagagatgaaggtggtcctggcgctcacgctgctgcgcttccgcatcctgcccgaccacagggagccacgcaggacgccggagattgttttgcgtgcggaggacggactttggct

SEQ ID NO: 644 - DNA, 475 bp, Artificial Sequence
  Synthetic
cgctccctgggccgtaggaaatgagcgataacgatgacatcgaggtggagagcgacgaagagcaaccgaggtttcaatctgcggctgacaaacgggctcatcataatgcactggaacgaaaacgtagggaccacatcaaagacagctttcacagtttgcgggactcagtcccatcactccaaggagagaaggcatcccgggcccaaatcctagacaaagccacagagtatatccagtatatgcgaaggaaaaaccacacacaccagcaagatattgacgacctcaagcggcagaatgctcttctggagcagcaagtccgtgcactggagaaggcgaggtcaagtgcccaactgcagaccaactacccctcctcagacaacagcctctacaccaacgccaagggcagcaccatctctgccttcgatgggggctcagactccagctcagagtctgagcctgaagagccccaaagcag

SEQ ID NO: 645 - DNA, 483 bp, Artificial Sequence
  Synthetic
gaagacactgaacgagcttcccttgttttgcctggaagcccacgctggctccctggctctgcccaggatgtgcagtccaaatcccaatccagcagtggggttatgtcgtcccgcttaccctcagagcccttctcctggtgctgcccagacgatcagccagtccctcctggagaggttctgcatggcctctaggagagaagttttcttggccccaggaaggcctggtggagggtggtggttgtgcactgttgctggacagatgcattcattcatgtgcacacacacacacacacatgcacacacaggggagcagatacctgcagagaagagccaaccaggtcctgattagtggcaagctgccccacaaagggctatgcctgtgtcttattgagacaccttggcaaagagatggctgattctgggtggtcctggacatggccgcacccaagggccctccaagccttaatggcaccctgaagcctc

SEQ ID NO: 646 - DNA, 357 bp, Artificial Sequence
  Synthetic
ctccacagcagaaatcgctgatggaccagctccagggagtggtcagcaacttcagtaccgcgatcccggattttcacgcggtgctggcaggccccgggggtcccgggaacgggctgcggtccctgtacccgcccccgccacctccgcagcacctgcagatgctgccgctgcagctgagcacctttggggaggagctggtctccccgcccgcggacgacgacgacgacagcgagaggtttaagctcctccaggagtacgtgtatgagcacgagcgggaagggaacaccgaagaagacgaactggaagaggaggaggaggacctgcaggcggccagcaaactgaccccggatgattcgc

SEQ ID NO: 647 - DNA, 448 bp, Artificial Sequence
  Synthetic
acaggactatttgccacgacatttcaaaggattccaagagagaatattggtgtccatgctgtgatgattcctcagctcctctcatctgatctccgtcctggcccccatgactttctttgcggtagttagggtgtggtatgtgccactgaggcccacacctattggcaatttatagcactgatctgtcatcaataccacttgctgtcttggatgtgaagatgatttttcctgcagggattccctctacaaaattaaaaacactgggcatgtggaaataatattcacgctttaaattgtcttttctattcactacaccaggggtccccgacccctaggcaacagactgtggccctagtgtagtgaatagaaaagacaatttaaagcatgaatattatttcctcatgcccagtgtttttaattttggtactggtctgtggcttgttagaaa

SEQ ID NO: 648 - DNA, 551 bp, Artificial Sequence
  Synthetic
gaagaaggcggcgttgtccatgtgcaagtctgtggtggcccctgccacggatggtggcctcaacctgacctccaccttcctcaggaaaaaccagtgtgagacccgaaccatgctgctgcagcccgcggggtccctcggctcctacagctaccggagtccccactggggcagcacctactccgtgtcagtggtggagaccgactacgaccagtacgcgctgctgtacagccagggcagcaagggccctggcgaggacttccgcatggccaccctctacagccgaacccagacccccagggctgagttaaaggagaaattcaccgccttctgcaaggcccagggcttcacagaggataccattgtcttcctgccccaaaccgataagtgcatgacggaacaataggactccccagggctgaagctgggatcccggccagccaggtgacccccacgctctggatgtctctgctctgttccttccccgagcccctgccccggctccccgccaaagcacccctgcccactcgggcttcatcctgcacaataaactc

SEQ ID NO: 649 - DNA, 579 bp, Artificial Sequence
  Synthetic
gacctcgggcttggtgaaacccactacctcccctctcatccccaccaccaccccagccaggcccctcctgcccttctccacggggcccggcagcccgctcttccagccactgagtttccagtcccagggcagccccccgaaggggcctgagctctccctggccagcatccacgtgcccctggaatcgatcaagcctagcagtgcccttcctgtgacagcctacgataaaaacggcttccgcatcctcttccactttgccaaggagtgtcccccaggacgacctgacgtgctggtggtggtggtgtccatgctgaacacggctcccttacctgtcaagagcatcgtgctgcaggctgcagtgcccaagtcaatgaaagtgaagctgcagccaccttctgggacagaactctctccatttagccccatccagccacctgcagccatcacccaggtcatgttgctggccaatccactgaaggagaaggtgcggcttcggtataagctgaccttcgccctgggggagcagctgagcacagaggtgggcgaggtggaccagttccctcctgtggaacagtgggg

SEQ ID NO: 650 - DNA, 302 bp, Artificial Sequence
  Synthetic
cagatcctcttaccacaatattactgatgtgtgtgagctggctgtgggccctgcaggtgcaccggccactctgttgaatgaagcaggtaaagatgctttaaaatcctctcaaaccattaagtctagagaagagggaaaggcaacccagcagagggaggtagaaagcttccattctgagactgaccaggatacgccctggctgctgccccaggagaaaaccccctttggctccgctaaacccttggattatgtggagattcacaaggtcaacaaagatggtgcattatcattgctaccaaaac

SEQ ID NO: 651 - DNA, 455 bp, Artificial Sequence
  Synthetic
aagcctttaggcagaatgtacaccttgttagtcatttgagaattcatactggtgaaaaaccctatgaatgtaaagaatgtggaaaagcttttagaatcagttcacagctggctactcatcagagaattcatactggagagaagccttatgaatgtattgaatgtggaaatgctttcaaacagagatcacaccttgcccaacatcagaaaactcatacaggagagaaaccttatgagtgtaatgaatgcgggaaagccttcagccaaacttccaatcttactcaacatcaaagaattcatactggagagaaaccctataaatgtactgaatgtggaaaggcttttagtgatagctcatcctgtgctcagcatcaaagactccacactggccaaaggccctatcagtgttttgaatgtgggaaggcgttcagaagaaagttatccttaatttgtcat

SEQ ID NO: 652 - DNA, 513 bp, Artificial Sequence
  Synthetic
  misc_feature (62)..(62): n is a, c, g, or t
  misc_feature (153)..(153): n is a, c, g, or t
ctaggctgactggatttgagaacaaatacccacccatttccaccatggtatggtaacttctntgagcttcagtttccaagtgaatttccatgtaataggacattcccattaaatacaagctgtttttactttttcgcctcccagggcctgtgngatctggtcccccagcctctcttgggctttcttacactaactctgtacctaccatctcctgcctcccttaggcaggcacctccaaccaccacacactccctgctgttttccctgcctggaactttccctcctgccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtggaaactaagccactaattgagtttctggctcccctcctggggttgtaagttttgttcattcatgagggccgac

SEQ ID NO: 653 - DNA, 460 bp, Artificial Sequence
  Synthetic
gccatcttcctgaatgttgacatgccagtgggtgtgactccttcatttttccttctcccttccctttggacagtgttacagtgaacacttagcatcctgtttttggttggtagttaagcaaactgacattacggaaagtgccttagacactacagtactaagacaatgttgaatatatcattcgcctctataacaatttaatgtattcagttttgactgtgcttcatatcatgtacctctctagtcaaagtggtattacagacattcagtgacaatgaatcagtgttaattctaaatccttgatcctctgcaatgtgcttgaaaacacaaaccttttgggttaaaagctttaacatctattaggaagaatttgtcctgtgggtttggaatcttggattttccccctttatgaactgtactggctgttgaccaccagacacctgaccgcaaatatcttttc

SEQ ID NO: 654 - DNA, 507 bp, Artificial Sequence
  Synthetic
tttccttatctgaggctacctggggattgtgggcagcaggcccctggactcccagaaatgctgagggtccctcttccaggggagttccctggggagcaagagtagaggggctattcccgagggtcctgtggtcagggtcctgctttgtccctggcaggctgtcagtccagctggagggggtcagtaggctccagggagtgggccctcccctccgatcttggagtctctgggtgttccttcagcctcagcctcactggtaccttctgccctttaggggaggctatgagtctgggacaggaggacaaattcagagcccagagggggaaatggagaaacctgggctgctgaggaaagccacgggtgttggcgtgaactgatgatgtctcatcccacgcccactatggccacattacctaacctctccatatctgggctattcaaggtcttggaggccagcacagacccagctagttgttgaagccagttcttttttgtcttggtcatgag

SEQ ID NO: 655 - DNA, 292 bp, Artificial Sequence
  Synthetic
ttagattactcatctgtccaccaagtgggacatttgcatggactgggggcttaaaggacttagaagagacctgtaagtaaatcctgaaaatgagccaatccccacttgaatggttactggagtaaacccacctttaccaccccaattacagcacccgaggccgataaaccaacttggctctggttcatttttcttttcttcatttgtgatgctcagattcaaaatgtgtgttctacactgttacaggcttctcttttgtttgattaaagattttagtcctacttttgtatgg

SEQ ID NO: 656 - DNA, 482 bp, Artificial Sequence
  Synthetic
agggtgtcactttttttgcactgcagcatatacactaactagcttattaaaatttacaaaatgtctttttgaatgtatcaaggatatatttagtttgagtggaatttgtcagcagatatcagtaacttattgccgcttatattgtacaatgttaaacttcaattcctgtaacctggttagtattaatgtcagtgactaaaaaacttagagttagttttagggcactttttattttgagagcatgaagtgtggaatgtgtcactacgattgttgataaagctgaggccacttgcaacttgattttttaaatgaaatagataaagtctttttgaataatatagtatgcactgctatttgcttgattatgtaatgtcaaaagtttaactatattccaagtacaaaaacatactggattacattgaggatgttgaatagcattcatgatggctttgttttggtttggggcagctgtcaccagctaa

SEQ ID NO: 657 - DNA, 484 bp, Artificial Sequence
  Synthetic
  misc_feature (395)..(395): n is a, c, g, or t
gtttcagattcttttgccattgcaaagtaccccttataaaccagcaatgtcatctgtgaggaagcaaattctcaagtgtctgtcatttacttggttctttttctttgtggtcttcacccttataccctggaaaagtctgtaattaccttagccaggaagatagatggtcatggcaagcgcacagcaccagacttactggctcaccaagatgatggaaaaaggcagatgattttttaaaaagccgtaatgactcctttagaccagccatttagcgtggtaattttgaaaggcctagctccattgcagacttccaaagggtcagctctgagactgccctccaggtgggcagttgattatttccaccagtgttttccagagccttaaactgtcctaangtgacaactacctcagttggcaggaaagagacatatagtagaaagtgaaaaatgagcagtatttgggcagatgctatgggttacagttg

SEQ ID NO: 658 - DNA, 247 bp, Artificial Sequence
  Synthetic
gcaaggctgttgtctgtatggaaggacacgctcgcggcaagggcagggcctggggagggtggcctgtccagtcctgcagacaaggggaggcctgacagagcccaagaatgaggacaccctcggcacgggaacccattcacttagcgtttgctccagtagctttccctctgctaccaatgcagataaacgcggcttgttttactcaggcaagagaatgtgaatagtgccaagaaaatcctttacatta

SEQ ID NO: 659 - DNA, 541 bp, Artificial Sequence
  Synthetic
gtcagttcttatcaaaaagctcggtactgcactacaggatgaaaaggaaaagaaaggcaaagacaaagaagaacactaaaaaagtaatttgatctgtgaacaaattatgattgtgtctgttttattacactggagtgtttttttagtataataatttgaaatataactttaaaataattctaaatttgtggctataattaaaagtttgtaagttaacctgttctagttccatcattctgtgtacagtgaagtattgcatgataatgtaaattttgtgaaaaactagattaaaatatataactgcttgttatggtttataattatataatgtgcaatacaattcctgcatctttaaaatgtctgcagaataactgtgaatttttttgttattggattggccgtaacttttagaaaaaaatcttgttgatgataatgtgattttggggaggtcattaattgctttttcttttttaaatgtagacttatataaatacctgtttgtatatagcttgagtaattgtgatatgattgtataccacta

SEQ ID NO: 660 - DNA, 502 bp, Artificial Sequence
  Synthetic
  misc_feature (55)..(55): n is a, c, g, or t
  misc_feature (105)..(130): n is a, c, g, or t
  misc_feature (465)..(465): n is a, c, g, or t
gattcatgagaagttctccccaagtccactgaacaaggctagttcctgcaccacncaggattcaaaggaaagacgaagggagcagaacttgtggcagcaacaggnnnnnnnnnnnnnnnnnnnnnnnnnnagttgtcaaggcagcagaaaggagtcacccagccaaaggaggagatggctcagcggggctgcaccaaggggccaagaggccctacccgtgtctaaaccctcctctcactcccctaagcctggtgaaagagtcagaagccccaggctcctttttctgtttcttaactcaacagctaaaaaatggctccaggtagtgagtcaatgaagttcagacatgttggtgtaaagtttctcctctgctcctgaaaacttcatcttcttggtgtctcatgtcctcattctcccctatatgacatgcaaaaacgatctttctttgaaatccctctgggaagaagncatgtttattgaaactgtccttcagccttaaatacaa

SEQ ID NO: 661 - DNA, 571 bp, Artificial Sequence
  Synthetic
  misc_feature (467)..(467): n is a, c, g, or t
tatcccgacctggaagctttgatgggtcatgggccatgccataccccctgtggcaatggagtgtgtggatgctcacctgtgccatctgtcctcctgtctgtgccaggaggcacctgagttctctgctgttatcctgccccaagggcctgggccgagcctctacctgaagcaactctgctcttcctgtcagtctcaaagcacaaggaggttcagcccaggaggaagccagctgcaatgtggagacacgtcctcctccccaacccacctcatgccaccgccaaccccctgccccaggagcgggcctgagccacgtcccctaggagcagctggagatggccaaaagagtgagctcaggactactggatcccatgcccaggtgtccagcagacctcaaggcagaagggtcacctaacccaggagtccacagactgatgtgacctcaggttcccacatcagtggccacaggncagggcccacctggtagaagtgttctggatatggccagggtgggtgtgtggctaagtgggcctgaacagagggaacctagggcccttggccaatgtgattaaag

SEQ ID NO: 662 - DNA, 452 bp, Artificial Sequence
  Synthetic
  misc_feature (59)..(59): n is a, c, g, or t
  misc_feature (81)..(81): n is a, c, g, or t
  misc_feature (99)..(99): n is a, c, g, or t
  misc_feature (109)..(111): n is a, c, g, or t
  misc_feature (113)..(114): n is a, c, g, or t
ctaggcttgctttatcatcacatcccaaggccagagggctgctgctgtccccatcttgngccctgctagaagagggatagngggtggctggcatgatgnggtgaggggnnnannagaatatgcagaggccttggaggaagaggactggcagttatgacaggaaggctctctatacctggctccccagtgttctgcccctggcactgagcatgaggagccaggctttggggagactttgcaatcacccccccaacctggtccattttccacaggtagctttcttgaactcaccttgacccctcctcagccagcagcccccacctccaggggcaaaggagctgaaagacagtcctgaactggggggagctgggatcacatcagccaggccctgtccctcacaggaagtgagatgaggtgataccatggatggtgactaaggccccaaagtccct

SEQ ID NO: 663 - DNA, 470 bp, Artificial Sequence
  Synthetic
  misc_feature (421)..(421): n is a, c, g, or t
tgcgtcttcccaagttagcaggtgtctctggtgttcagtgaggaggacatgtgagtcctggcagaagccatggcccatgtctgcacatccagggaggaggacagaaggcccagctcacatgtgagtcctggcagaagccatggcccatgtctgcacatccagggaggaggacagaaggcccagctcacatgtgagtcctggcagaagccatggcccatgtctgcacatccagggaggaggacagaaggcccagctcacatgtgagtcctggcagaagccatggcccatgtctgcacatccagggaggaggacagaaggcccagctcagtggcccccgccccccaccccccacgcccgaacagcaggggcagagcagccctccttcgaagtgtgtccaagtccgcatttgagccttgttctngggcccagcccaacacctggcttgggctcactgtcctgagttgcagtaa

SEQ ID NO: 664 - DNA, 288 bp, Artificial Sequence
  Synthetic
taaaccagtagaaaggctcctatttacatccaggttgtcagtttaagctcaagtaatatgcaaggcacaattcttcctttctccagctttagaaaaatcaaccacatggcccagccctgaaggactgaagggagcttgtgcgggatcccgtcatcctggactctccacacagcgtccacgcatggatggccatgggcatgagtctctggatctccctgagggtagcccagcctcggcctggagcacaggggcctgtgatctgagaatctcagatccagaatcaggagc

SEQ ID NO: 665 - DNA, 530 bp, Artificial Sequence
  Synthetic
  misc_feature (67)..(68): n is a, c, g, or t
  misc_feature (70)..(70): n is a, c, g, or t
  misc_feature (76)..(76): n is a, c, g, or t
  misc_feature (82)..(82): n is a, c, g, or t
  misc_feature (126)..(126): n is a, c, g, or t
  misc_feature (248)..(248): n is a, c, g, or t
tttatatctatttccaactgcacatagttgagctgtctattattcaatttttccaaagcctgaaagnntnctcgangaaatngggaaatgctactccttgtagtgtttgatgcttagtgtccacancccacgtcagtctgtacctcattgattttaatctgtcgtagttcttcctcagctgcagtcagtggggcaggggaagattgtgacagcaagtattttttatatccatgtaatgatacatcttncctttactgttccaaatacttcatctttaatgtggccacctccaaattccttcttcagagttgctcttgttgctgcatacaacattttttgacgaacatgagaatgatctggagaccatgcaatgaatatccattcatatccctgggcattctgagaatctaacctgaataatatatagcatggttgtttgtcctccaacaggggtaaaacaaaggaatcataatccttatcccaggaatctgaaggctgactatatgatccaatcacaagttgctcatttt

SEQ ID NO: 666 - DNA, 365 bp, Artificial Sequence
  Synthetic
aattccaagtgaagcagcacagtgctgagaaacagttcatccatggtgccatgttaaagaagttggaaatatatcttgaaaatcctatcttccttttaggcttgaatatgatgctgaacagtaagtttgttaaatcttggaacttaaaacaatcctgctttctcaagtactattctaacattgcgctttataagggatgatatttctaccacctcactcatatttttagctgaaatgattttcctggtatgtctgttattttgtggaaaaagaaatattgtgtaaaatgggtgctgccaaaattccaggccattttgcagggactctgaagtgacctttagtagtaatagtcttatgtgcagtaa

SEQ ID NO: 667 - DNA, 273 bp, Artificial Sequence
  Synthetic
acttaggtaattgtagggcgaggattataaatgaaatttgcaaaatcacttagcagcaactgaagacaattatcaaccacgtggagaaaatcaaaccgagcagggctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaat

SEQ ID NO: 668 - DNA, 445 bp, Artificial Sequence
  Synthetic
  misc_feature (155)..(155): n is a, c, g, or t
  misc_feature (193)..(193): n is a, c, g, or t
  misc_feature (224)..(224): n is a, c, g, or t
  misc_feature (233)..(233): n is a, c, g, or t
  misc_feature (258)..(258): n is a, c, g, or t
  misc_feature (273)..(273): n is a, c, g, or t
tgcgatacgaaactcacgagatgtgatatgaagctacttctacatgaaaattgcaatctaaagctttcataccaaatgtcacaggagctgtctcccagctcatttttaacactgaaatgacattaggatctaaaataatttgctgtcaattgtanatttgcatgagtacgtatgtttggctcattactggttnaccccttgagtgaatgcctgtttatgatganctgagagcnatattcatgtgtgatctgcgtgttnctggaatatgctttnatacgtaatgaaatctgtttgctgggaattcctgattcttgttatataagaagaacaactatttcgctccagaaaaaaaagatcaaagagctttcagaaacttgggagaactgggtatttagaacaagtgataatgggtcagtttctcagactgtagcattgaaattagatg

SEQ ID NO: 669 - DNA, 510 bp, Artificial Sequence
  Synthetic
  misc_feature (140)..(140): n is a, c, g, or t
gaagcaactttgtatctcaggtcttaatgaacacatatgaattcacagtggagaagaccctgcatcagggaatgtggaaatgactttgctgaattctcaagccttaccaaacacatcagaaatctcactggagagaaacngtatgaatgtagagaatctgggaatacctttctgaatcccacaaaccttaatgtgtgtatgtgaactcacattggagagaaaccctgcaatttaaatggtatggtctggatgatgccccactccatatttgtaagccctaagtcctagttccttacactataactgtatttggacatagggttttcaaacaggtgagtaacttcaaatgaggttgttgggttcgatccctaatctgacatcactggtgtccctataagggaaactgaaggaaggatacacatggagaagactgtgtggatccaccagaagatggccatctacaagccaaggacagagacctggaacagatgctttcattatggcctccag

SEQ ID NO: 670 - DNA, 528 bp, Artificial Sequence
  Synthetic
  misc_feature (50)..(50): n is a, c, g, or t
gaagaagctctgccaacaataaaagattttgttaagtggtataaaaattngtgaatgttgaaattcaacatgcttcatattataaccaacctgcaagaatattgtggtcaattgtgtgaataccaaatcagtatatactgtatactgtactatgaaaatttgtatttaaaggcaaagatacaagtaattgttggtataattggggaaaagggtattcaatatttattaagtaacaatgagaaatgcaacaaacttccttatttatgccttatgaataaaagagtgtggaatgtaaggtgaaattttatgtgcaagatcctgaaggaatgggtattcttgaaggcagggactgtcttttctatgtttgttctgtacagctctcagcacactcttggtgcttgataaatgttatgttatataataccaataactaagcacatttttagatcttttttcaaatggcttgtgtaaaatttggttaatgtacaatggttggtttgaagctatcatgtaaaattggcctctcca

SEQ ID NO: 671 - DNA, 279 bp, Artificial Sequence; identical to SEQ ID NO: 119
  Synthetic
  misc_feature (40)..(40): n is a, c, g, or t

SEQ ID NO: 672 - DNA, 269 bp, Artificial Sequence
  Synthetic
cggcgagatccagcatcgcatgcagcgcaaggttctctaccgcctcaagtgagtcgcgccgcctcactggactcatggactcgcccacgcctcgcccctcctgccgctgcctgccattgacaatgttgctgggacctctgcctccccactgcagccctcgaacttcccaggcaccctcctttctgccccagaggaaggagctgtagtgtcctgctgcctctgggcgcccgcctctagcgcggttctgggaagtgtgcttttggcatccg

SEQ ID NO: 673 - DNA, 539 bp, Artificial Sequence
  Synthetic
ggcctgcagcatctgagtggggtgagcctcatgggagagacatcgctgggcagcaggccacgggagctccgggcgggcccctctcagcaggctgtgtgtgccagggctgtggggcagaggacgtggtgcccttccagtgccctgcctgtgacttccagcgctgccaagcctgctggcaacggcaccttcaggcctctaggatgtgcccagcctgccacaccgcctccaggaagcagagcgtcatgcaggtcttctggccagagccccacaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgtgcccagtgccccccaggcacctttgtgcagcggccgtgccgccgagacagccccacgacgtgtggcccgtgtccaccgcgccactacacgcagttctggaactacctggagcgctg

SEQ ID NO: 674 - DNA, 533 bp, Artificial Sequence
  Synthetic
cggtgtgctgttgagccaggactgtgctggcaccccacagggagccttggagccctgcgtccaggaggccactgcactgctcacttgtggcccagcgcatccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcaggagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgc

SEQ ID NO: 675 - DNA, 537 bp, Artificial Sequence
  Synthetic
tattgtaaagacgtcaattttcccaaaactgattgatagctataataaattcccaaactatcaagtgcatcattgttgttggaattgacaagctgattttaaaacttatatgggagaaaaaccttacatgaaaatacaaagagccagaatagtcaagacactctcaaagaagaaaaataaggtggaaaagacttactttatcagatctcaatacatgattgataataatgaagacagcttgatattggcacaagaacagacagactaatggaacagaggaaagaatccaacaacagatttacatatgtcatatgtggacatatgacagaggtggcattgcagatgggcgcagaaagttagattacaaacaatgataggacaggtgtgtaaccctatggagagaaaaaaatgaaagtgaatccttacctcacaccatatgcaaaaaaaccaaatctaggtgaattaaaggcctaaatatgaaatttacatttataaggatttcagaatataatctaagagactatcttcatgaccttg

SEQ ID NO: 676 - DNA, 303 bp, Artificial Sequence
  Synthetic
ggggcagagacagaatcaggacacagctcgaggtcagggccagaggctggagctgagggcctagagtgagagggggcaaggcaagggggggagaggaagagaggcaggattagagagaggaggcaggccagaaagaggagagcaggagagacccaaagagaaacagaaggcagatagagagggagtgagaggcaggagctgagacacagatcctggaggaagaagaccaaaggaagggggcagagacagaaagggaggtgctaggacaaaactcgaaaggtggccctatcagggaagcagagg

SEQ ID NO: 677 - DNA, 567 bp, Artificial Sequence
  Synthetic
atgggaaaaccatagagaacttcctggtggtgaacaaggtctgtgggctgaaatacgtggtttattgtgaggacgacgtttgtcacttacctgtcctgagtctgggcgccaaggaggcatgggccgtgaaggagctgtgcaagctgacagacaacctggagacgcgcattgatgagctggagcgctggagccacaagctggccaagctgcggcggctcgacagcctcaagtccaccggcagctcgggcgccttcagccatgcagggagccagttcagtcgggcgggcagcgtcccccacaagaagaggccccccaaggtggccagcaaggtagggtcatcgtccgtggttccggaccaggcctgcatcagccagcgcttcctgcagggaaccatcattgccctggtggtggtcatggccttcaggcatcacctgggagcccagcctcaggagcaaggaccacagccttgtcctagagagagggcactggatggtggctccagctctaacggtcacccttttctgtggctccagcgtggtgtccatgtccacactgtacgtgctgagc

SEQ ID NO: 678 - DNA, 219 bp, Artificial Sequence
  Synthetic
  misc_feature (74)..(74): n is a, c, g, or t
tgaagattcttcgttgtcaagccgccaaagtggagagtgcgattgcagaagggggtgcttctcgtttcaggtanttgtagatttattttggggaatccttttgaaaaggccagtctcaaaaagtagttaacacagcaaataagtccgacttaatgggctgcaattttcatttgaaaagttcttaagttttcattgacagccttctaagaactgccatct

SEQ ID NO: 679 - DNA, 371 bp, Artificial Sequence
  Synthetic
cttccctccatcctgtaggattttgtagatgctggtaggggctggggctaccttgtttttaacatgagacttaattactaactccaaggggagggttcccctgctccaacaccccgttcctgagttaaaagtctgtttatttacttccttgttggagaagggcaggagagtacctgggaatcattacgatccctagcagctcatcctgccctttgaataccctcactttccaggcctggctcagaatctaacctatttattgactgtcctgagggccttgaaaacaggccgaacctggagggcctggatttctttttgggctggaatgctgcccctgagggtggggctggctcttactcaggaaactgctg

SEQ ID NO: 680 - DNA, 355 bp, Artificial Sequence; identical to SEQ ID NO: 84
  Synthetic

SEQ ID NO: 681 - DNA, 484 bp, Artificial Sequence
  Synthetic
cccacctccagatcacaagggcaggctttctattttaaaagtctgtacaaaaaccatgccaatagggcctgatgtctccttagaaaacctcgcagcagaaacctgttttttttctggagctgatcttagaaacctctgcacagaagctgctttgctggctctgcaagaaaatggactagacgcaactacagtgaaacaagagcactttctaaaatcacttaagactgtaaaaccgtcgttaagttgcaaggacttggctttatatgaaaacttatttaagaaagaaggattttctaacgtggaaggtatttaaaaatcaccttaaactcttgttcagttcacattaattgaaatgtgaacttgcctgtcgtttgcaacttcacacttttagaatttgtgtttatatttcctgtaagtgaataaataaaacaaaacaaaacaaaaaaaacttgtgcctgataagctaaggctcatttatttttaa

SEQ ID NO: 682 - DNA, 448 bp, Artificial Sequence
  Synthetic
aactcttttttaagcgatgagatctgtgtggcatttctagcgctctctaaattatgtctctggcatattttaatcactggaaactcaaagagtggaagagtggaagtgcgaaggaatctcaggtagctcttaactaattcgccagcagtgaagaaacttcaaccaatcatgttttccttgaattttgcaaagaaagaaaatccaaagcattgcttggatgttcttttaaggcattttatatcagttgaaccttaaactgaaatatgaagacaagctttataaactagttttttgatatgaagtatatcaattatgtttgccttttgtgaagaagtaaatgagttgaatttgtatttcacttactagattatactaatgagctaatggggtcattgccatcctccctactcatgactttgatgagttattgctgtaacatcacttcttg

SEQ ID NO: 683 - DNA, 450 bp, Artificial Sequence
  Synthetic
caccagtgcagcctcaggcatgaaagtgcaaggggacccaggtggaaagacggggactgagggcaagagcagaggcagcagcagcagcagcaggtgcctgtgaacatacctcagacgcctctggatccaagaaccaggggcctgaggatctgtggacaagagctggtttctaaaatcttgtaactcactagctagcggcggcctgagaactttagggtgactgatgctacccccacagaggaggcaagagccccaggactaacagctgactgaccaaagcagccccttgtaagcagctctgagtcttttggaggacagggacggtttgtggctgagataagtgtttcctggcaaaacatatgtggagcacaaagggtcagtcctctggcagaacagatgccacggagtatcacaggcaggaaagggtggccttcttgggtagcaggagtc

SEQ ID NO: 684 - DNA, 403 bp, Artificial Sequence
  Synthetic
ccacccaggatttcctctgtgagaagtgggggagatggacaggtgactgcttccgccagccttggtgcctcaggggaggccgactgaggggggctctgtggatggcattcggggagctacaggtttcccccaaaagctcagatgctcgttcttgaagagggaggtgctgcccctgccttcctgcgtaccgcgacaatacagcttcctccgcggcgctttaacacgcagggcgctgctgccaggggcgtcccgtgttctaactcgctcccacagcccctccggcttggtgagcagcgtctgaggggtgaggggcatagacttggagccagcctgctgcactagatgccagctgtgtcacttagcagcaaggtgacctcagcaaagttgttttacctctgtgcct

SEQ ID NO: 685 - DNA, 468 bp, Artificial Sequence
  Synthetic
ttgatgggattcgcacctgtgatgagtacgattccatacttgcggagcatcccttgaagctggtggtaactcgagcgttgatgattactgcagatattctagctgggtttggatttctcaccctgctccttggtcttgactgcgtgaaattcctccctgatgagccgtacattaaagtccgcatctgctttgttgctggagccacgttactaatagcaggtaccccaggaatcattggctctgtgtggtatgctgttgatgtgtatgtggaacgttctactttggttttgcacaatatatttcttggtatccaatataaatttggttggtcctgttggctcggaatggctgggtctctgggttgctttttggctggagctgttctcacctgctgcttatatctttttaaagatgttggacctgagagaaactatccttattccttgaggaaagcctattcagccgcgg

SEQ ID NO: 686 - DNA, 420 bp, Artificial Sequence
  Synthetic
agttttcatcgtctgctcaataccgtgggttcttcttcgtcctctgtcctctgcccagtgtgaggccatcaccatgtgagaagacatcttggcctgatttgctgccaccagcgtcccctccctcagtgggcccgaactcgccagccccagctttcagtggagaaagcggtcctctgaaatggtttcctcccaacccccgcatttaaagggactcaaggtgcctgccacttcctcagcgaagaagtctgtgttcctccccgtccttgccagtggcgatcatcccttcacaatcccagagtggcaggcgggaccagccccatggtctggctcctgtcacctgggtccgtgccagcacaatctgccaaagttctagagaccctgttcccttccccatcacctcacatgcttcttctgtgtgta

SEQ ID NO: 687 - DNA, 504 bp, Artificial Sequence
  Synthetic
gcaactgatagtactccggtggatatgcaaacctatgagctctgctgtgatatgatagatgtggttattgacatctcttgtatttatggtctcaaagaagatggagcaggaaccccctatgacaaggaatccacagccatcataaagcttaataatacaaccgtgctttatttaaaagaggtgacaaagttcctggctctcgtttgctttgtcagagaggaaagctttgaaagaaaagggctaattgactataattttcattgcttccggaaggccattcatgaagtttttgaggtgagaatgaaagtagtaaaatctcgaaaggttcagaatcggctgcagaagaaaaagagagccacccctaatgggacccctagagtgctgctgtaggtgaggtttcaggaatgtcttttgaaatcagaccttatccatgaggctgctgcgccatgttgcactaaaggaagaggaagaaggagattgggacacataccattgatttgttgt

SEQ ID NO: 688 - DNA, 541 bp, Artificial Sequence
  Synthetic
  misc_feature (40)..(40): n is a, c, g, or t
gggacatctggggactgtttttgtgtttaggggaaaaaanttctgctgcactctgcttgggccttgaggtctgtggcagggctcctctggcccgcagtggcctggatctatctgggccatgagtgacgggcagtgaccagagggactggaggccagcggtgtccacccttgccctcagcaagagagaatgcattcttaaaagaaagctgtacatgtatatatatgcatatatatatatgtggctctagcctcaggctccagccccagtggggtactgtacagttaactgaagaagaattttaaagacgatttgaacaagaaaatgaaggcagtgggaaagcaatgccaaatggttgtggagaaagtggccggagcctccctggagtggagcagccctgaagcctgtgccccccgacctgcgggccgctgttttggtttgacatgacaaggaaaggacttcctgctgaccctgagagcctctggggtgccgcggcaccacggggcatgcatgattgtgctagcgtttagtctgagttgatct

SEQ ID NO: 689 - DNA, 200 bp, Artificial Sequence
  Synthetic
ggacccagttttggctgctggttcccagcaggggactcgggggatatacagtggctgcaccaaattggaggtgtgggttcctccaacacaatttgcttctgcccgttgtcttcctgccagctgggtttggccaggatttctccgtgtgggggctacatgcgaccctctcccctcctccctgactttagaggctggtgctg

SEQ ID NO: 690 - DNA, 533 bp, Artificial Sequence
  Synthetic
gcgcagcatggaggactttgtcacttgggtggactcgtccaagatcaagcggcacgtgctagagtacaatgaggagcgcgatgacttcgatctggaagcctagcggatctcccactttgcatggctgtcttttacagatgggaaaactgaggcctgatgctggagattctatgagggtgctctcctcaagggtatcagacggtcgtaggttcttaagaatttgattcatcagtggcaggccatgcatagagccacgggaggtgcgtccttgttttccaggaaatgttcttagaacttggactactgattattaattgactgtgccttgggaaacagtgggaagtaacttggtgcagcactggggtattgttggactggttcaattcgtttaactcgaattcttgctcctggccgtggttaagctgtgtacagatgatggagagtttggcctcaagtttttataaactgagcgagactagtgttcaggatctcctcccttgtttaaatgtcaataaatgccccaactgctttgt

SEQ ID NO: 691 - DNA, 461 bp, Artificial Sequence
  Synthetic
aggatgaatatcacgcgtctcacatctttaatccagcctttcgtgacatgttggaaagatacatgtgaaacctacccagttaccctttctgaattgggaggaaaaccaaccaatgtatgtatgagaaactcagaagtctgaatagaaaaacaaagtaaatggcagaagattctcgagtttatgcccgcgtaggtttggagtgttgaaaaagctaaaatgtttagtttcacttggccctgaggtatggttgagaaggctgactgccagcagttgaggattgagtccgaccatgtttacatgcagggttcccaacaccagtggtgacactgggaagcagccccagcactttcctctcctgagtcctccagacccaaaatccttaatgtcaaaccaggtcagtgtttcttactgtgtttcaagtcgttaaaaagactgagagtagaggcactttatgctgctat

SEQ ID NO: 692 - DNA, 468 bp, Artificial Sequence
  Synthetic
  misc_feature (47)..(47): n is a, c, g, or t
  misc_feature (297)..(312): n is a, c, g, or t
  misc_feature (400)..(400): n is a, c, g, or t
  misc_feature (402)..(402): n is a, c, g, or t
gaagtcactcctccaatgtctgggacttgccagctcagcccgttagnatgagggtgctgagaggaaacaggaaacaagactgcgaatggcgctcaggcagggagcagggagtggcgtttggcttgcacgttcccatgtggccagatgctggggccactttccttctgtctgctggtgactgcagtgttccccctcctcctcaccacggggctcctgtgagtctggggggcacctctttctggcctgtgcacctctctctggcttataaaggtgcctggcctgtgccagcccctcctnnnnnnnnnnnnnnnnggggaccaggtgagccggctctcccacgtggttgtcccgggaaagctgccccacagcctcagcatcttcagcacttaccgatccagancntcccggccttctccggtgtcctgtaccaactcttctatttaagagaacctcagatgatgtacctga

SEQ ID NO: 693 - DNA, 445 bp, Artificial Sequence
  Synthetic
  misc_feature (164)..(164): n is a, c, g, or t
  misc_feature (168)..(168): n is a, c, g, or t
  misc_feature (174)..(174): n is a, c, g, or t
  misc_feature (188)..(188): n is a, c, g, or t
  misc_feature (191)..(211): n is a, c, g, or t
  misc_feature (214)..(214): n is a, c, g, or t
  misc_feature (391)..(391): n is a, c, g, or t
  misc_feature (393)..(394): n is a, c, g, or t
cccttggtgctgtgtggtcccagtggaaggaggggaagattttggaaacctggtagccaccagtaaggtgattctctgccctgttggggcctaaatttgggggcttttgggcaacctctccgtgtactgcgtctgtccacactcgattgggccccaggtgtgtnatgnaggcgnctctggtaaggtgnctnnnnnnnnnnnnnnnnnnnnntcnagtaacgaggcttttgatgtgttgagctggaggtgagtggaccgggggctgtgttttaagctgcttccttggcatttggcatcactgccttctgttcccgggggagcatggatcttttgtcctcactgctttctaatggggagggctgagggctccctgtccccacagcaggtatgntnngctctgccccagccccacacttgctctgaaaaccaagtgtcagagcccctt

SEQ ID NO: 694 - DNA, 512 bp, Artificial Sequence
  Synthetic
  misc_feature (97)..(97): n is a, c, g, or t
  misc_feature (176)..(176): n is a, c, g, or t
  misc_feature (229)..(239): n is a, c, g, or t
  misc_feature (386)..(386): n is a, c, g, or t
  misc_feature (411)..(485): n is a, c, g, or t
tcccctttgctttcttgtgttccagccacctgccatttcctccctgcctcccaccctccctccctgccccctgtcctgcttcccaccccctgcctgnccagcctgggccctgcagtgtggagagacacataagccttactgtcctctgggggcaggagccgagcctttttgttgcnccgctcccaggagagtgagggtgacgcaattgattaaaaccattttgttctannnnnnnnnnntggcatttgtggggtgcagatgggtcttggaaccaaagtgagggcacctggtccctcacctctgtagctctatgacttagtaaccacacctctactggacagaatgaggcctgccttgaagggtcaccgtgagcagcggccacccancccagcagggctgagtgcagagccnnnnnnnnnnnnnnnnnnnnnnnnnnnnnnnnnnnnnnnnnnnnnnnnnnnnnnnnnnnnnnnnnnnnnnnnnnntggaatcttagtggtttttcaaaccat

SEQ ID NO: 695 - DNA, 555 bp, Artificial Sequence
  Synthetic
  misc_feature (372)..(387): n is a, c, g, or t
  misc_feature (475)..(475): n is a, c, g, or t
  misc_feature (481)..(496): n is a, c, g, or t
  misc_feature (500)..(500): n is a, c, g, or t
  misc_feature (502)..(503): n is a, c, g, or t
  misc_feature (511)..(511): n is a, c, g, or t
gagttccacagacagctcggggcccctggaggaggcagaggaggccccccagctgatgcggaccaagagcgacgccagttgcatgagccagaggaggcccaagtgccgcgcccccggtgaggcccagcgcatccggcgacaccggttctctatcaacggccacttctacaatcataagacctccgtgtttactccagcctatggatccgtgaccaatgtgagggtcaacagcaccatgacaaccctgcaggtgctcaccctgctgctgaacaaatttagggtggaagatggccccagtgagttcgcactctacatcgttcacgagtctggggagcggacaaaattaaaagactgcgagtacccgctgatttnnnnnnnnnnnnnnnngccatgtgagaagatcgccaggatcttcctgatggaagctgacttgggcgtggaagtcccccatgaagtcgctcagtacattaagttngaaatnnnnnnnnnnnnnnnntttntnnaaaaattnaaaagaagaggaagaaagagaaataatcaaacttgaccatgaag